SEQUENCE LISTING (543 sequences)

SEQ ID NO: 1 - protein, 8 aa, Artificial Sequence
  peptide antagonist C-terminus of BLR-1
NATSLTTF

SEQ ID NO: 2 - protein, 4 aa, Artificial Sequence
  small molecule inhibitor
XTTF

SEQ ID NO: 3 - protein, 8 aa, Artificial Sequence
  peptide antagonist C-terminus of DOCK2
PDSLSTDL

SEQ ID NO: 4 - protein, 4 aa, Artificial Sequence
  PDZ motif
SSQL

SEQ ID NO: 5 - protein, 5 aa, Artificial Sequence
  PDZ motif
SSSQL

SEQ ID NO: 6 - protein, 6 aa, Artificial Sequence
  PDZ motif
SSSSQL

SEQ ID NO: 7 - protein, 7 aa, Artificial Sequence
  PDZ motif
PSSSSQL

SEQ ID NO: 8 - protein, 8 aa, Artificial Sequence
  PDZ motif
PPSSSSQL

SEQ ID NO: 9 - protein, 4 aa, Artificial Sequence
  PDZ motif
CSPI

SEQ ID NO: 10 - protein, 5 aa, Artificial Sequence
  PDZ motif
TCSPI

SEQ ID NO: 11 - protein, 6 aa, Artificial Sequence
  PDZ motif
KTCSPI

SEQ ID NO: 12 - protein, 7 aa, Artificial Sequence
  PDZ motif
QKTCSPI

SEQ ID NO: 13 - protein, 8 aa, Artificial Sequence
  PDZ motif
FQKTCSPI

SEQ ID NO: 14 - protein, 4 aa, Artificial Sequence
  PDZ motif
ISAA

SEQ ID NO: 15 - protein, 5 aa, Artificial Sequence
  PDZ motif
DISAA

SEQ ID NO: 16 - protein, 6 aa, Artificial Sequence
  PDZ motif
DDISAA

SEQ ID NO: 17 - protein, 7 aa, Artificial Sequence
  PDZ motif
YDDISAA

SEQ ID NO: 18 - protein, 8 aa, Artificial Sequence
  PDZ motif
DYDDISAA

SEQ ID NO: 19 - protein, 4 aa, Artificial Sequence
  PDZ motif
TSEI

SEQ ID NO: 20 - protein, 5 aa, Artificial Sequence
  PDZ motif
CTSEI

SEQ ID NO: 21 - protein, 6 aa, Artificial Sequence
  PDZ motif
SCTSEI

SEQ ID NO: 22 - protein, 7 aa, Artificial Sequence
  PDZ motif
SSCTSEI

SEQ ID NO: 23 - protein, 8 aa, Artificial Sequence
  PDZ motif
DSSCTSEI

SEQ ID NO: 24 - protein, 4 aa, Artificial Sequence
  PDZ motif
TSDA

SEQ ID NO: 25 - protein, 5 aa, Artificial Sequence
  PDZ motif
ATSDA

SEQ ID NO: 26 - protein, 6 aa, Artificial Sequence
  PDZ motif
PATSDA

SEQ ID NO: 27 - protein, 7 aa, Artificial Sequence
  PDZ motif
PPATSDA

SEQ ID NO: 28 - protein, 8 aa, Artificial Sequence
  PDZ motif
KPPATSDA

SEQ ID NO: 29 - protein, 4 aa, Artificial Sequence
  small molecule inhibitor
XTDL

SEQ ID NO: 30 - protein, 5 aa, Artificial Sequence
  PDZ motif
LTSDA

SEQ ID NO: 31 - protein, 6 aa, Artificial Sequence
  PDZ motif
RLTSDA

SEQ ID NO: 32 - protein, 7 aa, Artificial Sequence
  PDZ motif
ERLTSDA

SEQ ID NO: 33 - protein, 8 aa, Artificial Sequence
  PDZ motif
KERLTSDA

SEQ ID NO: 34 - protein, 4 aa, Artificial Sequence
  PDZ motif
TIGL

SEQ ID NO: 35 - protein, 5 aa, Artificial Sequence
  PDZ motif
QTIGL

SEQ ID NO: 36 - protein, 6 aa, Artificial Sequence
  PDZ motif
SQTIGL

SEQ ID NO: 37 - protein, 7 aa, Artificial Sequence
  PDZ motif
TSQTIGL

SEQ ID NO: 38 - protein, 8 aa, Artificial Sequence
  PDZ motif
KTSQTIGL

SEQ ID NO: 39 - protein, 4 aa, Artificial Sequence
  PDZ motif
FMSL

SEQ ID NO: 40 - protein, 5 aa, Artificial Sequence
  PDZ motif
DFMSL

SEQ ID NO: 41 - protein, 6 aa, Artificial Sequence
  PDZ motif
TDFMSL

SEQ ID NO: 42 - protein, 7 aa, Artificial Sequence
  PDZ motif
ATDFMSL

SEQ ID NO: 43 - protein, 8 aa, Artificial Sequence
  PDZ motif
QATDFMSL

SEQ ID NO: 44 - protein, 4 aa, Artificial Sequence
  PDZ motif
QSLV

SEQ ID NO: 45 - protein, 5 aa, Artificial Sequence
  PDZ motif
IQSLV

SEQ ID NO: 46 - protein, 6 aa, Artificial Sequence
  PDZ motif
EIQSLV

SEQ ID NO: 47 - protein, 7 aa, Artificial Sequence
  PDZ motif
NEIQSLV

SEQ ID NO: 48 - protein, 8 aa, Artificial Sequence
  PDZ motif
RNEIQSLV

SEQ ID NO: 49 - protein, 4 aa, Artificial Sequence
  PDZ motif
ESGI

SEQ ID NO: 50 - protein, 5 aa, Artificial Sequence
  PDZ motif
SESGI

SEQ ID NO: 51 - protein, 6 aa, Artificial Sequence
  PDZ motif
ASESGI

SEQ ID NO: 52 - protein, 7 aa, Artificial Sequence
  PDZ motif
RASESGI

SEQ ID NO: 53 - protein, 8 aa, Artificial Sequence
  PDZ motif
LRASESGI

SEQ ID NO: 54 - protein, 4 aa, Artificial Sequence
  PDZ motif
PYAA

SEQ ID NO: 55 - protein, 5 aa, Artificial Sequence
  PDZ motif
FPYAA

SEQ ID NO: 56 - protein, 6 aa, Artificial Sequence
  PDZ motif
RFPYAA

SEQ ID NO: 57 - protein, 7 aa, Artificial Sequence
  PDZ motif
LRFPYAA

SEQ ID NO: 58 - protein, 8 aa, Artificial Sequence
  PDZ motif
LLRFPYAA

SEQ ID NO: 59 - protein, 4 aa, Artificial Sequence
  PDZ motif
GCEL

SEQ ID NO: 60 - protein, 5 aa, Artificial Sequence
  PDZ motif
GGCEL

SEQ ID NO: 61 - protein, 6 aa, Artificial Sequence
  PDZ motif
EGGCEL

SEQ ID NO: 62 - protein, 7 aa, Artificial Sequence
  PDZ motif
EEGGCEL

SEQ ID NO: 63 - protein, 8 aa, Artificial Sequence
  PDZ motif
EEEGGCEL

SEQ ID NO: 64 - protein, 4 aa, Artificial Sequence
  PDZ motif
KTEA

SEQ ID NO: 65 - protein, 5 aa, Artificial Sequence
  PDZ motif
HKTEA

SEQ ID NO: 66 - protein, 6 aa, Artificial Sequence
  PDZ motif
NHKTEA

SEQ ID NO: 67 - protein, 7 aa, Artificial Sequence
  PDZ motif
NNHKTEA

SEQ ID NO: 68 - protein, 8 aa, Artificial Sequence
  PDZ motif
ENNHKTEA

SEQ ID NO: 69 - protein, 4 aa, Artificial Sequence
  PDZ motif
SSNL

SEQ ID NO: 70 - protein, 5 aa, Artificial Sequence
  PDZ motif
VSSNL

SEQ ID NO: 71 - protein, 6 aa, Artificial Sequence
  PDZ motif
NVSSNL

SEQ ID NO: 72 - protein, 7 aa, Artificial Sequence
  PDZ motif
VNVSSNL

SEQ ID NO: 73 - protein, 8 aa, Artificial Sequence
  PDZ motif
SVNVSSNL

SEQ ID NO: 74 - protein, 4 aa, Artificial Sequence
  PDZ motif
KTRV

SEQ ID NO: 75 - protein, 5 aa, Artificial Sequence
  PDZ motif
PKTRV

SEQ ID NO: 76 - protein, 6 aa, Artificial Sequence
  PDZ motif
RPKTRV

SEQ ID NO: 77 - protein, 7 aa, Artificial Sequence
  PDZ motif
RRPKTRV

SEQ ID NO: 78 - protein, 8 aa, Artificial Sequence
  PDZ motif
SRRPKTRV

SEQ ID NO: 79 - protein, 4 aa, Artificial Sequence
  PDZ motif
LYKL

SEQ ID NO: 80 - protein, 5 aa, Artificial Sequence
  PDZ motif
GLYKL

SEQ ID NO: 81 - protein, 6 aa, Artificial Sequence
  PDZ motif
FGLYKL

SEQ ID NO: 82 - protein, 7 aa, Artificial Sequence
  PDZ motif
FFGLYKL

SEQ ID NO: 83 - protein, 8 aa, Artificial Sequence
  PDZ motif
TFFGLYKL

SEQ ID NO: 84 - protein, 4 aa, Artificial Sequence
  PDZ motif
VTAL

SEQ ID NO: 85 - protein, 5 aa, Artificial Sequence
  PDZ motif
HVTAL

SEQ ID NO: 86 - protein, 6 aa, Artificial Sequence
  PDZ motif
LHVTAL

SEQ ID NO: 87 - protein, 7 aa, Artificial Sequence
  PDZ motif
GLHVTAL

SEQ ID NO: 88 - protein, 8 aa, Artificial Sequence
  PDZ motif
QGLHVTAL

SEQ ID NO: 89 - protein, 4 aa, Artificial Sequence
  PDZ motif
EFYA

SEQ ID NO: 90 - protein, 5 aa, Artificial Sequence
  PDZ motif
EEFYA

SEQ ID NO: 91 - protein, 6 aa, Artificial Sequence
  PDZ motif
QEEFYA

SEQ ID NO: 92 - protein, 7 aa, Artificial Sequence
  PDZ motif
KQEEFYA

SEQ ID NO: 93 - protein, 8 aa, Artificial Sequence
  PDZ motif
TKQEEFYA

SEQ ID NO: 94 - protein, 4 aa, Artificial Sequence
  PDZ motif
DSVF

SEQ ID NO: 95 - protein, 5 aa, Artificial Sequence
  PDZ motif
EDSVF

SEQ ID NO: 96 - protein, 6 aa, Artificial Sequence
  PDZ motif
LEDSVF

SEQ ID NO: 97 - protein, 7 aa, Artificial Sequence
  PDZ motif
TLEDSVF

SEQ ID NO: 98 - protein, 8 aa, Artificial Sequence
  PDZ motif
ETLEDSVF

SEQ ID NO: 99 - protein, 4 aa, Artificial Sequence
  PDZ motif
ESKA

SEQ ID NO: 100 - protein, 5 aa, Artificial Sequence
  PDZ motif
NESKA

SEQ ID NO: 101 - protein, 6 aa, Artificial Sequence
  PDZ motif
ENESKA

SEQ ID NO: 102 - protein, 7 aa, Artificial Sequence
  PDZ motif
KENESKA

SEQ ID NO: 103 - protein, 8 aa, Artificial Sequence
  PDZ motif
TKENESKA

SEQ ID NO: 104 - protein, 4 aa, Artificial Sequence
  PDZ motif
KIGV

SEQ ID NO: 105 - protein, 5 aa, Artificial Sequence
  PDZ motif
MKIGV

SEQ ID NO: 106 - protein, 6 aa, Artificial Sequence
  PDZ motif
DMKIGV

SEQ ID NO: 107 - protein, 7 aa, Artificial Sequence
  PDZ motif
VDMKIGV

SEQ ID NO: 108 - protein, 8 aa, Artificial Sequence
  PDZ motif
NVDMKIGV

SEQ ID NO: 109 - protein, 4 aa, Artificial Sequence
  PDZ motif
FTSL

SEQ ID NO: 110 - protein, 5 aa, Artificial Sequence
  PDZ motif
KFTSL

SEQ ID NO: 111 - protein, 6 aa, Artificial Sequence
  PDZ motif
VKFTSL

SEQ ID NO: 112 - protein, 7 aa, Artificial Sequence
  PDZ motif
EVKFTSL

SEQ ID NO: 113 - protein, 8 aa, Artificial Sequence
  PDZ motif
REVKFTSL

SEQ ID NO: 114 - protein, 4 aa, Artificial Sequence
  PDZ motif
KSLV

SEQ ID NO: 115 - protein, 5 aa, Artificial Sequence
  PDZ motif
LKSLV

SEQ ID NO: 116 - protein, 6 aa, Artificial Sequence
  PDZ motif
FLKSLV

SEQ ID NO: 117 - protein, 7 aa, Artificial Sequence
  PDZ motif
RFLKSLV

SEQ ID NO: 118 - protein, 8 aa, Artificial Sequence
  PDZ motif
GRFLKSLV

SEQ ID NO: 119 - protein, 4 aa, Artificial Sequence
  PDZ motif
GYIA

SEQ ID NO: 120 - protein, 5 aa, Artificial Sequence
  PDZ motif
NGYIA

SEQ ID NO: 121 - protein, 6 aa, Artificial Sequence
  PDZ motif
TNGYIA

SEQ ID NO: 122 - protein, 7 aa, Artificial Sequence
  PDZ motif
KTNGYIA

SEQ ID NO: 123 - protein, 8 aa, Artificial Sequence
  PDZ motif
GKTNGYIA

SEQ ID NO: 124 - protein, 4 aa, Artificial Sequence
  PDZ motif
QTLL

SEQ ID NO: 125 - protein, 5 aa, Artificial Sequence
  PDZ motif
LQTLL

SEQ ID NO: 126 - protein, 6 aa, Artificial Sequence
  PDZ motif
LLQTLL

SEQ ID NO: 127 - protein, 7 aa, Artificial Sequence
  PDZ motif
VLLQTLL

SEQ ID NO: 128 - protein, 8 aa, Artificial Sequence
  PDZ motif
SVLLQTLL

SEQ ID NO: 129 - protein, 4 aa, Artificial Sequence
  PDZ motif
PIDL

SEQ ID NO: 130 - protein, 5 aa, Artificial Sequence
  PDZ motif
PPIDL

SEQ ID NO: 131 - protein, 6 aa, Artificial Sequence
  PDZ motif
SPPIDL

SEQ ID NO: 132 - protein, 7 aa, Artificial Sequence
  PDZ motif
MSPPIDL

SEQ ID NO: 133 - protein, 8 aa, Artificial Sequence
  PDZ motif
EMSPPIDL

SEQ ID NO: 134 - protein, 4 aa, Artificial Sequence
  PDZ motif
YTMI

SEQ ID NO: 135 - protein, 5 aa, Artificial Sequence
  PDZ motif
SYTMI

SEQ ID NO: 136 - protein, 6 aa, Artificial Sequence
  PDZ motif
ASYTMI

SEQ ID NO: 137 - protein, 7 aa, Artificial Sequence
  PDZ motif
SASYTMI

SEQ ID NO: 138 - protein, 8 aa, Artificial Sequence
  PDZ motif
TSASYTMI

SEQ ID NO: 139 - protein, 4 aa, Artificial Sequence
  PDZ motif
HSVI

SEQ ID NO: 140 - protein, 5 aa, Artificial Sequence
  PDZ motif
EHSVI

SEQ ID NO: 141 - protein, 6 aa, Artificial Sequence
  PDZ motif
NEHSVI

SEQ ID NO: 142 - protein, 7 aa, Artificial Sequence
  PDZ motif
QNEHSVI

SEQ ID NO: 143 - protein, 8 aa, Artificial Sequence
  PDZ motif
EQNEHSVI

SEQ ID NO: 144 - protein, 4 aa, Artificial Sequence
  PDZ motif
TSVL

SEQ ID NO: 145 - protein, 5 aa, Artificial Sequence
  PDZ motif
ITSVL

SEQ ID NO: 146 - protein, 6 aa, Artificial Sequence
  PDZ motif
PITSVL

SEQ ID NO: 147 - protein, 7 aa, Artificial Sequence
  PDZ motif
FPITSVL

SEQ ID NO: 148 - protein, 8 aa, Artificial Sequence
  PDZ motif
LFPITSVL

SEQ ID NO: 149 - protein, 4 aa, Artificial Sequence
  PDZ motif
DTEL

SEQ ID NO: 150 - protein, 5 aa, Artificial Sequence
  PDZ motif
ADTEL

SEQ ID NO: 151 - protein, 6 aa, Artificial Sequence
  PDZ motif
VADTEL

SEQ ID NO: 152 - protein, 7 aa, Artificial Sequence
  PDZ motif
VVADTEL

SEQ ID NO: 153 - protein, 8 aa, Artificial Sequence
  PDZ motif
HVVADTEL

SEQ ID NO: 154 - protein, 4 aa, Artificial Sequence
  PDZ motif
VALI

SEQ ID NO: 155 - protein, 5 aa, Artificial Sequence
  PDZ motif
RVALI

SEQ ID NO: 156 - protein, 6 aa, Artificial Sequence
  PDZ motif
ARVALI

SEQ ID NO: 157 - protein, 7 aa, Artificial Sequence
  PDZ motif
LARVALI

SEQ ID NO: 158 - protein, 8 aa, Artificial Sequence
  PDZ motif
VLARVALI

SEQ ID NO: 159 - protein, 4 aa, Artificial Sequence
  PDZ motif
SSMA

SEQ ID NO: 160 - protein, 5 aa, Artificial Sequence
  PDZ motif
TSSMA

SEQ ID NO: 161 - protein, 6 aa, Artificial Sequence
  PDZ motif
STSSMA

SEQ ID NO: 162 - protein, 8 aa, Artificial Sequence
  PDZ motif
PCSTSSMA

SEQ ID NO: 163 - protein, 4 aa, Artificial Sequence
  PDZ motif
KSKV

SEQ ID NO: 164 - protein, 5 aa, Artificial Sequence
  PDZ motif
QKSKV

SEQ ID NO: 165 - protein, 6 aa, Artificial Sequence
  PDZ motif
AQKSKV

SEQ ID NO: 166 - protein, 7 aa, Artificial Sequence
  PDZ motif
EAQKSKV

SEQ ID NO: 167 - protein, 8 aa, Artificial Sequence
  PDZ motif
VEAQKSKV

SEQ ID NO: 168 - protein, 4 aa, Artificial Sequence
  PDZ motif
SYIL

SEQ ID NO: 169 - protein, 5 aa, Artificial Sequence
  PDZ motif
PSYIL

SEQ ID NO: 170 - protein, 6 aa, Artificial Sequence
  PDZ motif
KPSYIL

SEQ ID NO: 171 - protein, 7 aa, Artificial Sequence
  PDZ motif
QKPSYIL

SEQ ID NO: 172 - protein, 8 aa, Artificial Sequence
  PDZ motif
YQKPSYIL

SEQ ID NO: 173 - protein, 13 aa, Artificial Sequence
  Tat peptide
GYGRKKRRQRRRG

SEQ ID NO: 174 - protein, 21 aa, Artificial Sequence
  Tat-CD3 carboxyl terminus fusion peptide
GYGRKKRRQRRRGPPSSSSGL

SEQ ID NO: 175 - protein, 4 aa, Artificial Sequence
  PDZ ligand
SAEV

SEQ ID NO: 176 - protein, 21 aa, Artificial Sequence
  Tat-CLASP2 carboxyl terminus fusion peptide
GYGRKKRRQRRRGMTSSSSVV

SEQ ID NO: 177 - protein, 4 aa, Artificial Sequence
  PDZ motif
TELV

SEQ ID NO: 178 - protein, 5 aa, Artificial Sequence
  PDZ motif
KTELV

SEQ ID NO: 179 - protein, 6 aa, Artificial Sequence
  PDZ motif
HKTELV

SEQ ID NO: 180 - protein, 7 aa, Artificial Sequence
  PDZ motif
PHKTELV

SEQ ID NO: 181 - protein, 8 aa, Artificial Sequence
  PDZ motif
TPHKTELV

SEQ ID NO: 182 - protein, 4 aa, Artificial Sequence
  PDZ motif
SAQV

SEQ ID NO: 183 - protein, 5 aa, Artificial Sequence
  PDZ motif
SSAQV

SEQ ID NO: 184 - protein, 6 aa, Artificial Sequence
  PDZ motif
SSSAQV

SEQ ID NO: 185 - protein, 7 aa, Artificial Sequence
  PDZ motif
ISSSAQV

SEQ ID NO: 186 - protein, 8 aa, Artificial Sequence
  PDZ motif
SISSSAQV

SEQ ID NO: 187 - protein, 4 aa, Artificial Sequence
  PDZ motif
SSVV

SEQ ID NO: 188 - protein, 5 aa, Artificial Sequence
  PDZ motif
SSSVV

SEQ ID NO: 189 - protein, 6 aa, Artificial Sequence
  PDZ motif
SSSSVV

SEQ ID NO: 190 - protein, 7 aa, Artificial Sequence
  PDZ motif
TSSSSVV

SEQ ID NO: 191 - protein, 8 aa, Artificial Sequence
  PDZ motif
MTSSSSVV

SEQ ID NO: 192 - protein, 4 aa, Artificial Sequence
  PDZ motif
YAEV

SEQ ID NO: 193 - protein, 5 aa, Artificial Sequence
  PDZ motif
RYAEV

SEQ ID NO: 194 - protein, 6 aa, Artificial Sequence
  PDZ motif
PRYAEV

SEQ ID NO: 195 - protein, 7 aa, Artificial Sequence
  PDZ motif
SPRYAEV

SEQ ID NO: 196 - protein, 8 aa, Artificial Sequence
  PDZ motif
GSPRYAEV

SEQ ID NO: 197 - protein, 4 aa, Artificial Sequence
  PDZ motif
KSKV

SEQ ID NO: 198 - protein, 5 aa, Artificial Sequence
  PDZ motif
QKSKV

SEQ ID NO: 199 - protein, 6 aa, Artificial Sequence
  PDZ motif
AQKSKV

SEQ ID NO: 200 - protein, 7 aa, Artificial Sequence
  PDZ motif
EAQKSKV

SEQ ID NO: 201 - protein, 8 aa, Artificial Sequence
  PDZ motif
VEAQKSKV

SEQ ID NO: 202 - protein, 4 aa, Artificial Sequence
  PDZ motif
FTDV

SEQ ID NO: 203 - protein, 5 aa, Artificial Sequence
  PDZ motif
IFTDV

SEQ ID NO: 204 - protein, 6 aa, Artificial Sequence
  PDZ motif
KIFTDV

SEQ ID NO: 205 - protein, 7 aa, Artificial Sequence
  PDZ motif
KKIFTDV

SEQ ID NO: 206 - protein, 8 aa, Artificial Sequence
  PDZ motif
IKKIFTDV

SEQ ID NO: 207 - protein, 4 aa, Artificial Sequence
  PDZ motif
STDL

SEQ ID NO: 208 - protein, 5 aa, Artificial Sequence
  PDZ motif
LSTDL

SEQ ID NO: 209 - protein, 6 aa, Artificial Sequence
  PDZ motif
SLSTDL

SEQ ID NO: 210 - protein, 7 aa, Artificial Sequence
  PDZ motif
DSLSTDL

SEQ ID NO: 211 - protein, 8 aa, Artificial Sequence
  PDZ motif
PDSLSTDL

SEQ ID NO: 212 - protein, 4 aa, Artificial Sequence
  HPV66 carboxy terminus
ESTV

SEQ ID NO: 213 - protein, 5 aa, Artificial Sequence
  PDZ motif
KEFYA

SEQ ID NO: 214 - protein, 6 aa, Artificial Sequence
  PDZ motif
TKEFYA

SEQ ID NO: 215 - protein, 7 aa, Artificial Sequence
  PDZ motif
PTKEFYA

SEQ ID NO: 216 - protein, 8 aa, Artificial Sequence
  PDZ motif
APTKEFYA

SEQ ID NO: 217 - protein, 4 aa, Artificial Sequence
  PDZ motif
LTTF

SEQ ID NO: 218 - protein, 5 aa, Artificial Sequence
  PDZ motif
SLTTF

SEQ ID NO: 219 - protein, 6 aa, Artificial Sequence
  PDZ motif
TSLTTF

SEQ ID NO: 220 - protein, 7 aa, Artificial Sequence
  PDZ motif
ATSLTTF

SEQ ID NO: 221 - protein, 8 aa, Artificial Sequence
  PDZ motif
NATSLTTF

SEQ ID NO: 222 - protein, 7 aa, Artificial Sequence
  PDZ motif
CSTSSMA

SEQ ID NO: 223 - protein, 4 aa, Artificial Sequence
  PDZ motif
ESDV

SEQ ID NO: 224 - protein, 5 aa, Artificial Sequence
  PDZ motif
IESDV

SEQ ID NO: 225 - protein, 6 aa, Artificial Sequence
  PDZ motif
SIESDV

SEQ ID NO: 226 - protein, 7 aa, Artificial Sequence
  PDZ motif
PSIESDV

SEQ ID NO: 227 - protein, 8 aa, Artificial Sequence
  PDZ motif
MPSIESDV

SEQ ID NO: 228 - protein, 4 aa, Artificial Sequence
  PDZ motif
EYYV

SEQ ID NO: 229 - protein, 5 aa, Artificial Sequence
  PDZ motif
KEYYV

SEQ ID NO: 230 - protein, 6 aa, Artificial Sequence
  PDZ motif
DKEYYV

SEQ ID NO: 231 - protein, 7 aa, Artificial Sequence
  PDZ motif
KDKEYYV

SEQ ID NO: 232 - protein, 8 aa, Artificial Sequence
  PDZ motif
NKDKEYYV

SEQ ID NO: 233 - protein, 4 aa, Artificial Sequence
  PDZ motif
EYFI

SEQ ID NO: 234 - protein, 5 aa, Artificial Sequence
  PDZ motif
KEYFI

SEQ ID NO: 235 - protein, 6 aa, Artificial Sequence
  PDZ motif
RKEYFI

SEQ ID NO: 236 - protein, 7 aa, Artificial Sequence
  PDZ motif
SRKEYFI

SEQ ID NO: 237 - protein, 8 aa, Artificial Sequence
  PDZ motif
SSRKEYFI

SEQ ID NO: 238 - protein, 4 aa, Artificial Sequence
  PDZ motif
KTIA

SEQ ID NO: 239 - protein, 5 aa, Artificial Sequence
  PDZ motif
GKTIA

SEQ ID NO: 240 - protein, 6 aa, Artificial Sequence
  PDZ motif
FGKTIA

SEQ ID NO: 241 - protein, 7 aa, Artificial Sequence
  PDZ motif
TFGKTIA

SEQ ID NO: 242 - protein, 8 aa, Artificial Sequence
  PDZ motif
TTFGKTIA

SEQ ID NO: 243 - protein, 21 aa, Artificial Sequence
  Tat-CLASP1 carboxyl terminus fusion peptide
GYGRKKRRQRRRGSISSSAEV

SEQ ID NO: 244 - protein, 4 aa, Artificial Sequence
  PDZ motif
VSFV

SEQ ID NO: 245 - protein, 5 aa, Artificial Sequence
  PDZ motif
LVSFV

SEQ ID NO: 246 - protein, 6 aa, Artificial Sequence
  PDZ motif
SLVSFV

SEQ ID NO: 247 - protein, 7 aa, Artificial Sequence
  PDZ motif
QSLVSFV

SEQ ID NO: 248 - protein, 8 aa, Artificial Sequence
  PDZ motif
GQSLVSFV

SEQ ID NO: 249 - protein, 4 aa, Artificial Sequence
  PDZ ligand
TTRV

SEQ ID NO: 250 - protein, 4 aa, Artificial Sequence
  PDZ ligand
ETEV

SEQ ID NO: 251 - protein, 4 aa, Artificial Sequence
  PDZ ligand
AQRL

SEQ ID NO: 252 - protein, 4 aa, Artificial Sequence
  PDZ ligand
HDAL

SEQ ID NO: 253 - protein, 4 aa, Artificial Sequence
  PDZ motif
ITRL

SEQ ID NO: 254 - protein, 5 aa, Artificial Sequence
  PDZ motif
DITRL

SEQ ID NO: 255 - protein, 6 aa, Artificial Sequence
  PDZ motif
RDITRL

SEQ ID NO: 256 - protein, 7 aa, Artificial Sequence
  PDZ motif
GRDITRL

SEQ ID NO: 257 - protein, 8 aa, Artificial Sequence
  PDZ motif
QGRDITRL

SEQ ID NO: 258 - protein, 4 aa, Artificial Sequence
  PDZ motif
STTL

SEQ ID NO: 259 - protein, 5 aa, Artificial Sequence
  PDZ motif
TSTTL

SEQ ID NO: 260 - protein, 6 aa, Artificial Sequence
  PDZ motif
HTSTTL

SEQ ID NO: 261 - protein, 7 aa, Artificial Sequence
  PDZ motif
GHTSTTL

SEQ ID NO: 262 - protein, 8 aa, Artificial Sequence
  PDZ motif
SGHTSTTL

SEQ ID NO: 263 - protein, 4 aa, Artificial Sequence
  PDZ ligand
SAGF

SEQ ID NO: 264 - protein, 4 aa, Artificial Sequence
  PDZ ligand
SIVF

SEQ ID NO: 265 - protein, 4 aa, Artificial Sequence
  PDZ ligand
LGSF

SEQ ID NO: 266 - protein, 4 aa, Artificial Sequence
  PDZ ligand
DHWC

SEQ ID NO: 267 - protein, 90 aa, Artificial Sequence
  CASK PDZ domain 1
TRVRLVQFQKNTDEPMGITLKMNELNHCIVARIMHGGMIHRQGTLHVGDEIREINGISVANQTVEQLQKMLREMRGSITFKIVPSYRTQS

SEQ ID NO: 268 - protein, 86 aa, Artificial Sequence
  MPP1 PDZ domain 1
RKVRLIQFEKVTEEPMGITLKLNEKQSCTVARILHGGMIHRQGSLHVGDEILEINGTNVTNHSVDQLQKAMKETKGMISLKVIPNQ

SEQ ID NO: 269 - protein, 95 aa, Artificial Sequence
  LIMK1 PDZ domain 1
VTLVSIPASSHGKRGLSVSIDPPHGPPGCGTEHSHTVRVQGVDPGCMSPDVKNSIHVGDRILEINGTPIRNVPLDEIDLLIQETSRLLQLTLEHD

SEQ ID NO: 270 - protein, 91 aa, Artificial Sequence
  KIAA 0303 PDZ domain 1
PHQPIVIHSSGKNYGFTIRAIRVYVGDSDIYTVHHIVWNVEEGSPACQAGLKAGDLITHINGEPVHGLVHTEVIELLLKSGNKVSITTTPF

SEQ ID NO: 271 - protein, 89 aa, Artificial Sequence
  KIAA 0807 PDZ domain 1
PIIIHRAGKKYGFTLRAIRVYMGDSDVYTVHHMVWHVEDGGPASEAGLRQGDLITHVNGEPVHGLVHTEVVELILKSGNKVAISTTPLE

SEQ ID NO: 272 - protein, 203 aa, Artificial Sequence
  DLG1 PDZ domain 1
VNGTDADYEYEEITLERGNSGLGFSIAGGTDNPHIGDDSSIFITKIITGGAAAQDGRLRVNDCILQVNEVDVRDVTHSKAVEALKEAGSIVRLYVKRRKPVSEKIMEIKLIKGPKGLGFSIAGGVGNQHIPGDNSIYVTKIIEGGAAHKDGKLQIGDKLLAVNNVCLEEVTHEEAVTALKNTSDFVYLKVAKPTSMYMNDGYA

SEQ ID NO: 273 - protein, 338 aa, Artificial Sequence
  PSD95 PDZ domains 1-3
EGEMEYEEITLERGNSGLGFSIAGGTDNPHIGDDPSIFITKIIPGGAAAQDGRLRVNDSILFVNEVDVREVTHSAAVEALKEAGSIVRLYVMRRKPPAEKVMEIKLIKGPKGLGFSIAGGVGNQHIPGDNSIYVTKIIEGGAAHKDGRLQIGDKILAVNSVGLEDVMHEDAVAALKNTYDVVYLKVAKPSNAYLSDSYAPPDITTSYSQHLDNEISHSSYLGTDYPTAMTPTSPRRYSPVAKDLLGEEDIPREPRRIVIHRGSTGLGFNIVGGEDGEGIFISFILAGGPADLSGELRKGDQILSVNGVDLRNASHEQAAIALKNAGQTVTIIAQYKPE

SEQ ID NO: 274 - protein, 186 aa, Artificial Sequence
  NeDLG PDZ domains 1-2
YEEIVLERGNSGLGFSIAGGIDNPHVPDDPGIFITKIIPGGAAAMDGRLGVNDCVLRVNEVEVSEVVHSRAVEALKEAGPVVRLVVRRRQPPPETIMEVNLLKGPKGLGFSIAGGIGNQHIPGDNSIYITKIIEGGAAQKDGRLQIGDRLLAVNNTNLQDVRHEEAVASLKNTSDMVYLKVAKPGS

SEQ ID NO: 275 - protein, 94 aa, Artificial Sequence
  SNTa1 PDZ domain 1
QRRRVTVRKADAGGLGISIKGGRENKMPILISKIFKGLAADQTEALFVGDAILSVNGEDLSSATHDEAVQVLKKTGKEVVLEVKYMKDVSPYFK

SEQ ID NO: 276 - protein, 72 aa, Artificial Sequence
  TAX-IP 43 PDZ domain 1
QKRGVKVLKQELGGLGISIKGGKENKMPILISKIFKGLAADQTQALYVGDAILSVNGADLRDATHDEAVQAL

SEQ ID NO: 277 - protein, 43 aa, Artificial Sequence
  LDP PDZ domain 1
RGMTTQQIDLQGPGPWGFRLVGRKDFEQPLAISRVTPGSKAAL

SEQ ID NO: 278 - protein, 84 aa, Artificial Sequence
  LIM PDZ domain 1
SNYSVSLVGPAPWGFRLQGGKDFNMPLTISSLKDGGKAAQANVRIGDVVLSIDGINAQGMTHLEAQNKIKGCTGSLNMTLQRAS

SEQ ID NO: 279 - protein, 178 aa, Artificial Sequence
  MINT1 PDZ domains 1-2
SENCKDVFIEKQKGEILGVVIVESGWGSILPTVIIANMMHGGPAEKSGKLNIGDQIMSINGTSLVGLPLSTCQSIIKGLENQSRVKLNIVRCPPVTTVLIRRPDLRYQLGFSVQNGIICSLMRGGIAERGGVRVGHRIIEINGQSVVATPHEKIVHILSNAVGEIHMKTMPAAMYRLL

SEQ ID NO: 280 - protein, 187 aa, Artificial Sequence
  X11 beta PDZ domains 1-2
HFSNSENCKELQLEKHKGEILGVVVVESGWGSILPTVILANMMNGCPAARSGKLSIGDQIMSINGTSLVGLPLATCQGIIKGLKNQTQVKLNIVSCPPVTTVLIKRPDLKYQLGFSVQNGIICSLMRGGIAERGGVRVGHRIIEINGQSVVATAHEKIVQALSNSVGEIHMKTMPAAMFRLLTGQEN

SEQ ID NO: 281 - protein, 80 aa, Artificial Sequence
  KIAA 0440 PDZ domain 1
SSVEMTLRRNGLGQLGFHVNYEGIVADVEPYGYAWQAGLRQGSRLVEICKVAVATLSHEQMIDLLRTSVTVKVVIIPPHE

SEQ ID NO: 282 - protein, 83 aa, Artificial Sequence
  KIAA 0545 PDZ domain 1
SGWETVDMTLRRNGLGQLGFHVKYDGTVAEVEDYGFAWQAGLRQGSRLVEICKVAVVTLTHDQMIDLLRTSVTVKVVIIPPFE

SEQ ID NO: 283 - protein, 86 aa, Artificial Sequence
  TAX-IP2 PDZ domain 1
RKEVEVFKSEDALGLTITDNGAGYAFIKRIKEGSVIDHIHLISVGDMIEAINGQSLLGCRHYEVARLLKELPRGRTFTLKLTEPRK

SEQ ID NO: 284 - protein, 93 aa, Artificial Sequence
  TAX-IP 2-like PDZ domain 1
IRGETKEVEVTKTEDALGLTITDNGAGYAFIKRIKEGSIINRIEAVCVGDSIEAINDHSIVGCRHYEVAKMLRELPKSQPFTLRLVQPKRAFE

SEQ ID NO: 285 - protein, 91 aa, Artificial Sequence
  TAX-IP 33 PDZ domain 1
HSHPRVVELPKTDEGLGFNVMGGKEQNSPIYISRIIPGGVAERHGGLKRGDQLLSVNGVSVEGEHHEKAVELLKAAKDSVKLVVRYTPKVL

SEQ ID NO: 286 - protein, 89 aa, Artificial Sequence
  MPP2 PDZ domain 1
PVPPDAVRMVGIRKTAGEHLGVTFRVEGGELVIARILHGGMVAQQGLLHVGDIIKEVNGQPVGSDPRALQELLRNASGSVILKILPNYQ

SEQ ID NO: 287 - protein, 42 aa, Artificial Sequence
  MINT3 PDZ domain 1
PVTTAIIHRPHAREQLGFCVEDGIVRPRPLAPGWGGRAALST

SEQ ID NO: 288 - protein, 103 aa, Artificial Sequence
  TIP-1 PDZ domain 1
QRVEIHKLRQGENLILGFSIGGGIDQDPSQNPFSEDKTDKGIYVTRVSEGGPAEIAGLQIGDKIMQVNGWDMTMVTHDQARKRLTKRSEEVVRLLVTRQSLQK

SEQ ID NO: 289 - protein, 89 aa, Artificial Sequence
  PTN-4 PDZ domain 1
LIRMKPDENGRFGFNVKGGYDQKMPVIVSRVAPGTPADLCVPRLNEGDQVVLINGRDIAEHTHDQVVLFIKASCERHSGELMLLVRPNA

SEQ ID NO: 290 - protein, 214 aa, Artificial Sequence
  prIL16 PDZ domain 1
IHVTILHKEEGAGLGFSLAGGADLENKVITVHRVFPNGLASQEGTIQKGNEVLSINGKSLKGTTHHDALAILRQAREPRQAVIVTRKLTPEAMPDLNSSTDSAASASAASDVSVESTAEATVCTVTLEKMSAGLGFSLEGGKGSLHGDKPLTINRIFKGAASEQSETVQPGDEILQLGGTAMQGLTRFEAWNIIKALPDGPVTIVIRRKSLQSK

SEQ ID NO: 291 - protein, 92 aa, Artificial Sequence
  CBP PDZ domain 1
QRKLVTVEKQDNETFGFEIQSYRPQNQNACSSEMFTLICKIQEDSPAHCAGLQAGDVLANINGVSTEGFTYKQVVDLIRSSGNLLTIETLNG

SEQ ID NO: 292 - protein, 82 aa, Artificial Sequence
  KIAA 0751 PDZ domain 1
RDSGAMLGLKVVGGKMTESGRLCAFITKVKKGSLADTVGHLRPGDEVLEWNGRLLQGATFEEVYNIILESKPEPQVELVVSR

SEQ ID NO: 293 - protein, 105 aa, Artificial Sequence
  KIAA 0559 PDZ domain 1
HYIFPHARIKITRDSKDHTVSGNGLGIRIVGGKEIPGHSGEIGAYIAKILPGGSAEQTGKLMEGMQVLEWNGIPLTSKTYEEVQSIISQQSGEAEICVRLDLNML

SEQ ID NO: 294 - protein, 93 aa, Artificial Sequence
  AF6 PDZ domain 1
LRKEPEIITVTLKKQNGMGLSIVAAKGAGQDKLGIYVKSVVKGGAADVDGRLAAGDQLLSVDGRSLVGLSQERAAELMTRTSSVVTLEVAKQG

SEQ ID NO: 295 - protein, 90 aa, Artificial Sequence
  PICK1 PDZ domain 1
PTVPGKVTLQKDAQNLIGISIGGGAQYCPCLYIVQVFDNTPAALDGTVAAGDEITGVNGRSIKGKTKVEVAKMIQEVKGEVTIHYNKLQE

SEQ ID NO: 296 - protein, 69 aa, Artificial Sequence
  RGS12 PDZ domain 1
PPRVRSVEVARGRAGYGFTLSGQAPCVLSCVMRGSPADFVGLRAGDQILAVNEINVKKASHEDVVKLIG

SEQ ID NO: 297 - protein, 324 aa, Artificial Sequence
  PDZK1 PDZ domain 2-4
RLCYLVKEGGSYGFSLKTVQGKKGVYMTDITPQGVAMRAGVLADDHLIEVNGENVEDASHEKVVEKVKKSGSRVMFLLVDKETDKRHVEQKIQFKRETASLKLLPHQPRIVEMKKGSNGYGFYLRAGSEQKGQIIKDIDSGSPAEEAGLKNNDLVVAVNGESVETLDHDSVVEMIRKGGDQTSLLVVDKETDNMYRLAHFSPFLYYQSQELPNGSVKEAPAPTPTSLEVSSPPDTTEEVDHKPKLCRLAKGENGYGFHLNAIRGLPGSFIKEVQKGGPADLAGLEDEDVIIEVNGVNVLDEPYEKVVDRIQSSGKNVTLLVCGK

SEQ ID NO: 298 - protein, 88 aa, Artificial Sequence
  KIAA 0316 PDZ domain 1
IPPAPRKVEMRRDPVLGFGFVAGSEKPVVVRSVTPGGPSEGKLIPGDQIVMINDEPVSAAPRERVIDLVRSCKESILLTVIQPYPSPK

SEQ ID NO: 299 - protein, 241 aa, Artificial Sequence
  DLG5 PDZ domain 2
PYVEEPRHVKVQKGSEPLGISIVSGEKGGIYVSKVTVGSIAHQAGLEYGDQLLEFNGINLRSATEQQARLIIGQQCDTITILAQYNPHVHQLSSHSRSSSHLDPAGTHSTLQGSGTTTPEHPSVIDPLMEQDEGPSTPPAKQSSSRIAGDANKKTLEPRVVFIKKSQLELGVHLCGGNLHGVFVAEVEDDSPAKGPDGLVPGDLILEYGSLDVRNKTVEEVYVEMLKPRDGVRLKVQYRPE

SEQ ID NO: 300 - protein, 181 aa, Artificial Sequence
  SYNT aa 67-241
REIKQGIREVILCKDQDGKIGLRLKSIDNGIFVQLVQANSPASLVGLRFGDQVLQINGENCAGWSSDKAHKVLKQAFGEKITMTIRDRPFERTVIMHKDSSGHVGFIFKSGKITSIVKDSSAARNGLLTDHHICEINGQNVIGLKDAQIADILSTAGTVVTITIMPTFIFEHIIKRMAPSM

SEQ ID NO: 301 - protein, 263 aa, Artificial Sequence
  WWP3 (MAGI-1) PDZ domains 1-2
PSELKGKFIHTKLRKSSRGFGFTVVGGDEPDEFLQIKSLVLDGPAALDGKMETGDVIVSVNDTCVLGHTHAQVVKIFQSIPIGASVDLELCRGYPLPFDPDDPNTSLVTSVAILDKEPIIVNGQETYDSPASHSSKTGKVNGMKDARPSSPADVASNSSHGYPNDTVSLASSIATQPELITVHIVKGPMGFGFTIADSPGGGGQRVKQIVDSPRCRGLKEGDLIVEVNKKNVQALTHNQVVDMLVECPKGSEVTLLVQRGGLP

SEQ ID NO: 302 - protein, 103 aa, Artificial Sequence
  TAX-IP 40 PDZ domain 1
LLPETHRRVRLHKHGSDRPLGFYIRDGMSVRVAPQGLERVPGIFISRLVRGGLAESTGLLAVSDEILEVNGIEVAGKTLDQVTDMMVANSHNLIVTVKPANQR

SEQ ID NO: 303 - protein, 94 aa, Artificial Sequence
  KIAA 0858 PDZ domain 1
FSDMRISINQTPGKSLDFGFTIKWDIPGIFVASVEAGSPAEFSQLQVDDEIIAINNTKFSYNDSKEWEEAMAKAQETGHLVMDVRRYGKAGSPE

SEQ ID NO: 304 - protein, 88 aa, Artificial Sequence
  TIAM1 PDZ domain 1
HSIHIEKSDTAADTYGFSLSSVEEDGIRRLYVNSVKETGLASKKGLKAGDEILEINNRAADALNSSMLKDFLSQPSLGLLVRTYPELE

SEQ ID NO: 305 - protein, 88 aa, Artificial Sequence
  ConEn PDZ domain 1
LEQKAVLEQVQLDSPLGLEIHTTSNCQHFVSQVDTQVPTDSRLQIQPGDEVVQINEQVVVGWPRKNMVRELLREPAGLSLVLKKIPIP

SEQ ID NO: 306 - protein, 55 aa, Artificial Sequence
  SPsht PDZ domain 1
SSSGISGSQRRYIGVMMLTLSPSAGLRPGDVILAIGEQMVQNAEDVYEAVRTQSE

SEQ ID NO: 307 - protein, 93 aa, Artificial Sequence
  DVL1 PDZ domain 1
LNIVTVTLNMERHHFLGISIVGQSNDRGDGGIYIGSIMKGGAVAADGRIEPGDMLLQVNDVNFENMSNDDAVRVLREIVSQTGPISLTVAKCW

SEQ ID NO: 308 - protein, 98 aa, Artificial Sequence
  NSP (PRSS11) PDZ domain 1
IRQAKGKAITKKKYIGIRMMSLTSSKAKELKDRHRDFPDVISGAYIIEVIPDTPAEAGGLKENDVIISINGQSVVSANDVSDVIKRESTLNMVVRRGN

SEQ ID NO: 309 - protein, 108 aa, Artificial Sequence
  GEF PDZ domain 1
CSVMIFEVVEQAGAIILEDGQELDSWYVILNGTVEISHPDGKVENLFMGNSFGITPTLDKQYMHGIVRTKVDDCQFVCIAQQDYWRILNHVEKNTHKVEEEGEIVMVH

SEQ ID NO: 310 - protein, 89 aa, Artificial Sequence
  KIAA 0902 PDZ domain 1
ILNEMIAPVMRVNYGQSTDINAFVGAVSLSCSDSGLWAVEGGNKLVCSGLLQASKSNLISGSVMYIEEKTKTKYTGNPTKMYEVVYQIG

SEQ ID NO: 311 - protein, 90 aa, Artificial Sequence
  KIAA 0561 PDZ domain 1
PPSLSTALARSTASACGRSASTWVIATSTLCTTSSGVWRTEAPPRRRACGLGTSSPTSTGSQCWGWCTWTSWSCCZRAATRYPCGPQPWR

SEQ ID NO: 312 - protein, 90 aa, Artificial Sequence
  NOS1 PDZ domain 1
IQPNVISVRLFKRKVGGLGFLVKERVSKPPVIISDLIRGGAAEQSGLIQAGDIILAVNGRPLVDLSYDSALEVLRGIASETHVVLILRGP

SEQ ID NO: 313 - DNA, 25 bp, Artificial Sequence
  forward primer 6CAF
tcggatccatgtgaccagagttcgg

SEQ ID NO: 314 - DNA, 24 bp, Artificial Sequence
  reverse primer 7CAR
tcggaattcagactgagtgcggta

SEQ ID NO: 315 - DNA, 25 bp, Artificial Sequence
  forward primer 62MPF
gggatccggaaagtgcgactcatac

SEQ ID NO: 316 - DNA, 26 bp, Artificial Sequence
  reverse primer 63MPR
acggatccgctggttgggaattactt

SEQ ID NO: 317 - DNA, 27 bp, Artificial Sequence
  forward primer 52LIFP
ctgcccgggaccgtcaccctggtgtcc

SEQ ID NO: 318 - DNA, 24 bp, Artificial Sequence
  reverse primer 53LIRP
tcgcccgggtcatgctcgagggtc

SEQ ID NO: 319 - DNA, 27 bp, Artificial Sequence
  forward primer 152KIF
ctgggatcccacatcagccgattgtga

SEQ ID NO: 320 - DNA, 29 bp, Artificial Sequence
  reverse primer 153KIR
tgtgaattcaaatggggtagtagtgattg

SEQ ID NO: 321 - DNA, 26 bp, Artificial Sequence
  forward primer 281KIF
gcaggatccctcccatcatcatccac

SEQ ID NO: 322 - DNA, 25 bp, Artificial Sequence
  reverse primer 282KIR
gatgaattctccaggggagttgttg

SEQ ID NO: 323 - DNA, 26 bp, Artificial Sequence
  forward primer 1DF
tcggatccaggttaatggctcagatg

SEQ ID NO: 324 - DNA, 22 bp, Artificial Sequence
  reverse primer 2DR
cggaattcggtgcatagccatc

SEQ ID NO: 325 - DNA, 24 bp, Artificial Sequence
  forward primer 8PSF
tcggatccttgagggggagatgga

SEQ ID NO: 326 - DNA, 24 bp, Artificial Sequence
  reverse primer 11PSR
tcggaattcgctatactcttctgg

SEQ ID NO: 327 - DNA, 29 bp, Artificial Sequence
  forward primer 71NEDF
caggatccaatatgaggaaatcgtacttg

SEQ ID NO: 328 - DNA, 26 bp, Artificial Sequence
  reverse primer 72NEDR
ttgaattcgaggctgcctggcttggc

SEQ ID NO: 329 - DNA, 26 bp, Artificial Sequence
  forward primer 124SYF
tacggatccagcggccgccgcgtgac

SEQ ID NO: 330 - DNA, 26 bp, Artificial Sequence
  reverse primer 125SYR
gtagaattcttgaaatacggtgagac

SEQ ID NO: 331 - DNA, 27 bp, Artificial Sequence
  forward primer 97TAF
tctggatccagaagcgtggcgtgaagg

SEQ ID NO: 332 - DNA, 25 bp, Artificial Sequence
  reverse primer 98TAR
cggaattcaacgcctgcaccgcctc

SEQ ID NO: 333 - DNA, 27 bp, Artificial Sequence
  forward primer 146LIF
ccaggatccgcggaatgaccacccagc

SEQ ID NO: 334 - DNA, 27 bp, Artificial Sequence
  reverse primer 147LIR
catgaattcgctagagccgccttgctt

SEQ ID NO: 335 - DNA, 30 bp, Artificial Sequence
  forward primer 182LF
ttaggatcctgagcaagtacagtgtgtcac

SEQ ID NO: 336 - DNA, 31 bp, Artificial Sequence
  reverse primer 183LR
cttgaattcagcagatgctctttgcagagtc

SEQ ID NO: 337 - DNA, 25 bp, Artificial Sequence
  forward primer 34MIF
cggaattcggaaaactgtaaagatg

SEQ ID NO: 338 - DNA, 25 bp, Artificial Sequence
  reverse primer 20MR
tcggaattcagcagcctgtacatcg

SEQ ID NO: 339 - DNA, 26 bp, Artificial Sequence
  forward primer 133 XF
accggatccacttctcaaactcggag

SEQ ID NO: 340 - DNA, 27 bp, Artificial Sequence
  reverse primer 134 XR
agcgaattctcctgacccgtgaggagc

SEQ ID NO: 341 - DNA, 29 bp, Artificial Sequence
  forward primer 230KIF
agggaattcatcggtggagatgactctgc

SEQ ID NO: 342 - DNA, 29 bp, Artificial Sequence
  reverse primer 231KIR
cagaattcatgcgggggaatgatgacaac

SEQ ID NO: 343 - DNA, 29 bp, Artificial Sequence
  forward primer 293TF
ccggatcccgaggcgagaccaaggaggtg

SEQ ID NO: 344 - DNA, 27 bp, Artificial Sequence
  reverse primer 294TR
aatgaattcgaaggccctcttgggctg

SEQ ID NO: 345 - DNA, 29 bp, Artificial Sequence
  forward primer 197TF
aggggatccgcaaggaggtggaggtgttc

SEQ ID NO: 346 - DNA, 29 bp, Artificial Sequence
  reverse primer 198TR
tgtggaattccttgcgaggctccgtgagc

SEQ ID NO: 347 - DNA, 29 bp, Artificial Sequence
  forward primer 293TF
ccggatcccgaggcgagaccaaggaggtg

SEQ ID NO: 348 - DNA, 27 bp, Artificial Sequence
  reverse primer 294TR
aatgaattcgaaggccctcttgggctg

SEQ ID NO: 349 - DNA, 27 bp, Artificial Sequence
  forward primer 92TAF
gtgggatccactcccaccctcgagtag

SEQ ID NO: 350 - DNA, 30 bp, Artificial Sequence
  reverse primer 93TAR
catgaattccagaacttttgggtgtatcgc

SEQ ID NO: 351 - DNA, 28 bp, Artificial Sequence
  forward primer 142MF
tcaggatccagcctgtacctcccgatgc

SEQ ID NO: 352 - DNA, 28 bp, Artificial Sequence
  reverse primer 143MR
atggaattcctggtagttgggcaggatc

SEQ ID NO: 353 - DNA, 29 bp, Artificial Sequence
  forward primer 188MF
actggatccccgtcaccaccgccatcatc

SEQ ID NO: 354 - DNA, 28 bp, Artificial Sequence
  reverse primer 189MR
ctcgaattccgtgctcagggccgcccta

SEQ ID NO: 355 - DNA, 30 bp, Artificial Sequence
  forward primer 86TAF
cagggatccaaagagttgaaattcacaagc

SEQ ID NO: 356 - DNA, 27 bp, Artificial Sequence
  reverse primer 87TAR
acggaattctgcagcgactgccgcgtc

SEQ ID NO: 357 - DNA, 27 bp, Artificial Sequence
  forward primer 247PTF
atcggatcctaatcagaatgaaacctg

SEQ ID NO: 358 - DNA, 27 bp, Artificial Sequence
  reverse primer 248PTR
atcgaattcagcattaggtcgaactag

SEQ ID NO: 359 - DNA, 26 bp, Artificial Sequence
  forward primer 75PRF
acgggatccatgtcaccatcttacac

SEQ ID NO: 360 - DNA, 28 bp, Artificial Sequence
  reverse primer 76PRR
gtgaattccttggactggaggctttttc

SEQ ID NO: 361 - DNA, 29 bp, Artificial Sequence
  forward primer 235CYF
cctggatccaaagaaagcttgttactgtg

SEQ ID NO: 362 - DNA, 26 bp, Artificial Sequence
  reverse primer 236CYR
tcagaattccattaagagtctctatc

SEQ ID NO: 363 - DNA, 27 bp, Artificial Sequence
  forward primer 145HF
gtgggatccgagattcaggagcaatgc

SEQ ID NO: 364 - DNA, 28 bp, Artificial Sequence
  reverse primer 146HR
ctggaattcgccttgaaactacaagttc

SEQ ID NO: 365 - DNA, 27 bp, Artificial Sequence
  forward primer 130KIF
aaaggatccactacatctttcctcacg

SEQ ID NO: 366 - DNA, 29 bp, Artificial Sequence
  reverse primer 131KIR
tcacaattggatagcatattgaggtccag

SEQ ID NO: 367 - DNA, 25 bp, Artificial Sequence
  forward primer 66AFF
tcggatcctgaggaaagaacctgaa

SEQ ID NO: 368 - DNA, 26 bp, Artificial Sequence
  reverse primer 67AFR
tagaattcaccctgctttgctacttc

SEQ ID NO: 369 - DNA, 26 bp, Artificial Sequence
  forward primer 287PIF
tcgggatcccgactgtgcctgggaag

SEQ ID NO: 370 - DNA, 31 bp, Artificial Sequence
  reverse primer 288PIR
cttgaattcctcctgcagcttcttgttgtag

SEQ ID NO: 371 - DNA, 27 bp, Artificial Sequence
  forward primer 64RGF
tgggatcccgcccccaagggtgcggag

SEQ ID NO: 372 - DNA, 25 bp, Artificial Sequence
  reverse primer 65RGR
aggaattcccaattaatttcactac

SEQ ID NO: 373 - DNA, 27 bp, Artificial Sequence
  forward primer 238PDF
ccggatccggctctgctatctcgtgaa

SEQ ID NO: 374 - DNA, 28 bp, Artificial Sequence
  reverse primer 239PDR
taggaattctttcctcagactagaagtg

SEQ ID NO: 375 - DNA, 26 bp, Artificial Sequence
  forward primer 158KIF
aaaggatccctccggctcctcggaag

SEQ ID NO: 376 - DNA, 29 bp, Artificial Sequence
  reverse primer 159KIR
ttagaattctgatttgggagaagggtaag

SEQ ID NO: 377 - DNA, 27 bp, Artificial Sequence
  forward primer 81PDLGF
ataggatcccttatgtggaggagccac

SEQ ID NO: 378 - DNA, 30 bp, Artificial Sequence
  reverse primer 82PDLGR
ttgaattcctcagggcggtactgcaccttc

SEQ ID NO: 379 - DNA, 27 bp, Artificial Sequence
  forward primer 14SF
tcggatccttgaaattaagcaagggat

SEQ ID NO: 380 - DNA, 25 bp, Artificial Sequence
  reverse primer 15SR
tcggaattcatgcctggagccatcc

SEQ ID NO: 381 - DNA, 26 bp, Artificial Sequence
  forward primer 164WWF
cacggatcccttctgagttgaaaggc

SEQ ID NO: 382 - DNA, 28 bp, Artificial Sequence
  reverse primer 165WWR
cttgaattctggcagccctcctcgttgc

SEQ ID NO: 383 - DNA, 27 bp, Artificial Sequence
  forward primer 136TF
acgggatcctactgcctgagacccacc

SEQ ID NO: 384 - DNA, 29 bp, Artificial Sequence
  reverse primer 137TR
acggaattccgctggttggcgggcttgac

SEQ ID NO: 385 - DNA, 26 bp, Artificial Sequence
  forward primer 278KIF
aggagatcttcagtgatatgagaatc

SEQ ID NO: 386 - DNA, 26 bp, Artificial Sequence
  reverse primer 279KIR
cttgaattcaggtgaaccagcctttc

SEQ ID NO: 387 - DNA, 25 bp, Artificial Sequence
  forward primer 39TF
tcggatccacagcatccacattgag

SEQ ID NO: 388 - DNA, 23 bp, Artificial Sequence
  reverse primer 40TR
tcggaattcctccagctcggggt

SEQ ID NO: 389 - DNA, 29 bp, Artificial Sequence
  forward primer 296CF
aggggatcctggaacagaaggccgtgctc

SEQ ID NO: 390 - DNA, 28 bp, Artificial Sequence
  reverse primer 297CR
gggaattccggtatcgggatcttccttc

SEQ ID NO: 391 - DNA, 26 bp, Artificial Sequence
  forward primer 191SF
gaagaattcctcctccggaatcagtg

SEQ ID NO: 392 - DNA, 30 bp, Artificial Sequence
  reverse primer 192SR
tgcgaattcggattgggttcgaacagcttc

SEQ ID NO: 393 - DNA, 21 bp, Artificial Sequence
  1st PCR forward primer 55DVISF
tcatccagactcatccggaag

SEQ ID NO: 394 - DNA, 20 bp, Artificial Sequence
  1st PCR reverse primer 56DVISR
gctcatgtcactcttcaccg

SEQ ID NO: 395 - DNA, 25 bp, Artificial Sequence
  2nd PCR nested forward primer 37DVF
tcggatccaaacggtcactctcaac

SEQ ID NO: 396 - DNA, 26 bp, Artificial Sequence
  2nd PCR nested reverse primer 38DVR
tcggaattcccagcacttggctacag

SEQ ID NO: 397 - DNA, 28 bp, Artificial Sequence
  forward primer 194NSF
cccggatccgacaggccaaaggaaaagc

SEQ ID NO: 398 - DNA, 31 bp, Artificial Sequence
  reverse primer 195NSR
gatgaattcattacccctgcggaccaccatg

SEQ ID NO: 399 - DNA, 27 bp, Artificial Sequence
  forward primer 275GF
gagagatctgctcagtgatgatttttg

SEQ ID NO: 400 - DNA, 27 bp, Artificial Sequence
  reverse primer 276GR
ccggaattcatgtaccataacaatttc

SEQ ID NO: 401 - DNA, 25 bp, Artificial Sequence
  forward primer 290KIF
agaggatcctcaatgaaatgattgc

SEQ ID NO: 402 - DNA, 28 bp, Artificial Sequence
  reverse primer 291KIR
tctgaattccaatttggtagaccacttc

SEQ ID NO: 403 - DNA, 28 bp, Artificial Sequence
  forward primer 161KIF
cctggatccccccatcgttatccacagc

SEQ ID NO: 404 - DNA, 26 bp, Artificial Sequence
  reverse primer 162KIR
gaggaattctccagggctgtggtccg

SEQ ID NO: 405 - DNA, 25 bp, Artificial Sequence
  forward primer 155NOF
agcggatccagcccaatgtcatttc

SEQ ID NO: 406 - DNA, 25 bp, Artificial Sequence
  reverse primer 156NOR
gaagaattcagggcccctcagaatg

SEQ ID NO: 407 - protein, 4 aa, Artificial Sequence
  PDZ domain signature sequence
GLGF

SEQ ID NO: 408 - protein, 16 aa, Artificial Sequence
  MBPAc1-16 peptide
XSQKRPSQRHGSKYLA

SEQ ID NO: 409 - protein, 19 aa, Artificial Sequence
  PL peptide
ISKATPALPTVSISSSAEV

SEQ ID NO: 410 - protein, 20 aa, Artificial Sequence
  PL peptide
ISGTPTSTMVHGMTSSSSVV

SEQ ID NO: 411 - protein, 20 aa, Artificial Sequence
  PL peptide
CAISGTSSDRGYGSPRYAEV

SEQ ID NO: 412 - protein, 20 aa, Artificial Sequence
  PL peptide
SVFSIPTLWSPWPPSSSSQL

SEQ ID NO: 413 - protein, 19 aa, Artificial Sequence
  PL peptide
SEKKTSQSPHRFQKTCSPI

SEQ ID NO: 414 - protein, 18 aa, Artificial Sequence
  PL peptide
SPQPDSTDNDDYDDISAA

SEQ ID NO: 415 - protein, 20 aa, Artificial Sequence
  PL peptide
QATSRNGHSARQHVVADTEL

SEQ ID NO: 416 - protein, 20 aa, Artificial Sequence
  PL peptide
PDKFLQCVKNPEDSSCTSEI

SEQ ID NO: 417 - protein, 20 aa, Artificial Sequence
  PL peptide
QFMTADETRNLQNVDMKIGV

SEQ ID NO: 418 - protein, 19 aa, Artificial Sequence
  PL peptide
KKGTYLTDETHREVKFTSL

SEQ ID NO: 419 - protein, 19 aa, Artificial Sequence
  PL peptide
PYGTAMEKAQLKPPATSDA

SEQ ID NO: 420 - protein, 19 aa, Artificial Sequence
  PL peptide
HKAEIHAQPSDKERLTSDA

SEQ ID NO: 421 - protein, 20 aa, Artificial Sequence
  PL peptide
KDITSDSENSNFRNEIQSLV

SEQ ID NO: 422 - protein, 19 aa, Artificial Sequence
  PL peptide
TSGTGHNQTRALRASESGI

SEQ ID NO: 423 - protein, 19 aa, Artificial Sequence
  PL peptide
ERLKLEPHEGLLLRFPYAA

SEQ ID NO: 424 - protein, 20 aa, Artificial Sequence
  PL peptide
STNHSIGSTQSTPCSTSSMA

SEQ ID NO: 425 - protein, 20 aa, Artificial Sequence
  PL peptide
ARKANMKGSYSLVEAQKSKV

SEQ ID NO: 426 - protein, 20 aa, Artificial Sequence
  PL peptide
PKQANGGAYQKPTKQEEFYA

SEQ ID NO: 427 - protein, 17 aa, Artificial Sequence
  PL peptide
ENLAPVTTFGKTNGYIA

SEQ ID NO: 428 - protein, 20 aa, Artificial Sequence
  PL peptide
DLGNMEENKKLEENNHKTEA

SEQ ID NO: 429 - protein, 20 aa, Artificial Sequence
  PL peptide
QEEDGCSCRFPEEEEGGCEL

SEQ ID NO: 430 - protein, 20 aa, Artificial Sequence
  PL peptide
TREDIYVNYPTFSRRPKTRV

SEQ ID NO: 431 - protein, 19 aa, Artificial Sequence
  PL peptide
SSKSKSSEESQTFFGLYKL

SEQ ID NO: 432 - protein, 20 aa, Artificial Sequence
  PL peptide
YSATYSELEDPGEMSPPIDL

SEQ ID NO: 433 - protein, 20 aa, Artificial Sequence
  PL peptide
ISKLGISGDIDLTSASYTMI

SEQ ID NO: 434 - protein, 20 aa, Artificial Sequence
  PL peptide
LNFPLLQGIRVHGMEALGSF

SEQ ID NO: 435 - protein, 19 aa, Artificial Sequence
  PL peptide
EVICYIEKPGVETLEDSVF

SEQ ID NO: 436 - protein, 19 aa, Artificial Sequence
  PL peptide
ARHRVTSYTSSSVNVSSNL

SEQ ID NO: 437 - protein, 19 aa, Artificial Sequence
  PL peptide
KDSRPSFVGSSSGHTSTTL

SEQ ID NO: 438 - protein, 19 aa, Artificial Sequence
  PL peptide
AWDDSARAAGGQGLHVTAL

SEQ ID NO: 439 - protein, 20 aa, Artificial Sequence
  PL peptide
GTSDMKDLVGNIEQNEHSVI

SEQ ID NO: 440 - protein, 20 aa, Artificial Sequence
  PL peptide
SFPPCGHRENVPGQSLVSFV

SEQ ID NO: 441 - protein, 19 aa, Artificial Sequence
  PL peptide
TTNNNPNSAVNIKKIFTDV

SEQ ID NO: 442 - protein, 20 aa, Artificial Sequence
  PL peptide
LNSCSNRRVYKKMPSIESDV

SEQ ID NO: 443 - protein, 18 aa, Artificial Sequence
  PL peptide
TFAAGFNSTGLPHSTTRV

SEQ ID NO: 444 - protein, 19 aa, Artificial Sequence
  PL peptide
QGDPALQDAGDSSRKEYFI

SEQ ID NO: 445 - protein, 19 aa, Artificial Sequence
  PL peptide
SSAKSSNKNKKNKDKEYYV

SEQ ID NO: 446 - protein, 20 aa, Artificial Sequence
  PL peptide
GERKPSSAAYQKAPTKEFYA

SEQ ID NO: 447 - protein, 20 aa, Artificial Sequence
  PL peptide
LASKSAEEGKQIPDSLSTDL

SEQ ID NO: 448 - protein, 20 aa, Artificial Sequence
  PL peptide
LERVSSTSPSTGEHELSAGF

SEQ ID NO: 449 - protein, 20 aa, Artificial Sequence
  PL peptide
GKGKSIGRAPEASLQDKEGA

SEQ ID NO: 450 - protein, 20 aa, Artificial Sequence
  PL peptide
LERTSSVSPSTAEPELSIVF

SEQ ID NO: 451 - protein, 20 aa, Artificial Sequence
  PL peptide
DTPSSSYTQSTMDHDLHDAL

SEQ ID NO: 452 - protein, 20 aa, Artificial Sequence
  PL peptide
PSWRRSSLSESENATSLTTF

SEQ ID NO: 453 - protein, 19 aa, Artificial Sequence
  PL peptide
SSGGRARHSYHHPDQDHWC

SEQ ID NO: 454 - protein, 19 aa, Artificial Sequence
  PL peptide
VTSPNKHLGLVTPHKTELV

SEQ ID NO: 455 - protein, 19 aa, Artificial Sequence [sequence omitted]

SEQ ID NO: 456 - protein, 20 aa, Artificial Sequence
  PL peptide
SMQPDNSSDSDYDLHGAQRL

SEQ ID NO: 457 - protein, 17 aa, Artificial Sequence
  PL peptide
TIYENLAPVTTFGKTIA

SEQ ID NO: 458 - protein, 20 aa, Artificial Sequence
  PL peptide
QISPGGLEPPSEKHFRETEV

SEQ ID NO: 459 - protein, 19 aa, Artificial Sequence
  PL peptide
SWRRSSLSESENATSLTTF

SEQ ID NO: 460 - protein, 20 aa, Artificial Sequence
  PL peptide
KENDYESISDLQQGRDITRL

SEQ ID NO: 461 - protein, 4 aa, Artificial Sequence
  PDZ ligand
KEGA

SEQ ID NO: 462 - protein, 15 aa, Artificial Sequence
  linker
LQSTVPRARDPPVAT

SEQ ID NO: 463 - protein, 10 aa, Artificial Sequence
  linker
LDPGYPPVAT

SEQ ID NO: 464 - protein, 341 aa, Artificial Sequence
  DVL1 Construct  (N-P)
MAETKIIYHMDEEETPYLVKLPVAPERVTLADFKNVLSNRPVHAYKFFKSMDQDFGVVKEEIFDDNAKLPCFNGRVVSWLVLVEGAHSDAGSQGTDSHTDLPPPLERTGGIGDSRSPSFQPDVASSRDGMDNETGTESMVSHRRDRARRRNREEAARTNGHPRGDRRRDVGLPPDSASTALSSELESSSFVDSDEDDSTSRLSSSTEQSTSSRLIRKHKRRRRKQRLRQADRASSFSSMTDSTMSLNIITVTLNMERHHFLGICIVGQSNDRGDGGIYIGSIMKGGAVAADGRIEPGDMLLQVNDVNFENMSNDDAVRVLREIVSQTGPISLTVAKCWDPT

SEQ ID NO: 465 - protein, 198 aa, Artificial Sequence
  DVL1 Construct (N)
MAETKIIYHMDEEETPYLVKLPVAPERVTLADFKNVLSNRPVHAYKFFFKSMDQDFGVVKEEIFDDNAKLPCFNGRVVSWLVLVEGAHSDAGSQGTDSHTDLPPPLERTGGIGDSRSPSFQPDVASSRDGMDNETGTESMVSHRRDRARRRNREEAARTNGHPRGDRRRDVGLPPDSASTALSSELESSSFVDSDEDG

SEQ ID NO: 466 - protein, 97 aa, Artificial Sequence
  DVL1 Construct (P)
SLNIITVTLNMERHHFLGICIVGQSNDRGDGGIYIGSIMKGGAVAADGRIEPGDMLLQVNDVNFENMSNDDAVRVLREIVSQTGPISLTVAKCWDPT

SEQ ID NO: 467 - protein, 427 aa, Artificial Sequence
  KIAA 0751 Construct (N-J)
MMYFGGHSLEEDLEWSEPQIKDSGVDTCSSTTLNEEHSHSDKHPVTWQPSKDGDRLIGRILLNKRLKDGSVPRDSGAMLGLKVVGGKMTESGRLCAFITKVKKGSLADTVGHLRPGDEVLEWNGRLLQGATFEEVYNIILESKPEPQVELVVSRPIGDIPRIPDSTHAQLESSSSSFESQKMDRPSISVTSPMSPGMLRDVPQFLSGQLSIKLWFDKVGHQLIVTILGAKDLPSREDGRPRNPYVKIYFLPDRSDKNKRRTKTVKKTLEPKWNQTFIYSPVHRREFRERMLEITLWDQARVREEESEFLGEILIELETALLDDEPHWYKLQTHDVSSLPLPHPSPYMPRRQLHGESPTRRLQRSKRISDSEVSDYDCDDGIGVVSDYRHDGRDLQSSTLSVPEQVMSSNHCSPSGSPHRVDVIGRTT

SEQ ID NO: 468 - protein, 92 aa, Artificial Sequence
  KIAA 0751 Construct (P)
LKDGSVPRDSGAMLGLKVVGGKMTESGRLCAFITKVKKGSLADTVGHLRPGDEVLEWNGRLLQGATFEEVYNIILESKPEPQVELVVSRPIA

SEQ ID NO: 469 - protein, 251 aa, Artificial Sequence
  PAR6 Construct (N-P)
MARPQRTPARSPDSIVEVKSKFDAEFRRFALPRASVSGFQEFSRLLRAVHQIPGLDVLLGYTDAHGDLLPLTNDDSLHRALASGPPPLRLLVQKREADSSGLAFASNSLQRRKKGLLLRPVAPLRTRPPLLISLPQDFRQVSSVIDVDLLPETHRRVRLHKHGSDRPLGFYIRDGMSVRVAPQGLERVPGIFISRLVRGGLAESTGLLAVSDEILEVNGIEVAGKTLDQVTDMMVANSHNLIVTVKPANQR

SEQ ID NO: 470 - protein, 146 aa, Artificial Sequence
  PAR6 Construct (N)
MARPQRTPARSPDSIVEVKSKFDAEFRRFALPRASVSGFQEFSRLLRAVHQIPGLDVLLGYTDAHGDLLPLTNDDSLHRALASGPPPLRLLVQKREADSSGLAFASNSLQRRKKGLLLRPVAPLRTRPPLLISLPQDRQVSSVIDV

SEQ ID NO: 471 - protein, 97 aa, Artificial Sequence
  PAR6 Construct (P)
RRVRLHKHGSDRPLGFYIRDGMSVRVAPQGLERVPGIFISRLVRGGLAESTGLLAVSDEILEVNGIEVAGKTLDQVTDMMVANSHNLIVTVKPANQR

SEQ ID NO: 472 - protein, 442 aa, Artificial Sequence
  PSD95 Construct (N-P3)
MSQRPRAPRSALWLLAPPLLRWAPPLLTVLHSDLFQALLDILDYYEASLSESQKYRYQDEDTPPLEHSPAHLPNQANSPPVIVNTDTLEAPGYELQVNGTEGEMEYEEITLERGNSGLGFSIAGGTDNPHIGDDPSIFITKIIPGGAAAQDGRLRVNDSILFVNEVDVREVTHSAAVEALKEAGSIVRLYVMRRKPPAEKVMEIKLIKGPKGLGFSIAGGVGNQHIPGDNSIYVTKIIEGGAAHKDGRLQIGDKILAVNSVGLEDVMHEDAVAALKNTYDVVYLKVAKPSNAYLSDSYAPPDITTSYSQHLDNEISHSSYLGTDYPTAMTPTSPRRYSPVAKDLLGEEDIPREPRRIVIHRGSTGLGFNIVGGEDGEGIFISFILAGGPADLSGELRKGDQILSVNGVDLRNASHEQAAIALKNAGQTVTIIAQYKPEEYSR

SEQ ID NO: 473 - protein, 84 aa, Artificial Sequence
  CASK Construct (P)
RLVQFQKNTDEPMGITLKMNELNHCIVARIMHGGMIHRQGTLHVGDEIREINGISVANQTVEQLQKMLREMRGSITFKIVPSYR

SEQ ID NO: 474 - protein, 294 aa, Artificial Sequence
  MPP2/DLG2 Construct (N-SH3)
MPVAATNSETAMQQVLDNLGSLPSATGAAELDLIFLRGIMESPIVRSLAKAHERLEETKLEAVRDNNLELVQEILRDLAQLAEQSSTAAELAHILQEPHFQSLLETHDSVASKTYETPPPSPGLDPTFSNQPVPPDAVRMVGIRKTAGEHLGVTFRVEGGELVIARILHGGMVAQQGLLHVGDIIKEVNGQPVGSDPRALQELLRNASGSVILKILPSYQEPHLPRQVFVKCHFDYDPARDSLIPCKEAGLRFNAGDLLQIVNQDDANWWQACHVEGGSAGLIPSQLLEEKRKG

SEQ ID NO: 475 - protein, 121 aa, Artificial Sequence
  TIP1 Construct (N-C)
YIPGQPVTAVVQRVEIHKLRQGENLILGFSIGGGIDQDPSQNPFSEDKTDKGIYVTRVSEGGPAEIAGLQSGDKIMQVNGWDMTMVTHDQARKRLTKRSEEVVRLLVTRQSLQKAVQQSML

SEQ ID NO: 476 - DNA, 28 bp, Artificial Sequence
  primer 308 DVF (N 128 - N 155)
tcggaattcgtcgcgccatggcggagac

SEQ ID NO: 477 - DNA, 29 bp, Artificial Sequence
  primer 311 DVR (N 1004 - N 1032)
gggaattcggtcccagcacttggccacag

SEQ ID NO: 478 - DNA, 28 bp, Artificial Sequence
  primer 344 DVF (N 873 - N 900)
ccagaattctcaacatcgtcactgtcac

SEQ ID NO: 479 - DNA, 32 bp, Artificial Sequence
  primer 345 DVR (N713 - N744)
tcggaattccatcctcgtccgagtccacaaag

SEQ ID NO: 480 - DNA, 28 bp, Artificial Sequence
  primer 318 KIF (N 1366 - N 1393)
agacaattgaggaaatgatgtactttgg

SEQ ID NO: 481 - DNA, 28 bp, Artificial Sequence
  primer 319 KIR (N 1830 - N 1857)
gaacaattgcaataggccttgaaactac

SEQ ID NO: 482 - DNA, 28 bp, Artificial Sequence
  primer 320 KIR (N 2640 -N 2667)
acccaattgtagtccttcctataacatc

SEQ ID NO: 483 - DNA, 27 bp, Artificial Sequence
  primer 341 KIF (N 1567 - N 1593)
atagaattctaaaagatggaagtgtac

SEQ ID NO: 484 - DNA, 29 bp, Artificial Sequence
  primer 322 PAF (N 55 - N 82)
cccgaattcgccatggcccggccgcagag

SEQ ID NO: 485 - DNA, 28 bp, Artificial Sequence
  primer 324 PAR (N 798 - N 825)
cgtgaattcgctggttggcgggcttgac

SEQ ID NO: 486 - DNA, 30 bp, Artificial Sequence
  primer 342 PAF (N 519 - N 548)
gaggaattccgacgggtgcggctgcacaag

SEQ ID NO: 487 - DNA, 31 bp, Artificial Sequence
  primer 343 PAR (N 485 - N 516)
gcagaattcccacgtctatgactgaggaaac

SEQ ID NO: 488 - DNA, 32 bp, Artificial Sequence
  primer 315 PSF (N847 - N876)
agagaattcagagatatgtcccagagaccaag

SEQ ID NO: 489 - DNA, 29 bp, Artificial Sequence
  primer 304 PSR (N 2161 - N 2189)
cgagaattctgtactcttctggtttatac

SEQ ID NO: 490 - DNA, 29 bp, Artificial Sequence
  primer 336 CAF (N 1484 - N 1512)
ccagaattcggctggtacagtttcaaaag

SEQ ID NO: 491 - DNA, 29 bp, Artificial Sequence
  primer 325 CAR (N 1722 - N 1750)
actgaattcggtaacttggcacaatcttg

SEQ ID NO: 492 - DNA, 27 bp, Artificial Sequence
  primer 305 MF (N 58 - N 84)
agagaattcagagcccttgcctccttc

SEQ ID NO: 493 - DNA, 28 bp, Artificial Sequence
  primer 306 MR (N 798 - N 825)
tgagaattcctttccgcttctcctccag

SEQ ID NO: 494 - DNA, 21 bp, Artificial Sequence
  primer 1318 TIP R3-1 (N 336 - N 356)
cagtccatgctgtcggatccg

SEQ ID NO: 495 - DNA, 21 bp, Artificial Sequence
  primer 1317 TIP R5-1
gtcggaattccctacatcccg

SEQ ID NO: 496 - protein, 4 aa, Artificial Sequence
  PDZ consensus
ETQV

SEQ ID NO: 497 - protein, 4 aa, Artificial Sequence
  HPV57 E6 PL motif
RTSH

SEQ ID NO: 498 - protein, 4 aa, Artificial Sequence
  HPV2a E6 PL motif
RTLH

SEQ ID NO: 499 - protein, 4 aa, Artificial Sequence
  HPV63 E6 PL motif
LYII

SEQ ID NO: 500 - protein, 4 aa, Artificial Sequence
  HPV77 E6 PL motif
QSRQ

SEQ ID NO: 501 - protein, 4 aa, Artificial Sequence
  HPV80 E6 PL motif
GSIE

SEQ ID NO: 502 - protein, 10 aa, Artificial Sequence
  HPV61 E6 C-terminal
TGPCTARWQP

SEQ ID NO: 503 - protein, 12 aa, Artificial Sequence
  HPV60 E6 C-terminal
RQRSYCRNCIEK

SEQ ID NO: 504 - protein, 11 aa, Artificial Sequence
  HPV55 E6 C-terminal
CWTSCMETILP

SEQ ID NO: 505 - protein, 10 aa, Artificial Sequence
  HPV50 E6 C-terminal
CCRNCYEHEG

SEQ ID NO: 506 - protein, 10 aa, Artificial Sequence
  HPV48 E6 C-terminal
CRNCISHEGR

SEQ ID NO: 507 - protein, 14 aa, Artificial Sequence
  HPV44 E6 C-terminal
CFHCWTSCMETILP

SEQ ID NO: 508 - protein, 14 aa, Artificial Sequence
  HPV38 E6 C-terminal
GNWKGRCRHCKAIE

SEQ ID NO: 509 - protein, 12 aa, Artificial Sequence
  HPV37 E6 C-terminal
WKGLCRHCGSIG

SEQ ID NO: 510 - protein, 20 aa, Artificial Sequence
  HPV66 E6 C-terminal
TGSCLQCWRHTSRQATESTV

SEQ ID NO: 511 - protein, 20 aa, Artificial Sequence
  HPV57 E6 C-terminal
RCMNCAPRCMENAPALRTSH

SEQ ID NO: 512 - protein, 20 aa, Artificial Sequence
  HPV2a E6 C-terminal
HCMNCGSSCTATDPASRTLH

SEQ ID NO: 513 - protein, 20 aa, Artificial Sequence
  HPV16 E6 C-terminal
WTGRCMSCCRSSRTRRETQL

SEQ ID NO: 514 - protein, 20 aa, Artificial Sequence
  HPV18 E6 C-terminal
HSCCNRARQERLQRRRETQV

SEQ ID NO: 515 - protein, 20 aa, Artificial Sequence
  HPV31 E6 C-terminal
GRWTGRCIACWRRPRTETQV

SEQ ID NO: 516 - protein, 21 aa, Artificial Sequence
  HPV33 E6 C-terminal
CAACWRSARRRRLQRRRETAL

SEQ ID NO: 517 - protein, 21 aa, Artificial Sequence
  HPV51 E6 C-terminal
CANCWQRTRQRRLQRRNETQV

SEQ ID NO: 518 - protein, 21 aa, Artificial Sequence
  HPV52 E6 C-terminal
CSECWRPTRRPRLQRRRVTQV

SEQ ID NO: 519 - protein, 21 aa, Artificial Sequence
  HPV58 E6 C-terminal
CAVCWRPARRRRLQRRRQTQV

SEQ ID NO: 520 - protein, 20 aa, Artificial Sequence
  HPV70 E6 C-terminal
RHCWTSNREDRRRIRRETQV

SEQ ID NO: 521 - protein, 20 aa, Artificial Sequence
  HPV63 E6 C-terminal
VHKVRNKFKAKCSLCRLYII

SEQ ID NO: 522 - protein, 20 aa, Artificial Sequence
  HPV77 E6 C-terminal
GHWRGSCLHCWSRCMGQSRQ

SEQ ID NO: 523 - protein, 20 aa, Artificial Sequence
  HPV80 E6 C-terminal
QFHKVRRNWKGLCRHCGSIE

SEQ ID NO: 524 - protein, 12 aa, Artificial Sequence
  HPV21 E6 C-terminal
WKGICRLCKHFQ

SEQ ID NO: 525 - protein, 18 aa, Artificial Sequence
  HPV11 E6 C-terminal
WKGRCLHCWTTCMEDLLP

SEQ ID NO: 526 - protein, 15 aa, Artificial Sequence
  HPV36 E6 C-terminal
WKGICRQCKHFYNDW

SEQ ID NO: 527 - protein, 18 aa, Artificial Sequence
  HPV29 E6 C-terminal
WRGSCLYCWSRCMGQSPR

SEQ ID NO: 528 - protein, 14 aa, Artificial Sequence
  HPV28 E6 C-terminal
CQYCWLRCTVRIPQ

SEQ ID NO: 529 - protein, 16 aa, Artificial Sequence
  HPV24 E6 C-terminal
KVRRGWKGLCRQCKQI

SEQ ID NO: 530 - protein, 16 aa, Artificial Sequence
  HPV22 E6 C-terminal
VRDHWKGRCRHCKAIE

SEQ ID NO: 531 - protein, 18 aa, Artificial Sequence
  HPV21 E6 C-terminal
HKVRGSWKGICRLCKHFQ

SEQ ID NO: 532 - protein, 19 aa, Artificial Sequence
  HPV20 E6 C-terminal
FYLVRGSWKGICRLCKHFQ

SEQ ID NO: 533 - protein, 20 aa, Artificial Sequence
  HPV4 E6 C-terminal
TCYLIRGLWRGYCRNCIRKQ

SEQ ID NO: 534 - protein, 20 aa, Artificial Sequence
  HPV54 E6 C-terminal
RRFHCVRGYWKGRCLHCWKP

SEQ ID NO: 535 - protein, 20 aa, Artificial Sequence
  HPV5B E6 C-terminal
KVRNAWKGICRQCKHFYHDW

SEQ ID NO: 536 - protein, 20 aa, Artificial Sequence
  HPV74 E6 C-terminal
NTWKGRCFHCWTTCMENILP

SEQ ID NO: 537 - protein, 20 aa, Artificial Sequence
  HPV75 and HPV76 E6 C-terminal
EFHKVRNRWKGVCRHCRVIE

SEQ ID NO: 538 - protein, 20 aa, Artificial Sequence
  HPV47 E6 C-terminal
KVRNAWKGVCRQCKHFYNDW

SEQ ID NO: 539 - protein, 20 aa, Artificial Sequence
  HPV65 E6 C-terminal
ACYLIRGLWRGYCRNCIRKQ

SEQ ID NO: 540 - DNA, 33 bp, Artificial Sequence
  sequence unique to vector pDsRED1-N1(+ATG)
attgccaccatgggaattctggatccgggagat

SEQ ID NO: 541 - protein, 5 aa, Artificial Sequence
  flexible polylinker
GGGGS

SEQ ID NO: 542 - protein, 14 aa, Artificial Sequence
  linker
EGKSSGSGSESKVD

SEQ ID NO: 543 - protein, 18 aa, Artificial Sequence
  linker
KESGSVSSEQLAQFRSLD

(19) United States
(12) Patent Application Publication
(10) Pub. No.: US 2005/0037969 A1
(43) Pub. Date: Feb. 17, 2005

(54) Molecular interactions in hematopoietic cells

(75) Inventors: Peter S. Lu, Mountain View, CA (US); Joshua D. Rabinowitz, Mountain View, CA (US); Johannes Schweizer, Mountain View, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Arbor Vita Corporation, Sunnyvale, CA (US)

(21) Appl. No.: 10/938,249

(22) Filed: Sep. 10, 2004

Related U.S. Application Data
(63) Continuation of application No. 09/724,553, filed on Nov. 28, 2000.
Application No. 09/724,553 is a continuation-in-part of application No. 09/710,059, filed on Nov. 10, 2000, now abandoned.
Application No. 09/710,059 is a continuation-in-part of application No. 09/688,017, filed on Oct. 13, 2000.
Application No. 09/688,017 is a continuation-in-part of application No. 09/570,118, filed on May 12, 2000, now abandoned.
(63) Continuation-in-part of application No. 09/570,364, filed on May 12, 2000, now abandoned.
(63) Continuation-in-part of application No. 09/569,525, filed on May 12, 2000, now abandoned.
(63) Continuation-in-part of application No. 09/547,276, filed on Apr. 11, 2000, now abandoned.
(60) Provisional application No. 60/196,460, filed on Apr. 11, 2000.
(60) Provisional application No. 60/196,528, filed on Apr. 11, 2000.
(60) Provisional application No. 60/196,527, filed on Apr. 11, 2000.
(60) Provisional application No. 60/196,267, filed on Apr. 11, 2000.
(60) Provisional application No. 60/182,296, filed on Feb. 14, 2000.
(60) Provisional application No. 60/176,195, filed on Jan. 14, 2000.
(60) Provisional application No. 60/170,453, filed on Dec. 13, 1999.
(60) Provisional application No. 60/162,498, filed on Oct. 29, 1999.
(60) Provisional application No. 60/160,860, filed on Oct. 21, 1999.
(60) Provisional application No. 60/134,118, filed on May 14, 1999.
(60) Provisional application No. 60/134,117, filed on May 14, 1999.
(60) Provisional application No. 60/134,114, filed on May 14, 1999.

(51) Int. Cl. C12Q 1/68; A61K 39/395

(52) U.S. Cl. 514/12

(57) ABSTRACT

The invention provides reagents and methods for inhibiting or enhancing interactions between proteins in hematopoietic cells and other cells involved in the mediation of an immune response. Reagents and methods provided are useful for treatment of a variety of diseases and conditions mediated by immune system cells.

### REFERENCE TO RELATED APPLICATIONS

This application claims priority to U.S. patent application No. ______ (filed Nov. 10, 2000, attorney docket no. 020054-001120US), U.S. patent application No. ______ (filed Oct. 13, 2000, attorney docket no. 020054-0011OUS), U.S. patent applications Ser. Nos. 09/570118, 09/570364, 09/569525 (all filed May 12, 2000), 60/196,460, 60/196,528, and 60/196,527, 60/196267, 09/547,276 (all filed Apr. 11, 2000), Ser. No. 60/182,296 (filed Feb. 14, 2000); Ser. No. 60/176,195 (filed Jan. 14, 2000); Ser. No. 60/170,453 (filed Dec. 13, 1999); Ser. No. 60/162,498 (filed Oct. 29, 1999); Ser. No. 60/160,860 (filed Oct. 21, 1999); and Ser. Nos. 60/134,118; 60/134,117; and 60/134,114 (all filed May 14, 1999); the disclosures of each of which are incorporated herein in their entirety.

### FIELD OF THE INVENTION

The present invention relates to peptides and peptide analogues, and methods for using such compositions to regulate activities of cells of the hematopoietic system. In one aspect, the invention provides methods of modulating metabolism (e.g., activation) of hematopoietic cells (e.g., T cells and B cells) by antagonizing an interaction between a PDZ domain containing protein and a protein that binds a PDZ domain. In one aspect, it relates to fusion peptides containing an amino acid sequence corresponding to the carboxyl terminus of a surface receptor expressed by a hematopoietic cell and a transmembrane transporter sequence; such fusion peptides are useful in regulating hematopoietic cells by inhibiting cell activation.

### BACKGROUND OF THE INVENTION

PDZ domains of proteins are named after three prototypical proteins: PSD95, Drosophila large disc protein and Zonula Occludin I protein (Gomperts et al., 1996, Cell 84:659-662). PDZ domain-containing proteins are involved in synapse formation by organizing transmembrane neurotransmitter receptors through intracellular interactions. PDZ domains contain the signature sequence GLGF (SEQ ID NO: 402). In the nervous system, typical PDZ domain-containing proteins contain three PDZ domains, one SH3 domain and one guanylate kinase domain. Examples of intracellular PDZ domain-containing proteins include LIN-2, LIN-7 and LIN-10 at the pre-synapse, and PSD95 at the post-synapse.

PDZ domains have been shown to bind the carboxyl termini of transmembrane proteins in neuronal cells. Songyang et al. reported that proteins capable of binding PDZ domains contain a carboxyl terminal motif sequence of E-S/T-X-V/I (Songyang et al., 1997, Science 275:73). X-ray crystallography studies have revealed the contact points between the motif sequence and PDZ domains (Doyle et al., 1996, Cell 88:1067-1076). While the interaction between PDZ domains and ion channels in neurons have been studied extensively, such interactions have had limited studies in other biological systems, especially the hematopoietic system.

The hematopoietic system is composed of different cell types that perform distinct functions. Many of its diverse functions require coordinated movement of cell surface receptors including ion channels, adhesion surface molecules to coordinate cell-cell interaction, and cytokine receptors. Despite their diverse functional activities, all hematopoietic cells are believed to develop from a multipotent bone marrow hematopoietic stem cell. Such stem cell has been shown to express a surface marker termed CD34. During differentiation, the stem cell gives rise to progenitor cells in each of several specific hematopoietic cell lineages. The progenitor cells then undergo a series of morphological and functional changes to produce mature functionally committed hematopoietic cells.

Among the functions performed by hematopoietic cells, certain cell types are involved exclusively in immunity. For example, lymphocytes, which include T cells, B cells and natural killer (NK) cells, are effectors in immune responses. Monocytes and granulocytes (i.e., neutrophils, basophils and eosinophils) play a role in non-specific forms of defense.

Lymphocytes, monocytes and granulocytes are collectively referred to as white blood cells or leukocytes. On the other hand, other hematopoietic cells perform functions that are unrelated to the immune system. For example, erythrocytes are involved in gas transport, and cells of the thrombocytic series are involved in blood clotting.

T cells and B cells recognize antigens and generate an immune response. T cells recognize antigens by heterodimeric surface receptors termed the T cell receptor (TCR). The TCR is associated with a series of polypeptides collectively referred to as CD3 complex. B cells recognize antigens by surface immunoglobulins (Ig), which are also secretory molecules. In addition, a large number of co-stimulatory surface receptors have been identified in T cells and B cells, which augment cellular activation during antigen-induced activation.

In addition to the T cell antigen receptor/CD3 complex (TCR/CD3), other molecules expressed by T cells which mediate an activation signal, include but are not limited to, CD2, CD4, CD5, CD6, CD8, CD18, CD27, CD28, CD43, CD45, CD152 (CTLA-4), CD154, MHC class I, MHC class II, CDw137 (4-1BB), CDw150, and the like (Barclay et al., The Leucocyte Antigen Facts Book, 1997, Second edition, Academic Press; Leucocyte Typing, 1984, Bernard et al. (eds.), Springer-Verlag; Leukocyte Typing II, 1986, Reinherz et al. (eds.), Springer-Verlag; Leukocyte Typing II, 1987, McMichael (ed.), Oxford University Press; Leukocyte Typing IV, 1989, Knapp et al. (eds.), Oxford University Press; CD Antigens, 1996, VI Internat. Workshop and Conference on Human Leukocyte Differentiation Antigens. http://www.ncbi.nlm. nih.gov/prow); all incorporated by reference herein. Cell surface antigens that work together with TCR/CD3 are often referred to as co-receptors in the art.

Specific antibodies have been generated against all of the aforementioned T cell surface antigens. Other molecules that bind to the aforementioned T cell surface receptors include antigen-binding antibody derivatives such as variable domains, peptides, superantigens, and their natural ligands such as CD58 (LFA-3) for CD2, HIV gp120 for CD4, CD27L for CD27, CD80 or CD86 for CD28 or CD152, ICAM1, ICAM2 and ICAM3 for CD11a/CD18, 4-1BBL for CDw137.

Activation molecules expressed by B cells, include but are not limited to, surface Ig, CD18, CD19, CD20, CD21, CD22, CD23, CD40, CD45, CD80, CD86 and ICAM1.

Similarly, natural ligands of these molecules and antibodies directed to them as well as antibody derivatives may be used to deliver an activation signal to B cells.

However, prior to the present invention, it was not known that signal transduction following stimulation of any leukocyte receptor was mediated by receptor interactions with PDZ domain-containing proteins. Therefore, it was not even contemplated in the art that an interference of leukocyte surface receptor/PDZ domain interactions could regulate leukocyte activation.

### 3. SUMMARY OF THE INVENTION

In one aspect, the invention provides a method of modulating a biological function of a cell, e.g., an endothelial cell or hematopoietic cell (such as a leukocyte, e.g., T cell or B cell), by introducing into the cell an antagonist that inhibits binding of a PDZ protein and a PL protein in the cell, or a agonist that enhances binding of a PDZ protein and a PL protein in the cell. In various embodiments the PL protein is an adhesion protein, an adaptor protein, or an intracellular protein. In embodiments it is CD6, CD49E, CD49F, CD138, Clasp-1, Clasp4, VCAM1, Clasp-2, CD95, DNAM-1, CD83, CD44, CD4, CD97, CD3n, DOCK2, CD34, FceRIb, or FasLigand.

In an embodiment the PL protein is characterized by a carboxy-terminal amino acid motif that is X—S—X-A, X-A-D/E-V, X-V/I/L-X*-V, or X—S/T-X-F (where X is any amino acid and X* is any non-aromatic amino acid). In embodiments, the PL protein is expressed by T lymphocytes or B lymphocytes. In some embodiments of this method, the PDZ protein is CASK, MPP1, DLG1, PSD95, NeDLG, SYN1a, TAX43, LDP, LIM, LIMK, AF6, PTN4, prIL16, 41.8, RGS12, DVL1, TAX 40, TIAM1, MINT1, K303, TAX2, or KIAA561.

In some embodiments, the cell is a leukocyte and the biological function is cell activation, cell proliferation, maintenance of cell structure, cell metabolic activity, or cytokine production. In some embodiments, the method further includes detecting a change in leukocyte activation.

In preferred embodiments, the antagonist is an agent that inhibits the binding of a PL peptide to a PDZ domain polypeptide in an “A” assay, in a “G” assay, or in both an A assay and a G assay. The antagonist can be a polypeptide, such as a polypeptide having at the carboxyterminus at least two residues that are the same as the carboxy-terminal two residues of a PL protein, such as a PL protein is expressed in a hematopoietic or endothelial cell, and/or that is an adhesion protein, an adaptor protein, or an intracellular protein. In an embodiment, at least the carboxy-terminal four residues of the polypeptide are the same as the carboxy-terminal four residues of the PL protein. In an embodiment, the PL protein has a carboxy-terminal amino acid motif selected from X—S—X-A, X-A-D/E-V, X-V/I/L-X*-V, or X—S/T-X-F, where X is any amino acid and X* is any non-aromatic amino acid. In embodiment, the PL protein is CD6, CD49E, CD49F, CD138, Clasp-1, Clasp-4, VCAM1, Clasp-2, CD95, DNAM-1, CD83, CD44, CD97, CD3n, DOCK2, CD34, FceRlb, or FasLigand.

In a related aspect, the antagonist is a peptide mimetic of a PL inhibitor sequence peptide. In another related aspect the antagonist is a fusion polypeptide having a PL sequence and transmembrane transporter amino acid sequence (such as HIV tat, Drosophila antenapedia, herpes simplex virus VP22 or anti-DNA CDR 2 and 3).

In another aspect, the invention provides a method of determining whether a test compound is an inhibitor of binding between a PDZ protein and a PL protein by contacting a PDZ domain polypeptide having a sequence from the PDZ protein, and a PL peptide under conditions in which they form a complex, in the presence and in the absence of a test compound, and detecting the formation of the complex in the presence and absence of the test compound, where less complex formation in the presence of the test compound than in the absence of the compound indicates that the test compound is an inhibitor of a PDZ protein -PL protein binding. In embodiments the PL peptide has a sequence that includes the a C-terminal sequence of a PL protein, such as CD6, CD49E, CD49F, CD138, Clasp-1, Clasp-4, VCAM1, Clasp-2, CD95, DNAM-l, CD83, CD44, CD97, CD3n, DOCK2, CD34, FceRlb, or FasLigand. In some embodiments, the PDZ domain polypeptide is a fusion polypeptide.

In a related aspect, the invention provides a method of determining whether a test compound is an agonist of binding between a PDZ protein and a PL protein by contacting a PDZ domain polypeptide, and a PL peptide under conditions in which they form a complex, in the presence and in the absence of a test compound, and detecting the formation of the complex in the presence and absence of the test compound, where more complex formation in the presence of the test compound than in the absence of the compound indicates that the test compound is an agonist of a PDZ protein-PL protein binding.

The invention further provides an inhibitor of binding of a PDZ protein and a PL protein. In an embodiment, the inhibitor is characterized in that it reduces binding of a peptide selected from the group consisting of a PL peptide selected from the group consisting of CD6, CD49E, CD49F, CD138, Clasp-1, Clasp4, VCAM1, Clasp-2, CD95, DNAM-1, CD83, CD44, CD97, CD3n, DOCK2, CD34, FceRIb, and FasLigand and a PDZ domain polypeptide. In various embodiments, the inhibitor is a peptide comprising a sequence that is from 3 to about 20 residues of a C-terminal sequence of a PL protein selected from CD6, CD49E, CD49F, CD138, Clasp-1, Clasp4, VCAM1, Clasp-2, CD95, DNAM-1, CD83, CD44, CD97, CD3n, DOCK2, CD34, FceRIb, and FasLigand; a peptide having a motif X—S—X-A, X-A-D/E-V, X-V/I/L-X*-V, or X—S/T-X-F, (where X is any amino acid and X* is any non-aromatic amino acid); a peptide mimetic; or a small organic molecule. The invention also provides a pharmaceutical composition containing the inhibitor.

The invention also provides a method for treating a disease characterized by leukocyte activation by administering a therapeutically effective amount of an inhibitor of a PL-PDZ interaction. In embodiments, the disease is characterized by an inflammatory or humoral immune response or is an autoimmune disease. The invention further provides a method of reducing inflammation in a subject by administering an agent that inhibits binding of a PDZ protein and a PL protein, where the PL protein is an adhesion protein, an adaptor protein, or an intracellular protein.

The invention also provides use of an inhibitor of the binding of a PDZ protein and a PL protein to inhibit leukocyte activation or to treat a disease mediated by hematopoietic cells, such as a disease is characterized by an inflammatory or humoral immune response. The invention also provides use of an inhibitor of the binding of a PDZ protein and a PL protein in the preparation of a medicament for treatment of a disease mediated by hematopoietic cells.

The invention also provides a method of modulating a biological function of a hematopoietic cell, comprising introducing into the cell an antagonist that inhibits binding of a PDZ protein and a PL protein in the cell as deduced from Table 2, for example, where the PL protein is DNAM-l and the PDZ protein is MPP1, MPP2, DLG1, NeDLG, PSD95, LIM, AF6, 41.8 or RGS12, the PL protein is LPAP and the PDZ protein is DLG1 or MINT1, or the PL protein is DNAM-1 and the PDZ protein is PSD95 or MPP2.

The present invention also relates to peptides and peptide analogues that bind PDZ domains in hematopoietic cells. In particular, it relates to fusion peptides and peptide analogues containing a hematopoietic cell surface receptor carboxyl terminal sequence and a transmembrane transporter sequence which facilitates entry of the peptides into a target cell. The invention also relates to methods of using such compositions in inhibiting leukocyte activation as measured by cytokine production, cell proliferation, apoptosis and/or cytotoxicity.

It is an object of the invention to administer a therapeutically effective amount of the aforementioned fusion peptides, peptide analogues, small molecules and other mediators of PDZ-PL interactions as pharmaceutical compositions, e.g., to a subject to inhibit undesirable cell-mediated (e.g., leukocyte-mediated) events.

It is also an object of the invention to administer a therapeutically effective amount of the aforementioned fusion peptides, peptide analogues, small molecules and other mediators of PDZ-PL interactions as pharmaceutical compositions to a subject to treat an autoimmune disorder or to prevent transplantation rejection of a solid organ transplant.

In one aspect, the invention provides a method of determining the apparent affinity (Kd) of binding between a PDZ domain and a ligand by (a) immobilizing a polypeptide comprising the PDZ domain and at least one non-PDZ domain on a surface; (b) contacting the immobilized polypeptide with a plurality of different concentrations of the ligand; (c) determining the amount of binding of the ligand to the immobilized polypeptide at each of the concentrations of ligand; (d) calculating the apparent affinity of the binding from the binding determined in (c). In an embodiment, the polypeptide is immobilized by binding the polypeptide to an immobilized immunoglobulin that binds the non-PDZ domain. In an embodiment, the polypeptide comprising the PDZ domain is a fusion protein, for example a GST-PDZ domain fusion protein.

In one aspect, the invention provides a method of determining the Ki of an inhibitor or suspected inhibitor of binding between a PDZ domain and a ligand, by (a) immobilizing a polypeptide comprising the PDZ domain and a non-PDZ domain on a surface; (b) contacting the immobilized polypeptide with a plurality of different mixtures of the ligand and inhibitor, wherein the different mixtures comprise a fixed amount of ligand, at least a portion of which is detectably labeled, and different concentrations of the inhibitor; (c) determining the amount of ligand bound at the different concentrations of inhibitor; (d) calculating the Ki of the inhibitor from the binding determined in (c). In an embodiment, the polypeptide is immobilized by binding the polypeptide to an immobilized immunoglobulin that binds the non-PDZ domain. In an embodiment, the fixed amount of ligand is between about 0.01 Kd and about 2 Kd.

In another aspect, the invention provides a method of identifying an agent that enhances the binding of a PDZ domain to a ligand, by immobilizing a polypeptide comprising the PDZ domain and a non-PDZ domain on a surface; (b) contacting the immobilized polypeptide with the ligand in the presence of a test agent and determining the amount of ligand bound; and, (c) comparing the amount of ligand bound in the presence of the test agent with the amount of ligand bound by the polypeptide in the absence of the test agent, wherein at least two-fold greater binding in the presence of the test agent compared to the absence of the test agent indicates that the test agent is an agent that enhances the binding of the PDZ domain to the ligand. In an embodiment, the polypeptide is immobilized by binding the polypeptide to an immobilized immunoglobulin that binds the non-PDZ domain.

In another aspect, the invention provides a method of determining the potency (Ihaucer) of an enhancer of binding between a PDZ domain and a ligand, by (a) immobilizing a polypeptide comprising the PDZ domain and a non-PDZ domain on a surface; (b) contacting the immobilized polypeptide with a plurality of different mixtures of the ligand and enhancer, wherein the different mixtures comprise a fixed amount of ligand, at least a portion of which is detectably labeled, and different concentrations of the enhancer; (c) determining the amount of ligand bound at the different concentrations of enhancer; (d) calculating the potency (K_{enhancer}) of the enhancer from the binding determined in (c). In an embodiment, the polypeptide is immobilized by binding the polypeptide to an immobilized immunoglobulin that binds the non-PDZ domain. In an embodiment, the fixed amount of ligand is between about 0.01 Kd and about 0.5 Kd.

In another aspect, the invention provides a method of identifying a high specificity interaction between a particular PDZ domain and a ligand known or suspected of binding at least one PDZ domain, by (a) providing a plurality of different immobilized polypeptides, each of said polypeptides comprising a PDZ domain and a non-PDZ domain; (b) determining the affinity of the ligand for each of said polypeptides; (c) comparing the affinity of binding of the ligand to each of said polypeptides. An interaction between the ligand and a particular PDZ domain is deemed to have high specificity when the ligand binds an immobilized polypeptide comprising the particular PDZ domain with at least 2-fold higher affinity than to immobilized polypeptides not comprising the particular PDZ domain in (a). In an embodiment, the polypeptide is immobilized by binding the polypeptide to an immobilized immunoglobulin that binds the non-PDZ domain.

In another aspect, the invention provides a method for determining the PDZ-PL inhibition profile of a compound by (a) providing (i) a plurality of different immobilized polypeptides, each of said polypeptides comprising a PDZ domain and a non-PDZ domain; (ii) a plurality of corresponding ligands, wherein each ligand binds at least one PDZ domain in (i); (b) contacting each of said immobilized polypeptides in (i) with a corresponding ligand in (ii) in the presence and absence of a test compound; (c) determining for each polypeptide-ligand pair in (b) whether the test compound inhibits binding between the immobilized polypeptide and the corresponding ligand thereby determining the PDZ-PL inhibition profile of the test compound.

In another aspect, the invention provides an array comprising a plurality of different immobilized polypeptides, each of said polypeptides comprising a PDZ domain and a non-PDZ domain. In an embodiment, the array is situated in a plastic multiwell plate. In an embodiment, the array has at least 12 different polypeptides comprising at least 12 different PDZ domains, for example, at least 12 different PDZ domains are from PDZs expressed in lymphocytes. In an embodiment, the PDZs are selected from those listed in Table 2 or 6.

In an aspect, the invention provides an assay device comprising a plurality of different immobilized PDZ-containing proteins organized in an array. In one embodiment, the device has at least 25 different PDZ-containing proteins.

In a further aspect, the invention provides a method for identifying an interaction between a PDZ domain and a PL by contacting a PL to a plurality of PDZ containing polypeptides and detecting binding of at least one PL to a PDZ. In an embodiment, the contacting occurs on an assay device comprising a plurality of different immobilized PDZ-containing proteins organized in an array. In one embodiment, the device has at least 25 different PDZ-containing proteins. In embodiments, an interaction between a PDZ and more than one PL, or between a PL and more than one PDZ, is detected.

In a related aspect, the invention provides method for identifying a modulator of an interaction between a PDZ and a PL by conducting any of the aforementioned assays in the presence and absence of a test compound and detecting a difference in at least one PDZ-PL interaction in the presence and absence of the test compound. In embodiments, the the modulator is an enhancer of the interaction. In other embodiments, the modulator is an inhibitor of the interaction.

In an embodiment, any of the aforementioned methods or devices (as further described herein) comprising a plurality of PDZ-domain containing polypeptides (e.g., a PDZ domain fusion protein) comprises at least one, usually at least 2, typically at least 5 and often at least 10 different PDZ-containing polypeptides comprising PDZ sequences from proteins selected from: MPP1 (p55), K303, K807, DLG1, PSD95, NeDLG, TAX IP43, LDP, LIM, K545, TIP1, PTN-4, CBP, AF6, PDZK1, DLG5, Syntenin, WWP3, K561.

In an aspect, the invention provides a method of modulating a biological function of an endothelial cell or hematopoietic cell (e.g., a leukocyte such as a T cell or a B cell), comprising introducing into the cell an agent that inhibits binding of a PDZ protein and a PL protein in the cell, wherein any of the following (I)-( ) apply:
(I) the PL is CD105, VCAM1, CD95, Spectrin β, KV1.3, DNAM1, Neuroligin 3, CD44, CD38, CD3r, LPAP, CD46, CDw128B, DOCK2, PAG, CD34, or BLR-1;
(II) the PDZ is MPP1, K303, K807, DLG1, PSD95, NeDLG, IP43, LDP, LIM, K545, TIP1, PTN-4, CBP, AF6, PDZK1, DLG5, Syntenin, WWP3, or K561;
(III) the PDZ protein is MPP1 and the PL protein has a carboxy-terminal amino acid motif X—SIT/Y/I-X-V; the PDZ protein is LIMK1 and the PL protein has a carboxy-terminal amino acid motif X—S/T/Y-X-V; the PDZ protein is K303 and the PL protein has a carboxy-terminal amino acid motif X—S—X-V; the PDZ protein is K807 and the PL protein has a carboxy-terminal amino acid motif X1-S/T-X2-V/I/L/F; the PDZ protein is DLG1, PSD95, or NeDLG and the PL protein has a carboxy-terminal amino acid motif X—S/T/Y/A/E-X-V/I/L; the PDZ protein is SNTa1 and the PL protein has a carboxy-terminal amino acid motif X—S/T/Y-D/Y-V/I/L; the PDZ protein is DVL1 and the PL protein has a carboxy-terminal amino acid motif X—S/T/Y-X-V; the PDZ protein is LDP and the PL protein has a carboxy-terminal amino acid motif X-A/S—X2-V/I; the PDZ protein is LIM and the PL protein has a carboxy-terminal amino acid motif X—S/T-X2-A/V; the PDZ protein is K561 and the PL protein has a carboxy-terminal amino acid motif X—S/T/Y-X-V/I/L/F; the PDZ protein is K545 and the PL protein has a carboxy-terminal amino acid motif X-A/S/T/Y-M-A/S/V; the PDZ protein is TAX-IP2 and the PL protein has a carboxy-terminal amino acid motif X—S-D/E-V; the PDZ protein is MPP2 and the PL protein has a carboxy-terminal amino acid motif X—S/T/Y-X-A/V/I; the PDZ protein is TIP-I and the PL protein has a carboxy-terminal amino acid motif X—S/T-X2-V/I/L; the PDZ protein is PTN4 and the PL protein has a carboxy-terminal amino acid motif X1-S/T-X-V/F; the PDZ protein is prIL16 and the PL protein has a carboxy-terminal amino acid motif D/E/K/R-V/I/L/F/Y-X-V; the PDZ protein is CBP and the PL protein has a carboxy-terminal amino acid motif X—S/T-F/Y-V; the PDZ protein is protein 41 and the PL protein has a carboxy-terminal amino acid motif X-A/S/T/Y/F-X-A/V/I/L; the PDZ protein is AF6 and the PL protein has a carboxy-terminal amino acid motif X-A/S/T/Y-F/Y-V/I/L; the PDZ protein is RGS 12 and the PL protein has a carboxy-terminal amino acid motif X1-S/[[Y-X-V/F; the PDZ protein is PDZK1 and the PL protein has a carboxy-terminal amino acid motif X-T-X-F; the PDZ protein is DLG5 and the PL protein has a carboxy-terminal amino acid motif X—S/T-X-V; the PDZ protein is Synt and the PL protein has a carboxy-terminal amino acid motif X1-V/I/L-X2-V; the PDZ protein is WWP3 and the PL protein has a carboxy-terminal amino acid motif X—S/T-X2-V; or the PDZ protein is TAX-IP40 and the PL protein has a carboxy-terminal amino acid motif X-Y-X-V; where X is any amino acid, X1 is any amino acid, X2 is any amino acid;
(IV) the agent is a peptide comprising a sequence of at least the carboxy-terminal two or three residues of the PL protein;
(V) the agent is a small molecule or peptide mimetic of the carboxy-terminus of the PL protein;

In an aspect the invention provides a method for determining whether a test compound is an inhibitor of binding between a PDZ protein and a PL protein by contacting a PDZ domain polypeptide having a sequence from the PDZ protein, and a PL peptide, wherein the PL peptide comprises a C-terminal sequence of a PL protein under conditions in which they form a complex, where the contacting is carried out in the presence and in the absence of a test compound, and detecting the formation of the complex in the presence and absence of the test compound. In embodiments, the PL protein is CD105, VCAM1, CD95, Spectrin β, KV1.3, DNAM1, Neuroligin 3, TAX, CD44, CD38, CD3r, LPAP, CD46, CDw128B, DOCK2, PAG, CD34, or BLR-1 and less complex formation in the presence of the test compound than in the absence of the compound indicates that the test compound is an inhibitor of a PDZ protein-PL protein binding. The invention also contemplates the inhibitor identified by this method. In embodiments, the inhibitor is (a) a peptide comprising a sequence that is from 3 to about 20 residues of a C-terminal sequence of CD105, VCAM1, CD95, Spectrin β, KV1.3, DNAM1, Neuroligin 3, TAX, CD44, CD38, CD3rl, LPAP, CD46, CDw128B, DOCK2, PAG, CD34, or BLR-1; (b) a peptide mimetic of such a peptide; or (c) a small organic molecule with a molecular weight less than 1 kD. The invention further contemplates a pharmaceutical composition containing the inhibitor, as well as a method for treating a disease characterized by leukocyte activation by administering a therapeutically effective amount of the inhibitor. In embodiments, the disease is characterized by an inflammatory or humoral immune response, e.g., an autoimmune disease.

In an aspect, the invention provides a method of modulating a biological function in a cell (e.g., a hematopoietic cell) by introducing into the cell an antagonist that inhibits binding of a PDZ protein and a PL protein in the cell, wherein, the PDZ protein is MPP1 (p55) and the PL is Spectrin β; the PDZ protein is K303 and the PL is Spectrin P; the PDZ protein is K807 and the PL VCAM1, Spectrin β, KV1.3, Neuroligin 3, CD38, CD3η, LPAP, CD46 (form 1), CDw128B, DOCK2, PAG, CD34, or BLR-1; the PDZ protein is DLG1 and the PL is Spectrin; the PDZ protein is PSD95 and the PL is Spectrin β, CD34, or CD38; the PDZ protein is NeDLG and the PL is Spectrin β or CD38; the PDZ protein is TAX IP43 and the PL is Spectrin β or CD38; the PDZ protein is LDP and the PL is CD38; the PDZ protein is LIM and the PL is CD105; the PDZ protein is K545 and the PL is CD1 05; the PDZ protein is TIP1 and the PL is CD95, KV1.3, CD3η, LPAP; the PDZ protein is PTN-4 and the PL is Spectrin β; the PDZ protein is CBP and the PL is Spectrin β; the PDZ protein is AF6 and the PL is Spectrin β; the PDZ protein is PDZK1 and the PL is BLR-1; the PDZ protein is DLG5 and the PL is Spectrin; the PDZ protein is Syntenin and the PL is CD44; the PDZ protein is WWP3 and the PL is VCAM1, Spectrin β, DNAM1, Neuroligin 3; the PDZ protein is K561 and the PL is BLR-1.

The invention also provides the use of an inhibitor of the binding of a PDZ protein and a PL protein described herein or identified according to a method of the invention to inhibit leukocyte activation, or for preparation of a medicament for treatment of a disease mediated by a PDZ-PL interaction, e.g., in hematopoietic cells or in viral infection.

The PDZ and PL proteins referred to herein are known in the art and are described herein, e.g., at Tables 3, 4 and 7. For example, CD105 is described at GenBank accession no. X72012; VCAM1 is described at GenBank accession no. M73255; CD95 is described at GenBank accession no. M67454; Spectrin β is described at GenBank accession no. NM000347; KV1.3 is described at GenBank accession no. AAC31761; DNAM1 is described at GenBank accession no. U56102; Neuroligin 3 is described at GenBank accession no. NM018977; TAX is described at GenBank accession no. AB038239; CD44 is described at GenBank accession no. M69215; CD38 is described at GenBank accession no. NM004334; CD3η is described at GenBank accession no. M33158; LPAP is described at GenBank accession no. X81422; CD46 is described at GenBank accession no. M58050; CDw128B is described at GenBank accession no. M73969; DOCK2 is described at GenBank accession no. BAA13200; PAG is described at GenBank accession no. NMO18440; CD34 is described at GenBank accession no. M81104; BLR-1 is described at GenBank accession no. S56162; CD4 is described at GenBank accession no. M12807; CD6 is described at GenBank accession no. X60992; CD49E (4) is described at GenBank accession no. X06256; CD49F is described at GenBank accession no. X53586; CD97 is described at GenBank accession no. X84700; CD98 is described at GenBank accession no. J02939; CD138 is described at GenBank accession no. J05392; CD148 is described at GenBank accession no. D37781; CD166 is described at GenBank accession no. L38608; CDw137 (4-4BB) is described at GenBank accession no. NM001561; FasL is described at GenBank accession no. U11821; FceRIb is described at GenBank accession no. D10583; Galectin3 is described at GenBank accession no. J02921; CD114 is described at GenBank accession no. NM000760; CDW125 (IL5R) is described at GenBank accession no. X62156; CDW128A (IL8RA) is described at GenBank accession no. M68932; Mannose Receptor is described at GenBank accession no. NM002438; NMDA is described at GenBank accession no. NP000824; Glycophorin C is described at GenBank accession no. AAA52574; Neurexin is described at GenBank accession no. AB011150; Syndecan-2 is described at GenBank accession no. A33880; CC CKR-1R is described at GenBank accession no. L09230; CC CKR-2 is described at GenBank accession no. U03882; CC CKR-3 is described at GenBank accession no. HSU28694; CC CKR-4 is described at GenBank accession no. X85740; Volt. Gated Ca2+ is described at GenBank accession no. Q00975; CD83 is described at GenBank accession no. Z11697; CD62E is described at GenBank accession no. M30640; CD5 is described at GenBank accession no. X04391; and CD148 is described at GenBank accession no. D37781; BLR-1/CXCR5 NM001716.

### 4. BRIEF DESCRIPTION OF THE DRAWINGS

FIGS. 1A-1D show the results of exemplary assays in which the binding of biotinylated peptides having a sequence of the carboxyl-terminus (“c-terminus”) of various leukocyte proteins to PDZ domains (i.e., GST-PDZ domain fusion proteins) was determined using the “G” assay described infra. The PDZ domains are: PSD95 (FIG. 1A); NeDLG (FIG. 1B); DLG1 (FIG 1C); and 41.8 (FIG. 1D). These and other PDZ domain fusion proteins are described infia (e.g., TABLE 2). In the figure, peptides 1-31 refer to the biotinylated PL peptides used in the assay, and are identified in the Key, infra. “Peptide IDs” are defined in

TABLE 3
Key:

| # | Test Protein | Peptide IDs |
|---|---|---|
| 1 | Clasp-2 | AA2L |
| 2 | FceRlb | AA25L |
| 3 | CDW128B | AA29.2 |
| 4 | KV1.3 | AA33L |
| 5 | Neurexin | AA38L |
| 6 | DOCK2 | AA40L |
| 7 | CC CKR-1R | AA41L |
| 8 | CC CKR-2 | AA42L |
| 9 | CC CKR-4 | AA44L |
| 10 | BLR-1 | AA45L |
| 11 | CD49E | AA11L |
| 12 | CD97 | AA14L |
| 13 | VCAM1 | AA17L |
| 14 | CD138 | AA18L |
| 15 | DNAM-1 | AA22L |
| 16 | CDW128A | AA29.1L |
| 17 | CC CKR-3 | AA43L |
| 18 | Clasp-1 | AA1L-R |
| 19 | CD46 (Form 1) | AA10L |
| 20 | CD95 | AA13L |
| 21 | CDW125 | AA28L |
| 22 | CD83 | AA47L |
| 23 | CD62E | AA48L |
| 24 | CD3n | AA4L |
| 25 | Clasp-4 | AA3L-V |
| 26 | CD44 | AA9L |
| 27 | CD166 | AA20L |
| 28 | CD62E | AA48L |
| 29 | CD5 | AA49L |
| 30 | CD148 | AA55L |
| 31 | DOCK2 | AA40L |

FIGS. 2A and 2B show the Apparent Affinity Determination for PDZ-Ligand Interactions. Varying concentrations of biotinylated CLASP-2 (FIG. 2A; TABLE 4) or Fas (FIG. 2B; TABLE 4) C-terminal peptides were reacted with immobilized (plate bound) GST polypeptide or GST-PDZ fusion proteins (GST-DLG1, GST-NeDLG, and GST-PSD95). The binding to GST alone (<0.2 OD units) was subtracted from the binding to the fusion proteins to obtain the signal at each peptide concentration. This signal was then normalized by dividing the signal at each peptide concentration by the maximum signal observed for each peptide-PDZ pair (i.e. the signal obtained at 30 uM Clasp 2 peptide or 100 uM Fas peptide; 0.4-1.0 OD units for Clasp 2 and 1.2-2.0 OD units for Fas). The normalized signals were then plotted and fit to a saturation binding curve, yielding an apparent affinity of 21 uM for DLG1-Clasp 2 interaction, 7.5 uM for NeDLG-Clasp 2 interaction, 45 uM for PSD95-Clasp 2 interaction, 54 uM for DLG1-Fas interaction, 54 uM for NeDLG-Fas interaction, and 85 uM for PSD95-Fas interaction. Data are means of duplicate data points, with standard errors between duplicate data points <20%.

FIGS. 3A-3F show inhibition of PDZ-PL peptide interactions. A fixed concentration of biotinylated C-terminal peptide having a sequence based on the C-terminal sequence of a cell surface receptor protein (Clasp 2, CD46, Fas, and KV1.3; see TABLE 4) was bound to immobilized GST polypeptide or the GST-fusion protein indicated at the top left of each frame, in the presence or absence of the competitor peptides indicated in the legend of each flame and the level of inhibition determined. FIG. 3A—DLG1; FIG. 3B—PSD95; FIG. 3C NeDLG; FIG. 3D-DLG1, FIG. 3E, PSD95; FIG. F-41.8. In FIGS. 3A-B the competitor peptides are present at 100 uM; in FIGS. 3C-F the competitor is present at the indicated concentration.

FIGS. 4A and 4B shows the results of introduction of a Tat-CD3 fusion peptide on T cell activation. Antigen-specific T cell activation was measured by cytokine production. Fusion peptides containing tat and a T cell surface molecule carboxyl terminus inhibited γ-interferon (IFN) production by a T cell line in response to myelin basic protein (MBP) stimulation. The level of inhibition was determined by first subtracting the binding of the labeled peptide to GST alone from the binding to the fusion protein and dividing by the signal in the absence of competitor peptide.

FIGS. 5A and 5B show TIP1-RFP overexpression enhances anti-CD95 induced apoptosis in Jurkat T cells. Jurkat E6 T cells were transfected with either DsRED (RFP), TIP1-RFP, or PAR6(N-P)-RFP. 24 hours post-transfection, cells were treated for 2 hours with anti-CD95 and then incubated with annexin V-FITC and analyzed using flow cytometry. FIG. 5A shows the results of FACS analysis. FIG. 5B shows a 30% increase in apoptosis of RFP-TIP1 positive compared to RFP negative apoptotic (annexin V positive) cells.

FIG. 6 Binding of a 20-mer peptide (20 uM) corresponding to the C-terminus of CD95 (Fas) to TIP-1 can be inhibited by an 8-mer peptide corresponding to the C-terminus of TAX. 50% inhibition can be achieved by 20-100 uM of inhibitor.

FIG. 7 Binding of a 20-mer peptide (20 uM) corresponding to the C-terminus of TAX to TIP-1 can be inhibited by an 8-mer peptide corresponding to the C-terminus of CD95 (Fas). 50% inhibition can be achieved by 500 uM of inhibitor.

FIG. 8 Binding of a 20-mer peptide (1 uM) corresponding to the C-terminus of BLR-1 (CXCR5) to KIAA0807 (PDZ domain)-GST fusion protein can be inhibited by an 8-mer peptide corresponding to the C-terminus of BLR-1 and a small molecule inhibitor (acetyl-LTTF). 50% inhibition can be achieved by greater than 100 uM of the 8-mer peptide and 1 uM of the small molecule inhibitor.

FIG. 9 Binding of a 20-mer peptide (10 uM) corresponding to the C-terminus of DOCK2 to KIAA0807 (PDZ domain)-GST fusion protein can be inhibited by an 8-mer peptide corresponding to the C-terminus of DOCK2 and a small molecule inhibitor (acetyl-STDL). 50% inhibition can be achieved by 250 uM of the 8-mer peptide and less than 250 uM of the small molecule inhibitor.

### Tables

Tables

| | |
|---|---|
| Table 1 | Amino Acid Classification |
| Table 2 | Protein-Ligand Pairs |
| Table 3 | PDZ Domains |
| Table 3A | Note on Table 3 |
| Table 4 | PL Peptides |
| Table 5 | Exemplary PL Motifs |
| Table 6 | PL Motifs |
| Table 7 | PDZ Domain-Containing Genes Expressed |
| | in T Cells and B Cells |
| Table 8 | Linker sequences introduced in cloning |
| Table 9 | Correlation of HPV E6 PL Motif and Oncogenic Activity |

### 5. DEFINITIONS

5.1 A “fusion protein” or “fusion polypeptide” as used herein refers to a composite protein, i.e., a single contiguous amino acid sequence, made up of two (or more) distinct, heterologous polypeptides which are not normally fused together in a single amino acid sequence. Thus, a fusion protein can include a single amino acid sequence that contains two entirely distinct amino acid sequences or two similar or identical polypeptide sequences, provided that these sequences are not normally found together in the same configuration in a single amino acid sequence found in nature. Fusion proteins can generally be prepared using either recombinant nucleic acid methods, i.e., as a result of transcription and translation of a recombinant gene fusion product, which fusion comprises a segment encoding a polypeptide of the invention and a segment encoding a heterologous protein, or by chemical synthesis methods well known in the art.

5.2 A“fusion protein construct” as used herein is a polynucleotide encoding a fusion protein.

5.3 As used herein, the term “PDZ domain” refers to protein sequence (i.e., modular protein domain) of approximately 90 amino acids, characterized by homology to the brain synaptic protein PSD-95, the Drosophila septate junction protein Discs-Large (DLG), and the epithelial tight junction protein ZO1 (ZO1). PDZ domains are also known as Discs-Large homology repeats (“DHRs”) and GLGF (SEQ ID NO: 402) repeats. PDZ domains generally appear to maintain a core consensus sequence (Doyle, D. A., 1996, Cell 85: 1067-76).

PDZ domains are found in diverse membrane-associated proteins including members of the MAGUK family of guanylate kinase homologs, several protein phosphatases and kinases, neuronal nitric oxide synthase, and several dystrophin-associated proteins, collectively known as syntrophins.

Exemplary PDZ domain-containing proteins and PDZ domain sequences are shown in TABLE 3. The term “PDZ domain” also encompasses variants (e.g., naturally occuring variants) of the sequences of TABLE 3 (e.g., polymorphic variants, variants with conservative substitutions, and the like). Typically, PDZ domains are substantially identical to those shown in TABLE 3, e.g., at least about 70%, at least about 80%, or at least about 90% amino acid residue identity when compared and aligned for maximum correspondence.

5.4 As used herein, the term “PDZ protein” refers to a naturally occurring protein containing a PDZ domain, e.g., a human protein. Exemplary PDZ proteins include CASK, MPP1, DLG1, PSD95, NeDLG, TAX33, SYN1a, TAX43, LDP, LIM, LIMK1, LIMK2, MPP2, NOS1, AF6, PTN-4, prIL16, 41.8 kD, KIAA0559, RGS12, KIAA0316, DVL1, TAX40, TIAM1, MINT1, KIAA0303, CBP, MINT3, TAX2, KIAA0561. Exemplary PDZ proteins are listed in TABLE 2 and TABLE 3.

5.5 As used herein, the term “PDZ-domain polypeptide” refers to a polypeptide containing a PDZ domain, such as a fusion protein including a PDZ domain sequence, a naturally occurring PDZ protein, or an isolated PDZ domain peptide.

5.6 As used herein, the term “PL protein” or “PDZ Ligand protein” refers to a naturally occurring protein that forms a molecular complex with a PDZ-domain, or to a protein whose carboxy-terminus, when expressed separately from the full length protein (e.g., as a peptide fragment of 4-25 residues, e.g., 16 residues), forms such a molecular complex. The molecular complex can be observed in vitro using the “A assay” or “G assay” described infra, or in vivo. Exemplary PL proteins listed in TABLE 2 are demonstrated to bind specific PDZ proteins. This definition is not intended to include anti-PDZ antibodies and the like.

5.7 As used herein, a “PL sequence” refers to the amino acid sequence of the C-terminus of a PL protein (e.g., the C-terminal 2, 3, 4, 5, 6, 7, 8, 9, 10, 12, 14, 16, 20 or 25 residues) (“C-terminal PL sequence”) or to an internal sequence known to bind a PDZ domain (“internal PL sequence).

5.8 As used herein, a “PL peptide” is a peptide of having a sequence from, or based on, the sequence of the C-terminus of a PL protein. Exemplary PL peptides (biotinylated) are listed in TABLE 4.

5.9 As used herein, a “PL fusion protein” is a fusion protein that has a PL sequence as one domain, typically as the C-terminal domain of the fusion protein. An exemplary PL fusion protein is a tat-PL sequence fusion.

5.10 As used herein, the term “PL inhibitor peptide sequence” refers to PL peptide amino acid sequence that (in the form of a peptide or PL fusion protein) inhibits the interaction between a PDZ domain polypeptide and a PL peptide (e.g., in an A assay or a G assay).

5.11 As used herein, a “PDZ-domain encoding sequence” means a segment of a polynucleotide encoding a PDZ domain. In various embodiments, the polynucleotide is DNA, RNA, single stranded or double stranded.

5.12 As used herein, the terms “antagonist” and “inhibitor,” when used in the context of modulating a binding interaction (such as the binding of a PDZ domain sequence to a PL sequence), are used interchangeably and refer to an agent that reduces the binding of the, e.g., PL sequence (e.g., PL peptide) and the, e.g., PDZ domain sequence (e.g., PDZ protein, PDZ domain peptide).

5.13 As used herein, the terms “agonist” and “enhancer,” when used in the context of modulating a binding interaction (such as the binding of a PDZ domain sequence to a PL sequence), are used interchangeably and refer to an agent that increases the binding of the, e.g., PL sequence (e.g., PL peptide) and the, e.g., PDZ domain sequence (e.g., PDZ protein, PDZ domain peptide).

5.14 As used herein, the terms “peptide mimetic, ” “peptidomimetic,” and “peptide analog” are used interchangeably and refer to a synthetic chemical compound which has substantially the same structural and/or functional characteristics of an PL inhibitory or PL binding peptide of the invention. The mimetic can be either entirely composed of synthetic, non-natural analogues of amino acids, or, is a chimeric molecule of partly natural peptide amino acids and partly non-natural analogs of amino acids. The mimetic can also incorporate any amount of natural amino acid conservative substitutions as long as such substitutions also do not substantially alter the mimetic's structure and/or inhibitory or binding activity. As with polypeptides of the invention which are conservative variants, routine experimentation will determine whether a mimetic is within the scope of the invention, i.e., that its structure and/or function is not substantially altered. Thus, a mimetic composition is within the scope of the invention if it is capable of binding to a PDZ domain and/or inhibiting a PL-PDZ interaction.

Polypeptide mimetic compositions can contain any combination of nonnatural structural components, which are typically from three structural groups: a) residue linkage groups other than the natural amide bond (“peptide bond”) linkages; b) non-natural residues in place of naturally occurring amino acid residues; or c) residues which induce secondary structural mimicry, i.e., to induce or stabilize a secondary structure, e.g., a beta turn, gamma turn, beta sheet, alpha helix conformation, and the like.

A polypeptide can be characterized as a mimetic when all or some of its residues are joined by chemical means other than natural peptide bonds. Individual peptidomimetic residues can be joined by peptide bonds, other chemical bonds or coupling means, such as, e.g., glutaraldehyde, N-hydroxysuccinimide esters, bifunctional maleimides, N,N=-dicyclohexylcarbodiimide (DCC) or N,N=-diisopropylcarbodiimide (DIC). Linking groups that can be an alternative to the traditional amide bond (“peptide bond”) linkages include, e.g., ketomethylene (e.g., —C(═O)—CH₂— for —C(═O)—NH—), aminomethylene (CH₂—NH), ethylene, olefin (CH═CH), ether (CH₂—O), thioether (CH₂—S), tetrazole (CN₄—), thiazole, retroamide, thioamide, or ester (see, e.g., Spatola (1983) in Chemistry and Biochemistry of Amino Acids, Peptides and Proteins, Vol. 7, pp 267-357, A Peptide Backbone Modifications, Marcell Dekker, NY).

A polypeptide can also be characterized as a mimetic by containing all or some non-natural residues in place of naturally occurring amino acid residues. Nonnatural residues are well described in the scientific and patent literature; a few exemplary nonnatural compositions useful as mimetics of natural amino acid residues and guidelines are described below.

Mimetics of aromatic amino acids can be generated by replacing by, e.g., D- or L-naphylalanine; D- or L-phenylglycine; D- or L-2 thieneylalanine; D- or L-1, -2, 3-, or 4-pyreneylalanine; D- or L-3 thieneylalanine; D- or L-(2-pyridinyl)-alanine; D- or L-(3-pyridinyl)-alanine; D- or L-(2-pyrazinyl)-alanine; D- or L-(4-isopropyl)-phenylglycine; D-(trifluoromethyl)-phenylglycine; D-(trifluoromethyl)-phenylalanine; D-p-fluorophenylalanine; D- or L-p-biphenylphenylalanine; K- or L-p-methoxybiphenylphenylalanine; D- or L-2-indole(alkyl)alanines; and, D- or L-alkylainines, where alkyl can be substituted or unsubstituted methyl, ethyl, propyl, hexyl, butyl, pentyl, isopropyl, iso-butyl, sec-isotyl, iso-pentyl, or a non-acidic amino acids. Aromatic rings of a nonnatural amino acid include, e.g., thiazolyl, thiophenyl, pyrazolyl, benzimidazolyl, naphthyl, furanyl, pyrrolyl, and pyridyl aromatic rings.

Mimetics of acidic amino acids can be generated by substitution by, e.g., non-carboxylate amino acids while maintaining a negative charge; (phosphono)alanine; sulfated threonine. Carboxyl side groups (e.g., aspartyl or glutamyl) can also be selectively modified by reaction with carbodiimides (R—N—C—N—R═) such as, e.g., 1-cyclohexyl-3(2-morpholinyl-(4-ethyl) carbodiimide or 1-ethyl-3(4-azonia-4,4-dimetholpentyl) carbodiimide. Aspartyl or glutamyl can also be converted to asparaginyl and glutaminyl residues by reaction with ammonium ions.

Mimetics of basic amino acids can be generated by substitution with, e.g., (in addition to lysine and arginine) the amino acids ornithine, citrulline, or (guanidino)-acetic acid, or (guanidino)alkyl-acetic acid, where alkyl is defined above. Nitrile derivative (e.g., containing the CN-moiety in place of COOH) can be substituted for asparagine or glutamine. Asparaginyl and glutaminyl residues can be deaminated to the corresponding aspartyl or glutamyl residues.

Arginine residue mimetics can be generated by reacting arginyl with, e.g., one or more conventional reagents, including, e.g., phenylglyoxal, 2,3-butanedione, 1,2-cyclohexanedione, or ninhydrin, preferably under alkaline conditions.

Tyrosine residue mimetics can be generated by reacting tyrosyl with, e.g., aromatic diazonium compounds or tetranitromethane. N-acetylimidizol and tetranitromethane can be used to form 0-acetyl tyrosyl species and 3-nitro derivatives, respectively.

Cysteine residue mimetics can be generated by reacting cysteinyl residues with, e.g., alpha-haloacetates such as 2-chloroacetic acid or chloroacetamide and corresponding amines; to give carboxymethyl or carboxyamidomethyl derivatives. Cysteine residue mimetics can also be generated by reacting cysteinyl residues with, e.g., bromo-trifluoroacetone, alpha-bromo-beta-(5-irnidozoyl) propionic acid; chloroacetyl phosphate, N-alkylnaleimides, 3-nitro-2-pyridyl disulfide; methyl 2-pyridyl disulfide; p-chloromercuribenzoate; 2-chloromercuri-4 nitrophenol; or, chloro-7-nitrobenzo-oxa-1,3-diazole.

Lysine mimetics can be generated (and amino terminal residues can be altered) by reacting lysinyl with, e.g., succinic or other carboxylic acid anhydrides. Lysine and other alpha-amino-containing residue mimetics can also be generated by reaction with imidoesters, such as methyl picolinimidate, pyridoxal phosphate, pyridoxal, chloroborohydride, trinitrobenzenesulfonic acid, O-methylisourea, 2,4, pentanedione, and transamidase-catalyzed reactions with glyoxylate.

Mimetics of methionine can be generated by reaction with, e.g., methionine sulfoxide. Mimetics of proline include, e.g., pipecolic acid, thiazolidine carboxylic acid, 3- or 4-hydroxyproline, dehydroproline, 3- or 4-methylproline, or 3,3,-dimethylproline. Histidine residue mimetics can be generated by reacting histidyl with, e.g., diethylprocarbonate or para-bromophenacyl bromide.

Other mimetics include, e.g., those generated by hydroxylation of proline and lysine; phosphorylation of the hydroxyl groups of seryl or threonyl residues; methylation of the alpha-amino groups of lysine, arginine and histidine; acetylation of the N-terminal amine; methylation of main chain amide residues or substitution with N-methyl amino acids; or amidation of C-terminal carboxyl groups.

A component of a natural polypeptide (e.g., a PL polypeptide or PDZ polypetide) can also be replaced by an amino acid (or peptidomimetic residue) of the opposite chirality. Thus, any amino acid naturally occurring in the L-configuration (which can also be referred to as the R or S, depending upon the structure of the chemical entity) can be replaced with the amino acid of the same chemical structural type or a peptidomimetic, but of the opposite chirality, generally referred to as the D-amino acid, but which can additionally be referred to as the R- or S-form.

The mimetics of the invention can also include compositions that contain a structural mimetic residue, particularly a residue that induces or mimics secondary structures, such as a beta turn, beta sheet, alpha helix structures, gamma turns, and the like. For example, substitution of natural amino acid residues with D-amino acids; N-alpha-methyl amino acids; C-alpha-methyl amino acids; or dehydroamino acids within a peptide can induce or stabilize beta turns, gamma turns, beta sheets or alpha helix conformations. Beta turn mimetic structures have been described, e.g., by Nagai (1985) Tet. Lett. 26:647-650; Feigl (1986) J. Amer. Chem. Soc. 108:181-182; Kahn (1988) J. Amer. Chem. Soc. 110:1638-1639; Kemp (1988) Tet. Lett. 29:5057-5060; Kahn (1988) J. Molec. Recognition 1:75-79. Beta sheet mimetic structures have been described, e.g., by Smith (1992) J. Amer. Chem. Soc. 114:10672-10674. For example, a type VI beta turn induced by a cis amide surrogate, 1,5-disubstituted tetrazol, is described by Beusen (1995) Biopolymers 36:181-200. Incorporation of achiral omega-amino acid residues to generate polymethylene units as a substitution for amide bonds is described by Baneijee (1996) Biopolymers 39:769-777. Secondary structures of polypeptides can be analyzed by, e.g., high-field 1H NMR or 2D NMR spectroscopy, see, e.g., Higgins (1997) J. Pept. Res. 50:421435. See also, Hruby (1997) Biopolymers 43:219-266, Balaji, et al., U.S. Pat. No. 5,612,895.

5.15 As used herein, “peptide variants” and “conservative amino acid substitutions” refer to peptides that differ from a reference peptide (e.g., a peptide having the sequence of the carboxy-terminus of a specified PL protein) by substitution of an amino acid residue having similar properties (based on size, polarity, hydrophobicity, and the like). Thus, insofar as the compounds that are encompassed within the scope of the invention are partially defined in terms of amino acid residues of designated classes, the amino acids may be generally categorized into three main classes: hydrophilic amino acids, hydrophobic amino acids and cysteine-like amino acids, depending primarily on the characteristics of the amino acid side chain. These main classes may be further divided into subclasses. Hydrophilic amino acids include amino acids having acidic, basic or polar side chains and hydrophobic amino acids include amino acids having aromatic or apolar side chains. Apolar amino acids may be further subdivided to include, among others, aliphatic amino acids. The definitions of the classes of amino acids as used herein are as follows:

“Hydrophobic Amino Acid” refers to an amino acid having a side chain that is uncharged at physiological pH and that is repelled by aqueous solution. Examples of genetically encoded hydrophobic amino acids include Ile, Leu and Val. Examples of non-genetically encoded hydrophobic amino acids include t-BuA.

“Aromatic Amino Acid” refers to a hydrophobic amino acid having a side chain containing at least one ring having a conjugated i-electron system (aromatic group). The aromatic group may be further substituted with groups such as alkyl, alkenyl, alkynyl, hydroxyl, sulfanyl, nitro and amino groups, as well as others. Examples of genetically encoded aromatic amino acids include Phe, Tyr and Trp. Commonly encountered non-genetically encoded aromatic amino acids include phenylglycine, 2-naphthylalanine, β-2-thienylalanine, 1,2,3,4-tetrahydroisoquinoline-3-carboxylic acid, 4-chloro-phenylalanine, 2-fluorophenyl-alanine, 3-fluorophenylalanine and 4-fluorophenylalanine.

“Apolar Amino Acid” refers to a hydrophobic amino acid having a side chain that is generally uncharged at physiological pH and that is not polar. Examples of genetically encoded apolar amino acids include Gly, Pro and Met. Examples of non-encoded apolar amino acids include Cha.

“Aliphatic Amino Acid” refers to an apolar amino acid having a saturated or unsaturated straight chain, branched or cyclic hydrocarbon side chain. Examples of genetically encoded aliphatic amino acids include Ala, Leu, Val and Ile. Examples of non-encoded aliphatic amino acids include Nle.

“Hydrophilic Amino Acid” refers to an amino acid having a side chain that is attracted by aqueous solution. Examples of genetically encoded hydrophilic amino acids include Ser and Lys. Examples of non-encoded hydrophilic amino acids include Cit and hCys.

“Acidic Amino Acid” refers to a hydrophilic amino acid having a side chain pK value of less than 7. Acidic amino acids typically have negatively charged side chains at physiological pH due to loss of a hydrogen ion. Examples of genetically encoded acidic amino acids include Asp and Glu.

“Basic Amino Acid” refers to a hydrophilic amino acid having a side chain pK value of greater than 7. Basic amino acids typically have positively charged side chains at physiological pH due to association with hydronium ion. Examples of genetically encoded basic amino acids include Arg, Lys and His. Examples of non-genetically encoded basic amino acids include the non-cyclic amino acids ornithine, 2,3-diaminopropionic acid, 2,4-diaminobutyric acid and homoarginine.

“Polar Amino Acid” refers to a hydrophilic amino acid having a side chain that is uncharged at physiological pH, but which has a bond in which the pair of electrons shared in common by two atoms is held more closely by one of the atoms. Examples of genetically encoded polar amino acids include Asx and Glx. Examples of non-genetically encoded polar amino acids include citrulline, N-acetyl lysine and methionine sulfoxide.

“Cysteine-Like Amino Acid” refers to an amino acid having a side chain capable of forming a covalent linkage with a side chain of another amino acid residue, such as a disulfide linkage. Typically, cysteine-like amino acids generally have a side chain containing at least one thiol (SH) group. Examples of genetically encoded cysteine-like amino acids include Cys. Examples of non-genetically encoded cysteine-like amino acids include homocysteine and penicillamine.

As will be appreciated by those having skill in the art, the above classification are not absolute—several amino acids exhibit more than one characteristic property, and can therefore be included in more than one category. For example, tyrosine has both an aromatic ring and a polar hydroxyl group. Thus, tyrosine has dual properties and can be included in both the aromatic and polar categories. Similarly, in addition to being able to form disulfide linkages, cysteine also has apolar character. Thus, while not strictly classified as a hydrophobic or apolar amino acid, in many instances cysteine can be used to confer hydrophobicity to a peptide.

Certain commonly encountered amino acids which are not genetically encoded of which the peptides and peptide analogues of the invention may be composed include, but are not limited to, β-alanine (b-Ala) and other omega-amino acids such as 3-aminopropionic acid (Dap), 2,3-diaminopropionic acid (Dpr), 4-aminobutyric acid and so forth; α-aminoisobutyric acid (Aib); ε-aminohexanoic acid (Aha); δ-aminovaleric acid (Ava); N-methylglycine or sarcosine (MeGly); ornithine (Orm); citrulline (Cit); t-butylalanine (t-BuA); t-butylglycine (t-BuG); N-methylisoleucine (MeIle); phenylglycine (Phg); cyclohexylalanine (Cha); norleucine 25 (Nle); 2-naphthylalanine (2-Nal); 4-chlorophenylalanine (Phe(4-Cl)); 2-fluorophenylalanine (Phe(2-F)); 3-fluorophenylalanine (Phe(3-F)); 4-fluorophenylalanine (Phe(4-F)); penicillamine (Pen); 1,2,3,4-tetrahydroisoquinoline-3-carboxylic acid (Tic); β-2-thienylalanine (Thi); methionine sulfoxide (MSO); homoarginine (hArg); N-acetyl lysine (AcLys); 2,3-diaminobutyric acid (Dab); 2,3-diaminobutyric acid (Obu); p-aminophenylalanine (PhepNH₂)); N-methyl valine (MeVal); homocysteine (hCys) and homoserine (hSer). These amino acids also fall conveniently into the categories defined above.

The classifications of the above-described genetically encoded and non-encoded amino acids are summarized in TABLE 1, below. It is to be understood that TABLE 1 is for illustrative purposes only and does not purport to be an exhaustive list of amino acid residues which may comprise the peptides and peptide analogues described herein. Other amino acid residues which are useful for making the peptides and peptide analogues described herein can be found, e.g., in Fasman, 1989, CRC Practical Handbook of Biochemistry and Molecular Biology, CRC Press, Inc., and the references cited therein. Amino acids not specifically mentioned herein can be conveniently classified into the above-described categories on the basis of known behavior and/or their characteristic chemical and/or physical properties as compared with amino acids specifically identified.

TABLE 1

| | Genetically | |
|---|---|---|
| Classification | Encoded | Genetically Non-Encoded |
| Hydrophobic | | |
| Aromatic | F, Y, W | Phg, Nal, Thi, Tic, Phe(4-Cl), Phe(2-F), |
| | | Phe(3-F), Phe(4-F), Pyridyl Ala, |
| | | Benzothienyl Ala |
| Apolar | M, G, P | |
| Aliphatic | A, V, L, I | t-BuA, t-BuG, MeIle, Nle, MeVal, Cha, |
| | | bAla, MeGly, Aib |
| Hydrophilic | | |
| Acidic | D, E | |
| Basic | H, K, R | Dpr, Orn, hArg, Phe(p-NH₂), |
| | | DBU, A₂BU |
| Polar | Q, N, S, T, Y | Cit, AcLys, MSO, hSer |
| Cysteine-Like | C | Pen, hCys, p-methyl Cys |

5.16 As used herein, a “detectable label” has the ordinary meaning in the art and refers to an atom (e.g., radionuclide), molecule (e.g., fluorescein), or complex, that is or can be used to detect (e.g., due to a physical or chemical property), indicate the presence of a molecule or to enable binding of another molecule to which it is covalently bound or otherwise associated. The term “label” also refers to covalently bound or otherwise associated molecules (e.g., a biomolecule such as an enzyme) that act on a substrate to produce a detectable atom, molecule or complex. Detectable labels suitable for use in the present invention include any composition detectable by spectroscopic, photochemical, biochemical, immunochemical, electrical, optical or chemical means. Labels useful in the present invention include biotin for staining with labeled streptavidin conjugate, magnetic beads (e.g., Dynabeads™), fluorescent dyes (e.g., fluorescein, Texas red, rhodamine, green fluorescent protein, enhanced green fluorescent protein, and the like), radiolabels (e.g., ³H, ¹²⁵I, ³⁵S, ¹⁴C, or ³²P), enzymes ( e.g., hydrolases, particularly phosphatases such as alkaline phosphatase, esterases and glycosidases, or oxidoreductases, particularly peroxidases such as horse radish peroxidase, and others commonly used in ELISAs), substrates, cofactors, inhibitors, chemiluminescent groups, chromogenic agents, and colorimetric labels such as colloidal gold or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241. Means of detecting such labels are well known to those of skill in the art. Thus, for example, radiolabels and chemiluminescent labels may be detected using photographic film or scintillation counters, fluorescent markers may be detected using a photodetector to detect emitted light (e.g., as in fluorescence-activated cell sorting). Enzymatic labels are typically detected by providing the enzyme with a substrate and detecting the reaction product produced by the action of the enzyme on the substrate, and colorimetric labels are detected by simply visualizing the colored label. Thus, a label is any composition detectable by spectroscopic, photochemical, biochemical, immunochemical, electrical, optical or chemical means. The label may be coupled directly or indirectly to the desired component of the assay according to methods well known in the art. Non-radioactive labels are often attached by indirect means. Generally, a ligand molecule (e.g., biotin) is covalently bound to the molecule. The ligand then binds to an anti-ligand (e.g., streptavidin) molecule which is either inherently detectable or covalently bound to a signal generating system, such as a detectable enzyme, a fluorescent compound, or a chemiluminescent compound. A number of ligands and anti-ligands can be used. Where a ligand has a natural anti-ligand, for example, biotin, thyroxine, and cortisol, it can be used in conjunction with the labeled, naturally occurring anti-ligands. Alternatively, any haptenic or antigenic compound can be used in combination with an antibody. The molecules can also be conjugated directly to signal generating compounds, e.g., by conjugation with an enzyme or fluorophore. Means of detecting labels are well known to those of skill in the art. Thus, for example, where the label is a radioactive label, means for detection include a scintillation counter, photographic film as in autoradiography, or storage phosphor imaging. Where the label is a fluorescent label, it may be detected by exciting the fluorochrome with the appropriate wavelength of light and detecting the resulting fluorescence. The fluorescence may be detected visually, by means of photographic film, by the use of electronic detectors such as charge coupled devices (CCDs) or photomultipliers and the like. Similarly, enzymatic labels may be detected by providing the appropriate substrates for the enzyme and detecting the resulting reaction product. Also, simple colorimetric labels may be detected by observing the color associated with the label. It will be appreciated that when pairs of fluorophores are used in an assay, it is often preferred that they have distinct emission patterns (wavelengths) so that they can be easily distinguished.

5.17 As used herein, the term “substantially identical” in the context of comparing amino acid sequences, means that the sequences have at least about 70%, at least about 80%, or at least about 90% amino acid residue identity when compared and aligned for maximum correspondence. An algorithm that is suitable for determining percent sequence identity and sequence similarity is the FASTA algorithm, which is described in Pearson, W. R. & Lipman, D. J., 1988, Proc. Natl. Acad. Sci U.S.A. 85: 2444. See also W. R. Pearson, 1996, Methods Enzymol. 266: 227-258. Preferred parameters used in a FASTA alignment of DNA sequences to calculate percent identity are optimized, BL50 Matrix 15: −5, k-tuple=2; joining penalty=40, optimization=28; gap penalty −12, gap length penalty=−2; and width=16.

5.18 As used herein, “hematopoietic cells” include leukocytes including lymphocytes (T cells, B cells and NK cells), monocytes, and granulocytes (i.e., neutrophils, basophils and eosinophils), macrophages, dendritic cells, megakaryocytes, reticulocytes, erythrocytes, and CD34⁺ stem cells.

5.19 As used herein, the terms “test compound” or “test agent” are used interchangably and refer to a candidate agent that may have enhancer/agonist, or inhibitor/antagonist activity, e.g., inhibiting or enhancing an interaction such as PDZ-PL binding. The candidate agents or test compounds may be any of a large variety of compounds, both naturally occurring and synthetic, organic and inorganic, and including polymers (e.g., oligopeptides, polypeptides, oligonucleotides, and polynucleotides), small molecules, antibodies (as broadly defined herein), sugars, fatty acids, nucleotides and nucleotide analogs, analogs of naturally occurring structures (e.g., peptide mimetics, nucleic acid analogs, and the like), and numerous other compounds. In certain embodiment, test agents are prepared from diversity libraries, such as random or combinatorial peptide or non-peptide libraries. Many libraries are known in the art that can be used, e.g., chemically synthesized libraries, recombinant (e.g., phage display libraries), and in vitro translation-based libraries. Examples of chemically synthesized libraries are described in Fodor et al., 1991, Science 251:767-773; Houghten et al., 1991, Nature 354:84-86; Lam et al., 1991, Nature 354:82-84; Medynski, 1994, Bio/Technology 12:709-710; Gallop et al., 1994, J. Medicinal Chemistry 37(9):1233-1251; Ohlmeyer et al., 1993, Proc. Natl. Acad Sci. USA 90:10922-10926; Erb et al., 1994, Proc. Natl. Acad. Sci. USA 91:11422-11426; Houghten et al., 1992, Biotechniques 13:412; Jayawickreme et al., 1994, Proc. Natl. Acad Sci. USA 91:1614-1618; Salmon et al., 1993, Proc. Natl. Acad. Sci. USA 90:11708-11712; PCT Publication No. WO 93/20242; and Brenner and Lemer, 1992, Proc. Natl. Acad. Sci. USA 89:5381-5383. Examples of phage display libraries are described in Scott and Smith, 1990, Science 249:386-390; Devlin et al., 1990, Science, 249:404-406; Christian, R. B., et al., 1992, J. Mol. Biol. 227:711-718); Lenstra, 1992, J. Immunol. Meth. 152:149-157; Kay et al., 1993, Gene 128:59-65; and PCT Publication No. WO 94/18318 dated Aug. 18, 1994. In vitro translation-based libraries include but are not limited to those described in PCT Publication No. WO 91/05058 dated Apr. 18, 1991; and Mattheakis et al., 1994, Proc. Natl. Acad Sci. USA 91:9022-9026. By way of examples of nonpeptide libraries, a benzodiazepine library (see e.g., Bunin et al., 1994, Proc. Natl. Acad. Sci. USA 91:47084712) can be adapted for use. Peptoid libraries (Simon et al., 1992, Proc. Natl. Acad. Sci. USA 89:9367-9371) can also be used. Another example of a library that can be used, in which the amide functionalities in peptides have been permethylated to generate a chemically transformed combinatorial library, is described by Ostresh et al. (1994, Proc. Natl. Acad. Sci. USA 91:11138-11142).

5.20 The term “specific binding” refers to binding between two molecules, for example, a ligand and a receptor, characterized by the ability of a molecule (ligand) to associate with another specific molecule (receptor) even in the presence of many other diverse molecules, i.e., to show preferential binding of one molecule for another in a heterogeneous mixture of molecules. Specific binding of a ligand to a receptor is also evidenced by reduced binding of a detectably labeled ligand to the receptor in the presence of excess unlabeled ligand (i.e., a binding competition assay).

5.21 As used herein, a “plurality” of PDZ proteins (or corresponding PDZ domains or PDZ fusion polypeptides) has its usual meaning. In some embodiments, the plurality is at least 5, and often at least 25, at least 40, or at least 60 different PDZ proteins. In some embodiments, the plurality is selected from the list of PDZ polypeptides listed in Table 2 or Table 7. In some embodiments, the plurality of different PDZ proteins are from (i.e., expressed in) a particular specified tissue or a particular class or type of cell. In some embodiments, the plurality of different PDZ proteins represents a substantial fraction (e.g., typically at least 50%, more often at least 80%) of all of the PDZ proteins known to be, or suspected of being, expressed in the tissue or cell(s), e.g., all of the PDZ proteins known to be present in lymphocytes or hematopoetic cells. In some embodiments, the plurality is at least 50%, usually at least 80%, at least 90% or all of the PDZ proteins disclosed herein as being expressed in hematopoietic cells (see Tables 2 and 6). In an embodiment, the plurality includes at least 1, often at least 2, sometimes at least 5 or at least 10 and sometimes all of the following PDZ proteins: BAI I associated protein, Connector enhancer, DLG5 (pdlg), DVL3, GTPase, Guanin-exchange factor 1, PDZ domain containing prot., KIAA147, KIAA0300, KIAA0380, KIAA0440, KIAA0545, KIAA0807, KIAA0858, KIAA0902, novel serine protease, PDZK1, PICK8, PTN-3, RPIP8, serine protease, 26s subunit p27, hSYNTENIN, TAX1-IP, TAX2-like protein, wwp3, X11 prot. beta, ZO1. When referring to PL ligands or corresponding PL proteins (e.g., corresponding to those listed in Table 2, Table 4, Table 5, or elsewhere herein) a “plurality” may refer to at least 5, at least 10, and often at least 25 PLs such as those specifically listed herein, or to the classes and percentages set forth supra for PDZ domains.

### 6. DETAILED DESCRIPTION OF THE INVENTION

The present inventors have discovered that interactions between PDZ proteins and PL proteins play an important and extensive role in the biological function of hematopoietic cells and other cells involved in the immune response. Although PDZ-PL interactions were known in the nervous system (i.e., in neurons), their universal importance in hematopoietic cell function, especially in function of T cells and B cells, and their fundamental role in modulation of the immune response has not been recognized. In particular, the present inventors have surprisingly discovered that cell adhesion molecules that mediate cell-cell interaction in the hematopoietic system are PDZ-binding proteins (PL proteins) and bind to PDZ proteins. The inventors have identified numerous interactions between PDZ proteins and PL proteins present in immune system cells, and the invention provides reagents and methods for affecting biological function in the immune system by inhibiting these interactions. As used herein, the term “biological function” in the context of a cell, refers to a detectable biological activity normally carried out by the cell, e.g., a phenotypic change such as proliferation, cell activation (e.g., T cell activation, B cell activation, T-B cell conjugate formation), cytokine release, degranulation, tyrosine phosphorylation, ion (e.g., calcium) flux, metabolic activity, apoptosis, changes in gene expression, maintenance of cell structure, cell migration, adherance to a substrate, signal transduction, cell-cell interactions, and others described herein or known in the art.

In one aspect, the present invention relates to peptides, peptide analogues or mimetics, pharmaceutical compositions, and methods of using such compositions to regulate the biological activities of hematopoietic cells, e.g. T cells and B cells, or other cells (e.g., endothelial cells) that necessary for immune function. The invention further relates to methods of using the compositions to modulate hematopoietic cell activation and immune function, as well as assays for such inhibitors.

TABLE 2 summarizes an extensive analysis of protein interactions in T cells and B cells. PDZ proteins, the vast majority of which were not previously known to be expressed in immune system cells, are listed in the top row of TABLE 2. The first column of the table lists PL proteins. Positions in the matrix denoted by the letter “A,” “G,” “G′,” or “G″” indicate that an interaction between the PDZ protein and the PL has been detected in novel binding assays (described in detail infra). A blank cell indicates that no interaction was detected using the assays of the invention. (S)—Indicates “sticky” peptide ligand having high background signal (i.e., in one or more versions of the G assay, the signal of ligand binding in the GST alone background wells was repeatedly above 0.5 OD units). An asterisk (*) denotes a PL-PDZ interaction previously reported in the scientific literature.

TABLE 2

| | | | PDZ-GST fusion Protein: | | | | | | |
|---|---|---|---|---|---|---|---|---|---|
| PDZ LIGAND | CODE | SEQ | CASK | MPP1 | LIMK1 | K303 | K807 | DLG1 | PSD95 |
| CD6 | AA6L | ISAA | | | | | | | |
| CD49E (alpha-4) | AA11L | TSDA | | | | | | | |
| CD49F (Aform, alpha6) | AA12L | TSDA | | | | | | | |
| CD105 (endoglin) | AA16L | SSMA | | | | | | | |
| CD166 (CD6L) | AA20L | KTEA | | | | | | | |
| CC CKR-2 | AA42L | KEGA | | | | | | | |
| CD138 (syndecan-1) | AA18L | EFYA | * | | | | | | |
| Syndecan-2 (S) | AA39L | EFYA | | | | | | | |
| CD148 (DEP-1) | AA19L | GYIA | | | | | | | |
| CD98 (2F4) (S) | AA15L | PYAA | | | | | | | |
| CLASP-1 | AA1L | SAEV | | | | | | G | A |
| CLASP-4 | AA3L-V | YAEV | | | | | | A | A |
| NMDA | AA34.2L | ESDV | | A | A | | | A/G | A/G |
| VCAM1 | AA17L | KSKV | | A | | A | G′/G″ | A | |
| CLASP-2 | AA2L | SSVV | | | | | | A/G | A/G |
| CD95 (Apo-1/Fas) | AA13L | QSLV | | | | | | A/G/G′ | A/G/G′ |
| Spectrin beta (S) | AA32L | VSFV | | G″ | | G″ | G′/G″ | G′/G″ | G′/G″ |
| KV1.3 | AA33L | FTDV | | | A | | G′/G″ | *A/G/G′/G″ | *A/G/G′/G″ |
| DNAM-1 | AA22L | KTRV | | A | | | | A | A/G/G′ |
| Neuroligin 3 | AA36L | TTRV | | | | | G″ | | |
| TAX | AA56L | ETEV | | | | | G′ | G′/G″ | G′/G″ |
| CD83 | AA47L | TELV | | | | | | A | A |
| CD44 (long form) | AA9L | KIGV | | G | | | | | |
| Neurexin (S) | AA38L | EYYV | G* | A* | A | | | A/G | A/G |
| CD97 (CD55L) | AA14L | ESGI | | | | | | A | |
| CD38 (S) | AA8L | TSEI | | | | | G′ | | G′ |
| Mannose receptor | AA31L | HSVI | | | | | | | |
| Glycophorin C | AA37L | EYFI | | * | | | | G | G |
| Galectin3 | AA26L | YTMI | | | | | | | |
| CDw128A (IL8RA) | AA29.1L | SSNL | | | | | | A | |
| CD3n | AA4L | SSQL | | | | | G″ | A | A |
| LPAP | AA30L | VTAL | | | | | G′/G″ | A | |
| CD46 (form 1) | AA10L | FTSL | | | | | G′/G″ | A/G | A/G |
| CDw128B (IL8RB) | AA29.2L | STTL | | | | | G′/G″ | A/G | A |
| DOCK2 | AA40L | STDL | | | | | G′/G″ | A | A/G |
| PAG | AA58L | ITRL | | | | | G′ | | |
| CD34 | AA7L | DTEL | | | | | G′/G″ | A | A |
| CD5 | AA49L | AQRL | | | | | | | |
| CC CKR-4 | AA44L | HDAL | | | | | | | |
| FceRlb | AA25L | PIDL | | | | | | | |
| CDw137 (4-1BB ILA) (S) | AA21L | GCEL | | | | | | | |
| FasLigand | AA23L-M | LYKL | | | | | | | |
| CD62E | AA48L | SYIL | | | | | | | |
| CC CKR-1R | AA41L | SAGF | | | | | | | |
| CDw125 (IL5R) | AA28L | DSVF | | | | | | | |
| BLR-1 | AA45L | LTTF | | | | | G′ | | |
| CC CKR-3 | AA43L | SIVF | | | | | | | |
| CD114 (G-CSFR) | AA27L | LGSF | | | | | | | |
| V-gated Ca2+ channel (S) | AA46L | DHWC | | | | | | | |
| PDZ-GST fusion Protein: | | | CASK | MPP1 | LIMK1 | K303 | K807 | DLG1 | PSD95 |

| | PDZ-GST fusion Protein: | | | | | | | | | |
|---|---|---|---|---|---|---|---|---|---|---|
| PDZ LIGAND | NeDLG | SNTa1 | TAX-IP43 | LDP | LIM | MINT1 | X11β | K440 | K545 | TAX-IP2 |
| CD6 | | | | | | | | | | |
| CD49E (alpha-4) | | | | | | | | | | |
| CD49F (Aform, alpha6) | | | | | | | | | | |
| CD105 (endoglin) | | | | | G′ | | | | G″ | |
| CD166 (CD6L) | | | | | | | | | | |
| CC CKR-2 | | | | | | | | | | |
| CD138 (syndecan) | | | | | | | | | | |
| Syndecan-2 (S) | | | | | | | | | | |
| CD148 (DEP-1) | | | | | | | | | | |
| CD98 (2F4) (S) | | | | | | | | | | |
| CLASP-1 | G | | | | | | | | | |
| CLASP-4 | A | | | A | | A | | | | |
| NMDA | A/G | G | A | | | A/G | | | | A |
| VCAM1 | A | | | A | | | | | | |
| CLASP-2 | A/G/G′ | | | | | | | | | |
| CD95 (Apo-1/Fas) | A/G/G′ | | | | | | | | | |
| Spectrin beta (S) | G′/G″ | | G′ | | | | | | | |
| KV1.3 | A/G/G′/G″ | | | | | G/G′ | | | | |
| DNAM-1 | A | | | | A | | | | | |
| Neuroligin 3 | | | | | | | | | | |
| TAX | G′/G″ | | G′/G″ | | | | | | | |
| CD83 | A | | | | | | | | | |
| CD44 (long form) | | | | | | G | | | | |
| Neurexin (S) | G | A | A | | | A/G | | | | |
| CD97 (CD55L) | | | | | | | | | | |
| CD38 (S) | G′ | | G′ | G′ | | | | | | |
| Mannose receptor | | | | | | | | | | |
| Glycophorin C | G | | | | | A | | | | |
| Galectin3 | | | | | | | | | | |
| CDw128A (IL8RA) | A | | | | | | | | | |
| CD3n | | | | | | A/G/G′ | | | | |
| LPAP | | | | | | G | | | | |
| CD46 (form 1) | G | | | | | | | | | |
| CDw128B (IL8RB) | A/G/G′ | | | | | | | | | |
| DOCK2 | G | G | | | | | | | | |
| PAG | | | | | | | | | | |
| CD34 | G | | | | | | | | | |
| CD5 | | | | | | | | | | |
| CC CKR-4 | | | | | | | | | | |
| FceRlb | | | | | | A/G′ | | | | |
| CDw137 (4-1BB ILA) (S) | | | | | | | | | | |
| FasLigand | | | | | | | | | | |
| CD62E | | | | | | | | | | |
| CC CKR-1R | | | | | | | | | | |
| CDw125 (IL5R) | | | | | | | | | | |
| BLR-1 | | | | | | G | | | | |
| CC CKR-3 | | | | | | | | | | |
| CD114 (G-CSFR) | | | | | | | | | | |
| V-gated Ca2+ channel (S) | | | | | | | | | | |
| PDZ-GST fusion Protein: | NeDLG | SYN1a | TAX-IP43 | LDP | LIM | MINT1 | X11β | K440 | K545 | TAX-IP2 |

| | PDZ-GST fusion Protein: | | | | | | | | | |
|---|---|---|---|---|---|---|---|---|---|---|
| PDZ LIGAND | TAX-IP2L | TAX-IP33 | MPP2 | MINT3 | TIP1 | PTN-4 | prIL16 | CBP | 41.8 | K559 |
| CD6 | | | | | | | | | A | |
| CD49E (alpha-4) | | | | | | | | | A/G | |
| CD49F (Aform, alpha6) | | | | | | | | | A/G | |
| CD105 (endoglin) | | | | | | | | | | |
| CD166 (CD6L) | | | | | | | | | | |
| CC CKR-2 | | | | | | | | | | |
| CD138 (syndecan) | | | | | | | | | A/G | |
| Syndecan-2 (S) | | | | | | | | | | |
| CD148 (DEP-1) | | | | | | | | | | |
| CD98 (2F4) (S) | | | G | | | | | | | |
| CLASP-1 | | | | | | | | | | |
| CLASP-4 | | | | | | | | | A | |
| NMDA | | | G | | | A/G | | | A/G | |
| VCAM1 | | | | | | | | | A | |
| CLASP-2 | | | | | | | | | A | |
| CD95 (Apo-1/Fas) | | | | | G′ | | | | A/G | |
| Spectrin beta (S) | | | | | | G′ | | G′ | | |
| KV1.3 | | | | | G′ | | | | A | |
| DNAM-1 | | | G | | | | | | A | |
| Neuroligin 3 | | | | | | | | | | |
| TAX | | | | | G′ | G′ | | | | |
| CD83 | | | | | | | | | | |
| CD44 (long form) | | | | | | | G | | | |
| Neurexin (S) | | | | | | | A | | A | |
| CD97 (CD55L) | | | | | | | | | A | |
| CD38 (S) | | | | | | | | | | |
| Mannose receptor | | | | | | | | | | |
| Glycophorin C | | | A | | | | | | A | |
| Galectin3 | | | | | | | | | | |
| CDw128A (IL8RA) | | | | | | | | | | |
| CD3n | | | | | G′/G″ | | | | A/G | |
| LPAP | | | | | G′ | | | | | |
| CD46 (form 1) | | | | | | | | | | |
| CDw128B (IL8RB) | | | | | | | | | A | |
| DOCK2 | | | | | | | | | | |
| PAG | | | | | | | | | | |
| CD34 | | | | | | | | | | |
| CD5 | | | | | | | | | | |
| CC CKR-4 | | | | | | | | | | |
| FceRlb | | | | | | | | | | |
| CDw137 (4-1BB ILA) (S) | | | | | | | | | | |
| FasLigand | | | | | | | | | | |
| CD62E | | | | | | | | | | |
| CC CKR-1R | | | | | | | | | | |
| CDw125 (IL5R) | | | | | | G | | | | |
| BLR-1 | | | | | | | | | | |
| CC CKR-3 | | | | | | | | | | |
| CD114 (G-CSFR) | | | | | | | | | | |
| V-gated Ca2+ channel (S) | | | | | | | | | | |
| PDZ-GST fusion Protein: | TAX-IP2L | TAX-IP33 | MPP2 | MINT3 | TIP1 | PTN-4 | prIL16 | CBP | 41.8 | K559 |

| | PDZ-GST fusion Protein: | | | | | | | | | |
|---|---|---|---|---|---|---|---|---|---|---|
| PDZ LIGAND | AF6 | PICK1 | RGS12 | PDZK1 | K316 | DLG5 | Synt | WWP3 | TAX-IP40 | K858 |
| CD6 | | | | | | | | | | |
| CD49E (alpha-4) | | | | | | | | | | |
| CD49F (Aform, alpha6) | | | | | | | | | | |
| CD105 (endoglin) | | | | | | | | | | |
| CD166 (CD6L) | | | | | | | | | | |
| CC CKR-2 | | | | | | | | | | |
| CD138 (syndecan) | | | | | | | * | | | |
| Syndecan-2 (S) | | | | | | | | | | |
| CD148 (DEP-1) | | | | | | | | | | |
| CD98 (2F4) (S) | | | | | | | | | | |
| CLASP-1 | | | | | | | | | | |
| CLASP-4 | A | | | | | | | | | |
| NMDA | | | A/G | | | | | | | |
| VCAM1 | | | | | | | | G′ | | |
| CLASP-2 | | | | | | | | | | |
| CD95 (Apo-1/Fas) | | | | | | | | | | |
| Spectrin beta (S) | G′/G″ | | | | | G′ | | G′/G″ | | |
| KV1.3 | | | A | | | | | | | |
| DNAM-1 | A | | A | | | | | G′/G″ | | |
| Neuroligin 3 | | | | | | | | G″ | | |
| TAX | | | | | | G′/G″ | | G′ | | |
| CD83 | | | | | | | | | | |
| CD44 (long form) | | | | | | | G′ | | | |
| Neurexin (S) | A | | A | | | | | | A | |
| CD97 (CD55L) | | | | | | | | | | |
| CD38 (S) | | | | | | | | | | |
| Mannose receptor | | | | | | | | | | |
| Glycophorin C | A | | | | | | | | | |
| Galectin3 | | | | | | | | | | |
| CDw128A (IL8RA) | | | | | | | | | | |
| CD3n | | | | | | | | | | |
| LPAP | | | | | | | | | | |
| CD46 (form 1) | | | | | | | | | | |
| CDw128B (IL8RB) | | | * | | | | | | | |
| DOCK2 | | | | | | | | | | |
| PAG | | | | | | | | | | |
| CD34 | | | | | | | | | | |
| CD5 | | | | | | | | | | |
| CC CKR-4 | | | | | | | | | | |
| FceRlb | | | | | | | | | | |
| CDw137 (4-1BB ILA) (S) | | | | | | | | | | |
| FasLigand | | | | | | | | | | |
| CD62E | | | | | | | | | | |
| CC CKR-1R | | | | | | | | | | |
| CDw125 (IL5R) | | | G | | | | | | | |
| BLR-1 | | | | G′/G″ | | | | | | |
| CC CKR-3 | | | | | | | | | | |
| CD114 (G-CSFR) | | | | | | | | | | |
| V-gated Ca2+ channel (S) | | | | | | | | | | |
| PDZ-GST fusion Protein: | AF6 | PICK1 | RGS12 | PDZK1 | K316 | DLG5 | Synt | WWP3 | TAX-IP40 | K858 |

| | PDZ-GST fusion Protein: | | | | | | | | | |
|---|---|---|---|---|---|---|---|---|---|---|
| PDZ LIGAND | TIAM1 | SP short | ConEn | DVL1 | NSP | GEF | K902 | K561 | NOS1 | LIMK2 |
| CD6 | | | | | | | | | | |
| CD49E (alpha-4) | | | | | | | | | | |
| CD49F (Aform, alpha6) | | | | | | | | | | |
| CD105 (endoglin) | | | | | | | | | | |
| CD166 (CD6L) | | | | | | | | | | |
| CC CKR-2 | | | | | | | | | | |
| CD138 (syndecan) | A | | | | | | | | | |
| Syndecan-2 (S) | | | | | | | | | | |
| CD148 (DEP-1) | | | | | | | | | | |
| CD98 (2F4) (S) | | | | | | | | | | |
| CLASP-1 | | | | | | | | | | |
| CLASP-4 | | | | | | | | | | |
| NMDA | | | | A | | | | G | | |
| VCAM1 | A | | | | | | | | | |
| CLASP-2 | | | | | | | | | | |
| CD95 (Apo-1/Fas) | | | | | | | | | | |
| Spectrin beta (S) | | | | | | | | | | |
| KV1.3 | | | | A | | | | | | |
| DNAM-1 | | | | | | | | | | |
| Neuroligin 3 | | | | | | | | | | |
| TAX | | | | | | | | | | |
| CD83 | | | | | | | | | | |
| CD44 (long form) | | | | | | | | | | |
| Neurexin (S) | A | | | A | | | | | | |
| CD97 (CD55L) | | | | | | | | | | |
| CD38 (S) | | | | | | | | | | |
| Mannose receptor | | | | | | | | | | |
| Glycophorin C | | | | | | | | | | |
| Galectin3 | | | | | | | | | | |
| CDw128A (IL8RA) | | | | | | | | | | |
| CD3n | | | | | | | | | | |
| LPAP | | | | | | | | | | |
| CD46 (form 1) | | | | | | | | | | |
| CDw128B (IL8RB) | | | | | | | | | | |
| DOCK2 | | | | | | | | G | | |
| PAG | | | | | | | | | | |
| CD34 | | | | | | | | | | |
| CD5 | | | | | | | | | | |
| CC CKR-4 | | | | | | | | | | |
| FceRlb | | | | | | | | | | |
| CDw137 (4-1BB ILA) (S) | | | | | | | | | | |
| FasLigand | | | | | | | | G | | |
| CD62E | | | | | | | | | | |
| CC CKR-1R | | | | | | | | | | |
| CDw125 (IL5R) | | | | | | | | | | |
| BLR-1 | | | | | | | | G′ | | |
| CC CKR-3 | | | | | | | | | | |
| CD114 (G-CSFR) | | | | | | | | | | |
| V-gated Ca2+ channel (S) | | | | | | | | | | |
| PDZ-GST fusion Protein: | TIAM1 | SP short | ConEn | DVL1 | NSP | GEF | K902 | K561 | NOS1 | LIMK2 |

As discussed in detail herein, the PDZ proteins listed in TABLE 2 are naturally occurring proteins containing a PDZ domain. The present invention is particularly directed to the detection and modulation of interactions between PDZ proteins and PL proteins in hematopoietic cells. Exemplary PL proteins are listed in TABLE 2. Notably, as discussed infra, many of these PL proteins have not previously been recognized as such in any cell system. A variety of PL protein classes are known, and the PL proteins described herein can be characterized as (1) “PL adhesion proteins” (2) “PL ion channel proteins” (3) “PL adaptor proteins” (4) “PL intracellular proteins” and (5) “PL cytokine receptor proteins.”

As used herein, an adhesion protein is a cell surface protein involved in cell-cell interaction by direct contact with cell surface molecules (e.g., transmembrane proteins or surface proteins) on a different cell. Thus, when a cell expressing a PL adhesion protein contacts an appropriate other cell, the PL adhesion protein localizes at the interface of the two cells and directly contacts a cell surface molecule on the second cell. A cell-cell interface is a region where the plasma membranes of two different cells are in close (generally <10 nm, often about 1 nm) apposition. Typically, direct molecular contact means interaction of molecules at distances where Van der Walls forces are significant, generally less than about 1 nm. Exemplary PL adhesion proteins include CD6; CD49E (alpha4); CD49F (a form, alpha6); CD138 (syndecan); CLASP-1; CLASP4; VCAM1; CLASP-2; DNAM-1; CD83; CD44 (long form); CD97; (CD55L); CD3η; DOCK2; CD34; and FceRlb. Thus, in one embodiment, the PL proteins of the invention are PL adhesion proteins. In an embodiment, the invention provides methods and reagents, as detailed herein, for inhibiting interactions between PL adhesion proteins and PDZ proteins to modulate an immune response. In an embodiment, the inhibition or modulation occurs in a hematopoietic cell. In a related embodiment, the inhibition or modulation occurs in an endothelial cell. In a related embodiment, the inhibition or modulation occurs in an endothelial cell. In a related embodiment, the inhibition or modulation occurs in an epithelial cells, keratinocytes, hepatocytes, cardiac myocytes.

As used herein, an ion channel protein means a transmembrane protein that itself catalyzes the passage of an ion from aqueous solution on one side of a lipid bilayer membrane to aqueous solution on the other side (e.g., by forming a small pore in the membrane). One exemplary PL ion channel proteins is Kv1.3. Thus, in one embodiment, the PL proteins of the invention are PL ion channel proteins. In an embodiment, the invention provides methods and reagents, as detailed herein, for inhibiting interactions between PL ion channel proteins and PDZ proteins to modulate an immune response. In an embodiment, the inhibition or modulation occurs in a hematopoeitic cell. In a related embodiment, the inhibition or modulation occurs in an endothelial cell.

As used herein, an intercellular (i.e., cytosolic) protein has the normal meaning in the art and refers to a protein that is not membrane bound, e.g., has no transmembrane domain. Thus, in one embodiment, the PL proteins of the invention are PL intercellular proteins. Exemplary PL intercellular proteins include Glycophorin C and LPAP. In an embodiment, the invention provides methods and reagents, as detailed herein, for inhibiting interactions between PL cytoplasmic proteins and PDZ proteins to modulate an immune response. In an embodiment, the inhibition or modulation occurs in a hematopoeitic cell. In a related embodiment, the inhibition or modulation occurs in an endothelial cell.

As used herein a cytokine receptor has the normal meaning in the art and refers to a membrane protein with an extracellular domain that specifically binds a cytokine. Exemplary PL cytokine receptor proteins include CDW125 (IL5R), CDW128A (IL8RA), and BRL-1. Thus, in one embodiment, the PL proteins of the invention are PL cytokine proteins. In an embodiment, the invention provides methods and reagents, as detailed herein, for inhibiting interactions between PL cytokine proteins and PDZ proteins to modulate an immune response. In an embodiment, the inhibition or modulation occurs in a hematopoeitic cell. In a related embodiment, the inhibition or modulation occurs in an endothelial cell.

As used herein, an adaptor protein means a molecule (e.g., protein) that contributes to the formation of a multimolecular complex by binding two or more other biomolecuics. The binding of the two or more other molecules by the adaptor molecule/protein generally involves direct molecular contact between the adaptor protein and each of the two or more other molecules. One exemplary PL adaptor protein is LPAP. Thus, in one embodiment, the PL proteins of the invention are PL adaptor proteins. In an embodiment, the invention provides methods and reagents, as detailed herein, for inhibiting interactions between PL adaptor proteins and PDZ proteins to modulate an immune response. In an embodiment, the inhibition or modulation occurs in a hematopoeitic cell. In a related embodiment, the inhibition or modulation occurs in an endothelial cell.

In various embodiments, the PL proteins of the invention are characterized by specific C-terminal (i.e., PL domain) amino acid sequences or amino acid motifs, as described elsewhere in this disclosure.

In various embodiments of the invention, the PL proteins of the invention bind a PDZ protein expressed in T lymphocytes, B lymphocytes, or both T and B lymphocytes. In an embodiment, the PL protein binds a PDZ protein expressed in endothelial cells. In various embodiments, the PL proteins and/or the PDZ protein to which it binds are not expressed in the nervous system (e.g., neurons).

In various embodiments of the invention, the PL protein of the invention binds only one PDZ protein listed in TABLE 2. In other embodiments, the PL protein binds 1 to 3, 3 to 5, or more than 5 different PDZ proteins listed in TABLE 2.

In various embodiments of the invention, the PL protein is expressed or up-regulated upon cell activation (e.g., in activated B lymphocytes, T lymphocytes) or upon entry into mitosis (e.g., up-regulation in rapidly proliferating cell populations).

In various embodiments of the invention, the PL protein is (i) a protein that mediates immune cell (e.g., hematopoietic cell) activation or migration, (ii) a protein that does not mediate apoptosis in a cell type, (iii) a protein that is other than a G-protein coupled seven transmembrane helix receptor, (iv) a protein that is G-protein coupled seven transmembrane helix receptor but not a cytokine receptor, or (v) a protein that is not a G-protein coupled seven transmembrane helix receptor and is a cytokine receptor.

### 6.1 Detection of PDZ Domain-Containing Proteins Expressed in Hematopoietic Cells

As noted supra, the present inventors surprisingly discovered that numerous PDZ proteins are expressed in immune system cells, and play a fundamental biological role in modulation of the immune response. PDZ proteins DLG1 and TIAM-1 have been previously described to be in T cells. The present inventors discovered, using a BLAST search of the Human EST database and the experiments described infra, that several additional PDZ proteins are present in hematopoietic cells including MPP1, P-DLG, VELI-1, PSD95, syntenin in T cells and CASK, DLG1, DLG2, ZIP KINASE, syntrophin 2, P-dlg, PSD95, and syntenin in B cells.

To determine the full extent of involvement of PDZ proteins in hematopoietic function, the inventors embarked on a systematic investigation of PDZ proteins in T and B cells. A comprehensive list of PDZ domain-containing proteins was retrieved from the Sanger Centre database (Pfam) searching for the keyword, “PDZ”. The corresponding cDNA sequences were retrieved from GenBank using the NCBI “entrez” database (hereinafter, “GenBank PDZ protein cDNA sequences”). The DNA portion encoding PDZ domains was identified by alignment of cDNA and protein sequence using CLUSTALW. Based on the DNA/protein alignment information, primers encompassing the PDZ domains were designed. The expression of certain PDZ-containing proteins in immune cells was detected by polymerase chain reaction (“PCR”) amplification of cDNAs obtained by reverse transcription (“RT”) of immune cell derived RNA (i.e., “RT-PCR”). PCR, RT-PCR and other methods for analysis and manipulation of nucleic acids are well known and are described generally in Sambrook et al., (1989) MOLECULAR CLONING: A LABORATORY MANUAL, 2ND ED., VOLS. 1-3, Cold Spring Harbor Laboratory hereinafter, “Sambrook”); and Ausubel et al., CURRENT PROTOCOLS IN MOLECULAR BIOLOGY, Greene Publishing and Wiley-Interscience, New York (1997), as supplemented through January 1999 (hereinafter “Ausubel”).

In the experiments summarized in TABLE 2, T-cells (Jurkat E6 cell line) and B-cells (MV 4-11 cell line) were tested for expression of specific PDZ domain containing genes by RT-PCR. RNA was prepared using the “trizol” RNA preparation kit (GIBCO-BRL; Cat. #15596-018) according to the manufacturer's recommendations. Briefly, 1-5 x 10⁷ lymphoblasts were harvested by centrifugation at 200×g for 10 minutes at 20° C. Cells were resuspended in 100 μl PBS buffer and 1 ml of TRIZOL reagent was added per 5×10⁶ cells. The cells resuspension was mixed and after 5 minutes incubation at room temperature (RT), chloroform was added at 0.2 ml per ml TRIZOL. The resuspension was vigorously shaken and incubated for .3 more minutes at RT. Samples were then centrifuged at 12000×g for 15 minutes at 4° C., the aqueous phase was recovered and RNA was precipitated with 2-propanol. The precipitate was collected by centrifugation at 12000×g for 15 minutes at 4° C., washed with 75% ethanol, finally recollected by another spin at 12000×g for 15 minutes at 4° C., air dried and resuspended in an appropriate volume of DEPC treated water.

RNA concentration and purity were determined by the measurement of 260/280 nm light absorption by the nucleic acid. For cDNA synthesis, the SUPERSCRIPT II reverse transcriptase cDNA kit (GIBCO-BRL; Cat. #18064-014) was used. RNA input per 200 μl cDNA reaction sample was 10 μg. Prior to cDNA synthesis RNA was treated with 1 unit/μl DNAse I in 110 μl water at 37° C. for 20 minutes. DNase I was then inactivated by a 10 minutes incubation at 70 C. Random primer was used for cDNA priming; 10 μl of random hexamer primer (100 ng/μl) was added, samples were heated to 70° C. for 5 minutes and chilled on ice. Subsequently 40 μl SUPERSCRIPT II “first strand” buffer, 20 μl of 0.1 M DDT, 10 μl of a 10 mM of mix of deoxynucleotide triphosphates (DATP, dCTP, dGTP, dUTP) and 10 μl of SUPERSCRIPT II reverse transcriptase were added and cDNA synthesis was done for 45 minutes at 42° C. Reactions were stopped by a 5 minutes incubation at 95° C. and typically, 24 μl of such cDNA samples were used for PCR.

A portion of the cDNA (typically, ⅕ of a 20 μl reaction) was used for PCR. PCR was conducted using primers designed to amplify specifically PDZ domain-containing regions of PDZ proteins of interest Oligonucleotide primers were designed to amplify one or more PDZ-encoding domains. The DNA sequences encoding the various PDZ domains of interest were identified by inspection (i.e., conceptual translation of the PDZ protein cDNA sequences obtained from GenBank, followed by alignment with the PDZ domain amino acid sequence). TABLE 3 shows the PCR primers, the PDZ-encoded domains amplified, and the GenBank accession number of the PDZ-domain containing proteins. To facilitate subsequent cloning of PDZ domains, the PCR primers included endonuclease restriction sequences at their ends to allow ligation with pGEX-3X cloning vector (Pharmacia, GenBank XXI13852 ) in frame with glutathione-S transferase (GST).

TABLE 3 lists proteins detected in the aforementioned assays. The results showed that PDZ proteins are widely utilized in T and B cells in both lineage specific as well as lineage independent manner. For example INADL2/3 (PDZ dom.), KLAA0316, and 26s subunit p27 were detected in T cells, but not B cells. mCASK, KIAA0559, PTN-4, and X11 beta were detected in B cells, but not T cells. AF6, BAII associated prot., Cytohesin bind. Prot., DLG1, DLG5 (pdlg), DVL1, DVL3, GTPase, hypoth. 41.8 kd, KIAA147, KIAA0300, KIAA0303, KIAA0380, KIAA0440, KIAA0545, KIAA0561, LIMK1, LIMK2, LIM domain prot, LIM protein, MINT1, MINT3, MPP1, MPP2, NE-DLG, NOS1, novel serine protease, PTN-3, prIL 16, PSD95, RGS12, serine protease, SYNTENIN, SYNTR 1 alpha, TAX1, TAX2, TAX33, TAX40, Tax43 (SYN, Beta1), TIAM wwp3, and X11 prot. were detected in both T cells and B cells. Similar expression patterns will be apparent from inspection of the Table.

TABLE 3

| GENE | | | | CLON. | FORWARD | REVERSE |
|---|---|---|---|---|---|---|
| SYMBOL | PROTEIN | ACC. # | AMINO ACID SEQUENCE | SITES | PRIMER | PRIMER |
| CASK | Homo sapiens | Y17138 | AA 495-584 | BaM HI/ | 6CAF | 7CAR |
| | CASK protein | GI: | PDZ domain 1 (of 1) | Eco RI | 5′- | 5′- |
| | | 3087817 | TRVRLVQFQKNTDEPMGITLKMNELNHC | | TCGGATCCATGT | TCGGAATTCAGAC |
| | | | IVARIMHGGMIHRQGTLHVGDEIREING | | GACCAGAGTTCG | TGAGTGCGGTA- |
| | | | ISVANQTVEQLQKMLREMRGSITFKIVP | | G-3′ | 3′ |
| | | | SYRTQS | | N1471-1494 | N1761-1738 |
| MPP1 | 55 Kd | M64925 | AA 101-186 | Bam HI/ | 62MPF | 63MPR |
| | erythrocyte | GI: | PDZ domain 1 (of 1) | BaM HI | 5′- | 5′- |
| | membrane | 189785 | CRKVRLIQFEKVTEEPMGITLKLNEKQS | | GGGATCCGGAAA | ACGGATCCGCTGG |
| | protein | | CTVARILHGGMIHRQGSLHVGDEILEIN | | GTGCGACTCATA | TTGGGAATTACTT |
| | | | GTNVTNHSVDQLQKAMKETKGMISLKVI | | C-3′ | -3′ |
| | | | PNQ | | N296-320 | N568-543 |
| LIMK1 | human LIM | NM_002314 | AA 194-291 | SMA I | 52LIFP | 53LIRP |
| | domain | GI: | PDZ domain 1 (of 1) | | 5′- | 5′- |
| | kinase 1 | 8051616 | VTLVSIPASSHGKRGLSVSIDPPHGPPG | | CTGCCCGGGACC | TCGCCCGGGTCAT |
| | | | CGTEHSHTVRVQGVDPGCMSPDVKNSIH | | GTCACCCTGGTG | GCTCGAGGGTC- |
| | | | VGDRILEINGTPIRNVPLDEIDLLIQET | | TCC-3′ | 3′ |
| | | | SRLLQLTLEHD | | N570-597 | N874-851 |
| KIAA 0303 | KIAA 0303 | Ab002301 | AA 652-742 | Bam HI/ | 152KIF | 153KIR |
| (K303) | protein | GI: | PDZ domain 1 (of 1) | Eco RI | 5′- | 5′- |
| | | 2224546 | PHQPIVIHSSGKNYGFTIRAIRVYVGDS | | CTGGGATCCCAC | TGTGAATTCAAAT |
| | | | DIYTVHHIVWNVEEGSPACQAGLKAGDL | | ATCAGCCGATTG | GGGGTAGTAGTGA |
| | | | ITHINGEPVHGLVHTEVIELLLKSGNKV | | TGA-3′ | TTG-3′ |
| | | | SITTTPF | | N1948-1976 | N2237-2209 |
| KIAA 0807 | KIAA 0807 | AB018350 | AA 635-743 | Bam HI/ | 281KIF | 282KIR |
| (K807) | protein | GI: | PDZ domain 1 (of 1) | Eco RI | 5′- | 5′- |
| | | 3882334 | PIIIHRAGKKYGFTLRAIRVYMGDSDVY | | GCAGGATCCCTC | GATGAATTCTCCA |
| | | | TVHHMVWHVEDGGPASEAGLRQGDLITH | | CCATCATCATCC | GGGGAGTTGTTG- |
| | | | VNGEPVHGLVHTEVVELILKSGNKVAIS | | AC-3′ | 3′ |
| | | | TTPLE | | N1894-1919 | N2155-2179 |
| DLG1 | human | U13897 | AA 275-477 | Bam HI/ | 1DF | 2DR |
| | homolog of | GI: | PDZ domains 1-2 (of 3) | Eco RI | 5′- | 5′- |
| | Drosophila | 558437 | VNGTDADYEYEEITLERGNSGLGFSIAG | | TCGGATCCAGGT | CGGAATTCGGTGC |
| | discs large | | GTDNPHIGDDSSIFITKIITGGAAAQDG | | TAATGGCTCAGA | ATAGCCATC-3′ |
| | protein | | RLRVNDCILQVNEVDVRNVTHSKAVEAL | | TG-3′ | N1442-1421 |
| | | | KEAGSIVRLYVKRRKPVSEKIMEIKLIK | | N815-841 | |
| | | | GPKGLGFSIAGGVGNQHIPGDNSIYVTK | | | |
| | | | IIEGGAAHKDGKLQIGDKLLAVNNVCLE | | | |
| | | | EVTHEEAVTALKNTSDFVYLKVAKPTSM | | | |
| | | | YMNDGYA | | | |
| PSD95 | human post- | U83192 | AA 387-724 | Bam HI/ | 8PSF | 11PSR |
| | synaptic | GI: | PDZ domains 1-3 (of 3) | Eco RI | 5′- | 5′- |
| | density | 3318652 | EGEMEYEEITLERGNSGLGFSIAGGTDN | | TCGGATCCTTGA | TCGGAATTCGCTA |
| | protein 95 | | PHIGDDPSIFITKIIPGGAAAQDGRLRV | | GGGGGAGATGGA | TACTCTTCTGG- |
| | | | NDSILFVNEVDVREVTHSAAVEALKEAG | | -3′ | 3′ |
| | | | SIVRLYVMRRKPPAEKVMEIKLIKGPKG | | N1150-1173 | N2191-2168 |
| | | | LGFSIAGGVGNQHIPGDNSIYVTKIIEG | | | |
| | | | GAAHKDGRLQIGDKILAVNSVGLEDVMH | | | |
| | | | EDAVAALKNTYDVVYLKVAKPSNAYLSD | | | |
| | | | SYAPPDITTSYSQHLDNEISHSSYLGTD | | | |
| | | | YPTAMTPTSPRRYSPVAKDLLGEEDIPR | | | |
| | | | EPRRIVIHRGSTGLGFNIVGGEDGEGIF | | | |
| | | | ISFILAGGPADLSGELRKGDQILSVNGV | | | |
| | | | DLRNASHEQAAI | | | |
| | | | ALKNAGQTVTIIAQYKPE | | | |
| NeDLG | Pre-synaptic | U49089 | AA 205-389 | Bam HI/ | 71NEDF | 72NEDR |
| | protein | GI: | PDZ domains 1-2 (of 3) | Eco RI | 5′- | 5′- |
| | sap102 | 1515354 | YEEIVLERGNSGLGFSIAGGIDNPHVPD | | CAGGATCCAATA | TTGAATTCGAGGC |
| | (neuroendo- | | DPGIFITKIIPGGAAAMDGRLGVNDCVL | | TGAGGAAATCGT | TGCCTGGCTTGGC |
| | crine-dlg) | | RVNEVEVSEVVHSRAVEALKEAGPVVRL | | ACTTG-3′ | -3′ |
| | | | VVRRRQPPPETIMEVNLLKGPKGLGFSI | | N608-635 | N1186-1161 |
| | | | AGGIGNQHIPGDNSIYITKIIEGGAAQK | | | |
| | | | DGRLQIGDRLLAVNNTNLQDVRHEEAVA | | | |
| | | | SLKNTSDNVYLKVAKPGS | | | |
| Syn- | syn-trophin | U40571 | AA 96-189 | Bam HI/ | 124SYF | 125SYR |
| trophin | alpha 1 | GI: | PDZ domain 1 (of 1) | ECO RI | 5′- | 5′- |
| alpha1 | protein | 1145727 | QRRRVTVRKADAGGLGISIKGGRENKMP | | TACGGATCCAGC | GTAGAATTCTTGA |
| gene | | | ILISKIFKGLAADQTEALFVGDAILSVN | | GGCCGCCGCGTG | AATACGGTGAGAC |
| (SNTa1) | | | GEDLSSATHDEAVQVLKKTGEVVLEVKY | | AC-3′ | -3′ |
| | | | MKDVSPYFK | | N279-301 | N576-551 |
| TAX-IP 43 | human tax | AF028828 | AA 15-85 | Bam HI/ | 97TAF | 98TAR |
| | interaction | GI: | PDZ domain 1 (of 1) | Eco RI | 5′- | 5′- |
| | protein 43 | 2613011 | QKRGVKVLKQELGGLGISIKGGKENKMP | | TCTGGATCCAGA | CGGAATTCAACGC |
| | | | ILISKIFKGLAADQTQALYVGDAILSVN | | AGCGTGGCGTGA | CTGCACCGCCTC- |
| | | | GADLRDATHDEAVQAL | | AGG-3′ | 3′ |
| | | | | | N37-63 | N267-231 |
| Limdomain | Lim domain | U90878 | AA 46-88 | Bam HI/ | 146L1F | 147L1R |
| protein | protein clp- | GI: | PDZ domain 1 (of 1) | Eco RI | 5′- | 5′- |
| gene | 36 | 2957144 | RGMTTQQIDLQGPGPWGFRLVGRKDFEQ | | GAATGACCACCC | GAGCCGCCTTGCT |
| (LDP) | | | PLAISRVTPGSKAAL | | CCAGGATCCGCG | CATGAATTCGCTA |
| | | | | | AGC-3′ | T-3′ |
| | | | | | N129-155 | N276-239 |
| Lim | Human LIM | AF061258 | AA 29-112 | Bam HI/ | 182LF | 183LR |
| protein | protein | GI: | PDZ domain 1 (of 1) | Eco RI | 5′- | 5′- |
| gene | | 3108092 | SNYSVSLVGPAPWGFRLQGGKDFNMPLT | | TTAGGATCCTGA | CTTGAATTCAGCA |
| (LIM) | | | ISSLKDGGKAAQANVRIGDVVLSIDGIN | | GCAAGTACAGTG | GATGCTCTTTGCA |
| | | | AQGMTHLEAQNKIKGCTGSLNMTLQRAS | | TGTCAC-3′ | GAGTC-3′ |
| | | | | | N86-115 | N350-320 |
| MINT1 | human LIM | L04953 | AA 717-894 | Eco RI/ | 34MIF | 20MR |
| | protein | GI: | PDZ domains 1-2 (of 2) | Eco RI | 5′- | 5′- |
| | | 340408 | SENCKDVFIEKQKGEILGVVIVESGWGS | | CGGAATTCGGAA | TCGGAATTCAGCA |
| | | | ILPTVIIANMMHGGPAEKSGKLNIGDQI | | AACTGTAAAGAT | GCCTGTACATCG- |
| | | | MSINGTSLVGLPLSTCQSIIKGLENQSR | | G-3′ | 3′ |
| | | | VKLNIVRCPPVTTVLIRRPDLRYQLGFS | | N2149-2167 | N2690-2666 |
| | | | VQNGIICSLMRGGIAERGGVRVGHRIIE | | | |
| | | | INGQSVVATPHEKIVHILSNAVGEIHMK | | | |
| | | | TMPAAMYRLL | | | |
| X11 beta | Homo sapiens | AF047348 | AA 558-843 | Bam HI/ | 133 XF | 134 XR |
| | adaptor | GI: | PDZ domains 1-2 (of 2) | Eco RI | 5′- | 5′- |
| | protein X11- | 3005559 | HFSNSENCKELQLEKHKGEILGVVVVES | | ACCGGATCCACT | AGCGAATTCTCCT |
| | beta | | GWGSILPTVILANMMNGCPAARSGKLSI | | TCTCAAACTCGG | GACCCGTGAGGAG |
| | | | GDQIMSINGTSLVGLPLATCQGIIKGLK | | AG-3′ | C-3′ |
| | | | NQTQVKLNIVSCPPVTTVLIKRPDLKYQ | | N1865-1890 | N2422-2438 |
| | | | LGFSVQNGIICSLMRGGIAERGGVRVGH | | | |
| | | | RIIEINGQSVVATAHEKIVQ | | | |
| | | | ALSNSVGEIHMKTMPAAMFRLLTGQEN | | | |
| KIAA 0440 | KIAA 0440 | AB007900 | AA 285-362 | Eco RI/ | 230KIF | 231KIR |
| (K440) | protein | GI: | PDZ domain 1 (of 1) | Eco RI | 5′- | 5′- |
| | | 2662160 | SSVEMTLRRNGLGQLGFHVNYEGIVADV | | AGGGAATTCATC | CAGAATTCATGCG |
| | | | EPYGYAWQAGLRQGSRLVEICKVAVATL | | GGTGGAGATGAC | GGGGAATGATGAC |
| | | | SHEQMIDLLRTSVTVKVVIIPPHE | | TCTGC-3′ | AAC-3′ |
| | | | | | N843-871 | N1066-1094 |
| KIAA 0545 | KIAA 0545 | AB011117 | AA 308-390 | Eco RI/ | 293TF | 294TR |
| (K545) | protein | GI: | PDZ domain 1 (of 1) | Eco RI | 5′- | 5′- |
| | | 3043613 | SGWETVDMTLRRNGLGQLGFHVKYDGTV | | CCGGATCCCGAG | AATGAATTCGAAG |
| | | | ABVEDYGFAWQAGLRQGSRLVEICKVAV | | GCGAGACCAAGG | GCCCTCTTGGGCT |
| | | | VTLTHDQMIDLLRTSVTVKVVIIPPFE | | AGGTG-3′ | G-3′ |
| | | | | | N384-411 | N672-646 |
| TAX-1P2 | human tax | AF028824 | AA 54-140 | Bam HI/ | 197TF | 198TR |
| | interaction | GI: | PDZ domain 1 (of 1) | ECO RI | 5′- | 5′- |
| | protein 2 | 2613003 | RKEVEVFKSEDALGLTITDNGAGYAFIK | | AGGGGATCCGCA | TGTGGAATTCCTT |
| | | | RIKEGSVIDHIHLISVGDMIEAINGQSL | | AGGAGGTGGAGG | GCGAGGCTCCGTG |
| | | | LGCRHYEVARLLKELPRGRTFTLKLTEP | | TGTTC-3′ | AGC-3′ |
| | | | RK | | N154-182 | N429-401 |
| TAX-IP | human tax | AC005175 | AA 130-221 | Bam HI/ | 293TF | 294TR |
| 2-like | interaction | GI: | PDZ domain 1 of 1 | Eco RI | 5′- | 5′- |
| | 2-like | 3253116 | IRGETKEVEVTKTEDALGLTITDNGAGY | | CCGGATCCCGAG | AATGAATTCGAAG |
| | protein | | AFIKRIKEGSIINRIEAVCVGDSIEAIN | | GCGAGACCAAGG | GCCCTCTTGGGCT |
| | | | DHSIVGCRHYEVAKMLRELPKSQPFTLR | | AGGTG-3′ | G-3′ |
| | | | LVQPKRAFE | | N384-411 | N672-646 |
| TAX-IP33 | tax inter- | AF028826 | AA 73-162 | Bam HI/ | 92TAF | 93TAR |
| | action | GI: | PDZ domain 1 (of 1) | Eco RI | 5′- | 5′- |
| | protein 33 | 2613007 | HSHPRVVELPKTDEGLGFNVMGGKEQNS | | GTGGGATCCACT | CATGAATTCCAGA |
| | | | PIYISRIIPGGVAERHGGLKRGDQLLSV | | CCCACCCTCGAG | ACTTTTGGGTGTA |
| | | | NGVSVEGEHHEKAVELLKAAKDSVKLVV | | TAG-3′ | TCGC-3′ |
| | | | RYTPKVL | | N208-234 | N497-468 |
| MPP2 | maguk p55 | X82895 | AA 185-273 | Bam HI/ | 142MF | 143MR |
| | subfamily | GI: | PDZ domain 1 (of 1) | Eco RI | 5′- | 5′- |
| | member 2 | 939884 | PVPPDAVRMVGIRKTAGEHLGVTFRVEG | | TCAGGATCCAGC | ATGGAATTCCTGG |
| | (DLG2) | | GELVIARILHGGMVAQQGLLHVGDIIKE | | CTGTACCTCCCG | TAGTTGGGCAGGA |
| | | | VNGQPVGSDPRALQELLRNASGSYILKI | | ATGC-3′ | TC-3′ |
| | | | LPNYQ | | N542-569 | N828-801 |
| MINT3 | human MINT3 | AF029110 | AA 11-52 | Bam HI/ | 188MF | 189MR |
| | | GI: | PDZ domain 1 (of 1) | Eco RI | 5′- | 5′- |
| | | 3169808 | PVTTAIIHRPHAREQLGFCVEDGIVRPR | | ACTGGATCCCCG | CTCGAATTCCGTG |
| | | | PLAPGWGGRAALST | | TCACCACCGCCA | CTCAGGGCCGCCC |
| | | | | | TCATC-3′ | TA-3′ |
| | | | | | N23-51 | N165-138 |
| TIP-1 | Homo sapiens | AF028823 | AA 14-117 | Bam HI/ | 86TAF | 87TAR |
| | Tax | GI: | PDZ domain 1 (of 1) | ECO RI | 5′- | 5′- |
| | interaction | 2613001 | QRVEIHKLRQGENLILGFSIGGGIDQDP | | CAGGGATCCAAA | ACGGAATTCTGCA |
| | protein 1 | | SQNPFSEDKTDKGIYVTRVSEGGPAEIA | | GAGTTGAAATTC | GCGACTGCCGCGT |
| | | | GLQIGDKIMQVNGWDMTMVTHDQARKRL | | ACAAGC-3′ | C-3′ |
| | | | TKRSEEVVRLLVTRQSLQK | | N10-39 | N305-331 |
| PTN-4 | protein- | M68941 | AA 774-862 | Bam HI/ | 247PTF | 248PTR |
| | tyrosine | GI: | PDZ domain 1 (of 1) | Eco RI | 5′- | 5′- |
| | phosphatase | 190747 | LIRMKPDENGRFGFNVKGGYDQKMPVIV | | ATCGGATCCTAA | ATCGAATTCAGCA |
| | meg1 | | SRVAPGTPADLCVPRLNEGDQVVLINGR | | TCAGAATGAAAC | TTAGGTCGAACTA |
| | | | DIAEHTHDQVVLFIKASCERHSGELMLL | | CTG-3′ | G-3′ |
| | | | VRPNA | | N2312-2338 | N2595-2569 |
| prIL16 | putative | S81601 | AA 170-383 | Bam HI/ | 75PRF | 76PRR |
| | interleukin | GI: | PDZ domain 1-2 (of 2) | Eco RI | 5′- | 5′- |
| | 16 precursor | 1478492 | IHVTILHKEEGAGLGFSLAGGADLENKV | | ACGGGATCCATG | GTGAATTCCTTGG |
| | | | ITVHRVFPNGLASQEGTIQKGNEVLSIN | | TCACCATCTTAC | ACTGGAGGCTTTT |
| | | | GKSLKGTTHHDALAILRQAREPRQAVIV | | AC-3′ | TC-3′ |
| | | | TRKLTPEAMPDLNSSTDSAASASAASDV | | N503-528 | N1157-1129 |
| | | | SVESTAEATVCTVTLEKNSAGLGFSLEG | | | |
| | | | GKGSLHGDKPLTINRIFKGAASEQSETV | | | |
| | | | QPGDEILQLGGTAMQGLTRFEAWNIIKA | | | |
| | | | LPDGPVTIVIRRKSLQSK | | | |
| Cyto-hesin | Cytohesin | AF08836 | AA 85-76 | Bam HI/ | 235CYF | 236CYR |
| binding | binding | GI: | PDZ domain 1 (of 1) | ECO RI | 5′- | 5′- |
| Protein | protein HE | 3192908 | QRKLVTVEKQDNETFGFEIQSYRPQNQN | | CCTGGATCCAAA | TCAGAATTCCATT |
| gene | | | ACSSEMFTLICKIQEDSPAHCAGLQAGD | | GAAAGCTTGTTA | AAGAGTCTCTATC |
| (CBP) | | | VLANINGVSTEGFTYKQVVDLIRSSGNL | | CTGTG-3′ | -3′ |
| | | | LTIETLNG | | N246-274 | N535-510 |
| KIAA 0751 | Hypoth. | AF007156 | AA 4-85 | Bam HI/ | 145HF | 146HR |
| (41.8) | 41.8 kD | GI: | PDZ domain 1 (of 1) | Eco RI | 5′- | 5′- |
| | protein | 2852637 | RDSGAMLGLKVVGGKMTESGRLCAFITK | | GTGGGATCCGAG | CTGGAATTCGCCT |
| | | | VKKGSLADTVGHLRPGDEVLEWNGRLLQ | | ATTCAGGAGCAA | TGAAACTACAAGT |
| | | | GATFEEVYNIILESKPEPQVELVVSR | | TGC-3′ | TC-3′ |
| | | | | | N4-30 | N267-240 |
| KIAA 0559 | KIAA 0559 | AB011131 | AA 766-870 | Bam HI/ | 130KIF | 131KIR |
| (K559) | protein | GI: | PDZ domain 1 (of 1) | Eco RI | 5′- | 5′- |
| | | 3043641 | HYIFPHARIKITRDSKDHTVSGNGLGIR | | AAAGGATCCACT | TCACAATTGGATA |
| | | | IVGGKEIPGHSGEIGAYIAKILPGGSAE | | ACATCTTTCCTC | GCATATTGAGGTC |
| | | | QTGKLMEGMQVLEWNGIPLTSKTYEEVQ | | ACG-3′ | CAG-3′ |
| | | | SIISQQSGEAEICVRLDLNML | | N2290-2312 | N2623-2595 |
| AF6 | af-6 protein | U02478 | AA 985-1077 | Bam HI/ | 66AFF | 67AFR |
| | | GI: | PDZ domain 1 (of 1) | Eco RI | 5′- | 5′- |
| | | 430993 | LRKEPEIITVTLKKQNGMGLSIVAAKGA | | TCGGATCCTGAG | TAGAATTCACCCT |
| | | | GQDKLGIYVKSVVKGGAADVDGRLAAGD | | GAAAGAACCTGA | GCTTTGCTACTTC |
| | | | QLLSVDGRSLVGLSQERAABLMTRTSSV | | A-3′ | -3′ |
| | | | VTLEVAKQG | | N2946-2970 | N3239-3214 |
| PICK1 | Novel human | AL049654 | AA 16-AA 105 | Bam HI/ | 287PIF | 288PIR |
| | mRNA similar | GI: | PDZ domain 1 (of 1) | Eco RI | 5′- | 5′- |
| | to mouse | 4678411 | PTVPGKVTLQKDAQNLIGISIGGGAQYC | | TCGGGATCCCGA | CTTGAATTCCTCC |
| | gene | | PCLYIVQVFDNTPAALDGTVAAGDEITG | | CTGTGCCTGGGA | TGCAGCTTCTTGT |
| | | | VNGRSIKGKTKVEVAKMIQEVKGE | | AG-3′ | TGTAG-3′ |
| | | | VTIHYNKLQE | | N527-N554 | N268-N293 |
| RGS12 | human | AF035152 | AA 35-103 | Bam HI/ | 64RGF | 65RGR |
| | regulator of | GI: | PDZ domain 1 (of 1) | Eco RI | 5′- | 5′- |
| | G-protein | 3290015 | PPRVRSVEVARGRAGYGFTLSGQAPCVL | | TGGGATCCCGCC | AGGAATTCCCAAT |
| | signal-ling | | SCVMRGSPADFVGLRAGDQILAVNEINV | | CCCAAGGGTGCG | TAATTTCACTAC- |
| | 12 | | KKASHEDVVKLIG | | GAG-3′ | 3′ |
| | | | | | N93-119 | N316-291 |
| PDZK1 | Homo sapiens | AF012281 | AA 134-457 | Bam HI/ | 238PDF | 239PDR |
| | PDZ domain | GI: | PDZ domains 2-4 (of 4) | Eco RI | 5′- | 5′- |
| | contain-ing | 2944188 | RLCYLVKEGGSYGFSLKTVQGKKGVYMT | | CCGGATCCGGCT | TAGGAATTCTTTC |
| | protein | | DITPQGVAMRAGVLADDHLIEVNGENVE | | CTGCTATCTCGT | CTCAGACTAGAAG |
| | (PDZK1) | | DASHEKVVEKVKKSGSRVMFLLVDKETD | | GAA-3′ | TG-3′ |
| | | | KRHVEQKIQFKRETASLKLLPHQPRIVE | | N 426-452 | N 1385-1412 |
| | | | MKKGSNGYGFYLRAGSEQKGQIIKDIDS | | | |
| | | | GSPAEEAGIAWNDLVVAVNGESVETLDH | | | |
| | | | DSVVEMIRKGGDQTSLLVVDKETDNMYR | | | |
| | | | LAHFSPFLYYQSQELPNGSVKEAPAPTP | | | |
| | | | TSLEVSSPPDTTEEVDHKPKLCRLAKGE | | | |
| | | | NGYGFHLNAIRGLPGSFIKEVQKGGPAD | | | |
| | | | LAGLEDEDVIIEVNGVNVLDEPYEKVVD | | | |
| | | | RIQSSGKNVTLLVCGK | | | |
| KIAA 0316 | KIAA 0316 | AB002314 | AA 197-284 | Bam HI/ | 158KIF | 159KIR |
| (K316) | protein | GI: | PDZ domain 1 (of 1) | EC0 RI | 5′- | 5′- |
| | | 6683123 | IPPAPRKVEMRRDPVLGFGFVAGSEKPV | | AAAGGATCCCTC | TTAGAATTCTGAT |
| | | | VVRSVTPGGPSEGKLIPGDQIVMINDEP | | CGGCTCCTCGGA | TTGGGAGAAGGGT |
| | | | VSAAPRERVIDLVRSCKESILLTVIQPY | | AG-3′ | AAG-3′ |
| | | | PSPK | | N586-611 | N866-839 |
| DLG5 | Human discs | U61843 | AA 99-338 | Bam HI/ | 81PDLGF | 82PDLGR |
| | large | GI: | PDZ domains 2 (of 2) | ECO RI | 5′- | 5′- |
| | protein | 3650451 | PYVEEPRHVKVQKGSEPLGISIVSGEKG | | ATAGGATCCCTT | TTGAATTCCTCAG |
| | p-dlg | | GIYVSKVTVGSIAHQAGLEYGDQLLEFN | | ATGTGGAGGAGC | GGCGGTACTGCAC |
| | | | GINLRSATEQQARLIIGQQCDTITILAQ | | CAC-3′ | CTTC-3′ |
| | | | YNPHVHQLSSHSRSSSHLDPAGTHSTLQ | | N645-N671 | N1356-N1385 |
| | | | GSGTTTPEHPSVIDPLMEQDEGPSTPPA | | | |
| | | | KQSSSRIAGDANKKTLEPRVVFIKKSQL | | | |
| | | | ELGVHLCGGNLHGVFVAEVEDDSPAKGP | | | |
| | | | DGLVPGDLILEYGSLDVRNKTVEEVYVE | | | |
| | | | MLKPRDGVRLKVQYRPE | | | |
| Mouse | Mus musculus | AF077527 | AA 67-241 | Bam HI/ | 14SF | 15SR |
| Syntenin | Syntenin | GI: | REIKQGIREVILCKDQDGKIGLRLKSID | Eco RI | 5′- | 5′- |
| gene | | 3342559 | NGIFVQLVQANSPASLVGLRFGDQVLQI | | TCGGATCCTTGA | TCGGAATTCATGC |
| (SYNT) | | | NGENCAGWSSDKAHKVLKQAFGEKITMT | | AATTAAGCAAGG | CTGGAGCCATCC- |
| | | | IRDRPFERTVIMHKDSSGHVGFIFKSGK | | GAT-3′ | 3′ |
| | | | ITSIVKDSSAARNGLLTDHHICEINGQN | | N363-N390 | N896-N920 |
| | | | VIGLKDAQIADILSTAGTVVT | | | |
| | | | ITIMPTFIFEHIIKRMAPSM | | | |
| WWP3 | Homo sapiens | U80754 | AA 314-576 | Bam HI/ | 164WWF | 165WWR |
| | membrane | GI: | PDZ domains 1-2 (of 2) | Eco RI | 5′- | 5′- |
| | associated | 2695619 | PSELKGKFIHTKLRKSSRGFGFTWGGD | | CACGGATCCCTT | CTTGAATTCTGGC |
| | guanylate | | EPDEFLQIKSLVLDGPAALDGKMETGDV | CTGAGTTGAAAG | AGCCCTCCTCGTT | |
| | kinase 1 | | IVSVNDTCVLGHTHAQVVKIFQSIPIGA | | GC-3′ | GC-3′ |
| | (MAGI-1) | | SVDLELCRGYPLPFDPDDPNTSLVTSVA | | N932-N957 | N1710-N1737 |
| | | | ILDKEPIIVNGQETYDSPASHSSKTGKV | | | |
| | | | NGMKDARPSSPADVASNSSH | | | |
| | | | GYPNDTVSLASSIATQPELITVHIVKGP | | | |
| | | | MGFGFTIADSPGGGGQRVKQIVDSPRCR | | | |
| | | | GLKEGDLIVEVNKKNVQALTHNQVVDML | | | |
| | | | VECPKGSEVTLLVQRGGLP | | | |
| TAX-IP 40 | human tax | AF028827 | AA 35-137 | Bam HI/ | 136TF | 137TR |
| | inter-action | GI: | PDZ domain 1 (of 1) | Eco RI | 5′- | 5′- |
| | protein 40 | 2613009 | LLPETHRRVRLHKHGSDRPLGFYIRDGM | | ACGGGATCCTAC | ACGGAATTCCGCT |
| | | SVRVAPOGLERVPGIFISRLVRGGLAES | | TGCCTGAGACCC | GGTTGGCGGGCTT | |
| | | TGLLAVSDEILEVNGIEVAGKTLDQVTD | | ACC-3′ | GAC-3′ | |
| | | MMVANSHNLIVTVKPANQR | | N97-123 | N421-393 | |
| KIAA | KIAA 0858 | AB020665 | AA 66-159 | Bgl II/ | 278KIF | 279KIR |
| 0858 | protein | GI: | PDZ domain 1 (of 1) | Eco RI | 5′- | 5′- |
| (K858) | | 4240204 | FSDMRISINQTPGKSLDFGFTIKWDIPG | | AGGAGATCTTCA | CTTGAATTCAGGT |
| | | | IFVASVEAGSPAEFSQLQVDDEIIAINN | | GTGATATGAGAA | GAACCAGCCTTTC |
| | | | TKFSYNDSKEWEEAMAKAQETGHLVMDV | | TC-3′ | -3′ |
| | | | RRYGKAGSPE | | N190-N215 | N460-N485 |
| TIAM1 | T- lymphoma | NM_003253 | AA 1001-1088 | Bam HI/ | 39TF | 40TR |
| | invasion and | GI: | PDZ domain 1 (of 1) | Eco RI | 5′- | 5′- |
| | metastasis | 4507500 | HSIHIEKSDTAADTYGFSLSSVEEDGIR | | TCGGATCCACAG | TCGGAATTCCTCC |
| | inducing | | RLYVNSVKETGLASKKGLKAGDEILEIN | | CATCCACATTGA | AGCTCGGGGT-3′ |
| | protein 1 | | NRAADALNSSMLKDFLSQPSLGLLVRTY | | G-3′ | N3275-3253 |
| | | | PELE | | N2995-3019 | |
| Connector | Homo sapiens | AF100153 | AA 193-300 | Bam HI/ | 296CF | 297CR |
| Enhancer | connector | GI: | PDZ domain 1 (of 1) | Eco RI | 5′- | 5′- |
| gene | enhancer of | 3930780 | LEQKAVLEQVQLDSPLGLEIHTTSNCQH | | AGGGGATCCTGG | GGGAATTCCGGTA |
| (ConEn) | KSR-like | | FVSQVDTQVPTDSRLQIQPGDEVVQINE | | AACAGAAGGCCG | TCGGGATCTTCCT |
| | protein CNK1 | | QVVVGWPRKNMVRELLREPAGLSL | | TGCTC-3′ | TC-3′ |
| | | | VLKKIPIP | | N605-NE33 | N858-N884 |
| Serine | Homo sapiens | AF020760 | AA 421-506 | Eco RI/ | 191SF | 192SR |
| protease | serine | GI: | Splice variant: void of AA | Eco RI | 5′- | 5′- |
| (SPsht) | protease | 2738914 | 444-465 (ref. to GI: | | GAAGAATTCCTC | TGCGAATTCGGAT |
| | (omi) | | 2738914) | | CTCCGGAATCAG | TGGGTTCGAACAG |
| | | | PDZ domain 1 (of 1) | | TG-3′ | CTTC-3′ |
| | | | SSSGISGSQRRYIGVMMLTLSPSAGLRP | | N1S01-N1526 | N1774-N1803 |
| | | | GDVILAIGEQMVQNAEDVYEAVRTQSE | | | |
| DVL1 | human | AF00E011 | AA 248-340 | Bam HI/ | 1^{st} PCR: | 1^{st} PCR: |
| | dishevelled | GI: | PDZ domain 1 (of 1) | Eco RI | 55DVISF | 56DVISR |
| | segment | 2291005 | LNIVTVTLNMERHHFLGISIVGQSNDRG | | 5′- | 5′- |
| | polarity | | DGGIYIGSIMKGGAVAADGRIEPGDMLL | | TCATCCAGACTC | GCTCATGTCACTC |
| | protein | | QVNDVNFENMSNDDAVRVLREIVSQTGP | | ATCCGGAAG-3′ | TTCACCG-3′ |
| | homolog | | ISLTVAKCW | | NE52-673 | N1195-1174 |
| | | | | | 2^{nd} PCR, | 2^{nd} PCR, |
| | | | | | nested: | nested: |
| | | | | | 37DVF | 38DVR |
| | | | | | 5′- | 5′- |
| | | | | | TCGGATCCAAAC | TCGGAATTCCCAG |
| | | | | | GGTCACTCTCAA | CACTTGGCTACAG |
| | | | | | C-3′ | -3′ |
| | | | | | N723-747 | N1029-N1004 |
| Novel | Homo sapiens | Y07921 | AA 107-204 | Bam HI/ | 194NSF | 195NSR |
| serine | novel serine | GI: | PDZ domain 1 (of 2) | Eco RI | 5′- | 5′- |
| protease | protease | 1621243 | IRQAKGKAITKKKYIGIRMMSLTSSKAK | | CCCGGATCCGAC | GATGAATTCATTA |
| (NSP) | protein | | ELKDRHRDFPDVISGAYIIEVIPDTPAE | | AGGCCAAAGGAA | CCCCTGCGGACCA |
| | (PRSS 11) | | AGGLKENDVIISINGQSVVSANDVSDVI | | AAGC-3′ | CCATG-3′ |
| | | | KRESTLNMVVRRGN | | N1138-N1165 | N1415-N1445 |
| Guanin | Homo sapiens | AF117947 | AA 343-450 | Bgl II | 275GF | 276GR |
| Change | PDZ domain | GI: | PDZ domain 1 (of 1) | Eco RI | 5′- | 5′- |
| Factor | containing | 6650765 | CSVMIFEVVEQAGAIILEDGQELDSWYV | | GAGAGATCTGCT | CCGGAATTCATGT |
| gene | guanine | | ILNGTVEISHPDGKVENLFMGNSFGITP | | CAGTGATGATTT | ACCATAACAATTT |
| (GEF) | nucleotide | | TLDKQYMHGIVRTKVDDCQFVCIAQQDY | | TTG-3′ | C-3′ |
| | exchange | | WRILNHVEKNTHKEEEGEIVMVH | | N1088-N1114 | N1402-N1428 |
| | factor I | | | | | |
| KIAA 0902 | KIAA 0902 | AB020715 | AA 214-301 | Bam HI/ | 290KIF | 291KIR |
| (K902) | protein | GI: | PDZ domain 1 (of 1) | Eco RI | 5′- | 5′- |
| | | 4240304 | ILNEMIAPVMRVNYGQSTDINAFVGAVS | | AGAGGATCCTCA | TCTGAATTCCAAT |
| | | | LSCSDSGLWAVEGGNKLVCSGLLQASKS | | ATGAAATGATTG | TTGGTAGACCACT |
| | | | NLISGSVMYIEEKTKTKYTGNPTKMYEV | | C-3′ | TC-3′ |
| | | | VYQIG | | N633-N657 | N884-N991 |
| KIAA 0561 | KIAA 0561 | AB011133 | AA 948-1038 | Bam HI/ | 161KIF | 162KIR |
| (K561) | protein | GI: | PDZ domain 1 (of 1) | Eco RI | 5′- | 5′- |
| | | 3043645 | PPSLSTALARSTASACGRSASTWVIATS | | CCTGGATCCCCC | GAGGAATTCTCCA |
| | | | TLCTTSSGVWRTBAPPRRRACGLGTSSP | | CATCGTTATCCA | GGGCTGTGQTCCG |
| | | | TSTGSQCWGWCTWTSWSCCZRAATRYPC | | CAGC-3′ | -3′ |
| | | | GPQPWR | | N2836-2863 | N3120-3095 |
| NOS1 | human | U17327 | AA 239-329 | Bam HI/ | 155NOF | 156NOR |
| | neuronal | GI: | PDZ domain 1 (of 1) | Eco RI | 5′- | 5′- |
| | nitric oxide | 642525 | IQPNVISVRLFKRKVGGLGFLVKERVSK | | AGCGGATCCAGC | GAAGAATTCAGGG |
| | synthase | | PPVIISDLIRGGAAEQSGLIQAGDIILA | | CCAATGTCATTT | CCCCTCAGAATG- |
| | | | VNGRPLVDLSYDSALEVLRGIASETHVV | | C-3′ | 3′ |
| | | | LILRGP | | N711-733 | N994-970 |
| PA 3108375 v2 | | | | | | |
| *Note concerning TABLE 3 | | | | | | |
| In several cases, sequence analysis of the PDZ clones revealed differences to the DNA and/or protein sequence as published in the databases, summarized in TABLE 3A. | | | | | | |
| Key: | | | | | | |
| Gene names and corresponding gene products are provided. In some cases, cDNA sequences representing the same gene have several database entries under different accession numbers and names. Accession numbers shown correspond to the gene name used in this description, and numbering of nucleotides and amino acids correlates to the Genbank entry versions specified by the given accession number. Amino acid sequences shown correspond to the cloned DNA portions of PDZ domain containing genes. As is | | | | | | |
| # apparent from the primer sequences, in some constructs, the first N-terminal and/or last C-terminal amino acid corresponds to a linker amino acid introduced by the cloning process but is not represented at that position in the corresponding gene. PCR primers were designed such that restriction nuclease recognition sites were generated at the ends of the RT-PCR generated fragments. Therefore, 5′ primer sequences do not entirely match with the corresponding cDNA sequences. | | | | | | |

TABLE 3A

| GENE | GENBANK ENTRY*** | ACTUAL CONSTRUCT |
|---|---|---|
| AF6 | N 3060: C | N 3060: T* |
| DLG1 | N 1021: A, = AA 340: Gln | N 1021: G, = AA 340: Arg |
| Lim dom. | N 202: G | N 202: C* |
| | N 203: C, = AA 68: Arg | N 203: G, = AA 68: Gly |
| LIMK1 | N 855: C, = AA 285: Leu | N 855: A, = AA 285: Ile |
| MINT1 | N 2386: G, = AA 796: Glu** | N 2386: A, = AA 796: Lys** |
| NE-DLG | N 713: T | N 713: C* |
| | N 766: G, = AA 255: Gly | N 766: A, = AA 255: Glu |
| | N 803: G, = AA 267: Glu | N 803: C, = AA 267: Asp |
| | N 861: G, = AA 287: Val | N 861: A, = AA 287: Met |
| TIAM1 | N 3224: A | N 3224: G* |
| MPP2 | N 812 = A; AA = Asn | N 812 = G; AA = Ser |
| TIP-1 | N 196 = T; AA = Ile | N 196 = G; AA = Ser |

*= silent mutation, does not effect the AA sequence;
**= MINT1 is the same as X11a. The database entry for X11a shows the same sequence as our actual construct with regard to N 2386 of the MINT1 GenBank entry.
***= Nucleotide (“N”) and amino acid (“AA”) annotations correspond to the numbering as found in the GenBank files (for accession no., see Table 3).

### 6.2 Assays for Detection of Interactions Between PDZ-Domain Polypeptides and Candidate PDZ Ligand Proteins (PL Proteins)

Two complementary assays, termed “A′ and “G,”” were developed to detect binding between a PDZ-domain polypeptide and candidate PDZ ligand. In each of the two different assays, binding is detected between a peptide having a sequence corresponding to the C-terminus of a protein anticipated to bind to one or more PDZ domains (i.e. a candidate PL peptide) and a PDZ-domain polypeptide (typically a fusion protein containing a PDZ domain). In the “A” assay, the candidate PL peptide is immobilized and binding of a soluble PDZ-domain polypeptide to the immobilized peptide is detected (the “A″” assay is named for the fact that in one embodiment an avidin surface is used to immobilize the peptide). In the “G” assay, the PDZ-domain polypeptide is immobilized and binding of a soluble PL peptide is detected (The “G” assay is named for the fact that in one embodiment a GST-binding surface is used to immobilize the PDZ-domain polypeptide). Preferred embodiments of these assays are described in detail infra. However, it will be appreciated by ordinarily skilled practitioners that these assays can be modified in numerous ways while remaining useful for the purposes of the present invention.

6.2.1 Production of Fusion Proteins Containing PDZ-Domains

GST-PDZ domain fusion proteins were prepared for use in the assays of the invention. PCR products containing PDZ encoding domains (as described in §6.1 supra) were subcloned into an expression vector to permit expression of fusion proteins containing a PDZ domain and a heterologous domain (i.e., a glutathione-S transferase sequence, “GST”). PCR products (i.e., DNA fragments) representing PDZ domain encoding DNA was extracted from agarose gels using the “sephaglas” gel extraction system (Pharmacia) according to the manufacturer's recommendations.

As noted supra, PCR primers were designed to include endonuclease restriction sites to facilitate ligation of PCR fragments into a GST gene fusion vector (pGEX-3X; Pharmacia, GenBank accession no. XXU13852) in-frame with the glutathione-S transferase coding sequence. This vector contains a IPTG inducible lacZ promoter. The pGEX-3X vector was linearized using Bam HI and Eco RI or, in some cases, Eco RI or Sma I, as shown in TABLE 3, and dephosphorylated. For most cloning approaches, double digestion with Barn HI and Eco RI was performed, so that the ends of the PCR fragments to clone were Bam HI and Eco RI. In some cases, restriction endonuclease combinations used were Bgl II and Eco RI, Bam HI and Mfe I, or Eco RI only, Sma I only, or BamHil only (see TABLE 3). When more than one PDZ domain was cloned, the DNA portion cloned represents the PDZ domains and the cDNA portion located between individual domains. Precise locations of cloned fragments used in the assays are indicated in TABLE 3. DNA linker sequences between the GST portion and the PDZ domain containing DNA portion vary slightly, dependent on which of the above described cloning sites and approaches were used. As a consequence, the amino acid sequence of the GST-PDZ fusion protein varies in the linker region between GST and PDZ domain. Protein linkers sequences corresponding to different cloning sites/approaches are shown below. Linker sequences (vector DNA encoded) are bold, PDZ domain containing gene derived sequences are in italics.
1) GST-BamHI/BamHI—PDZ domain insert
   Gly-Ile-PDZ domain insert
2) GST-BamHI/BglII—PDZ domain insert
   Gly-Ile—PDZ domain insert
3) GST-EcoRI/EcoI—PDZ domain insert
   Gly-Ile-ProGly-Asn—PDZ domain insert (SEQ ID NO: 360)
4) GST-SmaI/SmaI—PDZ domain insert
   Gly-Ile-Pro—PDZ domain insert

The PDZ-encoding PCR fragment and linearized pGEX-3X vector were ethanol precipitated and resuspended in 10 ul standard ligation buffer. Ligation was performed for 4-10 hours at 7° C. using T4 DNA ligase. It will be understood that some of the resulting constructs include very short linker sequences and that, when multiple PDZ domains were cloned, the constructs included some DNA located between individual PDZ domains.

The ligation products were transformed in DH5α or BL-21 E.coli bacteria strains. Colonies were screened for presence and identity of the cloned PDZ domain containing DNA as well as for correct fusion with the glutathione S-transferase encoding DNA portion by PCR and by sequence analysis. Positive clones were tested in a small scale assay for expression of the GST/PDZ domain fusion protein and, if expressing, these clones were subsequently grown up for large scale preparations of GST/PDZ fusion protein.

GST-PDZ domain fusion protein was overexpressed following addition of IPTG to the culture medium and purified. Detailed procedure of small scale and large scale fusion protein expression and purification are described in “GST Gene Fusion System” (second edition, revision 2; published by Pharmacia). In brief, a small culture (3-5 mls) containing a bacterial strain (DH5α, BL21 or JM109) with the fusion protein construct was grown overnight in LB-media at 37° C. with the appropriate antibiotic selection (100 ug/ml ampicillin; a.k.a LB-amp). The overnight culture was poured into a fresh preparation of LB-amp (typically 250-500mls) and grown until the optical density (OD) of the culture was between 0.5 and 0.9 (approximately 2.5 hours). IPTG (isopropyl β-D-thiogalactopyranoside) was added to a final concentration of 1.0 mM to induce production of GST fusion protein, and culture was grown an additional 1.5-2.5 hours. Bacteria were collect by centrifugation (4500 g) and resuspended in Buffer A-(50 mM, Tris, pH 8.0, 50 mM dextrose, InmM EDTA, 200 uM phenylmethylsulfonylfluoride). An equal volume of Buffer A+ (Buffer A-, 4 mg/ml lysozyme) was added and incubated on ice for 3 min to lyse bacteria. An equal volume of Buffer B (10 mM Tris, pH 8.0,50 mM KCl, 1 mM EDTA. 0.5% Tween-20,0.5% NP40 (ak.a. IGEPAL CA-630), 200 uM phenylmethylsulfonylfluoride) was added and incubated for an additional 20 min. The bacterial cell lysate was centrifuged (×20,000g), and supernatant was added to glutathione Sepharose 4B (Pharmacia, cat no. 17-0765-01) previously swelled (rehydrated) in 1× phosphate-buffered saline (PBS). The supernatant-Sepharose slurry was poured into a column and washed with at least 20 bed volumes of 1×PBS. GST fusion protein was eluted off the glutathione sepharose by applying 0.5-1.0 ml aliquots of 5 mM glutathione and collected as separate fractions. Concentrations of fractions were determined using BioRad Protein Assay (cat no. 500-0006) according to manufacturer's specifications. Those fractions containing the highest concentration of fusion protein were pooled and dialyzed against 1×PBS/35% glycerol. Fusion proteins were assayed for size and quality by SDS gel electrophoresis (PAGE) as described in “Sambrook.” Fusion protein aliquots were stored at minus 80° C. and at minus 20° C.

6.2.2 Identification of Candidate PL Proteins and Synthesis of Peptides

In some non-hematopoietic cells (e.g., neurons, epithelial cells), certain PDZ domains are known to be bound by the C-terminal residues of PDZ-binding proteins. To identify PL proteins that function in hematopoietic and endothelial cells, cell surface receptor proteins were identified and peptides having the sequence corresponding to the C-terminus of each protein were synthesized. TABLE 4 lists these proteins, and provides corresponding C-terminal sequences and GenBank accession numbers. “Clasp 1” is described in WO 00/20434 (published 13 Apr. 2000). “Clasp 2” and “Clasp 4” are described in copending applications U.S. Ser. Nos. 09/547276, 60/196527, 60/240,503, 09/687837 and PCT/US00/10158 and have the C-terminal sequences shown in Table 4.

Synthetic peptides of defined sequence (e.g., corresponding to the carboxyl-termini of the indicated proteins) can be synthesized by any standard resin-based method (see, e.g., U.S. Pat. No. 4,108,846; see also, Caruthers et al., 1980, Nucleic Acids Res. Symp. Ser., 215-223; Horn et al., 1980, Nucleic Acids Res. Symp. Ser., 225-232; Roberge, et al., 1995, Science 269:202). The peptides used in the assays described herein were prepared by the FMOC (see, e.g., Guy and Fields, 1997, Meth. Enz. 289:67-83; Wellings and Atherton, 1997, Meth. Enz.289:44-67). In some cases (e.g., for use in the A and G assays of the invention), peptides were labeled with biotin at the amino-terninus by reaction with a four-fold excess of biotin methyl ester in dimethylsulfoxide with a catalytic amount of base. The peptides were cleaved from the resin using a halide containing acid (e.g. trifluoroacetic acid) in the presence of appropriate antioxidants (e.g. ethanedithiol) and excess solvent lyophilized.

Following lyophilization, peptides can be redissolved and purified by reverse phase high performance liquid chromatography (HPLC). One appropriate HPLC solvent system involves a Vydac C-18 semi-preparative column running at 5 mL per minute with increasing quantities of acetonitrile plus 0.1% trifluoroacetic acid in a base solvent of water plus 0.1% trifluoroacetic acid. After HPLC purification, the identities of the peptides are confirmed by MALDI cation-mode mass spectrometry. As noted, exemplary biotinylated peptides are provided in TABLE 4.

6.2.3 Detecting PDZ-PL Interactions

Based on the determination that immune system cells contain both many PDZ proteins and similarly many candidate PL proteins, it was apparent to the inventors that characterization of the specific PDZ-PL interactions among these proteins would require reliable and rapid assays for such interactions. A variety of assay formats known in the art can be used to select ligands that are specifically reactive with a particular protein. For example, solid-phase ELISA immunoassays, immunoprecipitation, Biacore, and Western blot assays can be used to identify peptides that specifically bind PDZ-domain polypeptides. As discussed supra, two different, complementary assays were developed to detect PDZ-PL interactions. In each, one binding partner of a PDZ-PL pair is immobilized, and the ability of the second binding partner to bind is determined. These assays, which are described infra, can be readily used to screen for hundreds to thousand of potential PDZ-Iigand interactions in a few hours. Thus these assays can be used to identify yet more novel PDZ-PL interactions in hematopoietic cells. In addition, they can be used to identify antagonists of PDZ-PL interactions (see infra). In various embodiments, fusion protein are used in the assays and devices of the invention. Methods for constructing and expressing fusion proteins are well known. Fusion proteins generally are described in Ausubel et al., supra, Kroll et al., 1993, DNA Cell. Biol. 12:441, and Imai et al., 1997, Cell 91:521-30. Usually, the fusion protein includes a domain to facilitate immobilization of the protein to a solid substrate (“an immobilization domain”). Often, the immobilization domain includes an epitope tag (i.e., a sequence recognized by a antibody, typically a monoclonal antibody) such as polyhistidine (Bush et al, 1991, J. Biol Chem 266:13811-14), SEAP (Berger et al, 1988, Gene 66:1-10), or M1 and M2 flag (see, e.g, U.S. Pat. Nos. 5,011,912; 4,851,341; 4,703,004; 4,782,137). In an embodiment, the immobilization domain is a GST coding region. It will be recognized that, in addition to the PDZ-domain and the particular residues bound by an immobilized.antibody, protein A, or otherwise contacted with the surface, the protein (e.g., fusion protein), will contain additional residues. In some embodiments these are residues naturally associated with the PDZ-domain (i.e., in a particular PDZ-protein) but they may include residues of synthetic (e.g., poly(alanine)) or heterologous origin (e.g., spacers of, e.g., between 10 and 300 residues).

PDZ domain-containing polypeptide used in the methods of the invention (e.g., PDZ fusion proteins) of the invention are typically made by (1) constructing a vector (e.g., plasmid, phage or phagemid) comprising a polynucleotide sequence encoding the desired polypeptide, (2) introducing the vector into an suitable expression system (e.g., a prokaryotic, insect, mammalian, or cell free expression system), (3) expressing the fusion protein and (4) optionally purifying the fusion protein.

(1) In one embodiment, expression of the protein comprises inserting the coding sequence into an appropriate expression vector (i.e., a vector that contains the necessary elements for the transcription and translation of the inserted coding sequence required for the expression system employed, e.g., control elements including enhancers, promoters, transcription terminators, origins of replication, a suitable initiation codon (e.g., methionine), open reading frame, and translational regulatory signals (e.g., a ribosome binding site, a termination codon and a polyadenylation sequence. Depending on the vector system and host utilized, any number of suitable transcription and translation elements, including constitutive and inducible promoters, can be used.

The coding sequence of the fusion protein includes a PDZ domain and an immobilization domain as described elsewhere herein. Polynucleotides encoding the amino acid sequence for each domain can be obtained in a variety of ways known in the art; typically the polynucleotides are obtained by PCR amplification of cloned plasmids, EDNA libraries, and cDNA generated by reverse transcription of RNA, using primers designed based on sequences determined by the practitioner or, more often, publicly available (e.g., through GenBank). The primers include linker regions (e.g., sequences including restriction sites) to facilitate cloning and manipulation in production of the fusion construct. The polynucleotides corresponding to the PDZ and immobilization regions are joined in-frame to produce the fusion protein-encoding sequence.

The fusion proteins of the invention may be expressed as secreted proteins (e.g., by including the signal sequence encoding DNA in the fusion gene; see, e.g., Lui et al, 1993, PNAS USA, 90:8957-61) or as nonsecreted proteins.

In some embodiments, the PDZ-containing proteins are immobilized on a solid surface. The substrate to which the polypeptide is bound may in any of a variety of forms, e.g., a microtiter dish, a test tube, a dipstick, a microcentrifuge tube, a bead, a spinnable disk, and the like. Suitable materials include glass, plastic (e.g., polyethylene, PVC, polypropylene, polystyrene, and the like), protein, paper, carbohydrate, lipip monolayer or supported lipid bilayer, and other solid supports. Other materials that may be employed include ceramics, metals, metalloids, semiconductive materials, cements and the like.

In some embodiments, the fusion proteins are organized as an array. The term “array,” as used herein, refers to an ordered arrangement of immobilized fusion proteins, in which particular different fusion proteins (i.e., having different PDZ domains) are located at different predetermined sites on the substrate. Because the location of particular fusion proteins on the array is known, binding at that location can be correlated with binding to the PDZ domain situated at that location. Immobilization of fusion proteins on beads (individually or in groups) is another particularly useful approach. In one embodiment, individual fusion proteins are immobilized on beads. In one embodiment, mixtures of distinguishable beads are used. Distinguishable beads are beads that can be separated from each other on the basis of a property such as size, magnetic property, color (e.g., using FACS) or affinity tag (e.g., a bead coated with protein A can be separated from a bead not coated with protein A by using IgG affinity methods). Binding to particular PDZ domain may be determined; similarly, the effect of test compounds (i.e., agonists and antagonists of binding) may be determined.

Methods for immobilizing proteins are known, and include covalent and non-covalent methods. One suitable immobilization method is antibody-mediated immobilization. According to this method, an antibody specific for the sequence of an “immobilization domain” of the PDZ-domain containing protein is itself immobilized on the substrate (e.g., by adsorption). One advantage of this approach is that a single antibody may be adhered to the substrate and used for immobilization of a number of polypeptides (sharing the same immobilization domain). For example, an immobilization domain consisting of poly-histidine (Bush et al, 1991, J. Biol Chem 266:13811-14) can be bound by an anti-histidine monoclonal antibody (R&D Systems, Minneapolis, Minn.); an immobilization domain consisting of secreted alkaline phosphatase (“SEAP”) (Berger et al, 1988, Gene 66:1-10) can be bound by anti-SEAP (Sigma Chemical Company, St. Louis, Mo.); an immobilization domain consisting of a FLAG epitope can be bound by anti-FLAG. Other ligand-antiligand immobilization methods are also suitable (e.g., an immobilization domain consisting of protein A sequences (Harlow and Lane, 1988, Antibodies A Laboratory Manual, Cold Spring Harbor Laboratory, Sigma Chemical Co., St. Louis, Mo.) can be bound by IgG; and an immobilization domain consisting of strepavidin can be bound by biotin (Harlow & Lane, supra; Sigma Chemical Co., St. Louis, Mo.). In a preferred embodiment, the immobilization domain is a GST moiety, as described herein.

When antibody-mediated immobilization methods are used, glass and plastic are especially useful substrates. The substrates may be printed with a hydrophobic (e.g., Teflon) mask to form wells. Preprinted glass slides with 3, 10 and 21 wells per 14.5 cm² slide “working area,” are available from, e.g., SPI Supplies, West Chester, Pa.; also see U.S. Pat. No. 4,011,350). In certain applications, a large format (12.4 cm×8.3 cm) glass slide is printed in a 96 well format is used; this format facilitates the use of automated liquid handling equipment and utilization of 96 well format plate readers of various types (fluorescent, colorimetric, scintillation). However, higher densities may be used (e.g., more than 10 or 100 polypeptides per cm²). See, e.g., MacBeath et al, 2000, Science 289:1760-63.

Typically, antibodies are bound to substrates (e.g., glass substrates) by adsorption. Suitable adsorption conditions are well known in the art and include incubation of 0.5-50 ug/ml (e.g., 10 ug/ml) mAb in buffer (e.g., PBS, or 50 to 300 mM Tris, MOPS, HEPES, PIPES, acetate buffers, pHs 6.5 to 8, at 4° C.) to 37° C. and from 1 hr to more than 24 hours.

Proteins may be covalently bound or noncovalently attached through nonspecific bonding. If covalent bonding between a the fusion protein and the surface is desired, the surface will usually be polyfunctional or be capable of being polyfunctionalized. Functional groups which may be present on the surface and used for linking can include carboxylic acids, aldehydes, amino groups, cyano groups, ethylenic groups, hydroxyl groups, mercapto groups and the like. The manner of linking a wide variety of compounds to various surfaces is well known and is amply illustrated in the literature.

6.2.3.1 “A Assay”Detection of PDZ-Ligand Binding Using Immobilized PL Peptide.

In one aspect, the invention provides an assay in which biotinylated candidate PL peptides are immobilized on an avidin coated surface. The binding of PDZ-domain fusion protein to this surface is then measured. In a preferred embodiment, the PDZ-domain fusion protein is a GST/PDZ fusion protein and the assay is carried out as follows:

(1) Avidin is bound to a surface, e.g. a protein binding surface. In one embodiment, avidin is bound to a polystyrene 96 well plate (e.g., Nunc Polysorb (cat #475094) by addition of 100 uL per well of 20 ug/mL of avidin (Pierce) in phosphate buffered saline without calcium and magnesium, pH 7.4 (“PBS”, GibcoBRL) at 4° C. for 12 hours. The plate is then treated to block nonspecific interactions by addition of 200 uL per well of PBS containing 2 g per 100 mL protease-free bovine serum albumin (“PBS/BSA”) for 2 hours at 4° C. The plate is then washed 3 times with PBS by repeatedly adding 200 uL per well of PBS to each well of the, plate and then dumping the contents of the plate into a waste container and tapping the plate gently on a dry surface.

(2) Biotinylated PL peptides (or candidate PL peptides, e.g. see TABLE 4) are immobilized on the surface of wells of the plate by addition of 50 uL per well of 0.4 uM peptide in PBS/BSA for 30 minutes at 4° C. Usually, each different peptide is added to at least eight different wells so that multiple measurements (e.g. duplicates and also measurements using different (3ST/PDZ-domain fusion proteins and a GST alone negative control) can be made, and also additional negative control wells are prepared in which no peptide is immobilized. Following immobilization of the PL peptide on the surface, the plate is washed 3 times with PBS.

(3) GST/PDZ-domain fusion protein (prepared as described supra) is allowed to react with the surface by addition of 50 uL per well of a solution containing 5 ug/mL GST/PDZ-domain fusion protein in PBS/BSA for 2 hours at 4° C. As a negative control, GST alone (i.e. not a fuision protein) is added to specified wells, generally at least 2 wells (i.e. duplicate measurements) for each immobilized peptide. After the 2 hour reaction, the plate is washed 3 times with PBS to remove unbound fusion protein.

(4) The binding of the GST/PDZ-domain fusion protein to the avidin-biotinylated peptide surface can be detected using a variety of methods, and detectors known in the art. In one embodiment, 50 uL per well of an anti-GST antibody in PBS/BSA (e.g. 2.5 ug/mL of polyclonal goat-anti-GST antibody, Pierce) is added to the plate and allowed to react for 20 minutes at 4° C. The plate is washed 3 times with PBS and a second, delectably labeled antibody is added. In one embodiment, 50 uL per well of 2.5 ug/mL of horseradish peroxidase (HRP)-conjugated polyclonal rabbit anti-goat immunoglobulin antibody is added to the plate and allowed to react for 20 minutes at 4° C. The plate is washed 5 times with 50 mM Tris pH 8.0 containing 0.2% Tween 20, and developed by addition of 100 uL per well of HRP-substrate solution (TMB, Dako) for 20 minutes at room temperature (RT). The reaction of the HRP and its substrate is terminated by the addition of 100 uL per well of IM sulfuric acid and the optical density (O.D.) of each well of the plate is read at 450 nm.

(5) Specific binding of a PL peptide and a PDZ-domain polypeptide is detected by comparing the signal from the well(s) in which the PL peptide and PDZ domain polypeptide are combined with the background signal(s). The background signal is the signal found in the negative controls. Typically a specific or selective reaction will be at least twice background signal, more typically more than 5 times background, and most typically 10 or more times the background signal. In addition, a statistically significant reaction will involve multiple measurements of the reaction with the signal and the background differing by at least two standard errors, more typically four standard errors, and most typically six or more standard errors. Correspondingly, a statistical test (e.g. a T-test) comparing repeated measurements of the signal with repeated measurements of the background will result in a p-value <0.05, more typically a p-value <0.01, and most typically a p-value <0.001 or less.

As noted, in an embodiment of the “A” assay, the signal from binding of a GST/PDZ-domain fusion protein to an avidin surface not exposed to (i.e. not covered with) the PL peptide is one suitable negative control (sometimes referred to as “B”). The signal from binding of GST polypeptide alone (i.e. not a fusion protein) to an avidin-coated surface that has been exposed to (i.e. covered with) the PL peptide is a second suitable negative control (sometimes referred to as “B2”). Because all measurements are done in multiples (i.e. at least duplicate) the arithmetic mean (or, equivalently, average) of several measurements is used in determining the binding, and the standard error of the mean is used in determining the probable error in the measurement of the binding. The standard error of the mean of N measurements equals the square root of the following: the sum of the squares of the difference between each measurement and the mean, divided by the product of (N) and (N−1). Thus, in one embodiment, specific binding of the PDZ protein to the plate-bound PL peptide is determined by comparing the mean signal (“mean S”) and standard error of the signal (“SE”) for a particular PL-PDZ combination with the mean B1 and/or mean B2. In TABLE 2, binding was detected to be specific (denoted by an “A” in the matrix) when (1) the, mean S was at least twice the mean B1 and at least twice the mean B2 and (2) the mean S was at least six standard errors (six SE) greater than both the mean B 1 and the mean B2. In addition, in the experiments summarized in TABLE 2, an additional criterion was used to ensure that none of the interactions defined as specific arose from a combined tendency of both the particular PDZ fusion protein and PL peptide tested to each give a higher than usual background. This criteria was that (3) the mean S was at least twenty times the product of the mean B1 and the mean B2. The factor twenty times reflects that at least one of B1 and B2 is generally less than 0.1 O.D. units, and therefore twenty times the product of the mean B1 and the mean B2 is generally less than twice the mean B1 and twice the mean B2, making criteria (3) less stringent than criteria (1). Only in a few cases where the mean B1 and the mean B2 are both greater than 0.1 O.D. units (i.e. both the particular PDZ fusion protein and PL peptide tested tend to give a higher than usual background) is criteria (3) more stringent than criteria (1).

TABLE 4
PL Peptides

| | | | |
|---|---|---|---|
| CODE | PROTEIN NAME | GENBANK ACCESS | SEQUENCE |

| | | | |
|---|---|---|---|
| AA1L | Clasp-1 | | ISKATPALPTVSISSSAEV |
| AA2L | Clasp-2 | | ISGTPTSTMVHGMTSSSSVV |
| AA3L | Clasp-4 | | CAISGTSSDRGYGSPRYAEV |
| AA4L | CD3n | M33158 | SVFSIPTLWSPWPPSSSSQL |
| AA5L-M* | CD4 | M12807 | SEKKSQSPHRFQKTCSPI |
| AA6L | CD6 | X60992 | SPQPDSTDNDDYDDISAA |
| AA7L | CD34 | M81104 | QATSENGHSARQHVVADTEL |
| AA8L | CD38 | NM004334 | PDKFLQCVKNPEDSSCTSKI |
| AA9L | CD44 | M69215 | QFMTADETRNLQNVDMKIGV |
| AA10L | CD46 (Form 1) | M58050 | KKGTYLTDETHREVKFTSL |
| AA11L | CD49E (4) | X06256 | PYGTAMEKAQLKPPATSDA |
| AA12L | CD49F | X53596 | HKAEIHAQPSDKERLTSDA |
| AA13L | CD95 | M67454 | KDITSDSENSNFRNEIQSLV |
| AA14L | CD97 | X84700 | TSGTGHNQTRALRASESGI |
| AA15L | CD98 | J02939 | ERLKLEPHEGLLLRPPYAA |
| AA16L | CD105 | X72012 | STNHSIGSTQSTPCSTSSMA |
| AA17L | VCAM1 | M73255 | ARKANMKGSYSLVEAQKSKV |
| AA18L | CD138 | J05392 | PKQANGGAYQKPTKQEEFYA |
| AA19L | CD148 | D37781 | ENLAPVTTFGKTNGYIA |
| AA20L | CD166 | L38608 | DLGNMEENKKLEENNHKTEA |
| AA21L | CDw137 (4-1BB) | NM001561 | QEEDGCSCRFPEEEEGGCEL |
| AA22L | DNAM-1 | U56102 | TREDIYVNYPTFSRRPKTRV |
| AA23L-M* | FasL | U11821 | SSKSKSSEESQTFFGLYKL |
| AA25L | FceRIb | D10583 | YSATYSELEDPGEMSPPIDL |
| AA26L | Galectin3 | J02921 | ISKLGISGDIDLTSASYTMI |
| AA27L | CD114 | N14000760 | LNFPLLQGIRVHGMEALGSF |
| AA28L | CDW125 (IL5R) | X62156 | EVICYIEKPGVETLEDSVF |
| AA29.1L | CDW128A (IL8RA) | M68932 | ARHRVTSYTSSSVNVSSNL |
| AA29.2L | CDW128E (IL8RB) | M73969 | KDSRPSFVGSSSGHTSTTL |
| AA30L | LPAP | X81422 | AWDDSARAAGGQGLHVTAL |
| AA31L | Mannose Receptor | NM002438 | GTSDMKDLVGNIEQNEHSVI |
| AA32L | Spectrin (beta) | NM000347 | SFPPCGHRENVPGQSLVSFV |
| AA33L | KV1.3 | AAC31761 | TTNNNPNSAVNIKKIFTDV |
| AA34.2L | NMDA | NP000824 | LNSCSNRRVYKKMPSIESDV |
| AA36L | Neuroligin | NM018977 | TFAAGFNSTGLPHSTTRV |
| AA37L | Glycophorin C | AAA52574 | QGDPALQDAGDSSRKEYFI |
| AA38L | Neurexin | AB011150 | SSAKSSNKNKKNKDKEYYV |
| AA39L | Syndecan-2 | A33880 | GERKPSSAAYQKAPTKEPYA |
| AA40L | DOCK2 | BAA13200 | LASKSAEEGKQIPDSLSTDL |
| AA41L | CC CKR-1R | L09230 | LERVSSTSPSTGEHELSAGF |
| AA42L | CC CKR-2 | U03882 | GKGKSIGRAPEASLQDKEGA |
| AA43L | CC CKR-3 | HSU28694 | LERTSSVSPSTAEPELSIVF |
| AA44L | CC CKR-4 | X85740 | DTPSSSYTQSTMDHDLHDAL |
| AA45L | BLR-1 | S56162 | PSWRRSSLSESENATSLTTF |
| AA46L | Volt. Gated Ca2+ | Q00975 | SSGGRARHSYHHPDQDHWC |
| AA47L | CD83 | Z11697 | VTSPNKHLGLVTPHKTELV |
| AA45L | CD62E | M30640 | SSSQSLESDGSYQKPSYIL |
| AA49L | CD5 | X04391 | SMQPDNSSDSDYDLHGAQRL |
| AA55L | CD148 | D37781 | TIYENLAPVTTFGKTIA |
| AA56L | TAX | AB038239 | QISPGGLEPPSEKHFRETEV |
| AA57L | BLR-1/CXCR5 | NM001716 | SWRRSSLSESENATSLTTF |
| AA58L | PAG | NM018440 | KENDYESISDLQQGRDITRL |

(PAG—Phosphoprotein Associated with GEMs)
*The Sequence studied is mutated at positions >10 amino acids from C-terminus to increase water solubility and/or eliminate intramolecular disulfides.

6.2.3.2 “G Assay”—Detection of PDZ-Ligand Binding Using Immobilized PDZ-Domain Fusion Polypeptide

In one aspect, the invention provides an assay in which a GST/PDZ fusion protein is immobilized on a surface (“G” assay). The binding of labeled PL peptide (as listed in TABLE 4) to this surface is then measured. In a preferred embodiment, the assay is carried out as follows:

(1) A PDZ-domain polypeptide is bound to a surface, e.g. a protein binding surface. In a preferred embodiment, a GST/PDZ fusion protein containing one or more PDZ domains is bound to a polystyrene 96-well plate. The GST/PDZ fusion protein can be bound to the plate by any of a variety of standard methods known to one of skill in the art, although some care must be taken that the process of binding the fusion protein to the plate does not alter the ligand-binding properties of the PDZ domain. In one embodiment, the GST/PDZ fusion protein is bound via an anti-GST antibody that is coated onto the 96-well plate. Adequate binding to the plate can be achieved when:
a. 100 uL per well of 5 ug/mL goat anti-GST polyclonal antibody (Pierce) in PBS is added to a polystyrene 96-well plate (e.g., Nunc Polysorb) at 4° C. for 12 hours.
b. The plate is blocked by addition of 200 uL per well of PBS/BSA for 2 hours at 4° C.
c. The plate is washed 3 times with PBS.
d. 50 uL per well of 5 ug/mL GST/PDZ fusion protein) or, as a negative control, GST polypeptide alone (i.e. not a fusion protein) in PBS/BSA is added to the plate for 2 hours at 4° C.
e. the plate is again washed 3 times with PBS.

(2) Biotinylated PL peptides (or candidate PL peptides, e.g. as shown in TABLE 4) are allowed to react with the surface by addition of 50 uL per well of 20 uM solution of the biotinylated peptide in PBS/BSA for 10 minutes at 4° C., followed by an additional 20 minute incubation at 25° C. The plate is washed 3 times with ice cold PBS.

(3) The binding of the biotinylated peptide to the GST/PDZ fusion protein surface can be detected using a variety of methods and detectors known to one of skill in the art. In one embodiment, 100 uL per well of 0.5 ug/mL streptavidin-horse radish peroxidase (HRP) conjugate dissolved in BSA/PBS is added and allowed to react for 20 minutes at 4° C. The plate is then washed 5 times with 50 mM Tris pH 8.0 containing 0.2% Tween 20, and developed by addition of 100 uL per well of HRP-substrate solution (TMB, Dako) for 20 minutes at room temperature (RT). The reaction of the HRP and its substrate is terminated by addition of 100 uL per well of I M sulftric acid, and the optical density (O.D.) of each well of the plate is read at 450 um.

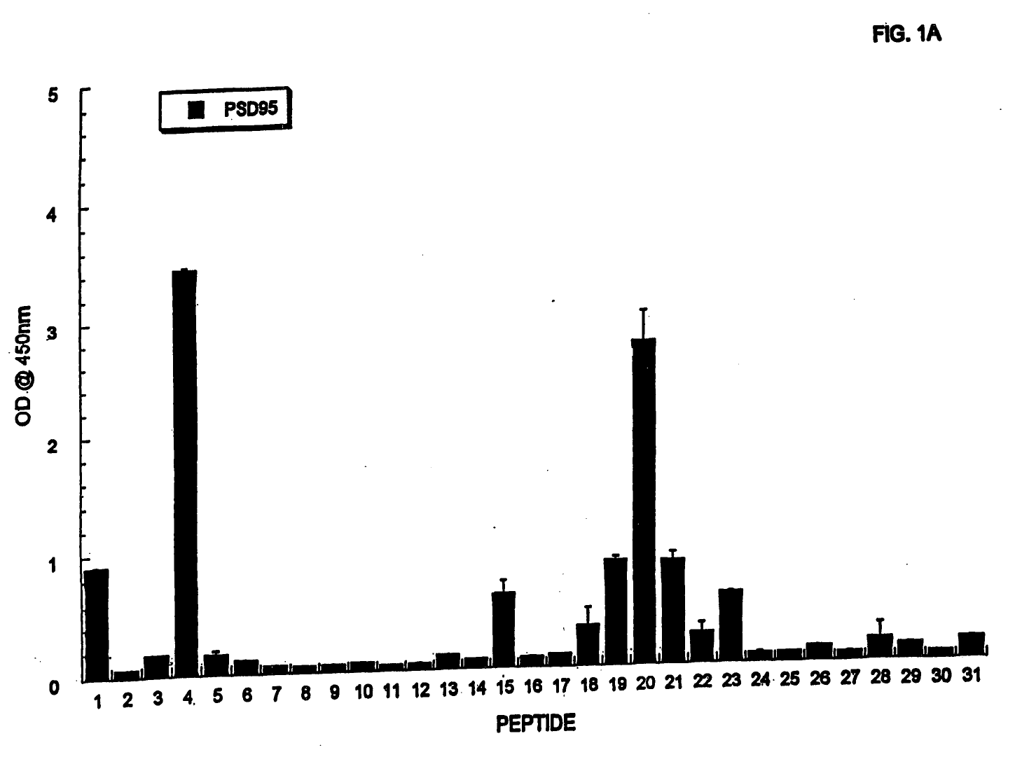

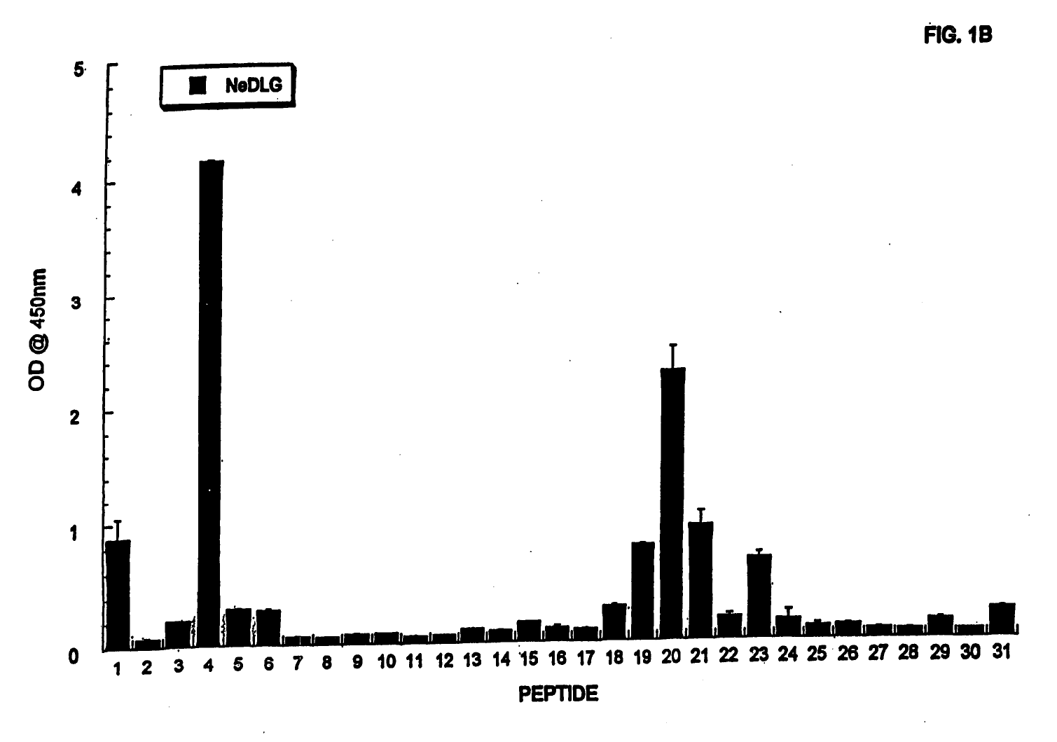

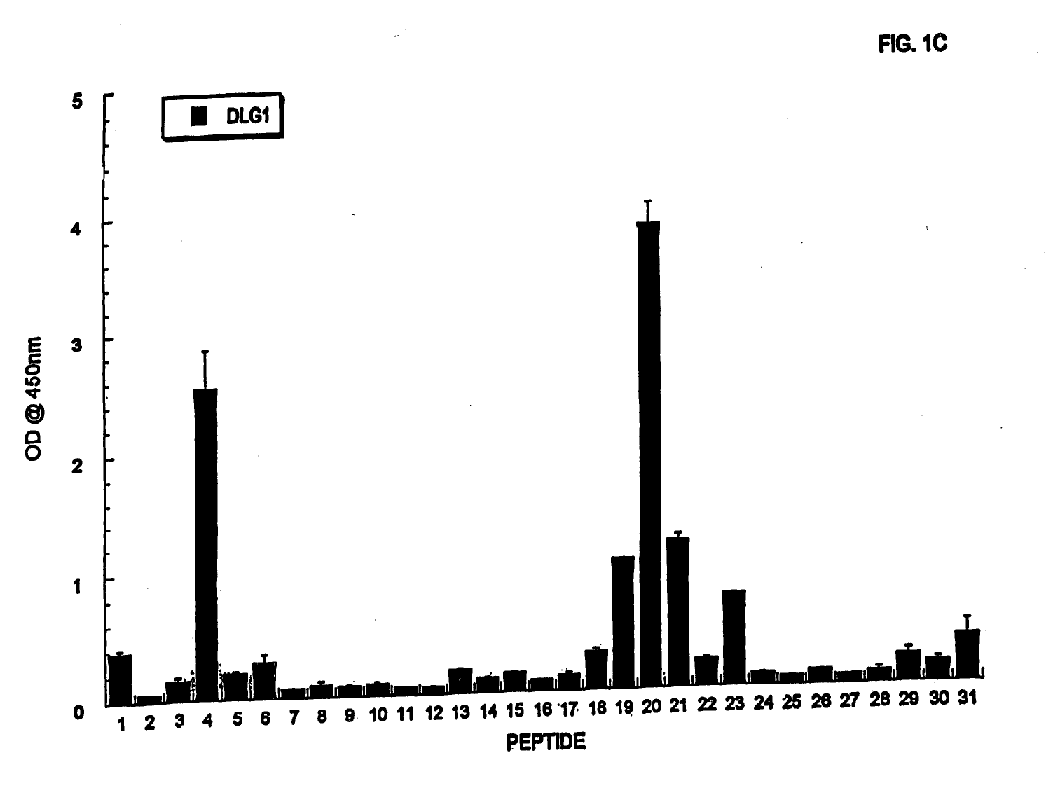

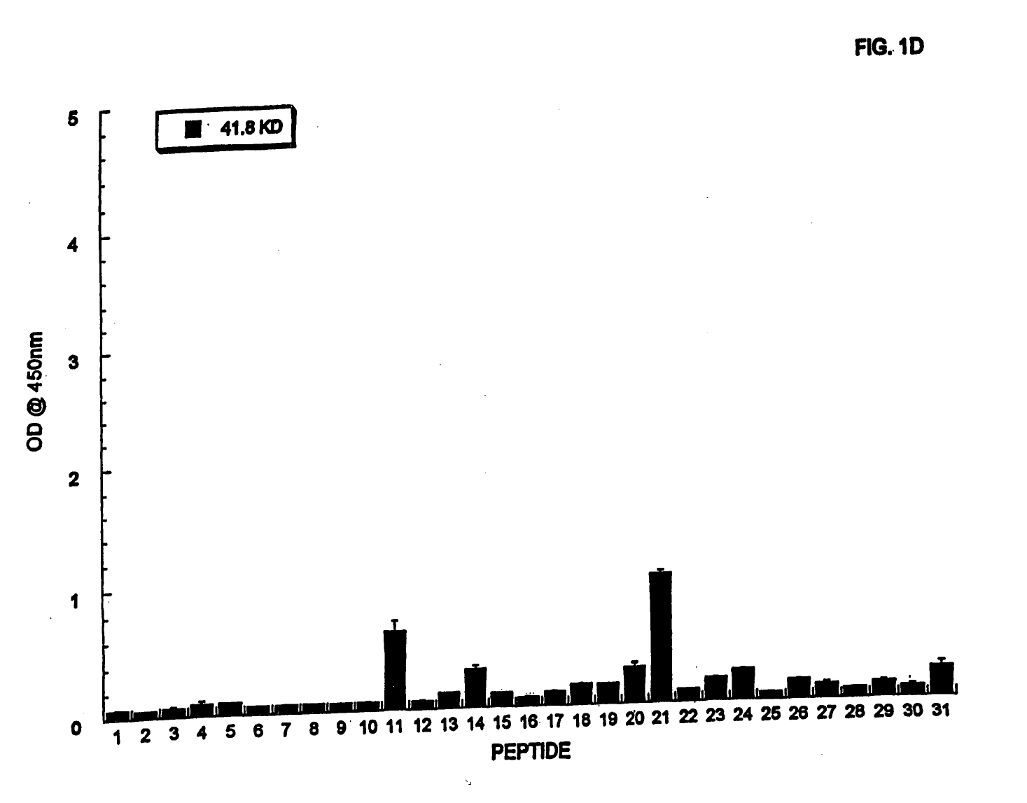

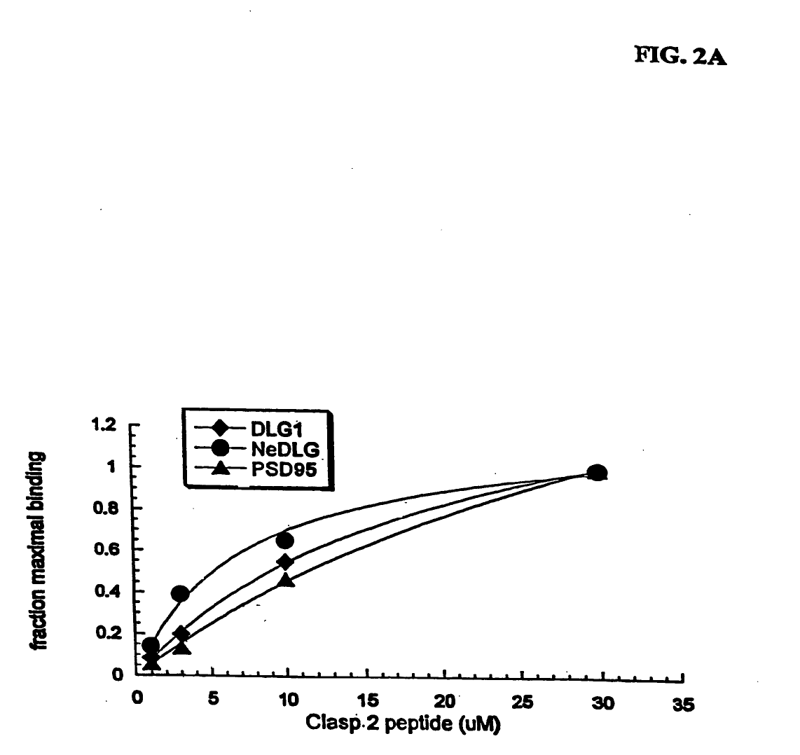

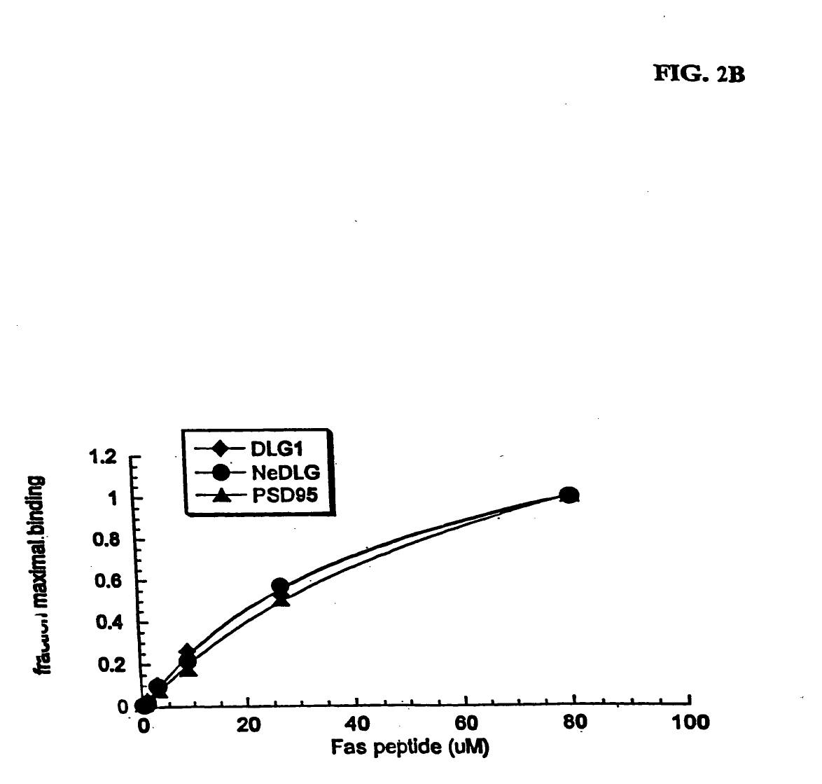

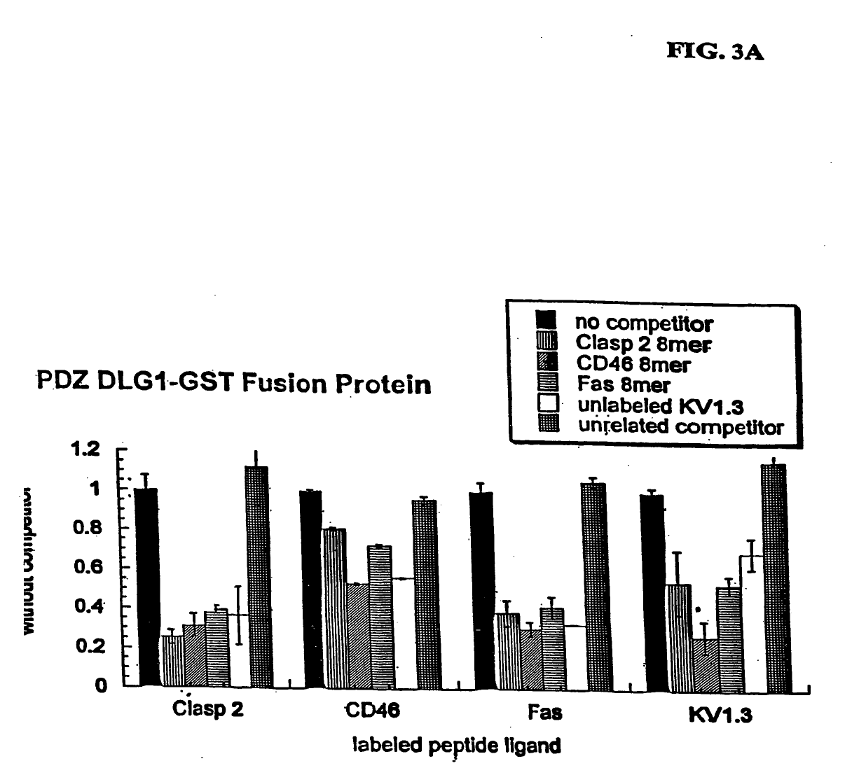

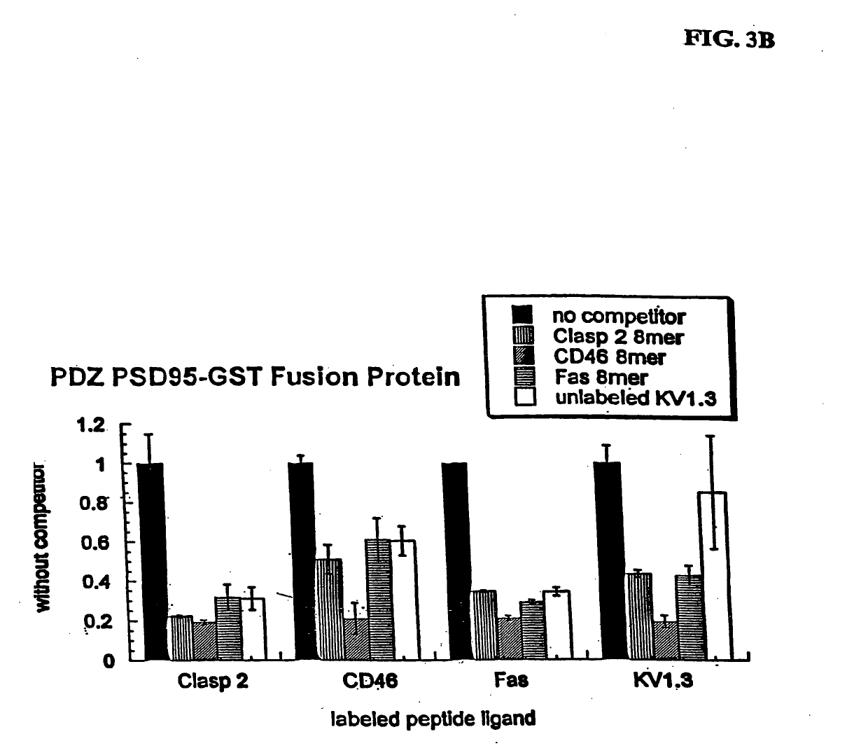

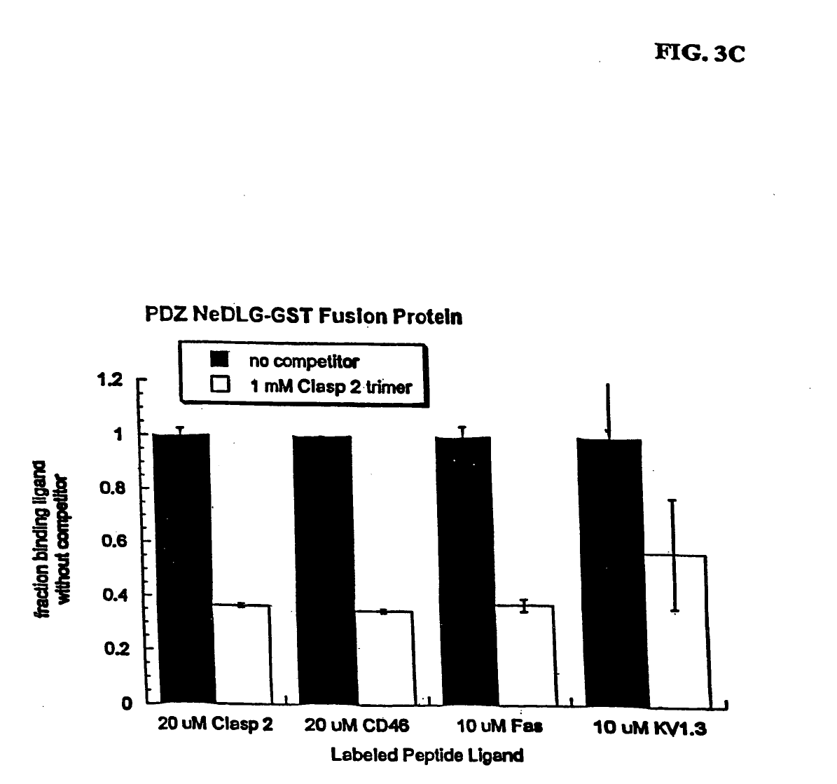

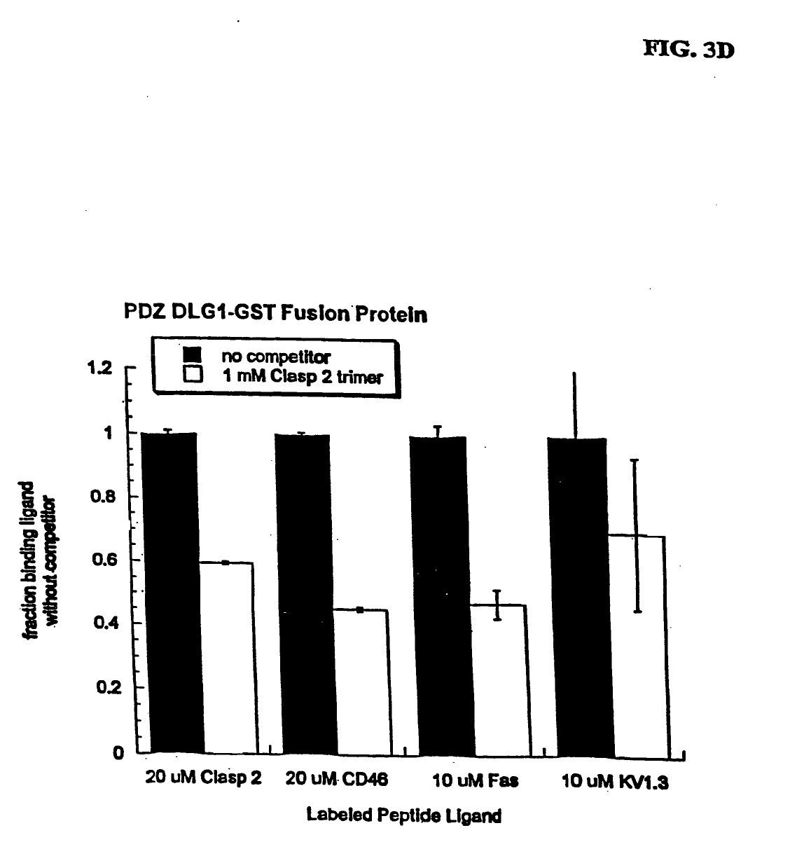

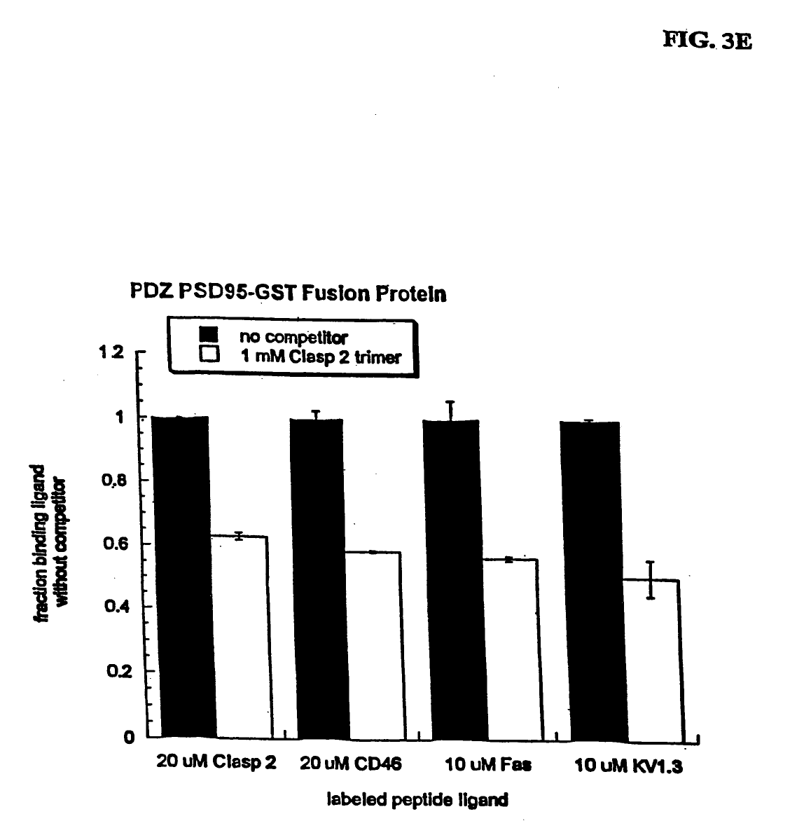

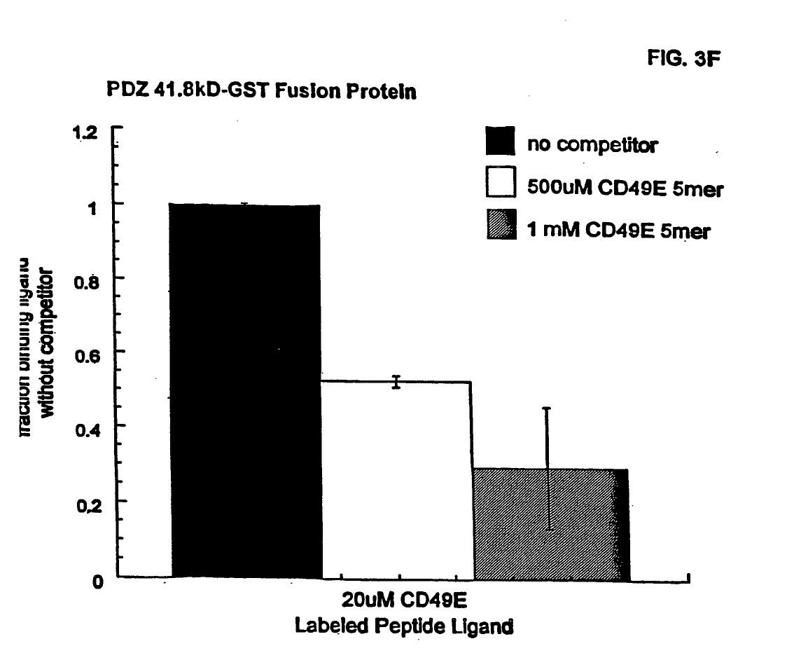

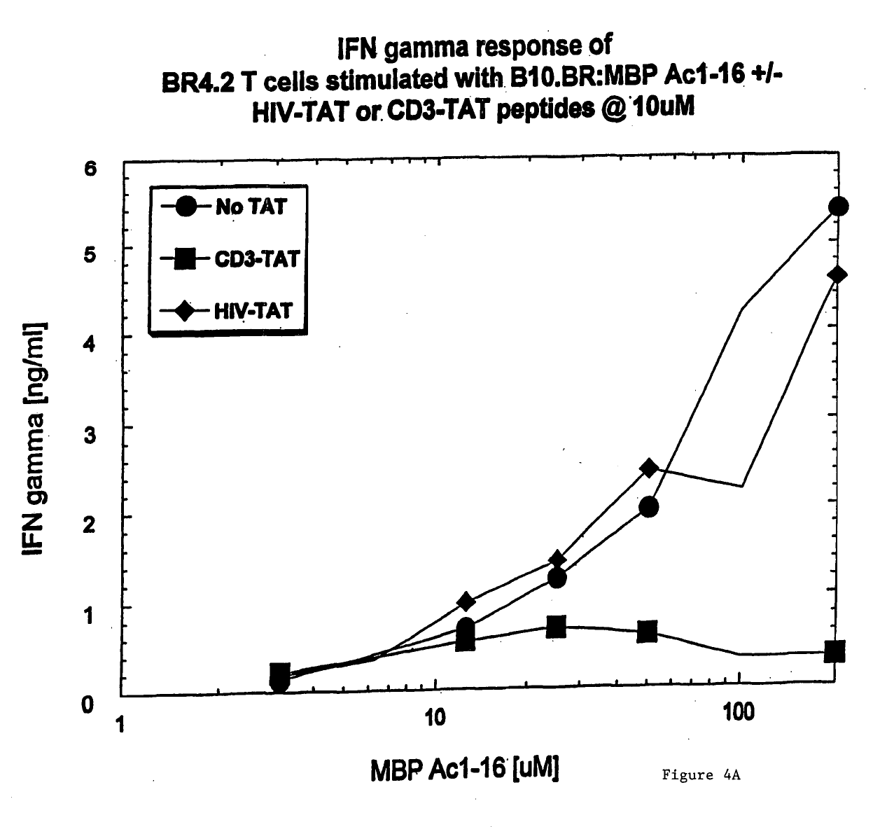

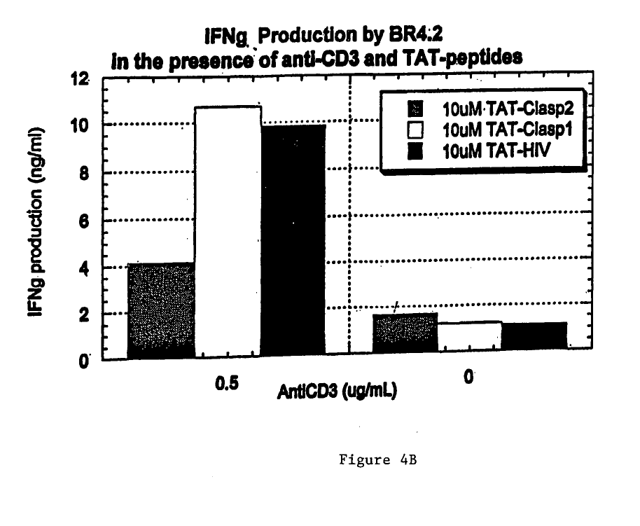

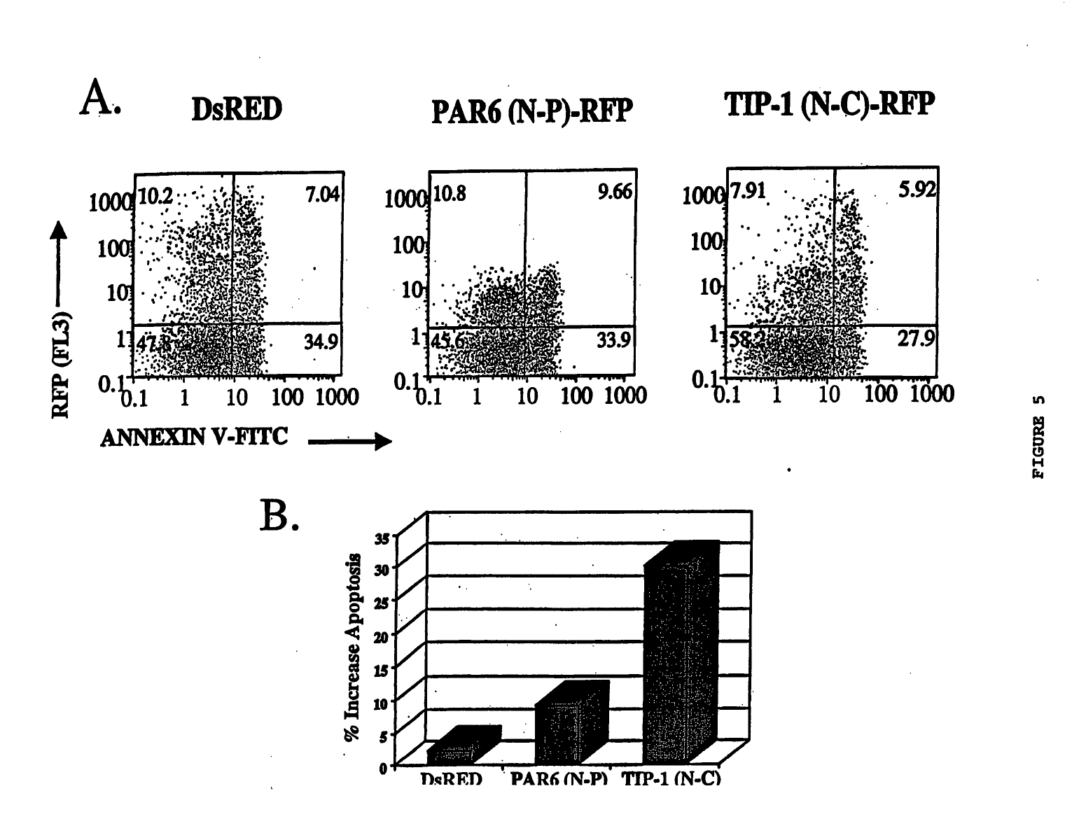

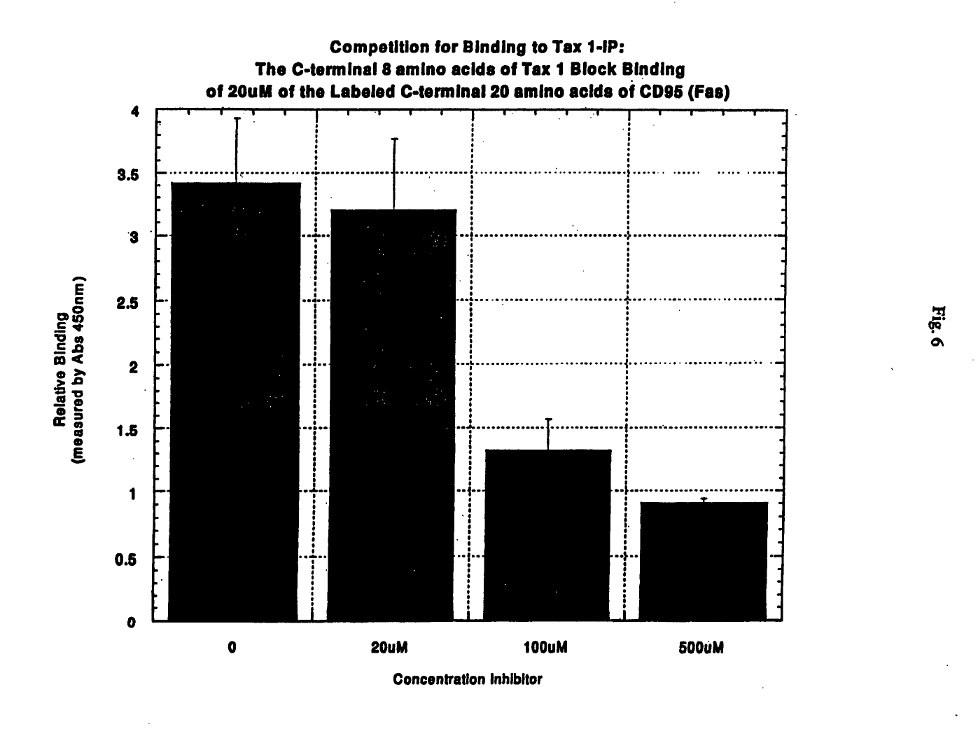

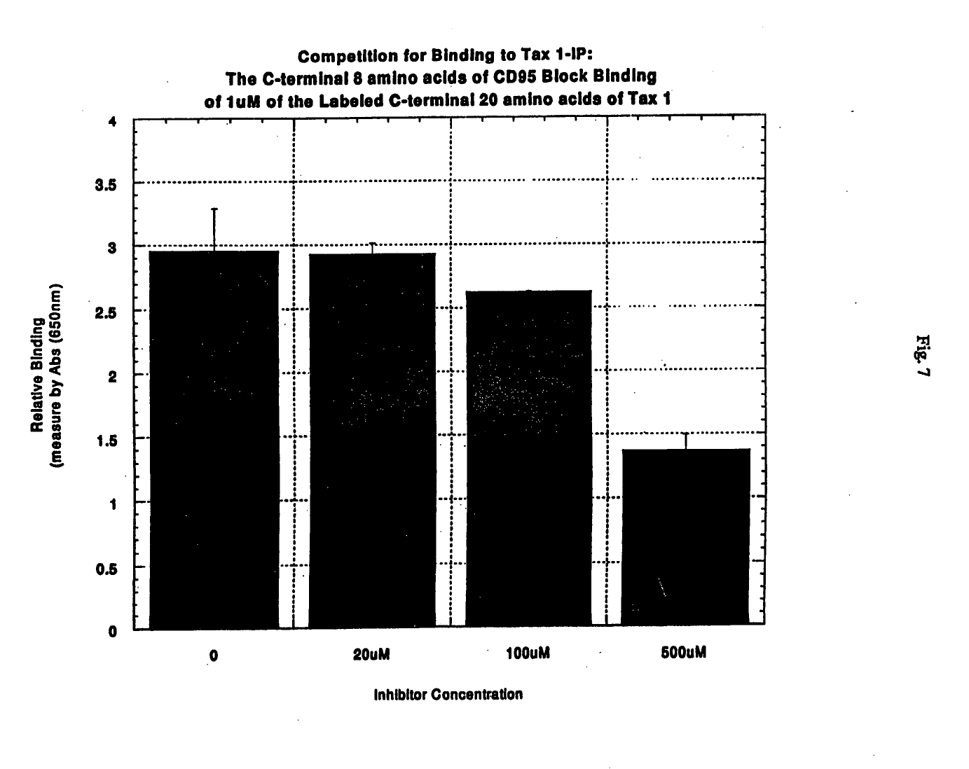

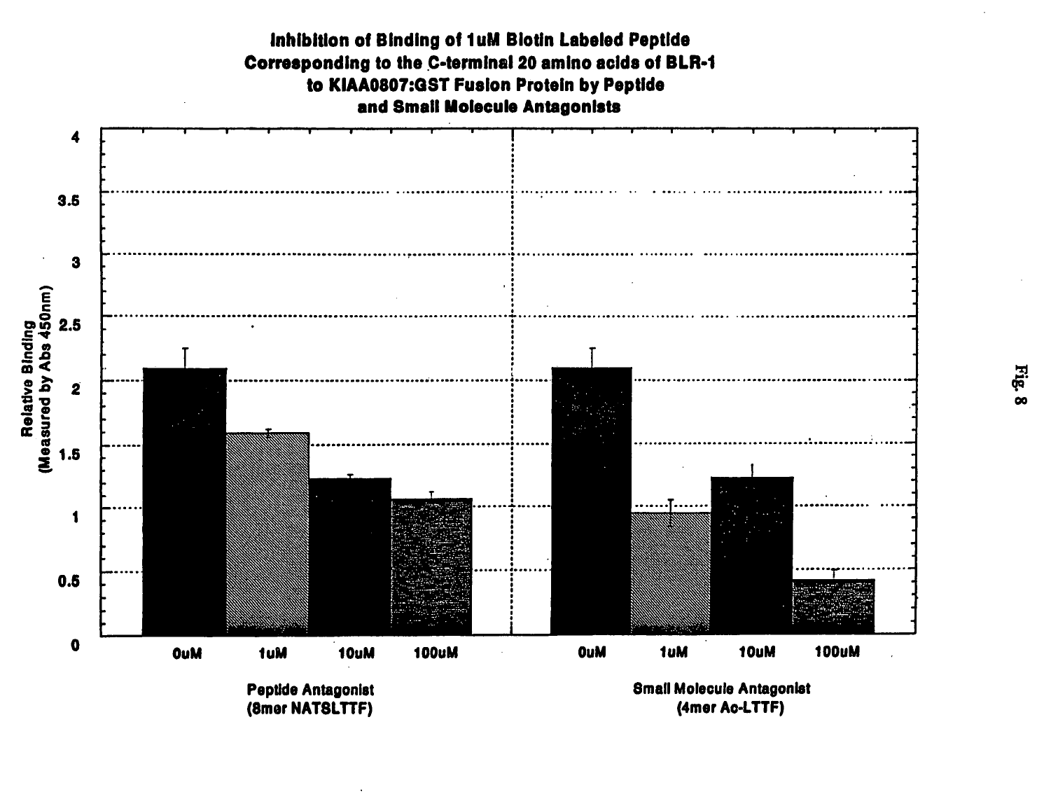

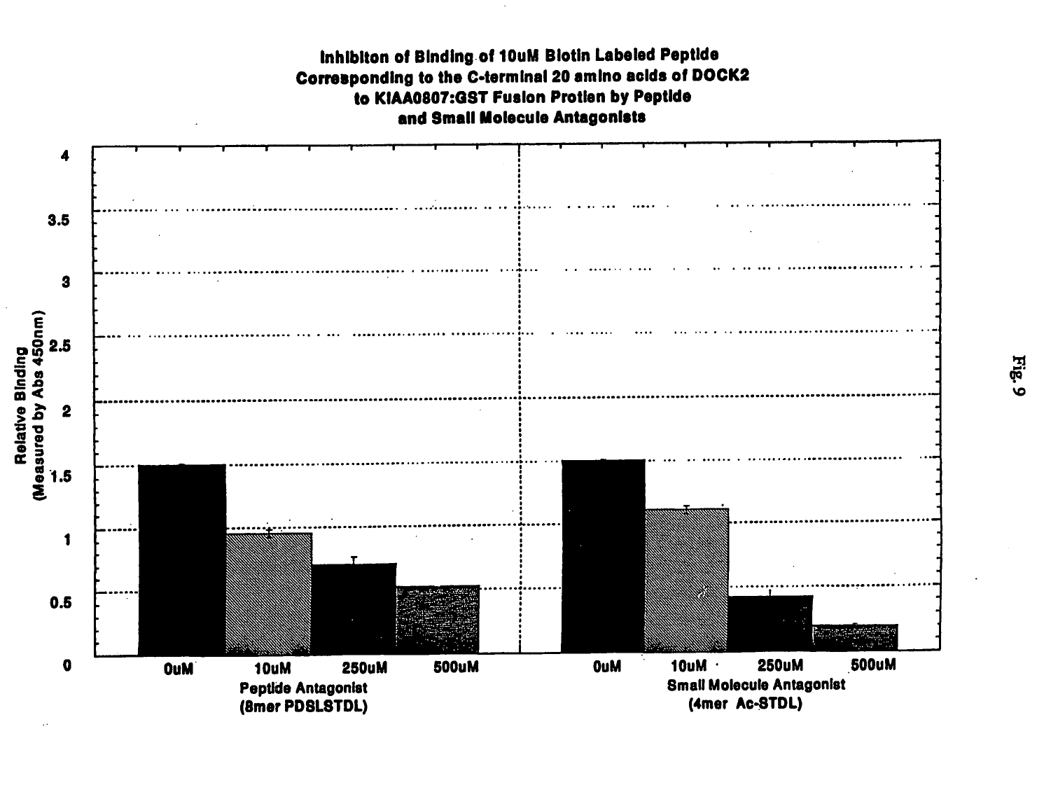

(4) Specific binding of a PL peptide and a PDZ domain polypeptide is determined by comparing the signal from the well(s) in which the PL peptide and PDZ domain polypeptide are combined, with the background signal(s). The background signal is the signal found in the negative control(s). Typically a specific or selective reaction will be at least twice background signal, more typically more than 5 times background, and most typically 10 or more times the background signal. In addition, a statistically significant reaction will involve multiple measurements of the reaction with the signal and the background differing by at least two standard errors, more typically four standard errors, and most typically six or more standard errors. Correspondingly, a statistical test (e.g. a T-test) comparing repeated measurements of the signal with -repeated measurements of the background will result in a p-value <0.05, more typically a p-value <0.01, and most typically a p-value <0.001 or less. As noted, in an embodiment of the “G” assay, the signal from binding of a given PL peptide to immobilized (surface bound) GST polypeptide alone is one suitable negative control (sometimes referred to as “B1”). Because all measurement are done in multiples (i.e. at least duplicate) the arithmetic mean (or, equivalently, average.) of several measurements is used in determining the binding, and the standard error of the mean is used in determining the probable error in the measurement of the binding. The standard error of the mean of N measurements equals the square root of the following: the sum of the squares of the difference between each measurement and the mean, divided by the product of (N) and (N−1). Thus, in one embodiment, specific binding of the PDZ protein to the platebound peptide is determined by comparing the mean signal (“mean S”) and standard error of the signal (“SE”) for a particular PL-PDZ combination with the mean B1. In experiments summarized in TABLE 2, binding was determined to be specific (denoted by a “G” in the matrix) when (1) the mean S was at least twice the mean B1 and (2) the mean S was at least six standard errors (six SE) greater than the mean B1. Results of exemplary “G” assays are shown in FIGS. 1A-1D.

6.2.3.3 “G′ assay” and “G″ assay”

Two specific modifications of the specific conditions described supra (§6.2.3.2) for the “G assay” are particularly useful. The modified assays use lesser quantities of labeled PL peptide and have slightly different biochemical requirements for detection of PDZ-ligand binding compared to the specific assay conditions described supra.

For convenience, the assay conditions described in this section are referred to as the “G′ assay” and the “G″ assay,” with the specific conditions described in §6.2.3.2 being referred to as the “G⁰ assay.” The “G′ assay” is identical to the “G⁰ assay” except at step (2) the peptide concentration is 10 uM instead of 20 uM. This results in slightly lower sensitivity for detection of interactions with low affinity and/or rapid dissociation rate. Correspondingly, it slightly increases the certainty that detected interactions are of sufficient affinity and half-life to be of biological importance and useful therapeutic targets.

The “G″ assay” is identical to the “G⁰ assay” except that at step (2) the peptide concentration is 1 uM instead of 20 uM and the incubation is performed for 60 minutes at 25° C. (rather than, e.g., 10 minutes at 4° C. followed by 20 minutes at 25° C.). This results in lower sensitivity for interactions of low affinity, rapid dissociation rate, and/or affinity that is less at 25° C. than at 4° C. Interactions will have lower affinity at 25° C. than at 4° C. if (as we have to be generally true for PDZ-ligand binding) the reaction entropy is negative (i.e. the entropy of the products is less than the entropy of the reactants). In contrast, the PDZ-PL binding signal may be similar in the “G″ assay” and the “G⁰ assay” for interactions of slow association and dissociation rate, as the PDZ-PL complex will accumulate during the longer incubation of the “G″ assay.” Thus comparison of results of the “G″ assay” and the “G⁰ assay” can be used to estimate the relative entropies, enthalpies, and kinetics of different PDZ-PL interactions. (Entropies and enthalpies are related to binding affinity by the equations delta G=RT In (Kd)=delta H−T delta S where delta G, H, and S are the reaction free energy, enthalpy, and entropy respectively, T is the temperature in degrees Kelvin, R is the gas constant, and Kd is the equilibrium dissociation constant). In particular, interactions that are detected only or much more strongly in the “G⁰ assay” generally have a rapid dissociation rate at 25° C. (t1/2<10 minutes) and a negative reaction entropy, while interactions that are detected similarly strongly in the “G″ assay” generally have a slower dissociation rate at 25° C. (t1/2>10 minutes). Rough estimation of the thermodynamics and kinetics of PDZ-PL interactions (as can be achieved via comparison of results of the “G⁰ assay” versus the “G″ assay” as outlined supra) can be used in the design of efficient inhibitors of the interactions. For example, a small molecule inhibitor based on the chemical structure of a PL that dissociates slowly from a given PDZ domain (as evidenced by similar binding in the “G″ assay” as in the “G⁰ assay”) may itself dissociate slowly and thus be of high affinity.

In this manner, variation of the temperature and duration of step (2) of the “G assay” can be used to provide insight into the kinetics and thermodynamics of the PDZ-Iigand binding reaction and into design of inhibitors of the reaction.

6.2.4 Assay Variations

As discussed supra, it will be appreciated that many of the steps in the above-described assays can be varied, for example, various substrates can be used for binding the PL and PDZ-containing proteins; different types of PDZ containing fusion proteins can be used; different labels for detecting PDZ/PL interactions can be employed; and different ways of detection can be used.

The PDZ-PL detection assays can employ a variety of surfaces to bind the PL and PDZ-containing proteins. For example, a surface can be an “assay plate” which is formed from a material (e.g. polystyrene) which optimizes adherence of either the PL protein or PDZ-containing protein thereto. Generally, the individual wells of the assay plate will have a high surface area to volume ratio and therefore a suitable shape is a flat bottom well (where the proteins of the assays are adherent). Other surfaces include, but are not limited to, polystyrene or glass beads, polystyrene or glass slides, and alike.

For example, the assay plate can be a “microtiter” plate. The term “microtiter” plate when used herein refers to a multiwell assay plate, e.g., having between about 30 to 200 individual wells, usually 96 wells. Alternatively, high density arrays can be used. Often, the individual wells of the microtiter plate will hold a maximum volume of about 250 ul. Conveniently, the assay plate is a 96 well polystyrene plate (such as that sold by Becton. Dickinson Labware, Lincoln Park, N.J.), which allows for automation and high throughput screening. Other surfaces include polystyrene microtiter ELISA plates such as that sold by Nune Maxisorp, Inter Med, Dernark. Often, about 50 ul to 300 ul, more preferably 100 ul to 200 ul, of an aqueous sample comprising buffers suspended therein will be added to each well of the assay plate.

The detectable labels of the invention can be any detectable compound or composition which is conjugated directly or indirectly with a molecule (such as described above). The label can be detectable by itself (e.g., radioisotope labels or fluorescent labels) or, in the case of an enzymatic label, can catalyze a chemical alteration of a substrate compound or composition which is detectable. The preferred label is an enzymatic one which catalyzes a color change of a non-radioactive color reagent.

Sometimes, the label is indirectly conjugated with the antibody. One of skill is aware of various techniques for indirect conjugation. For example, the antibody can be conjugated with biotin and any of the categories of labels mentioned above can be conjugated with avidin, or vice versa (see also “A” and “G” assay above). Biotin binds selectively to avidin and thus, the label can be conjugated with the antibody in this indirect manner. See, Ausubel, supra, for a review of techniques involving biotin-avidin conjugation and similar assays. Alternatively, to achieve indirect conjugation of the label with the antibody, the antibody is conjugated with a small hapten (e.g. digoxin) and one of the different types of labels mentioned above is conjugated with an anti-hapten antibody (e.g. anti-digoxin antibody). Thus, indirect conjugation of the label with the antibody can be achieved.

Assay variations can include different washing steps. By “washing” is meant exposing the solid phase to an aqueous solution (usually a buffer or cell culture media) in such a way that unbound material (e.g., non-adhering cells, non-adhering capture agent, unbound ligand, receptor, receptor construct, cell lysate, or HRP antibody) is removed therefrom. To reduce background noise, it is convenient to include a detergent (e.g., Triton X) in the washing solution. Usually, the aqueous washing solution is decanted from the wells of the assay plate following washing. Conveniently, washing can be achieved using an automated washing device. Sometimes, several washing steps (e.g., between about 1 to 10 washing steps) can be required.

Various buffers can also be used in PDZ-PL detection assays. For example, various blocking buffers can be used to reduce assay background. The term “blocking buffer” refers to an aqueous, pH buffered solution containing at least one blocking compound which is able to bind to exposed surfaces of the substrate which are not coated with a PL or PDZ-containing protein. The blocking compound is normally a protein such as bovine serum albumin (BSA), gelatin, casein or milk powder and does not cross-react with any of the reagents in the assay. The block buffer is generally provided at a pH between about 7 to 7.5 and suitable buffering agents include phosphate and TRIS.

Various enzyme-substrate combinations can also be utilized in detecting PDZ-PL interactions. Examples of enzyme-substrate combinations include, for example:
(i) Horseradish peroxidase (HRPO) with hydrogen peroxidase as a substrate, wherein the hydrogen peroxidase oxidizes a dye precursor (e.g. orthophenylene diamine [OPD] or 3,3′,5,5′-tetramethyl benzidine hydrochloride [TMB]) (as described above).
(ii) alkaline phosphatase (AP) with para-Nitrophenyl phosphate as chromogenic substrate.
(iii) β-D-galactosidase (β D-Gal) with a chromogenic substrate (e.g. p-nitrophenyl-β-D-galactosidase) or fluorogenic substrate 4-methylumbelliferyl-β-D-galactosidase.

Numerous other enzyme-substrate combinations are available to those skilled in the art. For a general review of these, see U.S. Pat. Nos. 4,275,149 and 4,318,980, both of which are herein incorporated by reference.

Further, it will be appreciated that, although, for convenience, the present discussion primarily refers antagonists of PDZ-PL interactions, agonists of PDZ-PL interactions can be identified using the methods disclosed herein or readily apparent variations thereof.

6.2.5 Results of PDL-PL Interaction Assays

TABLE 2, supra, shows the results of assays in which. specific binding was detected using the “A” and “G” assays described herein. The top row of the table specifies the source of the PDZ domain used in the GST-PDZ fusion proteins (see TABLE 3). The first column lists the cell surface proteins from which C-terminal peptide sequences were derived and the second column (“code”) identifies the peptide used in the assay (see TABLE 4). The third column, “Seq” provides the sequence of the four (4) C-terminal residues of the cell surface protein and peptide. In the matrix, “A” indicates specific binding as detected in the “A” assay. “G” indicates specific binding as shown in the “G” assay. A blank indicates that no specific binding was detected using the “A” or “G” assays. An asterisk (*) indicates that a pairwise interaction between the PDZ protein and the cell surface protein (or subdomains of either) has been described by others.

6.2.5.1 New PL Motifs

As noted supra, TABLE 2 shows the results of assays (referred to as “PRISM MATRIX”) to detect binding between PDZ proteins and candidate-PL peptides. A number of specific PDZ-PL interactions are identified by the MATRIX and key amino acids and positions important in PDZ binding (“PL motifs”) are deduced from these results. Not only is the MATRIX useful to catalog comprehensively PDZ-PL binding combinations, the assay can further aid in the rapid discovery and characterization of novel PL proteins and PL motifs to help in rational drug design and synthesis of PL-PDZ interaction inhibitors.

Other investigators have reported certain PL motifs important in PDZ binding, e.g., the C-terminal motifs S/T-X-V/I/L (for DLG1) and Y/F-Y/F-I/L/F for MPP1 (see, Doyle et al., 1996, Cell 85, 1067; Songyang et al., 1997, Science 275, 73). However, the reported motifs are not sufficiently specific (i.e. a large number of proteins meet these criteria yet are not necessarily actual PDZ ligands) and cover only a small number of PDZ proteins (approximately 10). The PRISM MATRIX can be used to determine ligand specificity and to deduce ligand binding motifs for any PDZ protein because it can precisely determine sequences of amnino acids that do or do not result in specific PDZ binding. In addition, the assay has revealed a significant of new PDZ domain binding motifs (i.e. PL motifs): C-terminal sequence of CD6, ISAA (SEQ ID NO: 14); C-terminal sequence of CD49E, TSDA (SEQ ID NO: 24); C-terminal sequence of CD49F, TSDA (SEQ ID NO: 24); C-terminal sequence of Clasp-1, SAEV (SEQ ID NO: 175); C-terminal sequence of CLASP-4, YAEV (SEQ ID NO: 192); C-terminal sequence of CD44, KIGV (SEQ ID NO: 104); C-terminal sequence of Fas Ligand, LYKL (SEQ ID NO: __); C-terminal sequence of IL5R, DSVF (SEQ ID NO: 94); C-terminal sequence of BLR-1, LTTF (SEQ ID NO: 217). Identification of these novel PL sequences allows the definition of novel PL motifs (See TABLE 5A, infra). The specificity with which these novel motifs are defined is enhanced by the fact that the MATRIX reports both positive results (i.e. PDZ-PL) combinations that result in specific binding interactions) and negative results (i.e. PDZ-PL combinations that do not result in specific binding). For example, the C-terminal sequence of CD6, SAA and the C-terminal sequence of CD49E, SDA bind to the PDZ-domain polypeptide 41.8 while the related C-terminal sequence of CD 166, TEA and C-terminal sequence of CD148, YIA do not. This identifies the novel PL motif (Motif 1, infra) of polypeptides terminating in alanine with serine at the −2 position and excludes polypeptides with threonine and tyrosine at the −2 position. This motif is therefore more specific than most previously identified motifs. Other novel motifs are described in TABLE 5.

TABLE 5

| | Position: | | | |
|---|---|---|---|---|
| | −3 | −2 | −1 | C-terminal |
| Motif 1 | X | S | X | A |
| Motif 2 | X | A | D/E | V |
| Motif 3 | X | V/I/L | X* | V |
| Motif 4 | X | S/T | X | F |
| Motif 5 | X | Y | X* | L |

X* is any non-aromatic amino acid (any residue other than Y, F or W).

6.2.5.2 PDZ-Specific PL Motifs

The invention provides a method for identifying a PDZ-domain binding protein (PDZ ligand or PL) that binds to a specified PDZ protein. According to the method, a plurality of putative PL peptides (e.g., peptides or polypeptides that include at or near their carboxy terminus a sequence of the C-terminus of a naturally occurring protein known or suspected of being a PL protein, i.e., binding to at least one PDZ domain) are provided. Binding assays, typically the A and G assays as described elsewhere herein, are carried out to identify peptides that do and do not bind the specified PDZ protein (e.g., by detecting binding to a PDZ domain sequence from the specified PDZ protein). Usually, a large number of putative PL peptides are screened, as shown in Table 2. Thus, typically at least 2, more often at least 3, PLs that bind the specified PDZ are identified, and typically at least a plurality (e.g., in this context, at least 10, more often at least 20, and typically at least 40 or more) PLs that do not bind the PDZ are identified.

The sequences of the binding and nonbinding peptides are compared, for example as described infra, and a motif(s) characteristic of peptides that bind the PDZ domain sequence and not characteristic of peptides that do not bind the PDZ domain sequence is determined. To identify a PL protein that binds the specified PDZ protein, known proteins are examined to identify sequences (typically at or near the c-terminus, e.g., within 1, 2 or 3 residues of the c-terminus) that match the motif identified. In addition, the search parameters may include other characteristics of the protein sequences being searched, such as an expression property (e.g., expression in a particular cell type, e.g., lymphocytes, or disease state), a functional property (e.g., receptor activity), and/or a structural property (e.g., similarity to a reference sequence). Usually this identification is carried out, at least in part, by a computer-implemented search of a database such as GenBank for proteins having the specified motif, although comparison can be made manually, particularly when the search is limited to a specific class of putative PLs.

In embodiments, the assay also includes the further step of characterizing or confirming the binding properties of the identified PL(s) and PDZ, typically by carrying out in vivo or in vitro binding assays described herein (e.g., the G assay) or known in the art (e.g., precipitation assays).

In an embodiment, a PRISM MATRIX (i.e., the representation of PL-PDZ binding interactions, e.g., interactions occuring in lymphocytes, e.g., as shown in TABLE 2) is used to identify C-terminal peptide sequence motifs characteristic of PLs (i.e., sequences that mediate binding to a particular PDZ domain-containing protein).

In an embodiment, the MATRIX is specifically arranged to facilitate identification of these motifs. To this end, the PL ligands in the MATRIX shown in TABLE 2 are ordered on the basis of C-terminal amino acid similarity, with weight given to residues reported to be important in PDZ binding (Doyle et al., 1996, Cell 85, 1067). In particular, in TABLE 2, peptides are first ordered based on the most C-terminal residues (zero position) in the following amino acid order: G, A, C, S, T, N, Q, D, E, H, K, R, V, I, L, M, P, F, Y, W. Among peptides with identifical C-termini, the same raking scheme was then applied to the next most important residue for peptide binding, the −2 position, followed by the −1 position and the −3 position.

(The PDZ domains of each of the GST-PDZ fusion proteins in the MATRIX are also ordered based on amino acid sequence similarity, in this case based on multiple sequence alignment using the CLUSTAL software package. In an alternative approach, the GST-PDZ fusions can also be arranged to give additional weight in alignment to residues known in the art to be important for ligand binding.)

Based on the peptide ligands being ordered in this structure-based manner, one approach to obtaining PDZ-specific PL motifs from the MATRIX is as follows:
1. Begin by examining a single column in the MATRIX (corresponding to a single PDZ protein).
2. Determine if there exist within this column one or a few areas in which several interactions are found, and other areas in which few or no interactions are found. For example, reading down the column of TABLE 2 corresponding to the PDZ protein DLG1, there are many interactions in the region starting with the ligand CLASP-1 and the ending with the ligand CD34, and no interactions outside of this region.
3. If such region(s) are found, note the amino acid(s) present at the C-terminus in the region. These amino acids constitute the motif at the C-terminus. For example, for the PDZ protein DLG1, the C-terminal amino acids from ligand CLASP-1 to ligand CD34 range from V to L (including positive interactions with ligands terminating in I, the amino acid separating V and L in the amino acid order supra). Thus in the C-terminal motif for DLG1 is V/I/L.
4. To define a preliminary motif at the −2 position, identify a subdivision of the overall region, such that the subdivision contains several interactions and is surrounded by areas of no or few interactions. Note the amino acid(s) present at the −2 position in the subdivision. For example, in the case of DLG1, one subdivision of many interactions is from the ligand CDW128A to the ligand DOCK2. The amino acid(s) present at the −2 position in this subdivision are S and T (adjacent in the amino acid ordering), defining the −2 position motif S/T.
5. To confirm the preliminary −2 position motif, test if it generalizes to most or all of the interactions found in the column. For example, in the case of DLG1, this process reveals that most ligands matching the C-terminal motif and the preliminary −2 position motif S/T do give detectable interactions. However, some positive interactions are also found with ligands having A, E, or Y at the −2 position. Thus a more general DLG1 −2 position motif is S/T/A/E/Y.
6. To define a preliminary motif at the −1 position, it is often helpful to identify vertically adjacent boxes which contain different results (i.e. one box contains interactions, the other does not). Frequently, these adjacent boxes will correspond to ligands that are identical at the C-terminal and −2 positions, but differ at the −1 position. If this is true, a motif is considered in which the amino acid found at the −1 position in the ligand that interacts with the PDZ protein is preferred at the −1 position of the motif, while the amino acid found at the −1 position in the ligand giving no interactions is disfavored in the motif. This process is repeated for all available such adjacent boxes. For example, in the case of DLG1, one appropriate set of adjacent boxes is CD97 versus CD38, with the preferred −1 position amino acid G and the disfavored E.
7. To confirm the −1 position motif, the set of preferred versus disfavored amino acids at the −1 position, identified in step 6 supra, is examined. If these amino acids fall into logical structural categories, e.g. the preferred amino acids are all hydrophilic while the disfavored amino acids are all hydrophobic, this confirms a −1 position motif. In the case of DLG1, preferred amino acids include R, E, and G, and disfavored include R and E. Since these sets overlap, there appears to be no −1 position motif.
8. Analogous steps to those preformed for the −1 position motif may also be preformed for the −3 position motif.

It will be apparent that the power of this analysis (or a computer implemented variation of this algorithm) increases with the size of the matrix (e.g., the number of PLs and PDZs tested). In TABLE 2, the PDZ domain-containing proteins (including MPP 1, K807, DLG1, PSD95, NeDLG, 41 kd, WWP3) bind a sufficient number of ligands in the current MATRIX for the above algorithm to be practical.

For PDZs that bind a fewer number of ligands in the MATRIX, an alternative approach is useful:
1. List the set of amino acids found at the −3, −2, −1, and C-terminal positions in ligands that do bind to the PDZ. For example, in the case of AF6, the set at the C-terminal position is {V, V, V, V, I}, at the −1 position {E, F, R, Y, F}, at the −2 position {A, S, T, Y, Y}, at the −3 position {Y, V, K, E, E}.
2. Examining the set at the C-terminal position for chemical similarity. Define one or more motifs based on observed chemical similarity at this position. For example, in the case of AF6, the set IV, V, V, V, I} strongly suggests a motif V/I/L, as these are all of the mid-sized, aliphatic, hydrophobic amino acids.
3. Examine the other positions similarly. For example, for AF6, the set {E, F, R, Y, F} at the −1 position suggests a −1 position motif F[Y as these are both single ring, aromatic amino acids. A less stringent motif F/Y/D/E/K/R is also suggested, that would include all of the single ring, aromatic amino acids as well as all of the charged amino acids. However, it is unclear why these chemically dissimilar groups of amino acids—including positively charged, negatively charged, and hydrophobic amino acids—would each be preferred at this position. At the −2 position, possible motifs are S/T/Y (all hydroxylated amino acids) and the less stringent motif A/S/T/Y. At the −3 position, possible motifs are D/E/K/R (all charged amino acids) or X (any amino acid) (as the set {Y, V, K, E, E} does not have a clear structural basis).
4. Revise the motifs at each position based on ligands that failed to bind to this PDZ. To do this, assemble a net motif using the least restrictive proposed motif at each position. Next determine how many ligands fitting this least restrictive overall motif fail to bind to this PDZ. Then see if the more restrictive proposed motif at each position would succeed in preventing these ligands from being predicted to bind. If so, the more restrictive motif is adopted. For example, for AF6 the least restrictive net motif (based on the above) is X-A/S/T/Y-F/Y/D/E/K/R-V/I/L. Fifteen different ligands in TABLE 2 (ten of which do not bind AF6) are included by this motif. Restricting the motif to F/Y at the −1 position eliminates all of the false predictions, while eliminating a minority (two of five) of the accurate predictions. As noted above, this also results in a more logical structural basis for the motif. Thus X-A/S/T/Y-F/Y-V/I/L appears to be a promising motif.
5. Check whether the promising motif includes accurately predicts all of the “strong” interactions. While every interaction in the MATRIX indicates a statistically significant interaction, not every interaction is of equal strength. (In this context, “strength” refers to the affinity/half-life of the interaction, e.g., as determined by the magnitude of the binding signal in the “G assay.”) In general, “strong” interactions result in positive interactions being detected in multiple assay formats (i.e. A, G, G′, and G″) and particularly in the “G′ or G” assays” (as these use the lower ligand concentration tand the G assay). For example, for AF6 the “strongest” interaction appears to be with Spectrin beta. Importantly, this ligand conforms to the promising motif supra. Therefore, this motif can be adopted.

PDZ domain-containing proteins in the MATRIX that bind to only one or two ligands provide a special challenge for defining PL motifs. However, in certain cases the combination of a PDZ domain binding to even a single ligand combined with the failure of that PDZ to bind related ligands allows prediction of a motif specific to that PDZ. Thus, the following process can be applied to define a PDZ-specific ligand motif based on only a single ligand that binds to that PDZ:
1. Let a preliminary guess at the C-terminal motif be X-X1X-X2, where X1 and X2 refer to amino acids chemically similar or identical to the amino acid found at the −2 position and zero positions respectively of the ligand that is known to bind (X is any amino acid). For example, in the case of K545 the C-terminal sequence known to bind is that of CD105, SSMA, therefore the preliminary motif is X-A/S/T/Y-X-A/S/V.
2. Place limits on the motif, such that ligands located immediately above and below the ligand that is known to bind (but do not themselves bind) are excluded from the motif In the case of K545, this means excluding the ligand CD49F (C-terminus TSDA) which is most easily accomplished by placing a limit on the −1 position (the zero and −2 positions are identical to SSMA, and the −3 position is very similar). A logical limit on the −1 position is to hydrophobic amino acids.
3. Check the more limited motif's ability to exclude other ligands that do not bind to the PDZ of interest. Add more restrictions as necessary, comparing the results of restrictions at different ligand positions as one proceeds. For example, in the case of K545, eight ligands that failed to bind K545 fit even the more restrictive motif. There were several possible ways to restrict the motif, e.g. by eliminating SN at the zero position, A/Y at the −2 position, hydrophobic residues other than M at the −1 position, or residues other than A/S/T/Y at the −3 position. Of these, eliminating residues other than M at the −1 position is the only single change that is sufficient at eliminating all ligands that fail to bind K545. Therefore, one sufficient motif (i.e. that only predicts the interaction which occurs) is X-A/S/T/Y-M-A/S/V.

It is important to note that motifs created based on a single ligand that binds using the above steps will often be too broad or too narrow. For example, in the case of K545 it is unclear if G or SN are tolerated at the zero position.

As will be apparent to one skilled in the art, the above algorithms are not mutually exclusive. Principles of each algorithm can be applied by one of experience in peptide chemistry and/or structural biology in unison to derive motifs somewhat superior to those that result from application of any one of these above algorithms in isolation. Motifs derived in this manner are provided in TABLE 6.

PDZ-specific PL motifs derived from the MATRIX can be applied to search genomic databases for other ligands (i.e. ligands not already in the MATRIX) that bind to a specific PDZ protein. Such searches can be performed using publicly available software such as BLAST (available at www.ncbi.nlm.nih.gov). Knowledge of these other ligands is of practical value in several respects:
1. It permits (based on knowledge of the function of these other ligands from the literature) prediction of desirable pharmacological effects of compounds that bind to the specified PDZ domain. Thereby it allows design of compounds that have these desirable pharmacological functions.
2. It permits (based on knowledge of the function of these other ligands from the literature) prediction of undesirable pharmacological effects of compounds that bind to the specified PDZ domain. Thereby it allows prediction of side effects of pharmaceutical compounds and thus facilitates design of pharmaceutical compounds without these side effects.
3. It permits biochemical assays to be performed that directly demonstrate the binding of these other ligands to the specified PDZ. Furthermore, it allows biochemical tests to be performed that determine the ability of pharmaceuticals or candidate pharmaceuticals to inhibit (or augment) differentially the binding of different ligands to the specified PDZ. Thus, knowledge of these others ligands allows drug screens to be performed for pharmaceuticals that effect the binding of specified ones of these other ligands to the given PDZ, without effecting the binding of other ligands to the same PDZ. Thus, it allows the discovery of pharmaceuticals that inhibit only the interaction of specified PDZ-PL pairs, and thus have highly specific biological effects.
4. It permits prediction of macromolecular complexes composed of the specified PDZ domain-containing protein and ligands that bind to different domains of that protein.

Table 6 shows examplary PL motifs derived from the PRISM MATRIX according to the invention.

TABLE 6
PL Motifs

| PDZ | Motif | Comments |
|---|---|---|
| CASK | X-Y-X-V | Previously known |
| MPP1 | X-S/T/Y/I-X-V | |
| LIMK1 | X-S/T/Y-X-V | Several ligands fitting this motif do |
| | | not bind LIMK1 |
| K303 | X-S-X-V | Several ligands fitting this motif do |
| | | not bind K303 |
| K807 | X1-S/T-X2-V/I/L/F | L preferred to V at C-terminus based |
| | Preferred: X1 = D/S/T; | on strength of responses (data not |
| | X2 = D/E/N/Q/S/T | shown) |
| DLG1, | X-S/T/Y/A/E-X-V/I/L | Generally consistent with scientific |
| PSD95, | | literature; however, A/E also |
| NeDLG | | acceptable at −2 position |
| SNTa1 | X-S/T/Y-D/Y-V/I/L | Both residues (D/Y) preferred at −1 |
| | | position interact productively with |
| | | positively charged groups |
| TAX- | X1-S/T/Y-X-V/I | D may be equivalent to E at −3 |
| IP43 | Preferred: X1 = E | position; D/E may be preferred at −1 |
| | | position |
| LDP | X-A/S-X2-V/I | |
| | Preferred: X2 = E | |
| LIM | X-S/T-X2-A/V | Residues with a long, flexible side |
| | Preferred: X2 = M/R/K | chain are referred at −1 position, |
| MINT1 | X-A/S/T/I/Y-X- | Many ligands give high signals with |
| | V/I/L/F | this PDZ for unclear reasons; |
| K545 | X-A/S/T/Y-M-A/S/V | |
| TAX- | X-S-D/E-V | |
| IP2 | | |
| MPP2 | X-S/T/Y-X-A/V/I | Several ligands fitting this motif do |
| | | not bind MPP2 |
| TIP-1 | X-S/T-X2-V/I/L | Several ligands fitting this motif do |
| | Possibly preferred: | not bind TIP-1 |
| | X2 = D/E/N/Q | |
| PTN-4 | X1-S/T-X-V/F | Several ligands fitting this motif do |
| | Preferred: X1 = D/E | not bind PTN-4 |
| prIL16 | D/E/K/R-V/I/L/F/Y-X-V | |
| CBP | X-S/T-F/Y-V | |
| 41 | X-A/S/T/Y/F-X- | Small hydrophobic residue |
| | A/V/I/L | (especially V) preferred at C-terminus |
| AF6 | X-A/S/T/Y-F/Y-V/I/L | Preferred C-terminal sequence is F-V |
| RGS12 | X1-S/T/Y-X-V/F | |
| | Preferred: X1 = D/E | |
| PDZK1 | X-T-X-F | |
| DLG5 | X-S/T-X-V | Several ligands fitting this motif do |
| | | not bind DLG5 |
| Synt | X1-V/I/L-X2-V | |
| | Possibly preferred: | |
| | X1 = K/R; X2 = G | |
| WWP3 | X-S/T-X2-V | Both residues (F/R) preferred at −1 |
| | Preferred: X2 = F/R | position have extensive hydrophobic |
| | | portions |
| TAX- | X-Y-X-V | |
| IP40 | | |
| TIAM1 | NONE | Few interactions found |
| DVL1 | X-S/T/Y-X-V | Several ligands fitting this motif do |
| | | not bind DVL1 |
| K561 | X-S/T/Y-X-V/I/L/F | Several ligands fitting this motif do |
| | | not bind K561 |

Key:
Capital letter are amino acids in standard single letter code;
X refers to any amino acid;
X1 or X2 refers to any amino acid, but with a preference for the amino acids stated in the table.
Sequences in the motif column refer to the C-terminal four amino acids of the ligand binding to the PDZ stated in the left-most column of the table.
Ligand amino acid positions (starting from the −3 position) are separated by hyphens;
slashes separate various possible amino acids at a given position.

### 6.3 Measurement of PDZ-Ligand Binding Affinity

The “A” and “G” assays of the invention can be used to determine the “apparent affinity” of binding of a PDZ ligand peptide to a PDZ-domain polypeptide. Apparent affinity is determined based on the concentration of one molecule required to saturate the binding of a second molecule (e.g., the binding of a ligand to a receptor). Two particularly useful approaches for quantitation of apparent affinity of PDZ-ligand binding are provided infra.

(1) A GST/PDZ fusion protein, as well as GST alone as a negative control, are bound to a surface (e.g., a 96-well plate) and the surface blocked and washed as described supra for the “G” assay.

(2) 50 uL per well of a solution of biotinylated PL peptide (e.g. as shown in TABLE 4) is added to the surface in increasing concentrations in PBS/BSA (e.g. at 0.1 uM, 0.33 uM, 1 uM, 3.3 uM, 10 uM, 33 uM, and 100 uM). In one embodiment, the PL peptide is allowed to react with the bound GST/PDZ fusion protein (as well as the GST alone negative control) for 10 minutes at 4° C. followed by 20 minutes at 25° C. The plate is washed 3 times with ice cold PBS to remove unbound labeled peptide.

(3) The binding of the PL peptide to the immobilized PDZ-domain polypeptide is detected as described supra for the “G” assay.

(4) For each concentration of peptide, the net binding signal is determined by subtracting the binding of the peptide to GST alone from the binding of the peptide to the GST/PDZ fusion protein. The net binding signal is then plotted as a function of ligand concentration and the plot is fit (e.g. by using the Kaleidagraph software package curve fitting algorithm) to the following equation, where “Signal_{[ligand]}” is the net binding signal at PL peptide concentration “[ligand],” “Kd” is the apparent affinity of the binding event, and “Saturation Binding” is a constant determined by the curve fitting algorithm to optimize the fit to the experimental data:
Signal_{[ligand]}=Saturation Binding×([ligand]/([ligand]+Kd))

For reliable application of the above equation it is necessary that the highest peptide ligand concentration successfully tested experimentally be greater than, or at least similar to, the calculated Kd (equivalently, the maximum observed binding should be similar to the calculated saturation binding). In cases where satisfying the above criteria proves difficult, an alternative approach (infra) can be used.

The results obtained when using approach 1 are demonstrated in FIGS. 2A and 2B. FIG. 2 shows varying concentrations of biotinylated CLASP-2 (FIG. 2A) or Fas (FIG. 2B). C-terminal peptides reacted with immobilized (plate bound) GST polypeptide or GST/PDZ fusion proteins (GST/DLG1, GST/NeDLG, and GDT/PSD95) in duplicate. The signals were normalized, plotted and fit to a saturation binding curve, yielding an apparent affinity of 21 uM for DLG1-CLASP-2 interaction, 7.5 uM for NeDLG-CLASP-2 interaction, 45 uM for PSD95-CLASP-2 interaction, and 54 uM for DLG1-Fas interaction, 54 uM for NeDLG-Fas interaction, and 85 uM for PSD95-Fas interaction.

Approach 2:

(1) A fixed concentration of a PDZ-domain polypeptide and increasing concentrations of a labeled PL peptide (labeled with, for example, biotin or fluorescein, see TABLE 4 for representative peptide amino acid sequences) are mixed together in solution and allowed to react. In one embodiment, preferred peptide concentrations are 0.1 uM, 1 uM, 10 uM, 100 uM, 1 mM. In various embodiments, appropriate reaction times can range from 10 minutes to 2 days at temperatures ranging from 4° C. to 37° C. In some embodiments, the identical reaction can also be carried out using a non-PDZ domain-containing protein as a control (e.g., if the PDZ-domain polypeptide is fusion protein, the fusion partner can be used).

(2) PDZ-ligand complexes can be separated from unbound labeled peptide using a variety of methods known in the art. For example, the complexes can be separated using higb performance size-exclusion chromatography (HPSEC, gel filtration) (Rabinowitz et al., 1998, Immunity 9:699), affinity chromatography(e.g. using glutathione Sepharose beads), and affinity absorption (e.g., by binding to an anti-GST-coated plate as described supra).

(3) The PDZ-ligand complex is detected based on presence of the label on the peptide ligand using a variety of methods and detectors known to one of skill in the art. For example, if the label is fluorescein and the separation is achieved using HPSEC, an in-line fluorescence detector can be used. The binding can also be detected as described supra for the G assay.

(4) The PDZ-ligand binding signal is plotted as a function of ligand concentration and the plot is fit. (e.g., by using the Kaleidagraph software package curve fitting algorithm) to the following equation, where “Signal_{[ligand]}” is the binding signal at PL peptide concentration “[ligand],” “Kd” is the apparent affinity of the binding event, and “Saturation Binding” is a constant determined by the curve fitting algorithm to optimize the fit to the experimental data:
Signal_{[Ligand]}=Saturation Binding×([ligand]/([ligand+Kd])

Measurement of the affinity of a labeled peptide ligand binding to a PDZ-domain polypeptide n is useful because knowledge of the affinity (or apparent affinity) of this interaction allows rational design of inhibitors of the interaction with known potency (See EXAMPLE 2). The potency of inhibitors in inhibition would be similar to (i.e. within one-order of magnitude of) the apparent affinity of the labeled peptide ligand binding to the PDZ-domain.

Thus, in one aspect, the invention provides a method of determining the apparent affinity of binding between a PDZ domain and a ligand by immobilizing a polypeptide comprising the PDZ domain and a non-PDZ domain on a surface, contacting the immobilized polypeptide with a plurality of different concentrations of the ligand, determining the amount of binding of the ligand to the immobilized polypeptide at each of the concentrations of ligand, and calculating the apparent affinity of the binding based on that data. Typically, the polypeptide comprising the PDZ domain and a non-PDZ domain is a fusion protein. In one embodiment, the e.g., fusion protein is GST-PDZ fusion protein, but other polypeptides can also be used (e.g., a fusion protein including a PDZ domain and any of a variety of epitope tags, biotinylation signals and the like) so long as the polypeptide can be immobilized In an orientation that does not abolish the ligand binding properties of the PDZ domain, e.g, by tethering the polypeptide to the surface via the non-PDZ domain via an anti-domain antibody and leaving the PDZ domain as the free end. It was discovered, for example, reacting a PDZ-GST fusion polypeptide directly to a plastic plate provided suboptimal results. The calculation of binding affinity itself can be determined using any suitable equation (e.g., as shown supra; also see Cantor and Schimmel (1980) BIOPHYSICAL CHEMISTRY W H Freeman & Co., San Francisco) or software.

Thus, in a preferred embodiment, the polypeptide is immobilized by binding the polypeptide to an immobilized immunoglobulin that binds the non-PDZ domain (e.g., an anti-GST antibody when a GST-PDZ fusion polypeptide is used). In a preferred embodiment, the step of contacting the ligand and PDZ-domain polypeptide is carried out under the conditions provided supra in the description of the “G” assay. It will be appreciated that binding assays are conveniently carried out in multiwell plates (e.g., 24-well, 96-well plates, or 384 well plates).

The present method has considerable advantages over other methods for measuring binding affinities PDZ-PL affinities, which typically involve contacting varying concentrations of a GST-PDZ fusion protein to a ligand-coated surface. For example, some previously described methods for determining affinity (e.g., using immobilized ligand and GST-PDZ protein in solution) did not account for oligomerization state of the fusion proteins used, resulting in potential errors of more than an order of magnitude.

Although not sufficient for quantitative measurement of PDZ-PL binding affinity, an estimate of the relative strength of binding of different PDZ-PL pairs can be made based on the absolute magnitude of the signals observed in the “G assay.” This estimate will reflect several factors, including biologically relevant aspects of the interaction, including the affinity and the dissociation rate. For comparisons of different ligands binding to a given PDZ domain-containing protein, differences in absolute binding signal likely relate primarily to the affinity and/or dissociation rate of the interactions of interest.

### 6.4 Assays to Identify Novel PDZ Domain Binding Moieties and to Identify Inhibitors of PDZ Protein-PL Protein Binding

Although described supra primarily in terms of identifying interactions between PDZ-domain polypeptides and PL proteins, the assays described supra and other assays can also be used to identify the binding of other molecules (e.g., peptide mimetics, small molecules, and the like) to PDZ domain sequences. For example, using the assays disclosed herein, combinatorial and other libraries of compounds can be screened, e.g., for molecules that specifically bind to PDZ domains in hematopoietic cells. Screening of libraries can be accomplished by any of a variety of commonly known methods. See, e.g., the following references, which disclose screening of peptide libraries: Parmley and Smith, 1989, Adv. Exp. Med Biol. 251:215-218; Scott and Smith, 1990, Science 249:386-390; Fowlkes et al., 1992; BioTechniques 13:422-427; Oldenburg et al., 1992, Proc. Natl. Acad Sci. USA 89:5393-5397; Yu et al., 1994, Cell 76:933-945; Staudt et al., 1988, Science 241:577-580; Bock et al., 1992, Nature 355:564-566; Tuerk et al., 1992, Proc. Natl. Acad Sci. USA 89:6988-6992; Ellington et al., 1992, Nature 355:850-852; U.S. Pat. No. 5,096,815, U.S. Pat. No. 5,223,409, and U.S. Pat. No. 5,198,346, all to Ladner et al.; Rebar and Pabo, 1993, Science 263:671-673; and PCT Publication No. WO 94/18318.

In a specific embodiment, screening can be carried out by contacting the library members with a hematopoietic cell PDZ-domain polypeptide immobilized on a solid support (e.g. as described supra in the “G” assay) and harvesting those library members that bind to the protein. Examples of such screening methods, termed “panning” techniques are described by way of example in Parmley and Smith, 1988, Gene 73:305-318; Fowlkes et al., 1992, BioTechniques 13:422-427; PCT Publication No. WO 94/18318; and in references cited hereinabove.

In another embodiment, the two-hybrid system for selecting interacting proteins in yeast (Fields and Song, 1989, Nature 340:245-246; Chien et al., 1991, Proc. Natl. Acad Sci. USA 88:9578-9582) can be used to identify molecules that specifically bind to a PDZ domain-containing protein. Furthermore, the identified molecules are further tested for their ability to inhibit transmembrane receptor interactions with a PDZ domain.

In one aspect of the invention, antagonists of an interaction between a PDZ protein and a PL protein are identified. In one embodiment, a modification of the “A” assay described supra is used to identify antagonists. In one embodiment, a modification of the “G” assay described supra is used to identify antagonists.

In one embodiment, screening assays are used to detect molecules that specifically bind to PDZ domains in hematopoietic cells. Such molecules are useful as agonists or antagonists of PDZ-protein-mediated cell function (e.g., cell activation, e.g., T cell activation, vesicle transport, cytokine release, growth factors, transcriptional changes, cytoskeletin rearrangement, cell movement, chemotaxis, and the like). In one embodiment, such assays are performed to screen for leukocyte activation inhibitors for drug development. The invention thus provides assays to detect molecules that specifically bind to PDZ domain-containing proteins in hematopoietic cells. For example, recombinant cells expressing PDZ domain-encoding nucleic acids can be used to produce PDZ domains in these assays and to screen for molecules that bind to the domains. Molecules are contacted with the PDZ domain (or fragment thereof) under conditions conducive to binding, and then molecules that specifically bind to such domains are identified. Methods that can be used to carry out the foregoing are commonly known in the art.

It will be appreciated by the ordinarily skilled practitioner that, in one embodiment, antagonists are identified by conducting the A or G assays in the presence and absence of a known or candidate antagonist. When decreased binding is observed in the presence of a compound, that compound is identified as an antagonist. Increased binding in the presence of a compound signifies that the compound is an agonist.

For example, in one assay, a test compound can be identified as an inhibitor (antagonist) of binding between a PDZ protein and a PL protein by contacting a PDZ domain polypeptide and a PL peptide in the presence and absence of the test compound, under conditions in which they would (but for the presence of the test compound) form a complex, and detecting the formation of the complex in the presence and absence of the test compound. It will be appreciated that less complex formation in the presence of the test compound than in the absence of the compound indicates that the test compound is an inhibitor of a PDZ protein-PL protein binding. In various embodiments, the PL peptide comprises an amino acid sequence substantially identical to the C-terminal sequence of a PL protein (e.g., CD6, CD49E, CD49F, CD138, Clasp-1, Clasp-4, VCAM1, Clasp-2, CD95, DNAM-1, CD83, CD44, CD4, CD97, Neurexin, CD3n, DOCK2, CD34, FceRIb, or FasLigand).

In one embodiment, the “G” assay is used in the presence or absence of an candidate inhibitor. In one embodiment, the “A” assay is used in the presense or absence of a candiate inhibitor.

In one embodiment (in which a G assay is used), one or more PDZ domain-containing GST-fusion proteins are bound to the surface of wells of a 96-well plate as described supra (with appropriate controls including nonfusion GST protein). All fusion proteins are bound in multiple wells so that appropriate controls and statistical analysis can be done. A test compound in BSA/PBS (typically at multiple different concentrations) is added to wells. Immediately thereafter, 30 uL of a detectably labeled (e.g., biotinylated) peptide known to bind to the relevant PDZ domain (see, e.g., TABLE 2) is added in each of the wells at a final concentration of, e.g., between about 2 uM and about 40 uM, typically 5 uM, 15 uM, or 25 uM. This mixture is then allowed to react with the PDZ fusion protein bound to the surface for 10 minutes at 4° C. followed by 20 minutes at 25° C. The surface is washed free of unbound peptide three times with ice cold PBS and the amount of binding of the peptide in the presence and absence of the test compound is determined. Usually, the level of binding is measured for each set of replica wells (e.g. duplicates) by subtracting the mean GST alone background from the mean of the raw measurement of peptide binding in these wells.

In an alternative embodiment, the A assay is carried out in the presence or absence of an test candidate to identify inhibitors of PL-PDZ interactions.

In one embodiment, a test compound is determined to be a specific inhibitor of the binding of the PDZ domain (P) and a PL (L) sequence when, at a test compound concentration of less than or equal to 1 mM (e.g., less than or equal to: 500 uM, 100 uM, 10 uM, 1 uM, 100 nM or 1 nM) the binding of P to L in the presence of the test compound less than about 50% of the binding in the absence of the test compound. (in various embodiments, less than about 25%, less than about 10%, or less than about 1%). Preferably, the net signal of binding of P to L in the presence of the test compound plus six (6) times the standard error of the signal in the presence of the test compound is less than the binding signal in the absence of the test compound.

In one embodiment, assays for an inhibitor are carried out using a single PDZ protein-PL protein pair (e.g., a PDZ domain fusion protein and a PL peptide). In a related embodiment, the assays are carried out using a plurality of pairs, such as a plurality of different pairs listed in TABLE 2.

In some embodiments, it is desirable to identify compounds that, at a given concentration, inhibit the binding of one PL-PDZ pair, but do not inhibit (or inhibit to a lesser degree) the binding of a specified second PL-PDZ pair. These antagonists can be identified by carrying out a series of assays using a candidate inhibitor and different PL-PDZ pairs (e.g., as shown in the matrix of TABLE 2) and comparing the results of the assays. All such pairwise combinations are contemplated by the invention (e.g., test compound inhibits binding of PL₁ to PDZ₁ to a greater degree than it inhibits binding of PL₁ to PDZ₂ or PL₂ to PDZ₂). Importantly, it will be appreciated that, based on the data provided in TABLE 2 and disclosed herein (and additional data that can be generated using the methods described herein) inhibitors with different specificities can readily be designed.

For example, according to the invention, the Ki (“potency”) of an inhibitor of a PDZ-PL interaction can be determined. Ki is a measure of the concentration of an inhibitor required to have a biological effect. For example, administration of an inhibitor of a PDZ-PL interaction in an amount sufficient to result in an intracellular inhibitor concentration of at least between about 1 and about 100 Ki is expected to inhibit the biological response mediated by the target PDZ-PL interaction. In one aspect of the invention, the Kd measurement of PDZ-PL binding as determined using the methods supra is used in determining Ki.

Thus, in one aspect, the invention provides a method of determining the potency (Ki) of an inhibitor or suspected inhibitor of binding between a PDZ domain and a ligand by immobilizing a polypeptide comprising the PDZ domain and a non-PDZ domain on a surface, contacting the immobilized polypeptide with a plurality of different mixtures of the ligand and inhibitor, wherein the different mixtures comprise a fixed amount of ligand and different concentrations of the inhibitor, determining the amount of ligand bound at the different concentrations of inhibitor, and calculating the Ki of the binding based on the amount of ligand bound in the presence of different concentrations of the inhibitor. In an embodiment, the polypeptide is immobilized by binding the polypeptide to an immobilized immunoglobulin that binds the non-PDZ domain. This method, which is based on the “G” assay described supra, is particularly suited for high-throughput analysis of the Ki for inhibitors of PDZ-ligand interactions. Further, using this method, the inhibition of the PDZ-ligand interaction itself is measured, without distortion of measurements by avidity effects.

Typically, at least a portion of the ligand is detectably labeled to permit easy quantitation of ligand binding.

It will be appreciated that the concentration of ligand and concentrations of inhibitor are selected to allow meaningful detection of inhibition. Thus, the concentration of the ligand whose binding is to be blocked is close to or less than its binding affinity (e.g., preferably less than the 5× Kd of the interaction, more preferably less than 2× Kd, most preferably less than 1× Kd). Thus, the ligand is typically present at a concentration of less than 2 Kd (e.g., between about 0.01 Kd and about 2 Kd) and the concentrations of the test inhibitor typically range from 1 nM to 100 uM (e.g. a 4-fold dilution series with highest concentration 10 uM or 1 mM). In a preferred embodiment, the Kd is determined using the assay disclosed supra.

The Ki of the binding can be calculated by any of a variety of methods routinely used in the art, based on the amount of ligand bound in the presence of different concentrations of the inhibitor. in an illustrative embodiment, for example, a plot of labeled ligand binding versus inhibitor concentration is fit to the equation:
S_{inhibitor}=S₀*Ki/([I]+Ki)
where S_{inhibitor} is the signal of labeled ligand binding to immobilized PDZ domain in the presence of inhibitor at concentration [I] and S₀ is the signal in the absence of inhibitor (i.e., [I]=0). Typically [I] is expressed as a molar concentration.

In another aspect of the invention, an enhancer (sometimes referred to as, augmentor or agonist) of binding between a PDZ domain and a ligand is identified by immobilizing a polypeptide comprising the PDZ domain and a non-PDZ domain on a surface, contacting the immobilized polypeptide with the ligand in the presence of a test agent and determining the amount of ligand bound, and comparing the amount of ligand bound in the presence of the test agent with the amount of ligand bound by the polypeptide in the absence of the test agent. At least two-fold (often at least 5-fold) greater binding in the presence of the test agent compared to the absence of the test agent indicates that the test agent is an agent that enhances the binding of the PDZ domain to the ligand. As noted supra, agents that enhance PDZ-ligand interactions are useful for disruption (dysregulation) of biological events requiring normal PDZ-ligand function (e.g., cancer cell division and metastasis, and activation and migration of immune cells).

The invention also provides methods for determining the “potency” or “K_{enhancer}” of an enhancer of a PDZ-ligand interaction. For example, according to the invention, the K_{enhancer} of an enhancer of a PDZ-PL interaction can be determined, e.g., using the Kd of PDZ-PL binding as determined using the methods described supra. K_{enhancer} is a measure of the concentration of an enhancer expected to have a biological effect. For example, administration of an enhancer of a PDZ-PL interaction in an amount sufficient to result in an intracellular inhibitor concentration of at least between about 0.1 and about 100 K_{enhancer} (e.g., between about 0.5 and about 50 K_{enhancer}) is expected to disrupt the biological response mediated by the target PDZ-PL interaction.

Thus, in one aspect the invention provides a method of determining the potency (K_{enhancer}) of an enhancer or suspected enhancer of binding between a PDZ domain and a ligand by immobilizing a polypeptide comprising the PDZ domain and a non-PDZ domain on a surface, contacting the immobilized polypeptide with a plurality of different mixtures of the ligand and enhancer, wherein the different mixtures comprise a fixed amount of ligand, at least a portion of which is detectably labeled, and different concentrations of the enhancer, determining the amount of ligand bound at the different concentrations of enhancer, and calculating the potency (K_{enhancer}) of the enhancer from the binding based on the amount of ligand bound in the presence of different concentrations of the enhancer. Typically, at least a portion of the ligand is detectably labeled to permit easy quantitation of ligand binding. This method, which is based on the “G” assay described supra, is particularly suited for high-throughput analysis of the K_{enhancer} for enhancers of PDZ-ligand interactions.

It will be appreciated that the concentration of ligand and concentrations of enhancer are selected to allow meaningful detection of enhanced binding. Thus, the ligand is typically present at a concentration of between about 0.01 Kd and about 0.5 Kd and the concentrations of the test agent/enhancer typically range from 1 nM to 1 mM (e.g. a 4-fold dilution series with highest concentration 10 uM or 1 mM). In a preferred embodiment, the Kd is determined using the assay disclosed supra.

The potency of the binding can be determined by a variety of standard methods based on the amount of ligand bound in the presence of different concentrations of the enhancer or augmentor. For example, a plot of labeled ligand binding versus enhancer concentration can be fit to the equation:
S([E])=S(0)+(S(0)*(D_{enhancer}−1)*[E]/([E]+K_{enhancer})
where “K_{enhancer}” is the potency of the augmenting compound, and “D_{enhancer}” is the fold-increase in binding of the labeled ligand obtained with addition of saturating amounts of the enhancing compound, [E] is the concentration of the enhancer. It will be understood that saturating amounts are the amount of enhancer such that further addition does not significantly increase the binding signal. Knowledge of “K_{enhancer}” is useful because it describes a concentration of the augmenting compound in a target cell that will result in a biological effect due to dysregulation of the PDZ-PL interaction. Typical therapeutic concentrations are between about 0.1 and about 100 K_{enhancer}.

6.4.1 Global Analysis of PDZ-PL Interactions

As described supra, the present invention provides powerful methods for analysis of PDZ-ligand interactions, including high-throughput methods such as the “G” assay and affinity assays described supra. In one embodiment of the invention, the affinity is determined for a particular ligand and a plurality of PDZ proteins. Typically the plurality is at least 5, and often at least 25, or at least 40 different PDZ proteins. In a preferred embodiment, the plurality of different PDZ proteins are from a particular tissue (e.g., central nervous system, spleen, cardiac muscle, kidney) or a particular class or type of cell, (e.g., a hematopoietic cell, a lymphocyte, a neuron) and the like. In a most preferred embodiment, the plurality of different PDZ proteins represents a substantial fraction (e.g., typically a majority, more often at least 80%) of all of the PDZ proteins known to be, or suspected of being, expressed in the tissue or cell(s), e.g., all of the PDZ proteins known to be present in lymphocytes. In an embodiment, the plurality is at least 50%, usually at least 80%, at least 90% or all of the PDZ proteins disclosed herein as being expressed in hematopoietic cells (see Table 7).

In one embodiment of the invention, the binding of a ligand to the plurality of PDZ proteins is determined. Using this method, it is possible to identify a particular PDZ domain bound with particular specificity by the ligand. The binding may be designated as “specific” if the affinity of the ligand to the particular PDZ domain is at least 2-fold that of the binding to other PDZ domains in the plurality (e.g., present in that cell type). The binding is deemed “very specific” if the affinity is at least 10-fold higher than to any other PDZ in the plurality or, alternatively, at least 10-fold higher than to at least 90%, more often 95% of the other PDZs in a defined plurality. Similarly, the binding is deemed “exceedingly specific” if it is at least 100-fold higher. For example, a ligand cound bind to 2 different PDZs with an affinity of 1 uM and to no other PDZs out of a set 40 with an affinty of less than 100 uM. This would constitute specific binding to those 2 PDZs. Similar measures of specifity are used to describe binding of a PDZ to a plurality of PLs.

It will be recognized that high specificity PDZ-PL interactions represent potentially more valuable targets for achieving a desired biological effect. The ability of an inhibitor or enhancer to act with high specificity is often desirable. In particular, the most specific PDZ-ligand interactions are also the best therapeutic targets, allowing specific inhibition of the interaction.

Thus, in one embodiment, the invention provides a method of identifying a high specificity interaction between a particular PDZ domain and a ligand known or suspected of binding at least one PDZ domain, by providing a plurality of different immobilized polypeptides, each of said polypeptides comprising a PDZ domain and a non-PDZ domain; determining the affinity of the ligand for each of said polypeptides, and comparing the affinity of binding of the ligand to each of said polypeptides, wherein an interaction between the ligand and a particular PDZ domain is deemed to have high specificity when the ligand binds an immobilized polypeptide comprising the particular PDZ domain with at least 2-fold higher affinity than to immobilized polypeptides not comprising the particular PDZ domain.

In a related aspect, the affinity of binding of a specific PDZ domain to a plurality of ligands (or suspected ligands) is determined. For example, in one embodiment, the invention provides a method of identifying a high specificity interaction between a PDZ domain and a particular ligand known or suspected of binding at least one PDZ domain, by providing an immobilized polypeptide comprising the PDZ domain and a non-PDZ domain; determining the affinity of each of a plurality of ligands for the polypeptide, and comparing the affinity of binding of each of the ligands to the polypeptide, wherein an interaction between a particular ligand and the PDZ domain is deemed to have high specificity when the ligand binds an immobilized polypeptide comprising the PDZ domain with at least 2-fold higher affinity than other ligands tested. Thus, the binding may be designated as “specific” if the affinity of the PDZ to the particular PL is at least 2-fold that of the binding to other PLs in the plurality (e.g., present in that cell type). The binding is deemed “very specific” if the affinity is at least 10-fold higher than to any other PL in the plurality or, alternatively, at least 10-fold higher than to at least 90%, more often 95% of the other PLs in a defined plurality. Similarly, the binding is deemed “exceedingly specific” if it is at least 100-fold higher. Typically the plurality is at least 5 different ligands, more often at lease 10. In an embodiment, the plurality of ligands comprises at least 1, typically at least 2, more often at least 5, and sometimes at least 10 ligands selected from CD105, VCAM1, CD95, Spectrin beta, KV1.3, DNAM1, Neuroligin 3, TAX, CD44 (long form), CD38, CD3n, LPAP, CD46 (form 1), CDw128B (IL-8 receptor B), DOCK2, PAG, CD34, BLR-1 (or a polypeptide comprising a C-terminal sequence (e.g., at least about 3, 4, 6, 8 or 10 residues) from such a ligand).

TABLE 7
PDZ Domain-Containing Genes Expressed in T Cells and B Cells

| | Expressed in T/B | Genebank |
|---|---|---|
| PDZ gene name | cells | acc. # |
| AF6 | T-/B-cells | 430993 |
| BAI I associated prot. | T-/B-cells | 3370997 |
| CASK (mouse) | T-/B-cells | 3087815 |
| Connector enhancer | B-cells | 3930780 |
| Cytohesin bind. Prot. | T-/B-cells | 3192908 |
| DLG1 | T-/B-cells | 475816 |
| DLG5 (pdlg) | T-/B-cells | 3650451 |
| DVL1 | T-/B-cells | 2291005 |
| DVL3 | T-/B-cells | 6806886 |
| GTPase | T-/B-cells | 3004860 |
| Guanin-exchange factor 1 | T-/B-cells | 6650765 |
| hypoth. 41.8 kd | T-/B-cells | 3882222 |
| PDZ domain containing | T cells only | 2370148 |
| prot. | | |
| KIAA147 | T-/B-cells | 1469875 |
| KIAA0300 | T-/B-cells | 2224540 |
| KIAA0303 | T-/B-cells | 2224546 |
| KIAA0316 | T-cells | 6683123 |
| KIAA0380 | T-/B-cells | 2224700 |
| KIAA0440 | T-/B-cells | 2662160 |
| KIAA0545 | T-/B-cells | 303617 |
| KIAA0561 | T-/B-cells | 3043645 |
| KIAA0559 | B-cells | 3043641 |
| KIAA0807 | T-/B-cells | 3882334 |
| KIAA0858 | T-/B-cells | 42402004 |
| KIAA0902 | T-/B-cells | 4240304 |
| LIMK1 | T-/B-cells | 4587498 |
| LIMK2 | T-/B-cells | 1805593 |
| LIM domain prot | T-/B-cells | 2957144 |
| LIM protein | T-/B-cells | 3108092 |
| MINT1 | T-/B-cells | 2625024 |
| MINT3 | T-/B-cells | 3169808 |
| MPP1 | T-/B-cells | 189785 |
| MPP2 | T-/B-cells | 939884 |
| NE-DLG | T-/B-cells | 1515354 |
| NOS1 | T-/B-cells | 642525 |
| novel serine protease | T-/B-cells | 1621243 |
| PDZK1 | T-/B-cells | 2944188 |
| PICK8 | T-/B-cells | 4678411 |
| PTN-3 | T-/B-cells | 179912 |
| PTN-4 | B cells | 190747 |
| prIL16 | T-/B-cells | 1478492 |
| PSD95 | T-/B-cells | 3318652 |
| RPIP8 | T-/B-cells | 5730014 |
| RGS12 | T-/B-cells | 3290015 |
| serine protease | T-/B-cells | 2738914 |
| 26s subunit p27 | T-cells | 9184389 |
| hSYNTENIN | T-/B-cells | 2795862 |
| SYNTR. 1 alpha | T-/B-cells | 1145727 |
| TAX1-IP | T-/B-cells | 2613001 |
| TAX2-IP | T-/B-cells | 2613003 |
| TAX2-like protein | T-/B-cells | 3253116 |
| TAX33-IP | T-/B-cells | 2613007 |
| TAX40-IP (PAR-6) | T-/B-cells | 2613011 |
| Tax43-IP (SYN. Beta1) | T-/B-cells | 2613011 |
| TIAM | T-/B-cells | 4507500 |
| wwp3 | T-/B-cells | 2695619 |
| X11 prot. beta | T-/B-cells | 3005559 |
| ZO1 | T-/B-cells | 292937 |

6.4.2 Use of Array for Global Predictions

One discovery of the present inventors relates to the important and extensive roles played by interactions between PDZ proteins and PL proteins, particularly in the biological function of hematopoietic cells and other cells involved in the immune response. Further, it has been discovered that valuable information can be ascertained by analysis (e.g., simultaneous analysis) of a large number of PDZ-PL interactions. In a preferred embodiment, the analysis encompasses all of the PDZ proteins expressed in a particular tissue (e.g., spleen) or type or class of cell (e.g., hematopoietic cell, neuron, lymphocyte, B cell, T cell and the like). Alternatively, the analysis encompasses at least about 5, or at least about 10, or at least about 12, or at least about 15 and often at least 50 different polypeptides, up to about 60, about 80, about 100, about 150, about 200, or even more different polypeptides; or a substantial fraction (e.g., typically a majority, more often at least 80%) of all of the PDZ proteins known to be, or suspected of being, expressed in the tissue or cell(s), e.g., all of the PDZ proteins known to be present in lymphocytes. In an embodiment, the plurality is at least 50%, usually at least 80%, at least 90% or all of the PDZ proteins disclosed herein as being expressed in hematopoietic cells (see Table 7).

It will be recognized that the arrays and methods of the invention are directed to analyis of PDZ and PL interactions, and involve selection of such proteins for analysis. While the devices and methods of the invention may include or involve a small number of control polypeptides, they typically do not include significant numbers of proteins or fusion proteins that do not include either PDZ or PL domains (e.g., typically, at least about 90% of the arrayed or immobilized polypeptides in a method or device of the invention is a PDZ or PL sequence protein, more often at least about 95%, or at least about 99%).

In an embodiment the array includes at least one, preferably at least 1, more often at least 5 or at least 10 and sometimes all of the following PDZ proteins present in lymphocytes: BAI I associated prot., Connector enhancer, DLG5 (pdlg), DVL3, GTPase, Guanin-exchange factor 1, PDZ domain containing prot., KIAA147, KIAA0300, KIAA0380, KIAA0440, KIAA0545, KIAA0807, KIAA0858, KIAA0902, novel serine protease, PDZK1, PICK8, PTN-3, RPIP8, serine protease, 26s subunit p27, hSYNTENIN, TAX1-IP, TAX2-like protein, wwp3, X11 prot. beta, ZO1.

It will be apparent from this disclosure that analysis of the relatively large number of different interactions preferably takes place simultaneously. In this context, “simultaneously” means that the analysis of several different PDZ-PL interactions (or the effect of a test agent on such interactions) is assessed at the same time. Typically the analysis is carried out in a highthroughput (e.g., robotic) fashion. One advantage of this method of simultaneous analysis is that it permits rigorous comparison of multiple different PDZ-PL interactions. For example, as explained in detail elsewhere herein, simultaneous analysis (and use of the arrays described infra) facilitates, for example, the direct comparison of the effect of an agent (e.g., an potential interaction inhibitor) on the interactions between a substantial portion of PDZs and/or PLs in a tissue or cell.

Accordingly, in one aspect, the invention provides an array of immobilized polypeptide comprising the PDZ domain and a non-PDZ domain on a surface. Typically, the array comprises at least about 5, or at least about 10, or at least about 12, or at least about 15 and often at least 50 different polypeptides. In one preferred embodiment, the different PDZ proteins are from a particular tissue (e.g., central nervous system, speen, cardiac muscle, kidney) or a particular class or type of cell, (e.g., a hematopoietic cell, a lymphocyte, a neuron) and the like. In a most preferred embodiment, the plurality of different PDZ proteins represents a substantial fraction (e.g., typically a majority, more often at least 80%) of all of the PDZ proteins known to be, or suspected of being, expressed in the tissue or cell(s), e.g., all of the PDZ proteins known to be present in lymphocytes. In an embodiment, the plurality is at least 50%, usually at least 80%, at least 90% or all of the PDZ proteins disclosed herein as being expressed in hematopoietic cells (see Table 7) e.g.; all of the PDZ proteins known to be present in lymphocytes. In an embodiment, the plurality is at least 50%, usually at least 80%, at least 90% or all of the PDZ proteins disclosed herein as being expressed in hematopoietic cells (see Table 7).

In an embodiment the array includes at least one, preferably at least 1, typically at least 5 and sometimes all of the following PDZ proteins present in lymphocytes: BAI I associated prot., Connector enhancer, DLG5 (pdlg), DVL3, GTPase, Guanin-exchange factor 1, PDZ domain containing prot., KIAA147, KIAA0300, KIAA0380, KIAA0440, KIAA0545, KIAA0807, KIAA0858, KIAA0902, novel serine protease, PDZK1, PICK8, PTN-3, RPIP8, serine protease, 26s subunit p27, hSyNTENIN, TAX1-IP, TAX2-like protein, wwp3, X11 prot. beta, ZO1. In this context, “array” refers to an ordered series of of immobilized polypeptides in which the identity of each polypeptide is associated with its location. In some embodiments the plurality of polypeptides are arrayed in a “common” area such that they can be simultaneously exposed to a solution (e.g., containing a ligand or test agent). For example, the plurality of polypeptides can be on a slide, plate or similar surface, which may be plastic, glass, metal, silica, beads or other surface to which proteins can be immobilized. In a different embodiment, the different immobilized polypeptides are situated in separate areas, such as different wells of multi-well plate (e.g., a 24-well plate, a 96-well plate, a 384 well plate, and the like). It will be recognized that a similar advantage can be obtained by using multiple arrays in tandem.

6.4.3 Analysis of PDZ-PL Inhibition Profile

In one aspect, the invention provides a method for determining if a test compound inhibits any PDZ-ligand interaction in large set of PDZ-ligand interaction (e.g., some or all of the PDZ-ligands interactions described in Table 2; a majority of the PDZ-ligands identified in a particular cell or tissue as described supra (e.g., lymphocytes) and the like. In one embodiment, the PDZ domains of interest are expressed as GST-PDZ fusion proteins and immobilized as described herein. For each PDZ domain, a labeled ligand that binds to the domain with a known affinity is identified as described herein.

As disclosed herein, numerous PDZ-PL interactions occur in cells of the hematopoietic system. For any known or suspected modulator (e.g., inhibitor) of a PDL-PL interaction(s), it is useful to know which interactions are inhibited (or augmented). For example, an agent that inhibits all PDZ-PL interactions in a cell (e.g., a lymphocyte) will have different uses than an agent that inhibits only one, or a small number, of specific PDZ-PL interactions. The profile of PDZ interactions inhibited by a particular agent is referred to as the “inhibition profile” for the agent, and is described in detail below. The profile of PDZ interactions enhanced by a particular agent is referred to as the “enhancement profile” for the agent. It will be readily apparent to one of skill guided by the description of the inhibition profile how to determine the enhancement profile for an agent. The present invention provides methods for determining the PDZ interaction (inhibition/enhancement) profile of an agent in a single assay.

In one aspect, the invention provides a method for determining the PDZ-PL inhibition profile of a compound by providing (i) a plurality of different immobilized polypeptides, each of said polypeptides comprising a PDZ domain and a non-PDZ domain and (ii) a plurality of corresponding ligands, wherein each ligand binds at least one PDZ domain in (i), then contacting each of said immobilized polypeptides in (i) with a corresponding ligand in (ii) in the presence and absence of a test compound, and determining for each polypeptide-ligand pair whether the test compound inhibits binding between the immobilized polypeptide and the corresponding ligand.

Typically the plurality is at least 5, and often at least 25, or at least 40 different PDZ proteins. In a preferred embodiment, the plurality of different ligands and the plurality of different PDZ proteins are from the same tissue or a particular class or type of cell, e.g., a hematopoietic cell, a lymphocyte, a neuron and the like. In a most preferred embodiment, the plurality of different PDZs represents a substantial fraction (e.g., at least 80%) of all of the PDZs known to be, or suspected of being, expressed in the tissue or cell(s), e.g., all of the PDZs known to be present in lymphocytes (for example, at least 80%, at least 90% or all of the PDZs disclosed herein as being expressed in hematopoietic cells).

In one embodiment, the inhibition profile is determined as follows: A plurality (e.g., all known) PDZ domains expressed in a cell (e.g., lymphocytes) are expressed as GST-fusion proteins and immobilized without altering their ligand binding properties as described supra. For each PDZ domain, a labeled ligand that binds to this domain with a known affinity is identified. If the set of PDZ domains expressed in lymphocytes is denoted by {P1 . . . Pn}, any given PDZ domain Pi binds a (labeled) ligand Li with affinity K_{d}i. To determine the inhibition profile for a test agent “compound X” the “G” assay (supra) can be performed as follows in 96-well plates with rows A-H and columns 1-12. Column 1 is coated with P1 and washed. The corresponding ligand L1 is added to each washed coated well of column 1 at a concentration 0.5 K_{d}1 with (rows B, D, F, H) or without (rows A, C, E, F) between about 1 and about 1000 uM) of test compound X. Column 2 is coated with P2, and L2 (at a concentration 0.5 K_{d}2) is added with or without inhibitor X. Additional PDZ domains and ligands are similarly tested.

Compound X is considered to inhibit the binding of Li to Pi if the average signal in the wells of column i containing X is less than half the signal in the equivalent wells of the column lacking X. Thus, in this single assay one determines the full set of lymphocyte PDZs that are inhibited by compound X.

In some embodiments, the test compound X is a mixture of compounds, such as the product of a combinatorial chemistry synthesis as described supra. In some embodiments, the test compound is known to have a desired biological effect, and the assay is used to determine the mechanism of action (i.e., if the biological effect is due to modulating a PDZ-PL interaction).

It will be apparent that an agent that modulates only one, or a few PDZ-PL interactions, in a panel (e.g., a panel of all known PDZs lymphocytes, a panel of at least 10, at least 20 or at least 50 PDZ domains) is a more specific modulator than an agent that modulate many or most interactions. Typically, an agent that modulates less than 20% of PDZ domains in a panel (e.g., Table 2) is deemed a “specific” inhibitor, less than 6% a “very specific” inhibitor, and a single PDZ domain a “maximally specific” inhibitor.

It will also be appreciated that “compound X” may be a composition containing mixture of compounds (e.g., generated using combinatorial chemistry methods) rather than a single compound.

Several variations of this assay are contemplated:

In some alternative embodiments, the assay above is performed using varying concentrations of the test compound X, rather than fixed concentration. This allows determination of the Ki of the X for each PDZ as described above.

In an alternative embodiment, instead of pairing each PDZ Pi with a specific labeled ligand Li, a mixture of different labeled ligands is created that such that for every PDZ at least one of the ligands in the mixture binds to this PDZ sufficiently to detect the binding in the “G” assay. This mixture is then used for every PDZ domain.

In one embodiment, compound X is known to have a desired biological effect, but the chemical mechanism by which it has that effect is unknown. The assays of the invention can then be used to determine if compound X has its effect by binding to a PDZ domain.

In one embodiment, PDZ-domain containing proteins are classified in to groups based on their biological function, e.g. into those that regulate chemotaxis versus those that regulate transcription. An optimal inhibitor of a particular function (e.g., including but not limited to an anti-chemotactic agent, an anti-T cell activation agent, cell-cycle control, vesicle transport, apoptosis, etc.) will inhibit multiple PDZ-ligand interactions involved in the function (e.g., chemotaxis, activation) but few other interactions. Thus, the assay is used in one embodiment in screening and design of a drug that specifically blocks a particular function. For example, an agent designed to block chemotaxis might be identified because, at a given concentration, the agent inhibits 2 or more PDZs involved in chemotaxis but fewer than 3 other PDZs, or that inhibits PDZs involved in chemotaxis with a Ki>10-fold better than for other PDZs. Thus, the invention provides a method for identifying an agent that inhibits a first selected PDZ-PL interaction or plurality of interactions but does not inhibit a second selected PDZ-PL interaction or plurality of interactions. The two (or more) sets of interactions can be selected on the basis of the known biological function of the PDZ proteins, the tissue specificity of the PDZ proteins, or any other criteria. Moreover, the assay can be used to determine effective doses (i.e., drug concentrations) that result in desired biological effects while avoiding undesirable effects.

6.4.4 Side Effects of PDZ-PL Modulator Interactions

In a related embodiment, the invention provides a method for determining likely side effects of a therapeutic that inhibits PDZ-ligand interactions. The method entails identifying those target tissues, organs or cell types that express PDZ proteins and ligands that are disrupted by a specified inhibitor. If, at a therapeutic dosage, a drug intended to have an effect in one organ system (e.g., hematopoietic system) disrupts PDZ-PL interactions in a different system (e.g., CNS) it can be predicted that the drug will have effects (“side effects”) on the second system. It will be apparent that the information obtained from this assay will be useful in the rational design and selection of drugs that do not have the side-effect.

In one embodiment, for example, a comprehensive PDZ protein set is obtained. A “perfectly comprehensive” PDZ protein set is defined as the set of all PDZ proteins expressed in the subject animal (e.g., humans). A comprehensive set may be obtained by analysis of, for example, the human genome sequence. However, a “perfectly comprehensive” set is not required and any reasonably large set of PDZ domain proteins (e.g., the set of all known PDZ proteins; or the set listed in Table 7) will provide valuable information.

In one embodiment, the method involves some of all of the following steps:
a) For each PDZ protein, determine the tissues in which it is highly expressed. This can be done experimentally although the information generally will be available in the scientific literature;
b) For each PDZ protein (or as many as possible), identify the cognate PL(s) bound by the PDZ protein;
c) Determine the Ki at which the test agent inhibits each PDZ-PL interaction, using the methods described supra;
d) From this information it is possible to calculate the pattern of PDZ-PL interactions disrupted at various concentrations of the test agent
   By correlating the set of PDZ-PL interactions disrupted with the expression pattern of the members of that set, it will be possible to identify the tissues likely affected by the agent.

Additional steps can also be carried out, including determining whether a specified tissue or cell type is exposed to an agent following a particular route of administration. This can be determined using basis pharmacokinetic methods and principles.

6.4.5 Modulation of Activities

The PDZ binding moieties and PDZ protein-PL protein binding antagonists of the invention are used to modulate biological activities or functions of cells (e.g., hematopoietic cells, such as T cells and B cells and the like), endothelial cells, and other immune system cells, as described herein, and for treatment of diseases and conditions in human and nonhuman animals (e.g., experimental models). Exemplary biological acitivities are listed supra.

When administered to patients, the compounds of the invention (e.g., PL-PDZ interaction inhibitors) are useful for treating (ameliorating symptoms of) a variety of diseases and conditions, including diseases characterized by inflammatory and humoral immune responses, e.g., inflammation, allergy (e.g., systemic anaphylaxis, hypersensitivity responses, drug allergies, insect sting allergies; inflammatory bowel diseases, ulcerative colitis, ileitis and enteritis; psoriasis and inflammatory dermatoses, scleroderma; respiratory allergic diseases such as asthma, allergic rhinitis, hypersensitivity lung diseases, and the like vasculitis, rh incompatibility, transfusion reactions, drug sensitivities, PIH, atopic dermatitis, eczema, rhinnitis; autoimmune diseases, such as arthritis (rheumatoid and psoriatic), multiple sclerosis, systemic lupus erythematosus, insulin-dependent diabetes, glomerulonephritis, scleroderma, MCTD, IDDM, Hashimoto thyroiditis, Goodpasture syndrome, psoriasis and the like, osteoarthritis, polyarthritis, graft rejection (e.g., allograft rejection, e.g., renal allograft rejection, graft-vs-host disease, transplantation rejection (cardiac, kidney, lung, liver, small bowel, cornea, pancreas, cadaver, autologous, bone marrow, xenotransplantation)), atherosclerosis, angiogenesis-dependent disorders, cancers (e.g., melanomas and breast cancer, prostrate cancer, leukemias, lymphomas, metastatic disease), infectious diseases (e.g., viral infection, such as HIV, measles, parainfluenza, virus-mediated cell fusion,), ischemia (e.g., post-myocardial infarction complications, joint injury, kidney, scleroderma).

The PL proteins and PDZ proteins listed in TABLE 2 are well characterized, and one of skill, guided by this disclosure (including the discovery of the interactions between PL proteins and PDZ proteins described herein), will recognize many uses for modulators (e.g., enhancers or inhibitors) of PDZ-PL interactions such as those described in TABLE 2. To further assist the reader, a discussion of the characteristics of selected PL proteins (and their function) is provided infra. It will be recognized that this discussion is not comprehensive and is not intended to limit the invention in any way. Moreover, nothing in this section should be construed as an intention by the inventors to be limited to a particular mechanism of action.

A. CD6

As shown supra, CD6 binds PDZ protein 41.8. CD6 is expressed on thymocytes, T cells, and B cell chronic lymphocytic leukemias. CD6 plays a role in T cell co-stimulation and CD6 negative T cells are less autoreactive than CD6 positive T cells. Inhibition of CD6 and CD6/41.8 interactions is predicted to reduce the symptoms of graft-versus-host disease (GVHD) or psoriasis. Thus, in one embodiment of the invention, GVHD is reduced in a patient receiving donor bone marrow cells by pre-treating the cells with an effective amount of an antagonist. In combination with post-transplantation immunosuppressive therapy such as FK506, Cellcept, or cyclosporin, CD6-PDZ interaction inhibitors will improve overall survival of transplantation patients (e.g., leukemia patients).

B. CD49e (ALPHA-4)

As shown by the experiments reported herein, the C-terminal end of CD49e binds to the PDZ-domain-containing protein41.8 kD. CD49e is a 110 kD transmembrane membrane protein of the integrin alpha family (integrin alpha 5). Paired with the integrin beta-1 subunit it forms VLA-5. VLA-5 is expressed predominantly on hematopoietic and lymphoid lineage cells including monocytes, basophils, T cells, and activated B cells. VLA-5 is the receptor for the ubiquitously-expressed adhesion molecule fibronectin. Tissue injury such as myocardial infarction releases soluble fragments of fibronectin. Binding of these soluble fragments to VLA-5 results in chemotaxis of immune cells including monocytes to the source of fibronectin, as well as down-modulation of VLA-5 expression on these cells. Such ligand-induced down-modulation is a common and required feature of chemotaxic receptors. Once immune cells migrate fully to the source of fibronectin, adhesion to the fibronectin surface is enhanced by fibronectin-VLA-5 interaction. Without intending to be bound by a particular mechanism, the 41.8/CD49e interaction is believed to be necessary for proper membrane distribution of CD49e and/or recycling of CD49e such that when it is disrupted, the migration and adherence to fibronectin -containing surfaces is similarly disrupted, resulting in an inability of immune system cells to effectively migrate toward a fibronectin source and adhere to fibronectin-containing surfaces. Such disruption would therefore result in desirable reduced inflammatory processes, including reduced post-myocardial infarction inflammation. Other diseases to be treated include but are not limited to joint inflamation, psoriasis, contact allergy, Crohn's Disease, inflammatory bowel disease, eczema, atopic dermatitis.

C. CD49F (VLA-6 α Subunit)

As shown supra, CD49F binds PDZ protein 41.8. CD49f is known as an integrin subunit that pairs either with the β1 integrin subunit (CD29), forming VLA-6, or with CD104 (β4 integrin subunit). The integrin supergene family consists of a number of cell surface αβ heterodimers important for many different physiologic processes, including embryogenesis, thrombosis, wound healing, tumorigenesis and immune responses. Each β chain can pair with various α chains. Both VLA-6 and CD49f/CD104 are widely expressed on epithelia in non-lymphoid tissues. VLA-6 is also expressed on platelets, monocytes, thymocytes and T lymphocytes, with an increased expression on activated and resting memory T cells.

Inhibition of interactions between VLA-6 and 41.8 has a number of therapeutic functions such as the prevention and treatment of metastatic cancers, and treatment of overactive immunity. For example, VLA-6 is associated with invasivion of prostrate carcinoma and plays a role in the metastasis of breast cancer. Blockage of VLA-6 function combined with conventional treatment for prostrate cancer, would be a more effective treatment by preventing metastatic disease (see, Cress et al., 1995, Cancer Metastasis R). Blockage of CD49f through PDZ interaction may also treat Rh incompatibility by blunting memory response or in the treatment of keloids.

D. CD138 (Syndecan-1)

CD138 is a transmembrane proteoglycan receptor with the extracellular domain functioning as a ligand binding domain for various extracellular matrix components and the intracellular portion functioning to alter cytoskeleton and transduce intracellular signals. CD138 also binds FGF2 and may be a co-receptor for FGF receptor (Yayon et al., 1991).

As shown supra, CD138 interacts with 41.8 kD protein and TLAM1. The c-terminus of CD138 has also been reported to bind the PDZ domains of syntenin and human CASK (Cohen et al., 1998,J. Cell. Biol. 142:129-138; Grootjans et al., 1997, PNAS94:13683-13688; Hsueh et al., 1998, J. Cell. Biol. 142:139-151).CD138 is expressed in pre-B cells, immature B cells, plasma cells, neural cells, the basolateral surface of epithelial cells, embryonic mesenchymal cells, vascular smooth muscle cells, endothelial cells and neural cells but not mature circulating B cells. The interaction between CD138 and the PDZ domains of the 41.8 kD protein and TIAM1 proteins is believed to be necessary for the proper distribution of CD138 on the cell surface. Disruption of the interaction by administration of an effective amount of an antagonist is expected to interfere with the migration and adherence of cells to the extracellular matrix, resulting in reduced inflammatory and humoral immune responses. Inhibition of CD138 may be used to treat without limitation diseases such as post-myocardial infarction inflamatory damage, joint injury, rheumatoid arthritis, vasculitis, drug reaction, scleraderma, SLE, Hashimoto thyroiditis, Goodpasture's syndrome, juvenile insulin-dependent diabetes, psoriasis.

E. CD98

As shown supra, CD98 interacts with MPP2. CD98 is expressed at high levels on monocytes and at low levels on T cells, B cells, splenocytes, NK cells, and granulocytes. CD98 plays roles in adhesion, fusion and is a L-type amino acid transporter. CD98 is also involved in virus-mediated cell fusion (e.g. paramyoviruses: parainfluenza virus type 2, Newcastle disease virus, and rubulaviruses) and antagonism of CD98 function is expected to treat )viral infections and limit viral spread. CD98 inhibitors can be an antiviral agent for, but not limited to paramyovirus, parainfluenza, Newcastle disease and rubula Other roles include treatment for acute leukemias.

F. CLASP-1

As shown supra, CLASP-1 interacts with DLG1, PSD95, and NeDLG. CLASP-1 is a member of a superfamily of immune-cell associated proteins with similar motifs (see PCT/US99/22996 published as WO 00/20434). CLASP-1 functions in the maintenance of the immune synapse. The CLASP-1 transcript is present in lymphoid organs and neural tissue, and the protein is expressed by T and B lymphocytes and macrophages in the MOMA-1 subregion of the marginal zone of the spleen, an area known to be important in T:B cell interaction. CLASP-1 staining of individual T and B cells exhibits a preactivation structural polarity, being organized as a “ball” or “cap” structure in B cells, and forming a “ring”, “ball” or “cap” structure in T cells. The placement of these structures is adjacent to the microtubule-organizing center (“MTOC”). CLASP-1 antibody staining indicates that CLASP-1 is at the interface of T-B cell conjugates that are fully committed to differentiation. Antibodies to the extracellular domain of CLASP-1 also block T-B cell conjugate formation and T cell activation.

Antagonism of CLASP-1 function is expected to interfere with immune responses (e.g., T and B cell activation), signal transduction, cell-cell interactions, and membrane organization. Diseases to be treatment by CLASP-1 agonists/antagonists include, but is not limited to, rheumaotoid arthritis, juvenile diabetes, organ rejection, graft-versus-host disease, scleroderma, multiple sclerosis.

G. CLASP4

As shown supra, CLASP4 interacts with DLG1, PSD95, NeDLG, LDP, AF6, 41.8, and MINT1. CLASP4 is a member of a superfamily of immune-cell associated proteins with similar motifs (see copending U.S. Pat. Application 60/196,527 filed Apr. 11, 2000). The CLASP4 protein is expressed primarily in peripheral blood lymphocytes. Inhibition of the interaction of CLASP-4 and PDZ domains will interfere with immune responses (e.g., T and B cell activation), signal transduction, cell-cell interactions, and membrane organization. Diesease to be treated bu CLASP4 agonists/antagonists include, but is not limited to, rheumaotoid arthritis, juvenile diabetes, organ rejection, graft-versus-host disease, scleroderma, multiple sclerosis, acute leukemias, leukemic blast crisis, post-infarction inflamation (cardiac, etc.), atherosclerosis.

H. VCAM1

The vascular cell adhesion molecule-1 (VCAM-1, CD106) is predominantly expressed by vascular endothelium (i.e., endothelial cells) but has been detected in macrophages, dendritic cells, bone marrow-derived cells, fibroblasts, cortical thymic epithelial cells, vascular smooth muscle cells, myoblasts and myotubes. VCAM-1 mediates adhesion through interacting with an integrin ligand, VLA-4, which is expressed by lymphocytes, monocytes and eosinophils. The interaction between VCAM-1 and VLA-4 is important for activation, flattening and extravasation of VLA4 expressing cells when the endothelium itself has become activated due to inflammation or injury (Salomon et al., 1997, Blood 89:2461-2471; St-Pierre et al., 1996, Eur. J. Immunol. 26:2050-2055; Bell et al., 1995, Int. Immunol. 7:1861-1871).

As discussed supra, the C-terminal region of VCAM-1 is a ligand for the PDZ domains of MPP1, DLG1, NeDLG1, LDP, 41.8 protein, TIAM1, K807, WWP3 and K303. These interactions are believed to mediate the function of endothelial cell interactions with integrin expressing leukocytes. When the PDZ-PL interactions are disrupted, the adherence of leukocytes to the endothelium will similarly be disrupted, resulting in, e.g., reduction of inflammation. Thus, inhibition of VCAM-1 binding to PDZ proteins is useful for reducing abnormal VCAM-1 inflammatory responses and associated pathologies such as (but not limited to) renal allograft rejection, insulin-dependent diabetes, rheumatoid arthritis, post-myocardial infarction complications and systemic lupus erythematosus (Pasloske et al., 1994, Ann Rev Med 45:283; Ockenhouse et al., 1992, J. Exp. Med. 176:1183; Solezk et al., 1997, Kidney Int. 51:1476; Tedla, et al., 1999, Clin. Exp. Immunol. 117:92-99; Kusterer et al., 1999, Exp Clin Endocrinol Diabetes 107:S102-107; Bonomini et al., 1998, Nephron 79:399; Suassuna et al., 1994, Kidney Int. 46:443; Ferri et al., 1999, Hypertension 34:568).

I. CLASP-2

As shown supra, CLASP-2 interacts with PSD-95, NeDLG, and DLG1. CLASP-2 is a member of a superfamily of immune-cell associated proteins with similar motifs (see copending U.S. patent Ser. No. 09/547,276 filed Apr. 11, 2000; WO 00/10158 filed Apr. 11, 2000; WO 00/10156 filed Apr. 11, 2000). The CLASP-2 transcript is present most strongly in placenta followed by lung, kidney and heart and the protein is expressed in T and B cells, and kidney epithelial cells.

Inhibition of the interaction of CLASP-2 and PDZ domains will interfere with CLASP-2 function resulting in interference with T and B cell function (e.g., T and B cell activation), signal transduction, cell-cell interactions, and membrane organization. In addition, since CLASP-2 is present in heart, blocking CLASP-2 function or expression can selectively block immune responses in the heart (for example, to selectively stop immune response in the heart compartment, e.g., following cardiac transplant rejection or post-MI inflammation, without compromising immunity elsewhere). Other diseases to be treatment by CLASP-1 agonists/antagonists include, but is not limited to, rheumaotoid arthritis, juvenile diabetes, organ rejection, graft-versus-host disease, scleroderma, multiple sclerosis.

J. CD95 (Apo-1/Fas)

CD95 (Fas/Apo-1) and Fas ligand (FasL) are a receptor-ligand pair involved in lymphocyte homeostasis and peripheral tolerance. Binding of Fas by its ligand results in apoptotic cell death, an important major mechanism for safe clearance of unwanted cell during resolution of the acute inflammatory response. As is shown supra, CD95 binds the PDZ domains DLG1, PSD95, NeDLG, TIP1, and 41.8. Agents that modulate (e.g., inhibit) the interaction of CD95 and PDZ domains are useful for treatment of diseases, e.g., organ transplantation, graft-versus-host disease, Crohn's Disease, Ulcerative colitis, inflammatory bowel disease, rheumatoid arthritis, osteoarthritis, multiple sclerosis, scleroderma, mixed connective tissue disease, leukemia and other malignancies.

K. KV1.3 (Shaker Type Kv1.3 Potassium Channel)

As shown supra, Kv1.3 binds DLG1, PSD95, NeDLG, LIMK, 41.8, RGS12, DVL1, and MINT1. Kv1.3 is a Shaker-related channel protein that is involved in modulating the membrane potential of T lymphocytes (Lewis and Cahalan, 1995, Ann. Rev. Immunol. 13:623). Inhibition of the Kv1.3 channel chronically depolarizes the T cell membrane, reduces calcium entry via calcium-activated release calcium channels in the plasma membrane, and consequently inhibits the calcium-signaling pathway essential for lymphocyte activation. Hanada et al., reported that Kv1.3 is associated with DLG1 and PSD95 in Jurkat T cells (J. Biol. Chem. 1997, 272:26899). Administration of Kv1.3-PDZ protein agonist/antagonists will disrupt T cell signaling and can be a useful therapeutic drug to treat, but not limited to, organ transplantation, graft-versus-host disease, Crohn's Disease, Ulcerative colitis, inflammatory bowel disease, rheumatoid arthritis, osteoarthritis, multiple sclerosis, scleroderma, mixed connective tissue disease.

L. DNAM-1

As shown supra, DNAM-1 binds several PDZ proteins, including MPP1, MPP2, DLG1, PSD95, NeDLG, LIM, AF6, 41.8, RGS12 and WWP3. DNAM-1 is associated with Fyn constitutively but required the presence of pervanadate (a tyrosine phosphatase inhibitor) (Shibuya, et al., 1999, Immunity. 1:615-623). Upon stimulation with anti-CD3 or cross-linking DNAM-1 with anti-DNAM-1, DNAM-1 is phosphorylated at Ser329 (Shibuya, et al., 1999, Immunity 1:1671-75) and associates with LFA-1. Furthermore, Fyn becomes associated with DNAM-1 independent of pervanadate. Fyn phosphorylates DNAM-1 at Tyr322, but does not require Tyr322 to continue binding to DNAM-1.

Since DNAM-1 itself does not have a SH3 binding domain but has a Src phosphorylation site at Tyr322, an adaptor molecule must be present to bridge DNAM-1 and Fyn. DLG1 has been described in the literature to be present in T cells (Hanada, et al. 1997, J. Biol. Chem. 272:26899), but does not bind to Fyn. Although PSD95 does not have a SH3 site, several of the other PDZ proteins do have SH3 binding domains including but not limited to NeDLG, RGS12, WWP3 and MPP2, and can fulfill this function. The adaptor PDZ list supra describes binding to DNAM-1 through the PDZ domain. Tyr139 is a candidate phosphorylation site to control association of Fyn to DNAM-1 and the adaptor PDZ. In addition, through WWP3, DNAM-1 may complex with beta-catenin, actin and cadherin (Dobrosotskaya and James, 2000, Biochem. Biophys. Res. Commun. 270:903-909).

Based on this analysis, inhibition of PDZ association with DNAM-1 using the reagents of the invention will inhibit Fyn association with DNAM-1 and the subsequent Tyr322 phosphorylation and activation of cytotoxic T cells. Disease that can be treated include but are not limited to Crohn's Disease, multiple sclerosis, ulcerative colitis, inflammatory bowel disease, graft-versus-host, juvenile diabetes, and Hashimoto's Disease.

M. CD83 (HB15)

CD83 is a transmembrane glycoprotein, expressed predominantly on activated dendritic cells (DCs), Langerhans cells in the skin, with some weak expression detected on activated peripheral lymphocytes, and interdigitating reticulum cells within the T cell zones of lymphoid organs (Zhou and Tedder, 1995, J. Immunol. 154:3821-3835; Zhou et al., 1992, J. Immunol. 149:735-742). CD83 is up-regulated de novo upon activation of an immature DCs, and is the major discriminating marker and characteristic for activated, mature DCs (Czerniecki et al., 1997, J. Immunol. 159:3823-3837). DCs function as antigen presenting cells (APCs). Upregulation and expression of CD83 thus appears to be required for DCs to mature and function as APCs.

As shown by experiments described supra, the CD83 binds to the PDZ domains of DLG1, PSD95, and NeDLG. These interactions between CD83 and PDZ domains, and between CD83 and DLG1, PSD95, and NeDLG are believed to be important for proper distribution and recycling of CD83. Disruption of CD83 and PDZ proteins with agonists and antagonist can be used to treat, but not limited to, psoriasis, cancers, allergies, autoimmune diseases such as multiple sclerosis, system lupus erythematosis.

N. CD44 (Phagocytic Glycoprotein 1, Lymphocyte Homing Receptor, p85 and HCAM)

CD44 is single pass transmembrane protein that has several different isoforms due to alternative splicing. It has a broad pattern of expression being detected on both hematopoietic and non-hematopoietic cell types including epithelial, endothelial, mesenchymal and neuronal cells. CD44H is a major isoform that is expressed in lymphoid, myeloid and erythroid cells (reviewed in Barclay et al., 1997, The Leukocyte Antigen Facts Book, 2ed, Academic Press). CD44 is a receptor for hyaluronate (HA), which is a constituent of the extracellular matrix (ECM). In the immune system, CD44 functions as an adhesion molecule on the surface of leukocytes and erythrocytes that binds HA polymers in the ECM, and it can also act as a signaling receptor when HA becomes soluble during inflammatory reactions or tissue damage. The cytoplasmic region of CD44 has been shown to bind or be associated with the actin cytoskeleton through interactions with spectrin and members of the ERM (ezrin, radixin, and meosin) family (reviewed in Lesley et al., 1993; Bajorath, 2000, Proteins 39:103-111). Additionally, CD44 is associated with the non-receptor tyrosine kinase p56Lck (Taher et al., 1996, J. Biol. Chem. 271:2863-2867. CD44 has been shown to be a co-stimulatory molecule with CD3/TCR engagement to activate T cells (reviewed in Aruffo, 1996, J. Clin. Invest. 98:2191-2192).

As described supra by experiments reported herein, the C-terminus of CD44 is a ligand for the PDZ domain contained in MPP1, prIL-16 and MINT1. It is believed that the interactions of CD44 with PDZ domains, and between CD44 with MPP1, prIL-16 and MINT1 function in maintenance of leukocyte structure and in leukocyte signaling. Thus, when a CD44-PDZ interaction is disrupted, CD44 will fail to transduce proper intracellular signals, and maintain proper distribution of CD44 on the surface, which will prevent adhesion of leukocytes to the endothelium during inflammation and tissue damage. Administration of agonists/antagonists of this interaction will thus result in, but not limited to, reduced inflammatory responses during tissue ischemia and cell lysis (e.g., rhabdomyosis), vascular insufficiencies (e.g. frostbite), psoriasis, eczema, graft-versus-host disease, granuloma annulare, scleroderma.

O. CD97 (CD55)

As discussed supra, CD97 binds the PDZ domains of DLG1 and 41.8. CD97 is a 79.7 kD seven-span transmembrane protein expressed on granulocytes and monocytes and at low levels on resting T cells and B cells. Upon T or B cell activation expression levels of CD97 in T cells and B cells increases rapidly (Eichler et al., 1994, Scand. J. Immunol. 39, 111-115; Pickl et al., 1995, Leukocyte Typing V:1151-1153). When expressed on COS cells, CD97 confers adhesion to lymphocytes and to erythrocytes.

According to the present invention, the interaction of CD97 with DLG1 and the 41.8 kd protein can be altered to interfere with proper membrane distribution of CD97 and/or recycling of CD97. Such modulation will affect CD97 dependent adherence of cells with therapeutic benefit. Without being limited, agonists and antagonists of CD97-PDZ protein interaction can be used to treat rheumatoid arthritis, osteoarthritis, Crohn's Disease, Ulcerative colitis, psoriasis.

P. Glycophorin C (GC)

As is shown supra, the c-terminus of Glycophorin C (GC) interacts with the PDZ domains of human DLG, PSD95, NeDLG, MMP2, AF6, 41.8, and MINT1 (with Mint-1 described previously). Glycophorin C is an integral membrane protein expressed in erythroid cells, thymus, stomach, breast, adult and fetal liver, monocytes, T and B cells (Le Van Kim et al., 1989, J. Biol. Chem. 264:20407-20414) and is known for its role in human erythrocytes where it interacts with MPP1 and protein 4.1 to regulate the shape, integrity and mechanical stability of red cells (Marfatia et al., 1997, J. Biol. Chem. 272:24191-24197; Reid et al., 1987, Blood 69:1068-1072.).

Interactions between Glycophorin C and PDZ proteins DLG, PSD95, NeDLG, MMP2, AF6, 41.8, and MINT1 are believed necessary for maintenance of the physical integrity of cells in which they are expressed. Modulation of GC-PDZ interactions will alter with the function of these and can be utilized to treat, but not limited to, polycythemia vera, spherocytosis.

Q. CDw128A (IL8RA)

As is described supra, CDW128A binds to the PDZ domains of DLG1 and NeDLG. There are two forms for the IL-8 receptor, IL-8RA (CDw128A) and IL-8RB (CDw128B) both of which are members of the G protein-coupled receptor superfamily and chemokine receptor branch of rhodopsin family. CDw128A and CDw128B both bind IL-8 with the same affinity but only CDw128B, binds three other IL-8-related CXC chemokines: melanoma growth-stimulating activity (GRO/MGSA), neutrophil-activating peptide 2 (NAP-2) and ENA-78. See, e.g., Ahuja and Murphy, 1996, J Biol Chem 271:20545-50.

CDw128A is expressed on all granulocytes, a subset of T cells, monocytes, endothelial cells, keratinocytes, erythrocytes, and melanoma cells. IL-8 induces chemotaxis of neutrophils, basophils, and T lymphocytes and increases neutrophil and monocyte adhesion to endothelial cells. The binding of IL-8 to IL8RA induces a transient increase in intracellular calcium levels, activation of phospholipase D, a respiratory burst of neutrophils and chemotaxis. This pro-inflammatory response is effective in normal immune responses. Inhibitors of CDw128A are useful for treatment of psoriasis, rheumatoid arthritis, polyarthritis, and for control of angiogenesis-dependent disorders such as melanomas and breast cancer.

R. CD3-eta(η)

CD3-η is a splice variant of CD3 zeta and a component of the CD3/TCR complex, which is required for antigen recognition, signal transduction and activation of T cells (Weiss and Littman, 1994, Cell 76:263-274.). See, Barclay et al., 1997, The Leucocyte antigen facts book, 2nd Ed, Academic Press. As shown by experiments reported herein, the C-terminal region of CD3-η is a ligand for the PDZ domains of MINT1, 41.8 protein, DLG1, and PSD95. The interactions of CD3-η with PDZ domains MINT1, 41.8 protein, DLG1, K807, TIP, and PSD95 are believed to be important activation of T cells, which is required for all cellular immune responses. Modulation of this interaction by agonists and antogonists can be used to treat, but is not limited to, acute and chronic allograft rejection, multiple sclerosis, graft-versus-host disease, rheumatoid arthritis.

S. LPAP (CD45-AP, LSM-1)

LPAP is a transmembrane protein expressed on resting T-and B-cells. LPAP has been shown to bind to CD45, a protein that is part of the T-cell receptor complex and has been found to co-localize with CD4, CD2 and Thy-1. LPAP has also been co-immune precipitated with p56(lck) and ZAP-70. The actual function of LPAP is unknown, but data obtained from LPAP deficient mice and Jurkat cell lines suggest that LPAP is an assembly molecule important for the organization of a functional CD45 complex.

As shown supra, LPAP binds to DLG-1, MINT-1, KIAA0807 and TIP-1 (sometimes “TAX Interacting Protein 1” or “TAX Interacting Protein” or “TAX IP-1”). Notably, DLG-1, MINT-1, KIAA0807 and TIP-1 are expressed in T-cells. It has been shown that DLG-1 coprecipitates with p56(lck) in T-cells (Hanada et al., 1997, J. Biol. Chem.272(43):26899-26904). The assay described herein also demonstrates that DLG-1 binds to CD95 and KV1.3, MINT-1 binds to KV1.3, TIP-1 binds to CD95, KV1.3, CD3η and HTLV-1 TAX oncoprotein. All these molecules are involved in signaling by the TCR or in the regulation of cell death by apoptosis. LPAP is believed to function in organizing the signaling of CD45 in T-cells by recruiting p56(LCD) as a substrate for CD45. Blocking the function of CD45 has been shown to severely impair the T-cell response. The human PDZ domain containing protein KIAA0807 shows a high degree of similarity to the mouse protein MAST205, a serine/threonine protein kinase, and computer based protein domain homology search predicts protein tyrosine kinase activity for KIAA0807. Binding of KLAA0807 to LPAP appears to be involved in, and possibly crucial for, recruiting KIAA0807 encoded protein into the CD45 complex. Further, protein kinase activity of the KIAA0807 protein may add specific kinase function to the CD45 complex and might tune phosphatase activity of CD45. Thus, inhibition of the PDZ domain-PDZ ligand mediated interaction between LPAP and KIAA0807 can be used to alter (e.g., diminish or abolish) immune response.

The assay described herein also shows that TIP-1 binds to LPAP and to CD95. CDp5 has been shown to be a pivotal component of programmed cell death (apoptosis). Binding of TIP-1 to CD95 might be regulated by changes in phosphorylation of either binding partner, and LPAP mediated recruitment of TIP-1 into the CD45 (phosphatase/kinase)-complex might ensure proximity of phosphatase/kinase activity when TIP-1 engages in CD95 binding. In addition, TIP-1/LPAP binding might compete for TIP-1/CD95 binding, thus being involved in switching from T-cell proliferation to apoptosis.

Studies with LPAP null mice have shown impaired lymphocyte responses to antigen receptor (Motoya, et al., 1999, J. Biol. Chem. 274(3):1407-1414) and have suggested that LPAP plays a role in regulation of lymphocyte expansion in particular lymphatic organs (Ding, et al., 1999, Eur. J. Immunol. 29(12):3956-3961). Therefore, inhibiting the interaction between LPAP and PDZ proteins is expected to alter the CD45-mediated path from the rest of the immune response and to change the pattern of lymphocyte distribution. Agonists and antagonist of PL-PDZ binding can be used to treat a variety of disease, including immune disorders such as (but not limited to) rheumatoid arthritis, transplant rejection, multiple sclerosis, scleroderma, graft-versus host disease.

T. CD46 (Complement Membrane Cofactor Protein (MCP))

CD46 is a membrane protein expressed on all nucleated cells, but not on erythrocytes. CD46 is a member of the regulator of complement activation protein family. Its primary function is the protection of cells from complement attack by inactivating membrane deposited C3b/C4b complement (Liszewski, et al., 1999, Adv. Immunol. 61:201-283). CD46 exists in more than 8 isoforms that are generated by differential splicing, with molecular weights ranging from 45 to 70 kD. In addition to the above function, CD46 also serves as the receptor for the measles virus and for other pathogenic microorganisms (e.g. Streptococcus pyogenes) (Manchester et al, 1994, Proc. Natl Acad. Sci. USA 91:2161; Okada et al., 1995, Proc. Natl Acad Sci. USA 92:2489-2493.). CD46 also appears to be over-expressed on certain tumors (Jurianz et al., 1999, Mol Immunol 36:929-939) thus rendering tumor cells insensitive to the action of complement. See, Barclay et al., (1997) The Leucocyte antigen facts book, 2^{nd} ed, Academic Press.

As shown supra, CD46 binds DLG1, PSD95 and Ne-DLG. This interaction is believed to be necessary for proper membrane distribution of CD46 and/or recycling of CD46. Alteration of the CD46-PDZ interaction can reduce the ability of measles virus and other pathogens to enter cells, renders CD46-expressing tumors susceptible to attack by complement. The administration of CD46-PDZ interaction agonists and antagonists is useful for the treatment of, but not limited to, cancers and viral infectious diseases.

U. CDw128B

As is described supra, CDw128B binds to the PDZ domains of DLG1, NeDLG, PSD95, K807 and 41.8 in the assays described supra. There are two forms for the IL-8 receptor, IL-8RA (CDw128A) and IL-8RB (CDw128B) both of which are members of the G protein-coupled receptor superfamily and chemokine receptor branch of rhodopsin family. CDw128A and CDw128B both binds IL-8 with equal affinity but only CDw128B, also binds three other IL-8-related CXC chemokines: melanoma growth-stimulating activity (GRO/MGSA), neutrophil-activating peptide 2 (NAP-2) and ENA-78. See, e.g., Ahuja, S K and Murphy, P M. 1996. J Biol Chem 271:20545-50.

CDw128B is expressed on all granulocytes, a subset of T cells, monocytes, endothelial cells, keratinocytes, erythrocytes, and melanoma cells. IL-8 induces chemotaxis of neutrophils, basophils, and T lymphocytes but diminished relative to IL8RA and increases neutrophil and monocyte adhesion to endothelial cells. The binding of IL-8A to its receptor induces a transient increase in intracellular calcium levelsand granule release but does not induce activation of phospholipase D or a respiratory burst in neutrophils. This pro-inflammatory response is effective in normal immune responses. Inhibitors of CDw128B are useful for treatment of psoriasis, rheumatoid arthritis, polyarthritis, and for control of angiogenesis-dependent disorders such as melanomas and breast cancer.

V. DOCK2

The DOCK family is a group of transmembrane proteins that interact with the cytoskeleton to affect cell shape, and maintain structural integrity of functional subdomains within a cell. Members of this new family include Drosophila myoblast city (mbc), DOCK180 (DOCK1), DOCK2, DOCK3, CED5, KIAA0209 and CLASP. The prototypical molecule, DOCK1 or DOCK180 is the human homologue of the C. elegans gene, CED5, which is involved in the engulfment and phagocytosis by macrophages of cells undergoing apoptosis (apoptotic cells). Of the human family members, DOCK2 is most closely related to DOCK1. DOCK2 expression appears to be confined to cells of leukocytic origin. DOCK2 is found in peripheral blood lymphocytes and can convert a flatten cell into a rounded morphology upon transfection (Nagase, et. al., 1996, DNA Res 3:321-29;, Nishihara, 1999, Hokkaido Igaku Zasshi 74:157). DOCK2 mRNA is highly expressed in peripheral blood lymphocytes, with lower expression in thymus and spleen and very weak expression in colon and small intestine. Immunohistochemistry with antibodies directed against DOCK2 detect expression of DOCK2 in macrophages in the interstitium and alveoli. DOCK2 protein is also detected in lymphocytes of the lymph node, and macrophage and lymphocytes in tonsillar tissue (Nishihara et al. 1999. Bioch. Biophys. Acta 1452:179). DOCK2, like DOCK1, has been shown to bind Rac1, a GTPase, which could account for it's ability to round-up flat NRK (normal rat kidney) cells (Nishihara, 1999, Hokkaido Igaku Zasshi 74:157).

As shown supra, DOCK2 is a PL and binds to the PDZ-containing proteins KIAA807, DLG1, PSD95, NeDLG, Syntrophin α1 (Syntα1) and KLAA0561. In addition to a PDZ domain, KLAA807 contains a kinase domain suggesting that the interaction between KIAA807 and DOCK may have a role in signal transduction. The function of KIAA0561 is unknown at this time. DLG1, PSD95, NeDLG and Syntrophin al have roles in cell signaling and structure (Gomperts, 1996, Cell 84:659; Shen and Wyszynski, 1997, Bioessays 19:847; Sheng and Kim, 1996, Curr. Opin. Neurobiol. 6:602; Leu et al., 1994, Proc. Nat. Acad. Sci. 91:9818). Most notably, PSD95, NeDLG and Syntrophin α1 are necessary for proper rganization of neuronal synapses (Adams et al., 2000, J. Cell Biol. 150:1385; Masuko et al., 1999, J. Biol. Chem. 274:5782). Using PDZ adaptor proteins, DOCK2 would be required to control cell shape of lymphocytes for their transit through the vascular circulation through its direct or indirection interaction with Rac1. The PDZ-PL interactions would be necessary for proper assembly and maintenance of the immunological synapse in a similar manner for their role in the neuronal synapse. These interactions would allow proper cell-to-cell interactions required to initiate immune reactions. Modulation of DOCK2-PDZ interactions by agonists and antagonists can be used to treat diseases such as, but not limited to, acute lymphoytic leukemias, leukemic blast crisis, post-myocardial infarction inflammation, and post-traumatic inflammation.

W. CD34

As is shown supra, CD34 binds DLG1, PSD95, K807, and NeDLG. CD34 is expressed on a small subpopulation of bone marrow cells which includes hematopoietic stem cells. CD34 is also present on bone marrow stomal cells and on endothelial cells. The selectins CD62L (L-selectin) and CD62E (E-selectin) bind CD34. CD34 mediates attachment and rolling of leukocytes. The hematopoietic stem cell properties of CD34 include myeloid differentiation of stem cells. Modulation of the CD34-PDZ interaction with agonists and/or antagonists can be used to treat, but is not limited to, myelodysplasia, leukemias, post-traumatic inflammation, post-myocardial infarction inflammation.

X. Fc Epsilon Receptor Beta I Chain (FcεRβI)

The high affinity receptor for human IgE, FcεRI, is composed of an α, β, and disulfide-linked γ homodimer. The α-chain binds the Fc portion of IgE, whereas the β-chain serves to amplify signals that are transduced through the γ-chain homodimer. Both αβγ2 tetramer and αγ2 trimer complexes exist, but the β-chain amplifies the signal 5- to 7-fold, as measured by Syk activation and calcium mobilization. Additionally, the FcεRβI is a PDZ ligand and is a member of the CD20/FcεRβI receptor family. As is shown supra, FcεRβI binds MINT1.

As the high-affinity receptor for IgE, FcβRI on basophils and mast cells plays a central role in the initiation of allergic responses. Signaling through the FcβRI begins by crosslinking of a multivalent allergen bound to IgE. The result is vesicular degranulation, release of histamine, leukotrienes and pro-inflammatory cytokines (IL-6 and TNFα), factors responsible for the symptoms of immediate hypersensitivity. Alteration of signaling by targeting PL/PDZ interaction with agonists and antagonists can be used to treat, but is not limited to, asthma, atopic dermatitis, eczema, drug reaction, mastocytosis, urticaria, eosinophilia myalgia syndrome (Turner, H., et. al., 1999, Nature 402 SUPP:B24).

Y. FAS Ligand (FasL)

CD95 (Fas/Apo-1) and Fas ligand (FasL) are a receptor-ligand pair critically involved in lymphocyte homeostasis and peripheral tolerance. Binding of Fas by its ligand results in apoptotic cell death, an important major mechanism for safe clearance of unwanted cell during resolution of the acute inflammatory response. FasL is mainly restricted to activated T lymphocytes and is rapidly induced. Fas ligand is frequently up-regulated in breast cancer, as compared with normal breast epithelial cells and benign breast disease. As is shown supra, FasL binds the KIAA0561 PDZ domain. The PDZ-PL modifiers are useful for treatment of, but not limited to, tumors, e.g., tumors unresponsive to conventional chemotherapy.

Z. CDW125 (IL5R)

As is shown supra, CDW125 binds PTN4 and RGS12. CDW125 is an IL-5 receptor expressed on eosinophils and basophils. IL5 promotes growth and differentiation of eosinophil precursors and actives mature eosinophils (Takatsu et al 1994, Adv. Immunol. 57:145-190). The secreted form of CDw125 has antagonistic properties and is able to inhibit IL-5-induced eosinophil proliferation and differentiation. Modulation of CDW125 binding to PDZ domains may be used to treat, but is not limited to, asthma, atopic dermatitis, eczema, drug reaction, urticaria, mastocytosis, eosinophilia.

AA. Burkitt's Lymphoma Receptor-1 (BLR-1; CXCR5)

BLR-1 is a transmembrane receptor detected primarily on B cells, and shown to be upregulated in stimulated T cells (Dobner et al., 1992, Eur J. Immunol. 22:2795-2799.; Flynn et al., 1998, J. Exp. Med 188:297-304). BLR-1 functions in chemotaxis of B and T cells into follicles of secondary lymphoid organs (e.g. spleen) for proper development and selection toward antigens (Forster et al., 1996, Cell 87:1037-1047. Its ligand is B-lymphocyte chemoattractant (BLC), which is strongly expressed in the follicles of Peyer's patches, spleen and lymph nodes (Gunn et al., 1998, Nature 391:799-803). Consistent with its chemotactic role is the demonstration that BLR-1 expression is downregulated in developed, activated B cells (plasma cells) to prevent them from being retained in follicles (Forster et al., 1994, Cell Mol Biol 40:381-387), and blr −/− B cells fail to migrate into B cell follicles (Forster et al., 1996).

It has been unclear how BLR-1 is organized on the cell surface and how the signaling occurs intracellularly. Through the PRISM MATRIX assay, we have deduced several intracellular proteins that interacts with BLR-1 that may play an important role in organizing intracellular and extracellular events related to BLR-1 function in B cell maturation. Disruption of PL/PDZ-mediated interactions between BLR-1 and PDZ domain molecules would be anticipated to interrupt BLR-1 signaling during B cell maturation, with implications for treatment and diagnosis of immune deficiency, allergy and autoimmunity.

PDZ domain containing proteins function by clustering proteins to organize macromolecular protein complexes, including modulation of protein phosphorylation. As shown supra, BLR-1 binds to the PDZ domain containing molecules MINT1, PDZK1, KIAA0807 and KLAA0561. Notably, MINT1, PDZK1, KIAA0807 and KLAA0561 are expressed in B-cells. Without intending to be bound by a particular mechanism, we suggest, that BLR-1 interactions with MINT1 and PDZK1 are crucial for proper BLR-1 clustering into functional macromolecular complexes whereas BLR-1 binding to KIAA0807 and KIAA0561 is likely to be associated with changes in the phosphorylation state of BLR-1 (or other molecules associated with BLR-1). In turn, BLR-1 phosphorylation may regulate BLR-1 binding to PDZK1 and MINT1.

MINT1 is known as part of a multi-protein complex that occurs in the neuroogical synapse. This complex functions as an intermediate for synaptic vesicle fusion and/or vesicle docking (Okamoto and Sudhof, 1997, J. Biol. Chem. 272(50):31459-31464). We suggest, that binding of MINT1 to BLR-1 as detected by the herein described assay might constitute a crucial step in recruitment, transport and/or functional organization of active BLR-1 complexes.

In several instances, the PDZK1 protein has been demonstrated to be a potent organizer of functional membrane bound multimeric protein complexes. PDZK1 is a four-PDZ domain molecule which is peculiar in that its carboxy terminal end constitutes a PL that has the property to interact with the PDZK1 most N-terminal PDZ domain (Kocher, et al., 1999, Lab Invest 79:1161-70). MAP17, cMOAT, CFTR, and scavenger receptor type B are four known examples of membrane bound and clustered proteins that engage via PDZ/PL mediated interactions with PDZ domains of PDZK1. MAP17 is expressed in normal kidney epithelium, but it is strongly upregulated in human kidney, colon, lung and breast carcinomas (Kocher, et al., Lab Invest., 1998, 78:117-125). cMOAT (canalicular multispecific anionic transporter) is a protein involved in multidrug resistance and is overexpressed in several carcinoma cells lines; (Kocher, et al., Lab Invest., 1999, 79:1161-1170). CFTR functions as a chloride channel which causes cystic fibrosis when mutated. CFTR binds to PDZK1 PDZ domains 2-4. The clustering of several CFTR molecules mediated by PDZK-1 has been shown to potentiate CFTR function (Wang et al., 2000, Cell, 103:169-179). Scavenger receptor type B is expressed in liver cells and is involved in the uptake of cholesterol from high density lipoproteins. The interaction between scavenger receptor type B and PDZK-1 seem to be associated with the regulation of atherogenesis (Ikemoto et al., 2000, Proc. Natl. Acad. Sci. 97:6538-6543).

The above described examples demonstrate that proper spatial organization and concentration of MAP17, cMOAT, CFTR, and scavenger receptor type B are vitally correlated with their proper physiological function. Furthermore, these observations strongly suggest that PDZK1 has a pivotal role in recruiting and in organizing these multimeric membrane associated protein complexes. We suggest, that PDZK1 is also crucial for recruiting and organizing BLR-1 into a functional membrane associated complex.

Intra-molecular binding of the PDZK1 PL to N-terminal PDZK1 PDZ domain might ensure proper orientation of up to three PL molecules (homo- or heterotrimer) bound to PDZK1 domains 2, 3, and 4. In addition, PDZK1 PDZ domain 1 might interact with the PL of another PDZK1 molecule in a head-to-tail fashion (inter-molecular), thus enabeling the local formation of stable high density homo- or heteromeric complexes. The equilibrium between intra-molecular and inter-molecular PDZK1 PL/PDZ binding might depend on the local PDZK1 concentration, whereas phosphorylation changes at the PDZK1 PL and/or the BLR-PL might function as an on/off switch for either interaction. BLR-1 PL phosphorylation might be regulated by KIAA0807 and KIAA0561 proteins when interacting with BLR-1.

KIAA0807 and KIAA0561 encode human PDZ domain containing proteins that are highly similar to the mouse protein MAST205, a serine/threonin protein kinase, and computer based protein domain homology search predicts protein thyrosine kinase activity for both, KIAA0807 and KLAA0561. PL/PDZ mediated binding of BLR-1 to KLAA0807 and to KIAA0561 proteins as detected by the herein described assay might change the phosphorylation status of BLR-1 and thus regulate its activity and binding properties; in addition, the BLR-1/KIAA0807 and the BLR-1/KIAA0561 interactions might associate protein kinase activity with BLR-1 which, in turn, might modulate the phosphorylation status of PDZK1 or any other of its PDZ domain containing interacting proteins in the course of (BLR-1) transport to and/or clustering in the cell membrane. The phosphorylation status of either binding partner might constitute the switch between engagement into and interruption of a PL/PDZ mediated protein-protein interaction.

As shown by experiments reported herein, the C-terminal end of BLR-1 binds to the PDZ domain containing proteins PDZK1, MINT1, KIAA0807 or KLAA0561. Without intending to be bound by a particular mechanism, the interaction between BLR-1 and the PDZ domain proteins is necessary for the proper distribution and signaling of BLR-1 on the cell surface. Normal function of the receptor is required for physiological B lymphocyte chemotaxis and thus for B lymphocyte function. This function of the receptor is disrupted by small molecule therapeutics that disrupt BLR-1/PDZ binding (see, e.g., Example 7, infra). When this interaction is disrupted, the chemotactic abilities of lymphoid cells expressing BLR-1 is similarly disrupted. Such a disruption results in a reduced immune response, interference with the ability of lymphocytes to properly circulate and develop responses to antigen. Such small molecule therapeutics are thus immunosupressive agents that specifically target B lymphocytes. These therapeutics are of use in the treatment of autoimmune disorders involving overactivity of B lymphocytes, such as rheumatoid arthritis, systemic lupus erythematosis, and pemphigus vulgaria. Agonists and antagonists of the BLR-PDZ interaction are used to treat immune system diseases including, but is not limited to, rheumatoid arthritis, transplant rejection, multiple sclerosis, scleroderma, graft-versus host disease, systemic lupus erythematosus, scleroderma and other autoimmune diseases.

BB. CD4

CD4 is a co-receptor with the T cell receptor (TCR) involved in antigen recognition. Both CD4 and TCR belong to the immunoglobulin supergene family. T cell activation is enhanced by increasing the avidity of T cells for effector and target cells. The cytoplasmic domain is involved in signal transduction and association with the tyrosine kinase p56^{lck}. CD4 is expressed on most thymocytes, two-thirds of peripheral blood T lymphocytes, monocytes and macrophages.

Human immunodeficiency virus type-1 (HIV-1) infects cells by membrane fusion mediated by its envelope glycoproteins (gp120-gp41) and is triggered by the interaction of CD4 and a chemokine co-receptor, CCR5 or CXCR4. Modulation of CD4-PDZ inhibitors with agonists and antagonists can be used to treat, but is not limited to, HIV infection immediately after exposure to HIV, rheumatoid arthritis, multiple sclerosis, scleroderma, systemic lupus erythematosis, psoriasis.

CC. PAG (Phosphoprotein Associated with GlycoSphingolipid-Enriched Microdomains)

PAG is a recently identified, transmembrane adaptor phosphoprotein. It is expressed in hematopoietic cells including peripheral blood lymphocytes, monocytes and neutrophils, and is a substrate for kinases including the Lck and Fyn (Brdicka et al. 2000 J. Exp. Med. 191:1592-1604). We have discovered using the methods of the inventon (see Table 2) that PAG contains a PDZ-ligand motif and have demonstrated that PAG binds the PDZ-containing protein kinase KIAA807. This PDZ-PL interaction is consistent with an adaptor or scaffolding role for PAG.

Subcellularly, PAG is found with glycosphingolipid-enriched microdomains (GEMs, also known as lipid rafts or detergent-insoluble glycolipid-rich membrane domains). GEMs are 70 nm detergent resistant membrane islands found within the bulk plasma membrane of a cell. Each GEM has a concentration of lipids with higher saturated fatty acid side-chains, which favors their association. Importantly, GEMs also contain vital signal transduction molecules including kinases (e.g. Lck, Fyn, LAT, and PI-3′-kinase), adaptor proteins (LAT) and G-proteins. In hematopoietic cells GEMs are a required, functional component of immune cell activation since disruption of GEMs attenuates their activation. Additionally, GEMs functionally interact with the lymphocyte cytoskeleton, which is necessary for microtubule organization and proper lymphocyte activation (Xavier and Seed,1999, Curr. Op. in Immunol. 11:265-269; Xavier et al., 1998, Immunity 8:723-732; Montixi et al., 1998, EMBO J. 17:5334-5348).

The identification of PAG as a component of GEMs and a target of kinases found within GEMs indicates that PAG is an important mediator of cellular signal transduction, and is able to recruit other signaling components, namely KIAA807, to GEMs and regulate signal transduction. PAG has been shown to be a negative regulator of T-cell activation (Brdicka et al., 2000, J. Exp. Med. 191:1592-1604), and the kinase activity of KIAA807 may transduce the negative regulatory effects of PAG. Such an association provides a means to modulate immune function. As a negative regulator of T-cell activation, enhancement of the PAG:KIAA807 interaction may reduce an overactive immune system in autoimmune disease states during transplantation rejection, rheumatoid arthritis, systemic lupus erythematosus (SLE) and multiple sclerosis. In contrast disruption of this interaction may stimulate the activation of lymphocytes, which could help patients with immune deficiencies such as HIV-induced AIDS.

### 6.5 Agonists and Antagonists of PDZ-PL Interactions

As described herein, interactions between PDZ proteins and PL proteins in cells (e.g., hematopoietic cells, e.g., T cells and B cells) may be disrupted or inhibited by the administration of inhibitors or antagonists. Inhibitors can be identified using screening assays described herein. In embodiment, the motifs disclosed herein are used to design inhibitors. In some embodiments, the antagonists of the invention have a structure (e.g., peptide sequence) based on the C-terminal residues of PL-domain proteins listed in TABLE 2. In some embodiments, the antagonists of the invention have a structure (e.g., peptide sequence) based on a PL motif disclosed herein.

The PDZ/PL antagonists and antagonists of the invention may be any of a large variety of compounds, both naturally occurring and synthetic, organic and inorganic, and including polymers (e.g., oligopeptides, polypeptides, oligonucleotides, and polynucleotides), small molecules, antibodies, sugars, fatty acids, nucleotides and nucleotide analogs, analogs of naturally occurring structures (e.g., peptide mimetics, nucleic acid analogs, and the like), and numerous other compounds. Although, for convenience, the present discussion primarily refers antagonists of PDZ-PL interactions, it will be recognized that PDZ-PL interaction agonists can also be use in the methods disclosed herein.

In one aspect, the peptides and peptide mimetics or analogues of the invention contain an amino acid sequence that binds a PDZ domain in hematopoietic cells such as T cells and B cells, or otherwise inhibits the association of PL proteins and PDZ proteins. In one embodiment, the antagonists comprise a peptide that has a sequence corresponding to the carboxy-terminal sequence of a PL protein listed in TABLE 2, e.g., a peptide listed TABLE 4. Typically, the peptide comprises at least the C-terminal two (3), three (3) or four (4) residues of the PL protein, and often the inhibitory peptide comprises more than four residues (e.g., at least five, six, seven, eight, nine, ten, twelve or fifteen residues) from the PL protein C-terminus. See, e.g. Section 6.5.1, infra Moreover, the C-terminal domains of specific surface receptors expressed by hematopoietic system and endothelial cells may themselves be used as inhibitors, and may be used as the basis for rational design of non-peptide inhibitors. See Section 6.6, infra.

In some embodiments, the inhibitor is a peptide, e.g., having a sequence of a PL C-terminal protein sequence. See, e.g. Section 6.5.i, infra

In some embodiments, the antagonist is a fusion protein comprising such a sequence. Fusion proteins containing a transmembrane transporter amino acid sequence are particularly useful. See, e.g. Section 6.9, infra.

In some embodiments, the inhibitor is conserved variant of the PL C-terminal protein sequence having inhibitory activity. See, e.g. Section 6.5.2, infra.

In some embodiments, the antagonist is a peptide mimetic of a PL C-terminal sequence. See, e.g. Section 6.5.3, infra.

In some embodiments, the inhibitor is a small molecule (i.e., having a molecular weight less than 1 kD). See, e.g. Section 6.5.4, infra.

6.5.1 Peptide Antagonists

In one embodiment, the antagonists comprise a peptide that has a sequence of a PL protein carboxy-terminus listed in TABLE 2. The peptide comprises at least the C-terminal two (2) residues of the PL protein, and typically, the inhibitory peptide comprises more than two residues (e.g, at least three, four, five, six, seven, eight, nine, ten, twelve or fifteen residues) from the PL protein C-terminus. The peptide may be any of a variety of lenghts (e.g., at least 2, at least 3, at least 4, at least 5, at least 6, at least 8, at least 10, or at least 20 residues) and may contain additional residues not from the PL protein. It will be recognized that short PL peptides are sometime used in the rational design of other small molecules with similar properties.

Although most often, the residues shared by the inhibitory peptide with the PL protein are found at the C-terminus of the peptide. However, in some embodiments, the sequence is internal. Similarly, in some cases, the inhibitory peptide comprises residues from a PL sequence that is near, but not at the c-terminus of a PL protein (see, Gee et al., 1998, J Biological Chem. 273:21980-87).

Sometime the PL protein carboxy-terminus sequence is referred to as the “core PDZ motif sequence” referring to the ability of the short sequence to interact with the PDZ domain. For example, in an embodiment, the “core PDZ motif sequence” of a hematopoietic cell surface receptor at its C-terminus contains the last four amino acids, this sequence may be used to target PDZ domains in hematopoietic cells. As described above, the four amino acid core of a PDZ motif sequence may contain additional amino acids at its amino terminus to further increase its binding affinity and/or stability. Thus, in one embodiment, the PDZ motif sequence peptide can be from four amino acids up to 15 amino acids. It is preferred that the length of the sequence to be 6-10 amino acids. More preferably, the PDZ motif sequence contains 8 amino acids. Additional amino acids at the amino terminal end of the core sequence may be derived from the natural sequence in each hematopoietic cell surface receptor or a synthetic linker. The additional amino acids may also be conservatively substituted. When the third residue from the C-terminus is S, T or Y, this residue may be phosphorylated prior to the use of the peptide.

In some embodiments, the peptide and nonpeptide inhibitors of the are small, e.g., fewer than ten amino acid residues in length if a peptide. Further, it is reported that a limited number of ligand amino acids directly contact the PDZ domain (generally less than eight) (Kozlov et al., 2000, Biochemistry 39, 2572; Doyle et al., 1996, Cell 85, 1067) and that peptides as short as the C-terminal three amino acids often retain similar binding properties to longer(>15) amino acids peptides (Yanagisawa et al., 1997, J. Biol. Chem. 272, 8539).

FIGS. 3A-H show the use of peptides to inhibit PL-PDZ interactions using the G assay described supra. In FIGS. 3A and B, the inhibiton assays were carried out using GST fusion proteins containing PDZ domains from DLG1 or PSD95 (see supra and TABLE 3). Binding of biotinylated PL peptides for Clasp 2, CD46, Fas, or KV1.3 (as listed in TABLE 4) was determined in the presence of various competitor peptides (at a concentration of 100 uM) or in the absence of a competitor (equalized as 100% binding). The competitor peptides were 8-mers peptides having the sequence of C-terminus of Clasp 2 (MTSSSSVV, SEQ ID NO: 191), CD46 (REVKFTSL, SEQ ID NO: 113), or Fas (RNEIQSLV, SEQ ID NO: 48), a unlabeled 19-mer having the sequence of c-terminus of KV1.3 (i.e., non-biotinylated AA33L as listed in TABLE 3), or a peptide having the sequence of residues 64-76 of hemoglobin (Vidal et al., 1999, J. Immunol. 163, 4811), i.e., an unrelated competitor. The binding of biotinylated peptide (10 uM for Fas and KV1.3, 20 uM for Clasp 2 and CD46) to GST alone was subtracted from the binding to the fusion proteins to obtain the net signal for each experimental condition. This net signal was then normalized by dividing by the signal in the absence of competitor peptide and the data were plotted. Error bars indicated the standard deviation of duplicate measurements. Specific inhibition of Clasp 2 PL-DLG PDZ binding was observed with the CLASP-2 8-mer, the CD46 8-mer, the FAS-8-mer, and the KV13 peptide, but not in the absence of peptide or using an unrelated peptide.

FIGS. 3C-F show similar assays using shorter peptides to inhibit (e.g., a 3-mer and a 5-mer). FIGS. 3C-E show binding of biotinylated PL peptides for Clasp 2, CD46, Fas, or KV1.3, at the indicated concentration (as listed in TABLE 3) to GST fusion proteins containing PDZ domains from NeDLG, DLG1, or PSD95 in the absence or presence of 1 mM 3-mer peptide having the sequence of the C-terminus of Clasp 2 (SVV). (Table 3). FIG. 3F shows the effect on binding of a 5-mer CD49E peptide (ATSDA, SEQ ID NO: 25) to GST fusion proteins containing a PDZ domain from 41.8 Kd

6.5.2 Peptide Variants

Having identified PDZ binding peptides and PDZ-PL interaction inhibitory sequences, variations of these sequences can be made and the resulting peptide variants can be tested for PDZ domain binding or PDZ-PL inhibitory activity. In embodiments, the variants have the same or a different ability to bind a PDZ domain as the parent peptide. Typically, such amino acid substitutions are conservative, i.e., the amino acid residues are replaced with other amino acid residues having physical and/or chemical properties similar to the residues they are replacing. Preferably, conservative amino acid substitutions are those wherein an amino acid is replaced with another amino acid encompassed within the same designated class.

6.5.3 Peptide Mimetics

Having identified PDZ binding peptides and PDZ-PL interaction inhibitory sequences, peptide mimetics can be prepared using routine methods, and the inhibitory activity of the mimetics can be confirmed using the assays of the invention. Thus, in some embodiments, the antagonist is a peptide mimetic of a PL C-terminal sequence. The skilled artisan will recognize that individual synthetic residues and polypeptides incorporating mimetics can be synthesized using a variety of procedures and methodologies, which are well described in the scientific and patent literature, e.g., Organic Syntheses Collective Volumes, Gilman et al. (Eds) John Wiley & Sons, Inc., NY. Polypeptides incorporating mimetics can also be made using solid phase synthetic procedures, as described, e.g., by Di Marchi, et al., U.S. Pat. No. 5,422,426. Mimetics of the invention can also be synthesized using combinatorial methodologies. Various techniques for generation of peptide and peptidomimetic libraries are well known, and include, e.g., multipin, tea bag, and split-couple-mix techniques; see, e.g., al-Obeidi (1998) Mol. Biotechnol. 9:205-223; Hruby (1997) Curr. Opin. Chem. Biol. 1:114-119; Ostergaard (1997) Mol. Divers. 3:17-27; Ostresh (1996) Methods Enzymol. 267:220-234.

6.5.4 Small Molecules

In some embodiments, the inhibitor is a small molecule (i.e., having a molecular weight less than 1 kD). Methods for screening small molecules are well known in the art and include those described supra at Section 6.4.

6.6 6.6 Cell Surface Receptors and PDZ-Domain Binding Sequences

The following sections describe specific surface receptors expressed by different cell types in the hematopoietic and immune response system. The C-termini of these receptors are used as inhibitors, or serve as the basis for designing PDZ motif sequence peptides, variants, fusion proteins, peptidomimetics, and small molecules for use in inhibiting PDZ-PL interactions. In a preferred embodiment, the peptides are tested in an assay of the invention for inhibitory or modulatory activity (also see, TABLE 4, and discussion supra).

6.6.1 PDZ Motif Sequences of T Cell Surface Receptors

A number of surface receptors expressed by T cells contain a PDZ motif sequence (PL sequence). These molecules include CD3η, CD4, CD6, CD38, CD49e, CD49f, CD53, CD83, CD90, CD95, CD97, CD98, CDw137 (41BB), CD166, CDw128 (IL8 R), DNAM-1, Fas ligand (FasL) and LPAP (Barclay et al., 1997, The Leucocyte Antigen Facts Book, second edition, Academic Press), CLASP-1, CLASP-2, CLASP-4, KV1.3, BLR-1 (CXCR5), PAG and DOCK2.

The C-terminal core sequence of CD3 is SSQL (SEQ ID NO:4). When naturally-occurring residues are added or removed from the core sequence, QL (SEQ ID NO: ), SQL (SEQ ID NO: ), SSSQL (SEQ ID NO:5), SSSSQL (SEQ ID NO:6), PSSSSQL (SEQ ID NO:7), and PPSSSSQL (SEQ ID NO:8) may also be used to target a PDZ domain-containing protein in T cells.

The C-terminal core sequence of CD4 is CSPI (SEQ ID NO:9). When naturally-occuring residues are added or removed from the core sequence, PI (SEQ ID NO: ), SPI (SEQ ID NO: ), TCSPI (SEQ ID NO:10), KTCSPI (SEQ ID NO:11), QKTCSPI (SEQ ID NO:12), and FQKTCSPI (SEQ ID NO:13) may also be used to target a PDZ domain-containing protein in T cells.

The C-terminal core sequence of CD6 is ISAA (SEQ ID NO:14). When naturally-occuring residues are added or removed from the core sequence, AA (SEQ ID NO: ), SAA (SEQ ID NO: ), DISAA (SEQ ID NO:15), DDISAA (SEQ ID NO:16), YDDISAA (SEQ ID NO:17), and DYDDISAA (SEQ ID NO:18) may also be used to target a PDZ domain-containing protein in T cells.

The C-terminal core sequence of CD38 is TSEI (SEQ ID NO:19). When naturally-occuring residues are added or removed from the core sequence, El (SEQ ID NO: ), SEI (SEQ ID NO: ), CTSEI (SEQ ID NO:20), SCTSEI (SEQ ID NO:21), SSCTSEI (SEQ ID NO:22), and DSSCTSEI (SEQ ID NO:23) may also be used to target a PDZ domain-containing protein in T cells.

The C-terminal core sequence of CD49e is TSDA (SEQ ID NO:24). When naturally-occuring residues are added or removed from the core sequence, DA (SEQ ID NO: ), SDA (SEQ ID NO: ), ATSDA (SEQ ID NO:25), PATSDA (SEQ ID NO:26), PPATSDA (SEQ ID NO:27), and KPPATSDA (SEQ ID NO:28) may also be used to target a PDZ domain-containing protein in T cells.

The C-terminal core sequence of CD49f is TSDA (SEQ ID NO:29). When naturally-occuring residues are added or removed from the core sequence, DA (SEQ ID NO: ), SDA (SEQ ID NO: ), LTSDA (SEQ ID NO:30), RLTSDA (SEQ ID NO:31), ERLTSDA (SEQ ID NO:32), and KERLTSDA (SEQ ID NO:33) may also be used to target a PDZ domain-containing protein in T cells.

The C-terminal core sequence of CD53 is TIGL (SEQ ID NO:34). When naturally-occuring residues are added or removed from the core sequence, GL (SEQ ID NO: ), IGL (SEQ ID NO: ), QTIGL (SEQ ID NO:35), SQTIGL (SEQ ID NO:36), TSQTIGL (SEQ ID NO:37), and KTSQTIGL (SEQ ID NO:38) may also be used to target a PDZ domain-containing protein in T cells.

The C-terminal core sequence of CD83 is TELV (SEQ. ID. NO: 177). When naturally-occuring residues are added or removed from the core sequence, LV (SEQ ID NO: ), ELV (SEQ ID NO: ), KTELV (SEQ. ID. NO: 178), HKTELV (SEQ. ID. NO: 179), PHKTELV (SEQ. ID. NO: 180), and TPHKTELV (SEQ. ID. NO: 181) may also be used to target a PDZ domain-containing protein in T cells.

The C-terminal core sequence of CD90 is FMSL (SEQ ID NO:39). When naturally-occuring residues are added or removed from the core sequence, SL (SEQ ID NO: ), MSL (SEQ ID NO: ), DFMSL (SEQ ID NO:40), TDFMSL (SEQ ID NO:41), ATDFMSL (SEQ ID NO:42), and QATDFMSL (SEQ ID NO:43) may also be used to target a PDZ domain-containing protein in T cells.

The C-terminal core sequence of CD95 is QSLV (SEQ ID NO:44). When naturally-occuring residues are added or removed from the core sequence, LV (SEQ ID NO: ), SLV (SEQ ID NO: ), IQSLV (SEQ ID NO:45), EIQSLV (SEQ ID NO:46), NEIQSLV (SEQ ID NO:47), and RNEIQSLV (SEQ ID NO:48) may also be used to target a PDZ domain-containing protein in T cells.

The C-terminal core sequence of CD97 is ESGI (SEQ ID NO:49). When naturally-occuring residues are added or removed from the core sequence, GI (SEQ ID NO: ), SGI (SEQ ID NO: ), SESGI (SEQ ID NO:50), ASESGI (SEQ ID NO:51), RASESGI (SEQ ID NO:52), and LRASESGI (SEQ ID NO:53) may also be used to target a PDZ domain-containing protein in T cells.

The C-terminal core sequence of CD98 is PYAA (SEQ ID NO:54). When naturally-occuring residues are added or removed from the core sequence, AA (SEQ ID NO: ), YAA (SEQ ID NO: ), FPYAA (SEQ ID NO:55), RFPYAA (SEQ ID NO:56), LRFPYAA (SEQ ID NO:57), and LLRFPYAA (SEQ ID NO:58) may also be used to target a PDZ domain-containing protein in T cells.

The C-terminal core sequence of CDw137 is GCEL (SEQ ID NO:59). When naturally-occuring residues are added or removed from the core sequence, EL (SEQ ID NO: ), CEL (SEQ ID NO: ), GGCEL (SEQ ID NO:60), EGGCEL (SEQ ID NO:61), EEGGCEL (SEQ ID NO:62), and EEEGGCEL (SEQ ID NO:63) may also be used to target a PDZ domain-containing protein in T cells.

The C-terminal core sequence of CD166 is KTEA (SEQ ID NO:64). When naturally-occuring residues are added or removed from the core sequence, EA (SEQ ID NO: ), TEA (SEQ ID NO: ), HKTEA (SEQ ID NO:65), NHKTEA (SEQ ID NO:66), NNHKTEA (SEQ ID NO:67), and ENNHKTEA (SEQ ID NO:68) may also be used to target a PDZ domain-containing protein in T cells.

The C-terminal core sequence of CDw128 is SSNL (SEQ ID NO:69). When naturally-occuring residues are added or removed from the core sequence, NL (SEQ ID NO: ), SNL (SEQ ID NO: ), VSSNL (SEQ ID NO:70), NVSSNL (SEQ ID NO:71), VNVSSNL (SEQ ID NO:72), and SVNVSSNL (SEQ ID NO:73) may also be used to target a PDZ domain-containing protein in T cells.

The C-terminal core sequence of DNAM-1 is KTRV (SEQ ID NO:74). When naturally-occuring residues are added or removed from the core sequence, RV (SEQ ID NO: ), TRV (SEQ ID NO: ), PKTRV (SEQ ID NO:75), RPKTRV (SEQ ID NO:76), RRPKTRV (SEQ ID NO:77), and SRRPKTRV (SEQ ID NO:78) may also be used to target a PDZ domain-containing protein in T cells.

The C-terminal core sequence of FasL is LYKL (SEQ ID NO:79). When naturally-occuring residues are added or removed from the core sequence, KL (SEQ ID NO: ), YKL (SEQ ID NO: ), GLYKL (SEQ ID NO:80), FGLYKL (SEQ ID NO:81), FFGLYKL (SEQ ID NO:82), and TFFGLYKL (SEQ ID NO:83) may also be used to target a PDZ domain-containing protein in T cells.

The C-terminal core sequence of LPAP is VTAL (SEQ ID NO:84). When naturally-occuring residues are added or removed from the core sequence, AL (SEQ ID NO: ), TAL (SEQ ID NO: ), HVTAL (SEQ ID NO:85), LHVTAL (SEQ ID NO:86), GLHVTAL (SEQ ID NO:87), and QGLHVTAL (SEQ ID NO:88) may also be used to target a PDZ domain-containing protein in T cells.

The C-terminal core sequence of CLASP-1 is SAQV (SEQ. ID. NO: 182). When naturally-occuring residues are added or removed from the core sequence, QV (SEQ ID NO: ), AQV (SEQ ID NO: ), SSAQV (SEQ. ID. NO: 183), SSSAQV (SEQ. ID. NO: 184), ISSSAQV (SEQ. ID. NO: 185), and SISSSAQV (SEQ. ID. NO: 186) may also be used to target a PDZ domain-containing protein in T cells.

The C-terminal core sequence of CLASP-2 is SSVV (SEQ. ID. NO: 187). When naturally-occuring residues are added or removed from the core sequence, VV (SEQ ID NO: ), SVV (SEQ ID NO: ), SSSVV (SEQ. ID. NO: 188), SSSSVV (SEQ. ID. NO: 189), TSSSSVV (SEQ. ID. NO: 190), and MTSSSSVV (SEQ. ID. NO: 191) may also be used to target a PDZ domain-containing protein in T cells.

The C-terminal core sequence of CLASP4 is YAEV (SEQ. ID. NO: 192). When naturally-occuring residues are added or removed from the core sequence, EV (SEQ ID NO: ), AEV (SEQ ID NO: ), RYAEV (SEQ. ID. NO: 193), PRYAEV (SEQ. ID. NO: 194), SPRYAEV (SEQ. ID. NO: 195), and GSPRYAEV (SEQ. ID. NO: 196) may also be used to target a PDZ domain-containing protein in T cells.

The C-terminal core sequence of KV1.3 is FTDV (SEQ. ID. NO: 202). When naturally-occuring residues are added or removed from the core sequence, DV (SEQ ID NO: ), TDV (SEQ ID NO: ), IFTDV (SEQ. ID. NO: 203), KIFTDV (SEQ. ID. NO: 204), KKIFTDV (SEQ. ID. NO: 205), and IKKIFTDV (SEQ. ID. NO: 206) may also be used to target a PDZ domain-containing protein in T cells.

The C-terminal core sequence of DOCK2 is STDL (SEQ. ID. NO: 207). When naturally-occurring residues are added or removed from the core sequence, DL (SEQ. ID. NO: ), TDL (SEQ. ID. NO: ), LSTDL (SEQ. ID. NO: 208), SLSTDL (SEQ. ID. NO: 209), DSLSTDL (SEQ. ID. NO: 210), and PDSLSTDL (SEQ. ID. NO: 211) may also be used to target a PDZ domain-containing protein in T cells.

The C-terminal core sequence of BLR-1 is LTTF (SEQ ID NO:). When naturally-occurring residues are added or removed from the core sequence, TF (SEQ ID NO: ), TTF (SEQ ID NO: ), SLTTF (SEQ ID NO:), TSLTIF (SEQ ID NO:), ATSLTTF (SEQ ID NO:), and NATSLTTF (SEQ ID NO:) may also be used to target a PDZ domain-containing protein in T cells.

The C-terminal core sequence of PAG is ITRL (SEQ ID NO:). When naturally-occurring residues are added or removed from the core sequence, RL (SEQ ID NO: ), TRL (SEQ ID NO: ), DITRL (SEQ ID NO:), RDITRL (SEQ ID NO:), GRDITRL (SEQ ID NO:), and QGRDITRL (SEQ ID NO:) may also be used to target a PDZ domain-containing protein in T cells.

6.6.2 PDZ Motif Sequences of B Cell Surface Receptors

A number of surface receptors expressed by B cells contain a PDZ domain motif sequence. These molecules include, but are not limited to, CD38, CD53, CD95, CD97, CD98, CDw137, CD138, CDw125 (IL5R), DNAM-1, LPAP, Syndecan-2 (Barclay et al., 1997, The Leucocyte Antigen Facts Book, second edition, Academic Press) and BLR-1. The specific motif sequences of CD38, CD53, CD83, CD95, CD97, CD98, CDw137, DNAM-1, DOCK2, LPAP, BLR-1 (CXCR5), PAG, CLASP-1, CLASP-2 and CLASP-4 have been described in the preceding paragraphs.

The C-terminal core sequence of CD138 is EFYA (SEQ ID NO:89). When naturally-occuring residues are added or removed from the core sequence, YA (SEQ ID NO: ), FYA (SEQ ID NO: ), EEFYA (SEQ ID NO:90), QEEFYA (SEQ ID NO:91), KQEEFYA (SEQ ID NO:92), and TKQEEFYA (SEQ ID NO:93) may also be used to target a PDZ domain-containing protein in B cells.

The C-terminal core sequence of CDw125 is DSVF (SEQ ID NO:94). When naturally-occuring residues are added or removed from the core sequence, VF (SEQ ID NO: ), SVF (SEQ ID NO: ), EDSVF (SEQ ID NO:95), LEDSVF (SEQ ID NO:96), TLEDSVF (SEQ ID NO:97), and ETLEDSVF (SEQ ID NO:98) may also be used to target a PDZ domain-containing protein in B cells.

The C-terminal core sequence of Syndecan-2 is EFYA (SEQ. ID. NO: 212). When naturally-occuring residues are added or removed from the core sequence, YA (SEQ ID NO: ), FYA (SEQ ID NO: ), KEFYA (SEQ. ID. NO: 213), TKEFYA (SEQ. ID. NO: 214), PTKEFYA (SEQ. ID. NO: 215), and APTKEFYA (SEQ. ID. NO: 216) may also be used to target a PDZ domain-containing protein in B cells.

The C-terminal core sequence of BLR-1 is LTTF (SEQ. ID. NO: 217). When naturally-occuring residues are added or removed from the core sequence, TF (SEQ ID NO: ), TFF (SEQ ID NO: ), SLTTF (SEQ. ID. NO: 218), TSLTTF (SEQ. ID. NO: 219), ATSLTTF (SEQ. ID. NO: 220), and NATSLTTF (SEQ. ID. NO: 221) may also be used to target a PDZ domain-containing protein in B cells.

6.6.3 PDZ motif sequences of Natural Killer Cell Surface Receptors

A number of surface receptors expressed by NK cells contain a PDZ domain motif sequence. These molecules include, but are not limited to CD38, CD56, CD98 and DNAM-1. The specific motif sequences of CD38, CD98 and DNAM-1 have been described in the preceding paragraphs.

The C-terminal core sequence of CD56 is ESKA (SEQ ID NO:99). When naturally-occuring residues are added or removed from the core sequence, KA (SEQ ID NO: ), SKA (SEQ ID NO: ), NESKA (SEQ ID NO:100), ENESKA (SEQ ID NO:101), KENESKA (SEQ ID NO:102), and TKENESKA (SEQ ID NO:103) may also be used to target a PDZ domain-containing protein in NK cells.

6.6.4 PDZ Motif Sequences of Monocyte Surface Receptors

A number of surface receptors expressed by cells of the monocytic lineage (monocytes and macrophages) contain a PDZ domain motif sequence. These molecules include, but are not limited to CD38, CD44, CD46, CD49e, CD49f, CD53, CD61, CD95, CD97, CD98, CD148, CDw128, CDw137, Ly-6, DNAM-1 and FcεRIβ. The specific motif sequences of CD38, CD49e, CD49f, CD53, CD95, CD97, CD98, CDw128, CDw137, DNAM-1, Galectin 3 (Mac-2), BLR-1 (CXCR5) and Mannose receptor have been described in the preceding paragraphs.

The C-terminal core sequence of CD44 is KIGV (SEQ ID NO:104). When naturally-occuring residues are added or removed from the core sequence, GV (SEQ ID NO: ), IGV (SEQ ID NO: ), MKIGV (SEQ ID NO:105), DMKIGV (SEQ ID NO:106), VDMKIGV (SEQ ID NO:107) and NVDMKIGV (SEQ ID NO:108) may also be used to target a PDZ domain-containing protein in monocytes.

The C-terminal core sequence of CD46 is FTSL (SEQ ID NO:109). When naturally-occuring residues are added or removed from the core sequence, SL (SEQ ID NO: ), TSL (SEQ ID NO: ), KFTSL (SEQ ID NO:110), VKFTSL (SEQ ID NO:111), EVKFTSL (SEQ ID NO:112) and REVKFTSL (SEQ ID NO:113) may also be used to target a PDZ domain-containing protein in monocytes.

The C-terminal core sequence of CD61 is KSLV (SEQ ID NO:114). When naturally-occuring residues are added or removed from the core sequence, LV (SEQ ID NO: ), SLV (SEQ ID NO: ), LKSLV (SEQ ID NO:115), FLKSLV (SEQ ID NO:116), RFLKSLV SEQ ID NO:117) and GRFLKSLV (SEQ ID NO:118) may also be used to target a PDZ domain-containing protein in monocytes.

The C-terminal core sequence of CD148 is GYIA (SEQ ID NO:119). When naturally-occuring residues are added or removed from the core sequence, IA (SEQ ID NO: ), YIA (SEQ ID NO: ), NGYIA (SEQ ID NO:120), TNGYIA (SEQ ID NO:121), KTNGYIA (SEQ ID NO:122) and GKTNGYIA (SEQ ID NO:123) may also be used to target a PDZ domain-containing protein in monocytes.

The C-terminal core sequence of Ly-6 is QTLL (SEQ ID NO:124). When naturally-occuring residues are added or removed from the core sequence, LL (SEQ ID NO: ), TLL (SEQ ID NO: ), LQTLL (SEQ ID NO:125), LLQTLL (SEQ ID NO:126), VLLQTLL (SEQ ID NO:127) and SVLLQTLL (SEQ ID NO:128) may also be used to target a PDZ domain-containing protein in monocytes.

The C-terminal core sequence of FcεRIβ is PIDL (SEQ ID NO:129). When naturally-occuring residues are added or removed from the core sequence, DL (SEQ ID NO: ), IDL (SEQ ID NO: ), PPIDL (SEQ ID NO:130), SPPIDL (SEQ ID NO:131), MSPPIDL (SEQ ID NO:132) and EMSPPIDL (SEQ ID NO:133) may also be used to target a PDZ domain-containing protein in monocytes.

The C-terminal core sequence of Galectin 3 is YTMI (SEQ ID NO:134). When naturally-occuring residues are added or removed from the core sequence, MI (SEQ ID NO: ), TMI (SEQ ID NO: ), SYTMI (SEQ ID NO:135), ASYTMI (SEQ ID NO:136), SASYTMI (SEQ ID NO:137) and TSASYTMI (SEQ ID NO:138) may also be used to target a PDZ domain-containing protein in monocytes.

The C-terminal core sequence of mannose receptor is HSVI (SEQ ID NO:139). When naturally-occuring residues are added or removed from the core sequence, VI (SEQ ID NO: ), SVI (SEQ ID NO: ), EHSVI (SEQ ID NO:140), NEHSVI (SEQ ID NO:141), QNEHSVI (SEQ ID NO:142) and EQNEHSVI (SEQ ID NO:143) may also be used to target a PDZ domain-containing protein in monocytes.

6.6.5 PDZ Motif Sequences of Granulocyte Surface Receptors

A number of surface receptors expressed by granulocytes contain a PDZ domain motif sequence. These molecules include, but are not limited to CD53, CD95, CD97, CD98, CD148, CDw125, CDw128, FcεRIβ and G-CSFR. The specific motif sequences of most of these molecules have been described in the preceding paragraphs.

The C-terminal core sequence of G-CSFR is TSVL (SEQ ID NO:144). When naturally-occuring residues are added or removed from the core sequence, VL (SEQ ID NO: ), SVL (SEQ ID NO: ), ITSVL (SEQ ID NO:145), PITSVL (SEQ ID NO:146), FPITSVL (SEQ ID NO:147) and LFPITSVL (SEQ ID NO:148) may also be used to target a PDZ domain-containing protein in monocytes.

6.6.6 PDZ Motif Sequences of Endothelial Cell Surface Receptors

While endothelial cells are not hematopoietic cells, they closely interact with the hematopoietic system as they form the lining of blood vessels. As such, endothelial cells come in contact with the cells of the hematopoietic system. Thus, the ability to regulate endothelial cell function provides for indirect regulation of hematopoietic cells.

A number of surface receptors expressed by endothelial cells contain a PDZ domain motif sequence. These molecules include, but are not limited to CD34, CD46, CD66b, CD66c, CD105, CD106, CD62e (E-selectin) and VCAM1.

The C-terminal core sequence of CD34 is DTEL (SEQ ID NO:149). When naturally-occuring residues are added or removed from the core sequence, EL (SEQ ID NO: ), TEL (SEQ ID NO: ), ADTEL (SEQ ID NO:150), VADTEL (SEQ ID NO:151), VVADTEL (SEQ ID NO:152) and HVVADTEL (SEQ ID NO:153) may also be used to target a PDZ domain-containing protein in endothelial cells.

The C-terminal core sequence of CD66b and CD66c is VALI (SEQ ID NO:154). When naturally-occuring residues are added or removed from the core sequence, LI (SEQ ID NO: ), ALI (SEQ ID NO: ), RVALI (SEQ ID NO:155), ARVALI (SEQ ID NO:156), LARVALI (SEQ ID NO:157) and VLARVALI (SEQ ID NO:158) may also be used to target a PDZ domain-containing protein in endothelial cells.

The C-terminal core sequence of CD105 is SSMA (SEQ ID NO:159). When naturally-occuring residues are added or removed from the core sequence, MA (SEQ ID NO: ), SMA (SEQ ID NO: ), TSSMA (SEQ ID NO:160), STSSMA (SEQ ID NO:161), CSTSSMA (SEQ ID NO: 222) and PCSTSSMA (SEQ ID NO: 162) may also be used to target a PDZ domain-containing protein in endothelial cells.

The C-terminal core sequence of CD106 is KSKV (SEQ ID NO:163). When naturally-occuring residues are added or removed from the core sequence, KV (SEQ ID NO: ), SKV (SEQ ID NO: ), QKSKV (SEQ ID NO:164), AQKSKV (SEQ ID NO:165), EAQKSKV (SEQ ID NO:166) and VEAQKSKV (SEQ ID NO:167) may also be used to target a PDZ domain-containing protein in endothelial cells.

The C-terminal core sequence of CD62e is SYIL (SEQ ID NO:168). When naturally-occuring residues are added or removed from the core sequence, IL (SEQ ID NO: ), YIL (SEQ ID NO: ), PSYIL (SEQ ID NO:169), KPSYIL (SEQ ID NO:170), QKPSYIL (SEQ ID NO:171) and YQKPSYIL (SEQ ID NO:172) may also be used to target a PDZ domain-containing protein in endothelial cells.

The C-terminal core sequence of VCAM1 is KSKV (SEQ. ID. NO: 197). When naturally-occuring residues are added or removed from the core sequence, KV (SEQ ID NO: ), SKV (SEQ ID NO: ), QKSKV (SEQ. ID. NO: 198), AQKSKV (SEQ. ID. NO: 199), EAQKSKV (SEQ. ID. NO: 200), and VEAQKSKV (SEQ. ID. NO: 201) may also be used to target a PDZ domain-containing protein in endothelial cells.

6.6.7 Mast Cell, Basophils and Eosinophil Cell Surface Receptors

FcεRIβ, CDw125, CDw128 and IL-8RB are transmembrane receptors expressed by mast cells, basophils and eosinophils. These receptors play a role in the activation of these cells to result in degranulation and histamine release in allergic reactions. The C-terminal core sequence of FcεRIβ is PIDL (SEQ ID NO:129). When naturally-occuring residues are added or removed from the core sequence, DL (SEQ ID NO: ), IDL (SEQ ID NO: ), PPIDL (SEQ ID NO:244), SPPIDL (SEQ ID NO:245), MSPPIDL (SEQ ID NO:246) and EMSPPIDL (SEQ ID NO:247) may also be used to target a PDZ domain-containing protein in mast cells. In addition, the residue E may be substituted with G to increase its binding affinity.

The C-terminal core sequence of CDw125 is DSVF (SEQ ID NO: 248). When naturally-occuring residues are added or removed from the core sequence, VF (SEQ ID NO: ), SVF (SEQ ID NO: ), EDSVF (SEQ ID NO:249), LEDSVF (SEQ ID NO:250), TLEDSVF (SEQ ID NO:251), and ETLEDSVF (SEQ ID NO:252) may also be used to target a PDZ domain-containing protein in mast cells.

The C-terminal core sequence of CDw128 is SSNL (SEQ ID NO:253). When naturally-occuring residues are added or removed from the core sequence, NL (SEQ ID NO: ), SNL (SEQ ID NO: ), VSSNL (SEQ ID NO:254), NVSSNL (SEQ ID NO:255), VNVSSNL (SEQ ID NO:256), and SVNVSSNL (SEQ ID NO:257) may also be used to target a PDZ domain-containing protein in mast cells.

The C-terminal core sequence of IL-8RB is STTL (SEQ ID NO:258). When naturally-occuring residues are added or removed from the core sequence, TL (SEQ ID NO: ), TTL (SEQ ID NO: ), TSTTL (SEQ ID NO:259), HTSTTL (SEQ ID NO:260), GHTSTTL (SEQ ID NO:261) and SGHTSTTL (SEQ ID NO:262) may also be used to target a PDZ domain-containing protein in mast cells.

6.6.8 Other PDZ Motif Sequences

The C-terminal core sequence of NMDA is ESDV (SEQ. ID. NO: 223). When naturally-occuring residues are added or removed from the core sequence, DV (SEQ ID NO: ), SDV (SEQ ID NO: ), IESDV (SEQ. ID. NO: 224), SIESDV (SEQ. ID. NO: 225), PSIESDV (SEQ. ID. NO: 226), and MPSIESDV (SEQ. ID. NO: 227) may also be used to target a PDZ domain-containing protein in neuronal cells.

The C-terminal core sequence of neurexin is EYYV (SEQ. ID. NO: 228). When naturally-occuring residues are added or removed from the core sequence, YV (SEQ ID NO: ), YYV (SEQ ID NO: ), KEYYV (SEQ. ID. NO: 229), DKEYYV (SEQ. ID. NO: 230), KDKEYYV (SEQ. ID. NO: 231), and NKDKEYYV (SEQ. ID. NO: 232) may also be used to target a PDZ domain-containing protein in neuronal cells.

The C-terminal core sequence of Glycophorin C is EYFI (SEQ. ID. NO: 233). When naturally-occuring residues are added or removed from the core sequence, FI (SEQ ID NO: ), YFI (SEQ ID NO: ), KEYFI (SEQ. ID. NO: 234), RKEYFI (SEQ. ID. NO: 235), SRKEYFI (SEQ. ID. NO: 236), and SSRKEYFI (SEQ. ID. NO: 237) may also be used to target a PDZ domain-containing protein.

The C-terminal core sequence of CD148 is KTIA (SEQ. ID. NO: 238). When naturally-occuring residues are added or removed from the core sequence, IA (SEQ ID NO: ), TIA (SEQ ID NO: ), GKTIA (SEQ. ID. NO: 239), FGKTIA (SEQ. ID. NO: 240), TFGKTIA (SEQ. ID. NO: 241), and TTFGKTIA (SEQ. ID. NO: 242) may also be used to target a PDZ domain-containing protein in epithelial or myeloid cells.

The C-terminal core sequence of beta-spectrin is VSFV (SEQ. ID. NO: ). When naturally-occuring residues are added to the core sequence, FV (SEQ. ID. NO: ), SFV (SEQ. ID. NO: ), LVSFV (SEQ. ID. NO: ), SLVSFV (SEQ. ID. NO: ), QSLVSFV (SEQ. ID. NO: ) AND GQSLVSFV (SEQ. ID. NO: ) may also be used to target a PDZ domain-containing protein.

### 6.7. Preparation of Peptides

6.7.1. Chemical Synthesis

The peptides of the invention or analogues thereof, may be prepared using virtually any art-known technique for the preparation of peptides and peptide analogues. For example, the peptides may be prepared in linear form using conventional solution or solid phase peptide syntheses and cleaved from the resin followed by purification procedures (Creighton, 1983, Protein Structures And Molecular Principles, W. H. Freeman and Co., N.Y.). Suitable procedures for synthesizing the peptides described herein are well known in the art. The composition of the synthetic peptides may be confirmed by amino acid analysis or sequencing (e.g., the Edman degradation procedure and mass spectroscopy).

In addition, analogues and derivatives of the peptides can be chemically synthesized. The linkage between each amino acid of the peptides of the invention may be an amide, a substituted amide or an isostere of amide. Nonclassical amino acids or chemical amino acid analogues can be introduced as a substitution or addition into the sequence. Non-classical amino acids include, but are not limited to, the D-isomers of the common amino acids, α-amino isobutyric acid, 4-aminobutyric acid, Abu, 2-amino butyric acid, γ-Abu, ε-Ahx, 6-amino hexanoic acid, Aib, 2-amino isobutyric acid, 3-amino propionic acid, ornithine, norleucine, norvaline, hydroxyproline, sarcosine, citrulline, cysteic acid, t-butylglycine, t-butylalanine, phenylglycine, cyclohexylalanine, β-alanine, fluoro-amino acids, designer amino acids such as β-methyl amino acids, Cα-methyl amino acids, Nα-methyl amino acids, and amino acid analogues in general. Furthermore, the amino acid can be D (dextrorotary) or L (levorotary).

6.7.2. Recombinant Synthesis

If the peptide is composed entirely of gene-encoded amino acids, or a portion of it is so composed, the peptide or the relevant portion may also be synthesized using conventional recombinant genetic engineering techniques. For recombinant production, a polynucleotide sequence encoding a linear form of the peptide is inserted into an appropriate expression vehicle, i.e., a vector which contains the necessary elements for the transcription and translation of the inserted coding sequence, or in the case of an RNA viral vector, the necessary elements for replication and translation. The expression vehicle is then transfected into a suitable target cell which will express the peptide. Depending on the expression system used, the expressed peptide is then isolated by procedures well-established in the art. Methods for recombinant protein and peptide production are well known in the art (see, e.g., Maniatis et al, 1989, Molecular Cloning A Laboratory Manual, Cold Spring Harbor Laboratory, N.Y.; and Ausubel et al., 1989, Current Protocols in Molecular Biology, Greene Publishing Associates and Wiley Interscience, N.Y.).

A variety of host-expression vector systems may be utilized to express the peptides described herein. These include, but are not limited to, microorganisms such as bacteria transformed with recombinant bacteriophage DNA or plasmid DNA expression vectors containing an appropriate coding sequence; yeast or filamentous fungi transformed with recombinant yeast or fungi expression vectors containing an appropriate coding sequence; insect cell systems infected with recombinant virus expression vectors (e.g., baculovirus) containing an appropriate coding sequence; plant cell systems infected with recombinant virus expression vectors (e.g., cauliflower mosaic virus or tobacco mosaic virus) or transformed with recombinant plasmid expression vectors (e.g., Ti plasmid) containing an appropriate coding sequence; or animal cell systems.

The expression elements of the expression systems vary in their strength and specificities. Depending on the host/vector system utilized, any of a number of suitable transcription and translation elements, including constitutive and inducible promoters, may be used in the expression vector. For example, when cloning in bacterial systems, inducible promoters such as pL of bacteriophage λ, plac, ptrp, ptac (ptrp-lac hybrid promoter) and the like may be used; when cloning in insect cell systems, promoters such as the baculovirus polyhedron promoter may be used; when cloning in plant cell systems, promoters derived from the genome of plant cells (e.g., heat shock promoters; the promoter for the small subunit of RUBISCO; the promoter for the chlorophyll a/b binding protein) or from plant viruses (e.g., the 35S RNA promoter of CaMV; the coat protein promoter of TMV) may be used; when cloning in mammalian cell systems, promoters derived from the genome of mammalian cells (e.g., metallothionein promoter) or from mammalian viruses (e.g., the adenovirus late promoter; the vaccinia virus 7.5 K promoter) may be used; when generating cell lines that contain multiple copies of expression product, SV40-, BPV- and EBV-based vectors may be used with an appropriate selectable marker.

In cases where plant expression vectors are used, the expression of sequences encoding the peptides of the invention may be driven by any of a number of promoters. For example, viral promoters such as the 35S RNA and 19S RNA promoters of CaMV (Brisson et al., 1984, Nature 310:511-514), or the coat protein promoter of TMV (Takamatsu et al., 1987, EMBO J. 6:307-311) may be used; alternatively, plant promoters such as the small subunit of RUBISCO (Coruzzi et al., 1984, EMBO J. 3:1671-1680; Broglie et al., 1984, Science 224:838-843) or heat shock promoters, e.g., soybean hsp17.5-E or hsp17.3-B (Gurley et al., 1986, Mol. Cell. Biol. 6:559-565) may be used. These constructs can be introduced into planleukocytes using Ti plasmids, Ri plasmids, plant virus vectors, direct DNA transformation, microinjection, electroporation, etc. For reviews of such techniques see, e.g., Weissbach & Weissbach, 1988, Methods for Plant Molecular Biology, Academic Press, NY, Section VIII, pp. 421-463; and Grierson & Corey, 1988, Plant Molecular Biology, 2d Ed., Blackie, London, Ch. 7-9.

In one insect expression system that may be used to produce the peptides of the invention, Autographa californica nuclear polyhidrosis virus (AcNPV) is used as a vector to express the foreign genes. The virus grows in Spodoptera frugperda cells. A coding sequence may be cloned into non-essential regions (for example the polyhedron gene) of the virus and placed under control of an AcNPV promoter (for example, the polyhedron promoter). Successful insertion of a coding sequence will result in inactivation of the polyhedron gene and production of non-occluded recombinant virus (i.e., virus lacking the proteinaceous coat coded for by the polyhedron gene). These recombinant viruses are then used to infect Spodoptera frugiperda cells in which the inserted gene is expressed. (e.g., see Smith et al., 1983, J. Virol. 46:584; Smith, U.S. Pat. No. 4,215,051). Further examples of this expression system may be found in Current Protocols in Molecular Biology, Vol. 2, Ausubel et al., eds., Greene Publish. Assoc. & Wiley Interscience.

In mammalian host cells, a number of viral based expression systems may be utilized. In cases where an adenovirus is used as an expression vector, a coding sequence may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing peptide in infected hosts. (e.g., See Logan & Shenk, 1984, Proc. Natl. Acad. Sci. USA 81:3655-3659). Alternatively, the vaccinia 7.5 K promoter may be used, (see, e.g., Mackett et al., 1982, Proc. Natl. Acad. Sci. USA 79:7415-7419; Mackett et al., 1984, J. Virol. 49:857-864; Panicali et al., 1982, Proc. Natl. Acad. Sci. USA 79:4927-4931).

Other expression systems for producing linear peptides of the invention will be apparent to those having skill in the art.

6.7.3. Purification of the Peptides and Peptide Analogues

The peptides and peptide analogues of the invention can be purified by art-known techniques such as high performance liquid chromatography, ion exchange chromatography, gel electrophoresis, affinity chromatography and the like. The actual conditions used to purify a particular peptide or analogue will depend, in part, on factors such as net charge, hydrophobicity, hydrophilicity, etc., and will be apparent to those having skill in the art. The purified peptides can be identified by assays based on their physical or functional properties, including radioactive labeling followed by gel electrophoresis, radioimmuno-assays, ELISA, bioassays, and the like.

For affinity chromatography purification, any antibody which specifically binds the peptides or peptide analogues may be used. For the production of antibodies, various host animals, including but not limited to rabbits, mice, rats, etc., may be immunized by injection with a peptide. The peptide may be attached to a suitable carrier, such as BSA or KLH, by means of a side chain functional group or linkers attached to a side chain functional group. Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacilli Calmette-Guerin) and Corynebacterium parvum.

Monoclonal antibodies to a peptide may be prepared using any technique which provides for the production of antibody molecules by continuous cell lines in culture. These include but are not limited to the hybridoma technique originally described by Koehler and Milstein, 1975, Nature 256:495-497, the human B-cell hybridoma technique, Kosbor et al., 1983, Immunology Today 4:72; Cote et al., 1983, Proc. Natl. Acad. Sci. U.S.A. 80:2026-2030 and the EBV-hybridoma technique (Cole et al, 1985, Monoclonal Antibodies and Cancer Therapy, Alan R. Liss, Inc., pp. 77-96 (1985)). In addition, techniques developed for the production of “chimeric antibodies” (Morrison et al, 1984, Proc. Natl. Acad. Sci. U.S.A. 81:6851-6855; Neuberger et al., 1984, Nature 312:604-608; Takeda et al., 1985, Nature 314:452-454) by splicing the genes from a mouse antibody molecule of appropriate antigen specificity together with genes from a human antibody molecule of appropriate biological activity can be used. Alternatively, techniques described for the production of single chain antibodies (U.S. Pat. No. 4,946,778) can be adapted to produce peptide-specific single chain antibodies.

Antibody fragments which contain deletions of specific binding sites may be generated by known techniques. For example, such fragments include but are not limited to F(ab)₂ fragments, which can be produced by pepsin digestion of the antibody molecule and Fab fragments, which can be generated by reducing the disulfide bridges of the F(ab′)₂ fragments. Alternatively, Fab expression libraries may be constructed (Huse et al., 1989, Science 246:1275-1281) to allow rapid and easy identification of monoclonal Fab fragments with the desired specificity for the peptide of interest.

The antibody or antibody fragment specific for the desired peptide can be attached, for example, to agarose, and the antibody-agarose complex is used in immunochromatography to purify peptides of the invention. See, Scopes, 1984, Protein Purification: Principles and Practice, Springer-Verlag New York, Inc., NY, Livingstone, 1974, Methods Enzymology: Immunoaffinity Chromatography of Proteins 34:723-731.

### 6.8. Uses of PDZ Domain Binding and Antagonist Compounds

In one aspect of the invention, the PDZ domain binding and PDZ-PL inhibitory compounds of the present invention are useful in regulating diverse activities of hematopoietic cells (e.g., T cells and B cells) and other cells involved in the immune response.

In one embodiment of the invention, the compounds of the invention are used to inhibit leukocyte activation, which is manifested in measurable events including but not limited to, cytokine production, cell adhesion, expansion of cell numbers, apoptosis and cytotoxicity. As a corollary, the compounds of the invention may be used to treat diverse conditions associated with undesirable leukocyte activation, including but not limited to, acute and chronic inflammation, graft-versus-host disease, transplantation rejection, hypersensitivities and autoimmunity such as multiple sclerosis, rheumatoid arthritis, peridontal disease, systemic lupus erythematosis, juvenile diabetes mellitis, non-insulin-dependent diabetes, and allergies, and other conditions listed herein (see, e.g., Section 6.4, supra).

Thus, the invention also relates to methods of using such compositions in modulating leukocyte activation as measured by, for example, cytotoxicity, cytokine production, cell proliferation, and apoptosis. Assays for activation are well known. For example, PDZ/PL interaction antagonists can be evaluated in the following: (1) cytotoxic T lymphocytes can be incubated with radioactively labeled target cells and the antigen-specific lysis of these target cells detected by the release of radioactivity, (2) helper T lymphocytes can be incubated with antigens and antigen presenting cells and the synthesis and secretion of cytokines measured by standard methods (Windhagen A; et al., 1995, Immunity 2(4): 373-80), (3) antigen presenting cells can be incubated with whole protein antigen and the presentation of that antigen on MHC detected by either T lymphocyte activation assays or biophysical methods (Harding et al., 1989, Proc. Natl. Acad. Sci., 86: 4230-4), (4) mast cells can be incubated with reagents that cross-link their Fc-epsilon receptors and histamine release measured by enzyme immunoassay (Siraganian, et al., 1983, TIPS 4: 432-437).

Similarly, the effect of PDZ/PL interaction antagonists on products of leukocyte activation in either a model organism (e.g., mouse) or a human patient can also be evaluated by various methods that are well known. For example, (1) the production of antibodies in response to vaccination can be readily detected by standard methods currently used in clinical laboratories, e.g., an ELISA; (2) the migration of immune cells to sites of inflammation can be detected by scratching the surface of skin and placing a sterile container to capture the migrating cells over scratch site (Peters et al., 1988, Blood 72: 1310-5); (3) the proliferation of peripheral blood mononuclear cells in response to mitogens or mixed lymphocyte reaction can be measured using ³H-thymidine; (4) the phagocytic capacity of granulocytes, macrophages, and other phagocytes in PBMCs can be measured by placing PMBCs in wells together with labeled particles (Peters et al., 1988); and (5) the differentiation of immune system cells can be measured by labeling PBMCs with antibodies to CD molecules such as CD4 and CD8 and measuring the fraction of the PBMCs expressing these markers.

In one exemplary assay, human peripheral blood mononuclear cells (PBMC), human T cell clones (e.g., Jurkat E6, ATCC TIB-152), EBV-transformed B cell clones (e.g., 9D10, ATCC CRL-8752), antigen-specific T cell clones or lines can be used to examine PDZ/PL interaction antagonists in vitro. Inhibition of activation of these cells or cell lines can be used for the evaluation of potential PDZ/PL interaction antagonists.

Standard methods by which hematopoietic cells are stimulated to undergo activation characteristic of an immune response are, for example:

A) Antigen specific stimulation of immune responses. Either pre-immunized or naïve mouse splenocytes can be generated by standard procedures. In addition, antigen-specific T cell clones and hybridomas (e.g., MBP-specific), and numerous B cell lymphoma cell lines (e.g., CH27), have been previously characterized and are available for the assays discussed below. Antigen specific splenocytes or B-cells can be mixed with antigen specific T-cells in the presence of antigen to generate an immune response. This can be performed in the presence or absence of PDZ/PL interaction antagonists to assay whether PDZ/PL interaction antagonists modulate the immune response infra.

B) Non-specific T cell activation. The following methods can be used to activate T cells in the absence of antigen: 1) cross-linking T cell receptor (TCR) by addition of antibodies against receptor activation molecules (e.g., TCR, CD3, or CD2) together with antibodies against co-stimulator molecules, for example anti-CD28; 2) activating cell surface receptors in a non-specific fashion using lectins such as concanavalin A (con A) and phytohemagglutinin (PHA); 3) mimicking cell surface receptor-mediated activation using pharmacological agents that activate protein kinase C (e.g., phorbol esters) and increase cytoplasmic Ca²⁺ (e.g., ionomycin).

C) Non-specific B cell activation: 1) application of antibodies against cell surface molecules such as IgM, CD20, or CD21. 2) Lipopolysaccharide (LPS), phorbol esters, calcium ionophores and ionomycin can also be used to by-pass receptor triggering.

D) Mixed lymphocyte reaction (MLR). Mix donor PBMC with recipient PBMC to activate lymphocytes by presentation of mismatched tissue antigens, which occurs in all cases except identical twins.

E) Generation of a specific T cell clone or line that recognizes a particular antigen. A standard approach is to generate tetanus toxin-specific T cells from a donor that has recently been boosted with tetanus toxin. Major histocompatability complex—(MHC—) matched antigen presenting cells and a source of tetanus toxin are used to maintain antigen specificity of the cell line or T cell clone (Lanzavecchia, A., et al., 1983, Eur. J. Immun. 13: 733-738).

Assay Quantitation

The assays described above can be quantitated by a variety of well known quantitation methods. For example:

(A) Tyrosine Phosphorylation

Tyrosine phosphorylation of early response proteins such as HS1, PLC-r, ZAP-76, and Vav is an early biochemical event following leukocyte activation. The tyrosine phosphorylated proteins can be detected by Western blot using antibodies against phosphorylated tyrosine residues. Tyrosine phosphorylation of these early response proteins can be used as a standard assay for leukocyte activation (J. Biol. Chem., 1997, 272(23): 14562-14570). Any change in the phosphorylation pattern of these or related proteins when immune responses are generated in the presence of potential PDZ/PL interaction antagonists is indicative of a potential PDZ/PL interaction antagonists.

(B) Intracellular Calcium Flux

The kinetics of intracellular Ca²⁺ concentrations are measured over time after stimulation of cells preloaded with a calcium sensitive dye. Upon binding Ca²⁺ the indicator dye (e.g., Fluor4 (Molecular Probes)), exhibits an increase in fluorescence level using flow cytometry, solution fluorometry, and confocal microscopy. Any change in the level or timing of calcium flux when immune responses are generated in the presence of PDZ/PL interaction antagonists is indicative of an inhibition of this response.

(C) Regulation of Early Activation Markers

Increased and diminished expression/regulation of early lymphocyte activation marker levels such as CD69, IL-2R, MHC class II, B7, and TCR are commonly measured with fluorescently labeled antibodies using flow cytometry. All antibodies are commercially available. Any change in the expression levels of lymphocyte activation markers when immune responses are generated in the presence of the PDZ/PL interaction antagonists is indicative of an inhibition of this response.

(D) Increased Metabolic Activity/Acid Release

Activation of most known signal transduction pathways trigger increases in acidic metabolites. This reproducible biological event is measured as the rate of acid release using a microphysiometer (Molecular Devices), and is used as an early activation marker when comparing the treatment of cells with potential biological therapeutics (McConnell, H. M. et al., 1992, Science 257: 1906-1912 and McConnell, H. M., 1995, Proc. Natl. Acad. Sci. 92: 2750-2754). Any statistically significant increase or decrease in acid release of the PDZ/PL interaction antagonist-treated sample, as compared to control sample (no treatment), suggest an effect of the PDZ/PL interaction antagonist on biological function.

(E) Cell Proliferation/Cell Viability Assays

(1) ³H-Thimidine Incorporation

Exposure of lymphocytes to antigen or mitogen in vitro induces DNA synthesis and cellular proliferation. The measurement of mitotic activity by ³H-thimidine incorporation into newly synthesized DNA is one of the most frequently used assays to quantitative T cell activation. Depending on the cell population and form of stimulation used to activate the T cells, mitotic activity can be measured within 24-72 hrs. in vitro, post 3H-thimidine pulse (Mishell, B. B. and S. M. Shiigi, 1980, Selected Methods in Cellular Immunology, W. H. Freeman and Company and Dutton, R. W. and Pearce, J. D., 1962, Nature 194: 93). Any statistically significant increase or decrease in CPM of the PDZ/PL interaction antagonist-treated sample, as compared to control sample (no treatment), suggest and effect of the PDZ/PL interaction antagonist on biological function.

(2) MTS [5-(3-carboxymethoxyphenyl)-2-(4,5-dimethylthiazolyl)-3(4-sulfophenyl)tetrazolium, inner salt] is a colorimetric method for determining the number of viable cells in proliferation or cytotoxicity assays (Barltrop, J. A. et al., 1991, Bioorg. & Med. Chem. Lett. 1: 611). 1-5 days after lymphocyte activation, MTS tetrazolium compound, Owen's reagent, is bioreduced by cells into a colored formazan product that is soluble in tissue culture media Color intensity is read at 490 nm minus 650 nm using a microplate reader. Any statistically significant increase or decrease in color intensity of the PDZ/PL interaction antagonist-treated sample, as compared to control sample (no treatment), can suggest an effect of the PDZ/PL interaction antagonist on biological function (Mosmann, T., 1983, J. Immunol. Methods 65: 55 and Barltrop, J. A. et al. (1991)).

(3) Bromodeoxyuridine (BrdU), a thymidine analogue, readily incorporates into cells undergoing DNA synthesis. BrdU-pulsed cells are labeled with an enzyme-conjugated anti-BrdU antibody (Gratzner, H. G., 1982, Science 218: 474-475.). A colorimetric, soluble substrate is used to visualize proliferating cells that have incorporated BrdU. Reaction is stopped with sulfuric acid and plate is read at 450 nm using a microplate reader. Any statistically significant increase or decrease in color intensity of the PDZ/PL interaction antagonist-treated sample, as compared to control sample (no treatment), suggest an effect of the PDZ/PL interaction antagonist on biological function.

(F) Apoptosis by Annexin V

Programmed cell death or apoptosis is an early event in a cascade of catabolic reactions leading to cell death. A lose in the integrity of the cell membrane allows for the binding of fluorescently conjugated phosphatidylserine. Stained cells can be measured by fluorescence microscopy and flow cytometry (Vermes, I., 1995, J. Immunol. Methods. 180: 39-52). In one embodiment, any statistically significant increase or decrease in apoptotic cell number of the PDZ/PL interaction antagonist-treated sample, as compared to control sample (no treatment), suggest an effect of the PDZ/PL interaction antagonist on biological function.

For evaluating apoptosis in situ, assays for evaluating cell death in tissue samples can also be used in vivo studies.

(G) Quantitation of Cytokine Production

Cell supernatants harvested after cell stimulation for 1648 hrs are stored at −80° C until assayed or directly tested for cytokine production. Multiple cytokine assays can be performed on each sample. IL-2, IL-3, IFN-γ and other cytokine ELISA Assays are available for mouse, rat, and human (Endogen, Inc. and BioSource). Cytokine production is measured using a standard two-antibody sandwich ELISA protocol as described by the manufacturer. The presence of horseradish peroxidase is detected with 3, 3′5, 5′ tertamethyl benziidine (TMB) substrate and the reaction is stopped with sulfuric acid. The absorbency at 450 nm is measured using a microplate reader. Any statistically significant increase or decrease in color intensity of the PDZ/PL interaction antagonist-treated sample, as compared to control sample (no treatment), suggest an effect of the PDZ/PL interaction antagonist on biological function. See also Example 1, infra. Detection of intracellular cytokines using anti-cytokine antibodies provides the additional advantage of measuring cytokines fore mixed cell populations. This allows for phenotyping measuring frequency of cytokine producing cell types in suspension or in tissues.

(H) NF-AT can be Visualized by Immunostaining

T cell activation requires the import of nuclear factor of activated T cells (NF-AT) to the nucleus. This translocation of NF-AT can be visualized by immunostaining with anti-NF-AT antibody (Cell 1998, 93: 851-861). Therefore, NF-AT nuclear translocation has been used to assay T cell activation. Similarly, NF-AT/luciferase reporter assays have been used as a standard measurement of T cell activation (MCB 1996, 12: 7151-7160). Any statistically significant increase or decrease in the nuclear translocation of NF-AT brought about by the PDZ/PL interaction antagonist-treated sample, as compared to control sample (no treatment), suggest an effect of the PDZ/PL interaction antagonist on biological function. In order to optimize the use of the peptides and peptide analogues disclosed herein in a human subject, various animal models may be used to define certain clinical parameters. For example, the compounds of the invention may be tested in different dosages, formulations and route of administration in a cardiac transplant mouse model to optimize their ability to inhibit rejection responses to solid organ transplants (Fulmer et al., 1963, Am. J. Anat. 113:273; Jockusch et al., 1983, Exp. Neurol. 81:749).

In situations where inhibition of a T cell response is desired, the compounds of the inventions may be used to inhibit PDZ domain interactions with CD3, CD4, CD6 and CDw137. In addition, the compounds of the invention may be used to inhibit PDZ domain interactions with CD53 and CD138 in B cells. In order to inhibit IgE-mediated allergic reactions, the compounds of the invention may be used to inhibit PDZ domain interactions with FcεRIβ, CDw125 and CDw128. Furthermore, a PDZ motif sequence (PL sequence) of CD95 may be used to induce apoptosis of lymphomas.

(I) Inflammatory Mediator Release Assays

Assays are well known in the art for inflammatory mediator release to access the effect of compounds or treatments IgE-mediated degranulation. See, e.g. Berger et al., 1997, Measuring Cell Degranulation e.g., Ch 19.6 Immunology Method Manual. Academic Press, Ltd. 1436-1440 and Siraganian, 1983, Histamine Secretion from Mast Cells and Basophil. TIPS 4:432-437, both incorporated by reference herein.

### 6.9. Formulation and Route of Administration

6.9.1 Introduction of Agonists or Antagonists (e.g. Peptides and Fusion Proteins) into Cells

In one aspect, the PDZ-PL antagonists of the invention are introduced into a cell to modulate (i.e., increase or decrease) a biological function or activity of the cell. Many small organic molecules readily cross the cell membranes (or can be modified by one of skill using routine methods to increase the ability of compounds to enter cells, e.g., by reducing or eliminating charge, increasing lipophilicity, conjugating the molecule to a moiety targeting a cell surface receptor such that after interacting with the receptor). Methods for introducing larger molecules, e.g., peptides and fusion proteins are also well known, including, e.g., injection, liposome-mediated fusion, application of a hydrogel, conjugation to a targeting moiety conjugate endocytozed by the cell, electroporation, and the like).

In one embodiment, the antagonist or agent is a fusion polypeptide or derivatized polypeptide. A fusion or derivatized protein may include a targeting moiety that increases the ability of the polypeptide to traverse a cell membrane or causes the polypeptide to be delivered to a specified cell type (e.g., liver cells or tumor cells) preferentially or cell compartment (e.g., nuclear compartment) preferentially. Examples of targeting moieties include lipid tails, amino acid sequences such as antennapoedia peptide or a nuclear localization signal (NLS; e.g., Xenopus nucleoplasmin Robbins et al., 1991, Cell 64:615).

In one embodiment of the invention, a peptide sequence or peptide analog determined to inhibit a PDZ domain-PL protein binding, in an assay of the invention is introduced into a cell by linking the sequence to an amino acid sequence that facilitates its transport through the plasma membrane (a “transmembrane transporter sequence”). The peptides of the invention may be used directly or fused to a transmembrane transporter sequence to facilitate their entry into cells. In the case of such a fusion peptide, each peptide may be fused with a heterologous peptide at its amino terminus directly or by using a flexible polylinker such as the pentamer G-G-G-G-S (SEQ ID NO:1) repeated 1 to 3 times. Such linker has been used in constructing single chain antibodies (scFv) by being inserted between V_{H} and V_{L} (Bird et al., 1988, Science 242:423-426; Huston et al., 1988, Proc. Natl. Acad. Sci. U.S.A. 85:5979-5883). The linker is designed to enable the correct interaction between two beta-sheets forming the variable region of the single chain antibody. Other linkers which may be used include Glu-Gly-Lys-Ser-Ser-Gly-Ser-Gly-Ser-Glu-Ser-Lys-Val-Asp (SEQ ID NO:2) (Chaudhary et al., 1990, Proc. Natl. Acad. Sci. U.S.A. 87:1066-1070) and Lys-Glu-Ser-Gly-Ser-Val-Ser-Ser-Glu-Gln-Leu-Ala-Gln-Phe-Arg-Ser-Leu-Asp (SEQ ID NO:3) (Bird et al., 1988, Science 242:423-426).

A number of peptide sequences have been described in the art as capable of facilitating the entry of a peptide linked to these sequences into a cell through the plasma membrane (Derossi et al., 1998, Trends in Cell Biol. 8:84). For the purpose of this invention, such peptides are collectively referred to as transmembrane transporter peptides. Examples of these peptide include, but are not limited to, tat derived from HIV (Vives et al., 1997, J. Biol. Chem. 272:16010; Nagahara et al., 1998, Nat. Med. 4:1449), antennapedia from Drosophila (Derossi et al., 1994, J. Biol. Chem. 261:10444), VP22 from herpes simplex virus (Elliot and D'Hare, 1997, Cell 88:223-233), complementarity-determining regions (CDR) 2 and 3 of anti-DNA antibodies (Avrameas et al., 1998, Proc. Natl Acad. Sci. U.S.A., 95:5601-5606),70 KDa heat shock protein (Fujihara, 1999, EMBO J. 18:411-419) and transportan (Pooga et al., 1998, FASEB J. 12:67-77). In a preferred embodiment of the invention, a truncated HIV tat peptide having the sequence of GYGRKKRRQRRRG (SEQ ID NO:173) is used.

It is preferred that a transmembrane transporter sequence is fused to a hematopoietic cell surface receptor carboxyl terminal sequence at its amino-terminus with or without a linker. Generally, the C-terminus of a PDZ motif sequence (PL sequence) must be free in order to interact with a PDZ domain. The transmembrane transporter sequence may be used in whole or in part as long as it is capable of facilitating entry of the peptide into a cell.

In an alternate embodiment of the invention, a hematopoietic cell surface receptor C-terminal sequence may be used alone when it is delivered in a manner that allows its entry into cells in the absence of a transmembrane transporter sequence. For example, the peptide may be delivered in a liposome formulation or using a gene therapy approach by delivering a coding sequence for the PDZ motif alone or as a fusion molecule into a target cell.

The compounds of the of the invention may also be administered via liposomes, which serve to target the conjugates to a particular tissue, such as lymphoid tissue, or targeted selectively to infected cells, as well as increase the half-life of the peptide composition. Liposomes include emulsions, foams, micelles, insoluble monolayers, liquid crystals, phospholipid dispersions, lamellar layers and the like. In these preparations the peptide to be delivered is incorporated as part of a liposome, alone or in conjunction with a molecule which binds to, e.g., a receptor prevalent among lymphoid cells, such as monoclonal antibodies which bind to the CD45 antigen, or with other therapeutic or immunogenic compositions. Thus, liposomes filled with a desired peptide or conjugate of the invention can be directed to the site of lymphoid cells, where the liposomes then deliver the selected inhibitor compositions. Liposomes for use in the invention are formed from standard vesicle-forming lipids, which generally include neutral and negatively charged phospholipids and a sterol, such as cholesterol. The selection of lipids is generally guided by consideration of, e.g., liposome size, acid lability and stability of the liposomes in the blood stream. A variety of methods are available for preparing liposomes, as described in, e.g., Szoka et al., Ann. Rev. Biophys. Bioeng. 9:467 (1980), U.S. Pat. Nos. 4,235,871, 4,501,728 and 4,837,028.

The targeting of liposomes using a variety of targeting agents is well known in the art (see, e.g., U.S. Pat. Nos. 4,957,773 and 4,603,044). For targeting to the immune cells, a ligand to be incorporated into the liposome can include, e.g., antibodies or fragments thereof specific for cell surface determinants of the desired immune system cells. A liposome suspension containing a peptide or conjugate may be administered intravenously, locally, topically, etc. in a dose which varies according to, inter alia, the manner of administration, the conjugate being delivered, and the stage of the disease being treated.

In order to specifically deliver a PDZ motif sequence (PL sequence) peptide into a specific cell type, the peptide may be linked to a cell-specific targeting moiety, which include but are not limited to, ligands for diverse leukocyte surface molecules such as growth factors, hormones and cytokines, as well as antibodies or antigen-binding fragments thereof. Since a large number of cell surface receptors have been identified in leukocytes, ligands or antibodies specific for these receptors may be used as cell-specific targeting moieties. For example, interleukin-2, B7-1 (CD80), B7-2 (CD86) and CD40 or peptide fragments thereof may be used to specifically target activated T cells (The Leucocyte Antigen Facts Book, 1997, Barclay et al. (eds.), Academic Press). CD28, CTLA4 and CD40L or peptide fragments thereof may be used to specifically target B cells. Furthermore, Fc domains may be used to target certain Fc receptor-expressing cells such as monocytes.

Antibodies are the most versatile cell-specific targeting moieties because they can be generated against any cell surface antigen. Monoclonal antibodies have been generated against leukocyte lineage-specific markers such as certain CD antigens. Antibody variable region genes can be readily isolated from hybridoma cells by methods well known in the art. However, since antibodies are assembled between two heavy chains and two light chains, it is preferred that a scFv be used as a cell-specific targeting moiety in the present invention. Such scFv are comprised of V_{H} and V_{L} domains linked into a single polypeptide chain by a flexible linker peptide.

The PDZ motif sequence (PL sequence) may be linked to a transmembrane transporter sequence and a cell-specific targeting moiety to produce a tri-fusion molecule. This molecule can bind to a leukocyte surface molecule, passes through the membrane and targets PDZ domains. Alternatively, a PDZ motif sequence (PL sequence) may be linked to a cell-specific targeting moiety that binds to a surface molecule that internalizes the fusion peptide.

In an other approach, microspheres of artificial polymers of mixed amino acids (proteinoids) have been used to deliver pharmaceuticals. For example, U.S. Pat. No. 4,925,673 describes drug-containing proteinoid microsphere carriers as well as methods for their preparation and use. These proteinoid microspheres are useful for the delivery of a number of active agents. Also see, U.S. Pat. Nos. 5,907,030 and 6,033,884, which are incorporated herein by reference.

6.9.2 Introduction of Polynucleotides into Cells

A polynucleotide encoding a surface receptor C-terminal peptide may be useful in the treatment of various leukocyte activation-associated abnormal conditions. By introducing gene sequences into cells, gene therapy can be used to treat conditions in which leukocytes are activated to result in deleterious consequences. In one embodiment, a polynucleotide that encodes a PL sequence peptide of the invention is introduced into a cell where it is expressed. The expressed peptide then inhibits the interaction of PDZ proteins and PL proteins in the cell.

Thus, in one embodiment, the polypeptides of the invention are expressed in a cell by introducing a nucleic acid (e.g., a DNA expression vector or mRNA) encoding the desired protein or peptide into the cell. Expression may be either constitutive or inducible depending on the vector and choice of promoter. Methods for introduction and expression of nucleic acids into a cell are well known in the art and described herein.

In a specific embodiment, nucleic acids comprising a sequence encoding a peptide disclosed herein, are administered to a human subject. In this embodiment of the invention, the nucleic acid produces its encoded product that mediates a therapeutic effect by inhibiting leukocyte activation. Any of the methods for gene therapy available in the art can be used according to the present invention. Exemplary methods are described below.

For general reviews of the methods of gene therapy, see Goldspiel et al., 1993, Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May, 1993, TIBTECH 11(5):155-215. Methods commonly known in the art of recombinant DNA technology which can be used are described in Ausubel et al. (eds.), 1993, Current Protocols in Molecular Biology, John Wiley & Sons, NY; and Kriegler, 1990, Gene Transfer and Expression, A Laboratory Manual, Stockton Press, NY.

In a preferred embodiment of the invention, the therapeutic composition comprises a coding sequence that is part of an expression vector. In particular, such a nucleic acid has a promoter operably linked to the coding sequence, said promoter being inducible or constitutive, and, optionally, tissue-specific. In another specific embodiment, a nucleic acid molecule is used in which the coding sequence and any other desired sequences are flanked by regions that promote homologous recombination at a desired site in the genome, thus providing for intrachromosomal expression of the nucleic acid (Koller and Smithies, 1989, Proc. Natl. Acad. Sci. USA 86:8932-8935; Zijlstra et al., 1989, Nature 342:435-438).

Delivery of the nucleic acid into a patient may be either direct, in which case the patient is directly exposed to the nucleic acid or nucleic acid-carrying vector, or indirect, in which case, cells are first transformed with the nucleic acid in vitro, then transplanted into the patient. These two approaches are known, respectively, as in vivo or ex vivo gene therapy.

In a specific embodiment, the nucleic acid is directly administered in vivo, where it is expressed to produce the encoded product. This can be accomplished by any methods known in the art, e.g., by constructing it as part of an appropriate nucleic acid expression vector and administering it so that it becomes intracellular, e.g., by infection using a defective or attenuated retroviral or other viral vector (see U.S. Pat. No. 4,980,286), by direct injection of naked DNA, by use of microparticle bombardment (e.g., a gene gun; Biolistic, Dupont), by coating with lipids or cell-surface receptors or transfecting agents, by encapsulation in liposomes, microparticles, or microcapsules, by administering it in linkage to a peptide which is known to enter the nucleus, or by administering it in linkage to a ligand subject to receptor-mediated endocytosis (see e.g., Wu and Wu, 1987, J. Biol. Chem. 262:4429-4432) which can be used to target cell types specifically expressing the receptors. In another embodiment, a nucleic acid-ligand complex can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., PCT Publications WO 92/06180 dated Apr. 16, 1992; WO 92/22635 dated Dec. 23, 1992; WO92/20316 dated Nov. 26, 1992; WO93/14188 dated Jul. 22, 1993; WO 93/20221 dated Oct. 14, 1993). Alternatively, the nucleic acid can be introduced intracellularly and incorporated within host cell DNA for expression, by homologous recombination (Koller and Smithies, 1989, Proc. Natl. Acad. Sci. USA 86:8932-8935; Zijlstra et al., 1989, Nature 342:435-438).

In a preferred embodiment of the invention, adenoviruses as viral vectors can be used in gene therapy. Adenoviruses have the advantage of being capable of infecting non-dividing cells (Kozarsky and Wilson, 1993, Current Opinion in Genetics and Development 3:499-503). Other instances of the use of adenoviruses in gene therapy can be found in Rosenfeld et al., 1991, Science 252:431-434; Rosenfeld et al., 1992, Cell 68:143-155; and Mastrangeli et al., 1993, J. Clin. Invest. 91:225-234. Furthermore, adenoviral vectors with modified tropism may be used for cell specific targeting (WO98/40508). Adeno-associated virus (AAV) has also been proposed for use in gene therapy (Walsh et al., 1993, Proc. Soc. Exp. Biol. Med. 204:289-300).

In addition, retroviral vectors (see Miller et al., 1993, Meth. Enzymol. 217:581-599) have been modified to delete retroviral sequences that are not necessary for packaging of the viral genome and integration into host cell DNA. The coding sequence to be used in gene therapy is cloned into the vector, which facilitates delivery of the gene into a patient. More detail about retroviral vectors can be found in Boesen et al., 1994, Biotherapy 6:291-302, which describes the use of a retroviral vector to deliver the mdrl gene to hematopoietic stem cells in order to make the stem cells more resistant to chemotherapy. Other references illustrating the use of retroviral vectors in gene therapy are: Clowes et al., 1994, J. Clin. Invest. 93:644-651; Kiem et al., 1994, Blood 83:1467-1473; Salmons and Gunzberg, 1993, Human Gene Therapy 4:129-141; and Grossman and Wilson, 1993, Curr. Opin. in Genetics and Devel. 3:110-114.

Another approach to gene therapy involves transferring a gene to cells in tissue culture. Usually, the method of transfer includes the transfer of a selectable marker to the cells. The cells are then placed under selection to isolate those cells that have taken up and are expressing the transferred gene. Those cells are then delivered to a patient.

In this embodiment, the nucleic acid is introduced into a cell prior to administration in vivo of the resulting recombinant cell. Such introduction can be carried out by any method known in the art, including but not limited to transfection, electroporation, lipofection, microinjection, infection with a viral or bacteriophage vector containing the nucleic acid sequences, cell fusion, chromosome-mediated gene transfer, microcell-mediated gene transfer, spheroplast fusion, etc. Numerous techniques are known in the art for the introduction of foreign genes into cells (see e.g., Loeffler and Behr, 1993, Meth. Enzymol. 217:599-618; Cohen et al., 1993, Meth. Enzymol. 217:618-644; Cline, 1985, Pharmac. Ther. 29:69-92) and may be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny. In a preferred embodiment, the cell used for gene therapy is autologous to the patient.

In a specific embodiment, the nucleic acid to be introduced for purposes of gene therapy comprises an inducible promoter operably linked to the coding sequence, such that expression of the nucleic acid is controllable by controlling the presence or absence of the appropriate inducer of transcription.

Oligonucleotides such as anti-sense RNA and DNA molecules, and ribozymes that function to inhibit the translation of a leukocyte surface receptor mRNA, especially its C-terminus are also within the scope of the invention. Anti-sense RNA and DNA molecules act to directly block the translation of mRNA by binding to targeted mRNA and preventing protein translation. In regard to antisense DNA, oligodeoxyribonucleotides derived from the translation initiation site, e.g., between −10 and +10 regions of a nucleotide sequence, are preferred.

The antisense oligonucleotide may comprise at least one modified base moiety which is selected from the group including, but not limited to, 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Within the scope of the invention are engineered hammerhead motif ribozyme molecules that specifically and efficiently catalyze endonucleolytic cleavage of leukocyte surface receptor RNA sequences.

Specific ribozyme cleavage sites within any potential RNA target are initially identified by scanning the target molecule for ribozyme cleavage sites which include the following sequences, GUA, GUU and GUC. Once identified, short RNA sequences of between 15 and 20 ribonucleotides corresponding to the region of the target gene containing the cleavage site may be evaluated for predicted structural features such as secondary structure that may render the oligonucleotide sequence unsuitable. The suitability of candidate targets may also be evaluated by testing their accessibility to hybridization with complementary oligonucleotides, using ribonuclease protection assays.

The anti-sense RNA and DNA molecules and ribozymes of the invention may be prepared by any method known in the art for the synthesis of nucleic acid molecules. These include techniques for chemically synthesizing oligodeoxyribonucleotides well known in the art such as for example solid phase phosphoramidite chemical synthesis. Alternatively, RNA molecules may be generated by in vitro and in vivo transcription of DNA sequences encoding the RNA molecule. Such DNA sequences may be incorporated into a wide variety of vectors which contain suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

Various modifications to the DNA molecules may be introduced as a means of increasing intracellular stability and half-life. Possible modifications include, but are not limited to, the addition of flanking sequences of ribo- or deoxy-nucleotides to the 5′ and/or 3′ ends of the molecule or the use of phosphorothioate or 2′O-methyl rather than phospho-diesterase linkages within the oligodeoxyribonucleotide backbone.

6.9.3 Other Pharmaceutical Compositions

The compounds of the invention, may be administered to a subject per se or in the form of a sterile composition or a pharmaceutical composition. Pharmaceutical compositions comprising the compounds of the invention may be manufactured by means of conventional mixing, dissolving, granulating, dragee-making, levigating, emulsifying, encapsulating, entrapping or lyophilizing processes. Pharmaceutical compositions may be formulated in conventional manner using one or more physiologically acceptable carriers, diluents, excipients or auxiliaries that facilitate processing of the active peptides or peptide analogues into preparations which can be used pharmaceutically. Proper formulation is dependent upon the route of administration chosen.

For topical administration the compounds of the invention may be formulated as solutions, gels, ointments, creams, suspensions, etc. as are well-known in the art.

Systemic formulations include those designed for administration by injection, e.g subcutaneous, intravenous, intramuscular, intrathecal or intraperitoneal injection, as well as those designed for transdermal, transmucosal, oral or pulmonary administration.

For injection, the compounds of the invention may be formulated in aqueous solutions, preferably in physiologically compatible buffers such as Hanks's solution, Ringer's solution, or physiological saline buffer. The solution may contain formulatory agents such as suspending, stabilizing and/or dispersing agents.

Alternatively, the compounds may be in powder form for constitution with a suitable vehicle, e.g., sterile pyrogen-free water, before use.

For transmucosal administration, penetrants appropriate to the barrier to be permeated are used in the formulation. Such penetrants are generally known in the art. This route of administration may be used to deliver the compounds to the nasal cavity.

For oral administration, the compounds can be readily formulated by combining the active peptides or peptide analogues with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. For oral solid formulations such as, for example, powders, capsules and tablets, suitable excipients include fillers such as sugars, such as lactose, sucrose, mannitol and sorbitol; cellulose preparations such as maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP); granulating agents; and binding agents. If desired, disintegrating agents may be added, such as the cross-linked polyvinylpyrrolidone, agar, or alginic acid or a salt thereof such as sodium alginate.

If desired, solid dosage forms may be sugar-coated or enteric-coated using standard techniques.

For oral liquid preparations such as, for example, suspensions, elixirs and solutions, suitable carriers, excipients or diluents include water, glycols, oils, alcohols, etc. Additionally, flavoring agents, preservatives, coloring agents and the like may be added.

For buccal administration, the compounds may take the form of tablets, lozenges, etc. formulated in conventional manner.

For administration by inhalation, the compounds for use according to the present invention are conveniently delivered in the form of an aerosol spray from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

The compounds may also be formulated in rectal or vaginal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

Alternatively, other pharmaceutical delivery systems may be employed. Liposomes and emulsions are well known examples of delivery vehicles that may be used to deliver peptides and peptide analogues of the invention. Certain organic solvents such as dimethylsulfoxide also may be employed, although usually at the cost of greater toxicity. Additionally, the compounds may be delivered using a sustained-release system, such as semipermeable matrices of solid polymers containing the therapeutic agent. Various of sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

As the compounds of the invention may contain charged side chains or termini, they may be included in any of the above-described formulations as the free acids or bases or as pharmaceutically acceptable salts. Pharmaceutically acceptable salts are those salts which substantially retain the biologic activity of the free bases and which are prepared by reaction with inorganic acids. Pharmaceutical salts tend to be more soluble in aqueous and other protic solvents than are the corresponding free base forms.

### 6.10. Effective Dosages

The compounds of the invention will generally be used in an amount effective to achieve the intended purpose. For use to inhibit leukocyte activation-associated disorders, the compounds of the invention or pharmaceutical compositions thereof, are administered or applied in a therapeutically effective amount. By therapeutically effective amount is meant an amount effective ameliorate or prevent the symptoms, or prolong the survival of, the patient being treated. Determination of a therapeutically effective amount is well within the capabilities of those skilled in the art, especially in light of the detailed disclosure provided herein. An “inhibitory amount” or “inhibitory concentration” of a PL-PDZ binding inhibitor is an amount that reduces binding by at least about 40%, preferably at least about 50%, often at least about 70%, and even as much as at least about 90%. Binding can as measured in vitro (e.g., in an A assay or G assay) or in situ.

For systemic administration, a therapeutically effective dose can be estimated initially from in vitro assays. For example, a dose can be formulated in animal models to achieve a circulating concentration range that includes the IC₅₀ as determined in cell culture (i.e., the concentration of test compound that inhibits 50% of leukocyte surface receptor-PDZ domain-containing protein interactions). Such information can be used to more accurately determine useful doses in humans.

Initial dosages can also be estimated from in vivo data, e.g., animal models, using techniques that are well known in the art. One having ordinary skill in the art could readily optimize administration to humans based on animal data.

Dosage amount and interval may be adjusted individually to provide plasma levels of the compounds that are sufficient to maintain therapeutic effect. Usual patient dosages for administration by injection range from about 0.1 to 5 mg/kg/day, preferably from about 0.5 to 1 mg/kg/day. Therapeutically effective serum levels may be achieved by administering multiple doses each day.

In cases of local administration or selective uptake, the effective local concentration of the compounds may not be related to plasma concentration. One having skill in the art will be able to optimize therapeutically effective local dosages without undue experimentation.

The amount of compound administered will, of course, be dependent on the subject being treated, on the subject's weight, the severity of the affliction, the manner of administration and the judgment of the prescribing physician.

The therapy may be repeated intermittently while symptoms detectable or even when they are not detectable. The therapy may be provided alone or in combination with other drugs. In the case of conditions associated with leukocyte activation such as transplantation rejection and autoimmunity, the drugs that may be used in combination with the compounds of the invention include, but are not limited to, steroid and non-steroid anti-inflammatory agents.

6.10.1 Toxicity

Preferably, a therapeutically effective dose of the compounds described herein will provide therapeutic benefit without causing substantial toxicity.

Toxicity of the compounds described herein can be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. Compounds which exhibit high therapeutic indices are preferred. The data obtained from these cell culture assays and animal studies can be used in formulating a dosage range that is not toxic for use in human. The dosage of the compounds described herein lies preferably within a range of circulating concentrations that include the effective dose with little or no toxicity. The dosage may vary within this range depending upon the dosage form employed and the route of administration utilized. The exact formulation, route of administration and dosage can be chosen by the individual physician in view of the patient's condition. (See, e.g., Fingl et al., 1975, In: The Pharmacological Basis of Therapeutics, Ch. 1, p. 1).

### 7. EXAMPLES

### 7.1 Example 1

### Tat-T Cell Surface Receptor Carboxyl Terminus Fusion Peptides Inhibited T Cell Activation

7.1.1. Materials And Methods

7.1.1.1. Peptide Synthesis

All peptides were chemically synthesized by standard procedures. The Tat-CD3 carboxyl terminus fusion peptide, (GYGRKKRRQRRRGPPSSSSGL, SEQ ID NO:174); Tat-CLASP1 carboxyl terminus fusion peptide, (GYGRKKRRQRRRGSISSSAEV, SEQ ID NO:243); Tat-CLASP2 carboxyl terminus fusion peptide, (GYGRKKRRQRRRGMTSSSSVV, SEQ ID NO:176); and Tat peptide, (GYGRKKRRQRRRG, SEQ ID NO:173); were dissolved at 1 mM in PBS, pH 7, or dH2O. Stock MBPAc1-16 peptide, (AcASQKRPSQRHGSKYLA, SEQ ID NO: 403), was dissolved at 5 mM. All peptides were aliquoted and stored at −80° C. until tested.

7.1.1.2 Cell Cultures

Cells were maintained and tested in RPMI 1640 media supplemented with 10% fetal calf serum (HyClone), 2 mM glutamine, 10 mM Hepes, 100 U/ml penicillin, 100 μg/ml streptomycin, 0.1 mM non-essential amino acids, 1 mM sodium pyruvate, and 50 μM beta mercaptoethanol.

7.1.1.3 T Cell Stimulation Assay

Supernatants were assayed for cytokine production following activation of T cell lines. Mouse T cell lines were stimulated using two different methods, either with antigen and antigen presenting cells or anti-mouse CD3.

Antigen-specific mouse T cells, BR4.2, were activated with the N-terminal 16 amino acid sequences of myelin basic protein (MBPAc1-16) and syngenic mouse splenocytes in 96-well plates. Mitomycin C-treated antigen presenting cells, 2×10⁵ B10.BR, were added to each row of serially diluted MBPAc1-16 ranging from 0 to 200 μM. Next, 10 μM Tat-peptides or media alone was added to each row. Finally, 2×10⁴ MBPAc1-16-specific T cell, pre-loaded with 10 μM Tat-peptides (see above), were added to all wells (Rabinowitz et al.,1997,. Proc. Natl. Acad. Sci. U.S.A., 94:8702-8707). Cells were activated during an overnight incubation at 5% CO2, 37° C. Cell supernatant was collected and stored at −80° C. until assayed for cytokine production. The final volume was 200 μl/well.

Antibody against mouse CD3 (Pharmigen #145-2C11) was coated overnight at 4° C. using 96-well flat bottom Elisa plates at a final concentration of 0.5 μg/ml, diluted in PBS. Just prior to use, plates were washed three times with 200 μl/well PBS to remove excess anti-CD3. To ensure that cells were given sufficient time to transduce Tat-peptides before activation, T cells (5×10⁵ cells/ml) were pre-treated with or without 10 μM Tat-peptides for two hours at 5% CO2, 37° C. and then diluted in media with or without 10 μM Tat-peptides to a final concentration of 2×10⁴ cells per well in a final volume of 200 μl. Cells were then treated as described above.

7.1.1.4 Cytokine ELISA

IFNγ was measured from cell supernatants, described above, at ambient temperature using the Endogen, Inc. ELISA protocol 3. Briefly, 96-well, flat bottom, high binding ELISA plates were preincubated overnight with coating antibody (MM700). Plates were washed with 50 mM TRIS, 0.2% tween-20, pH 8 and they blocked for one hour with PBS plus 2% BSA. Washed plates were then incubated one hour with 25 μl of cell supernatant and 25 μl blocking buffer, or with 50 μl IFNγ standard. The presence of IFNγ was detected with a biotin-labeled anti-mouse IFNγ monoclonal antibody (MM700B, Endogen, Inc.,). Quantitative amounts of detection antibody are revealed with horseradishperoxidase-conjugated streptavidin. The enzymatic, color, substrate for HRP, tetramethylbenzidine (TMB), was developed for up to 30 minutes and stopped with 1.0 M H₂SO₄. The absorbance at 450 nm was measured using a microtiter plate reader (Thermo Max, Molecular Devices) and the concentration of unknown IFNγ from cell supernatants was calculated from a standard curve generated by Softmax Pro. software (Molecular Devices).

7.1.1.2 Results

Peptides containing Tat transporter sequences linked to C-terminal sequences of various PLs were testing for their ability to inhibit T cell activation. FIG. 4A shows that the Tat-CD3 fusion peptide inhibits T cell activation mediated by peptide:MHC as compared to controls of Tat-peptide alone or no peptide. FIG. 4B shows that Tat-CLASP2 carboxyl terminus fusion peptide inhibited T cell activation mediated by monoclonal anti-CD3 as compared to Tat-peptide alone. Tat-CLASP1 fusion peptide did not inhibit T cell activation in this experiment. These results indicate that peptides containing potential inhibitory sequences can be transported into T cells through transporter peptide such as Tat to disrupt surface receptor organization mediated by PDZ proteins. Disruption of PDZ-mediated surface receptor organization leads to blockage of T cell activation in response to antigen.

### 7.2. Example 2

### Design of an Inhibitor of DLG 1-Ligand Binding with Greater than 100 uM Potency

A GST/DLG1 fusion protein (See TABLE 3) and a biotin-labeled peptide corresponding to the C-terminal 20 amino acids of the CLASP-2 protein, peptide AA2L (see TABLE 4), were synthesized and purified by standard techniques well known in the art as described supra. This PDZ-ligand combination was then shown to bind specifically using both the “A” assay and the “G” assay (See TABLE 2). Once specific binding was demonstrated, the apparent affinity of the binding interaction was determined using Approach 1 of the section entitled “Measurement of PDZ-ligand binding affinity” (see FIG. 2A). The measured apparent affinity was 21 uM. This implies that 21 uM labeled CLASP-2 peptide AA2L filled 50% of the binding sites for CLASP-2 on DLG1. Thus, 21 uM unlabeled CLASP-2 peptide should be able to block the binding of a given ligand to DLG1 by approximately 50%, assuming that the given ligand (1) binds to the same site(s) on DLG1 as Qasp˜2 and (2) is not added at sufficient concentration to reduce significantly the binding of the CLASP-2 peptide (i.e. cannot out-compete the CLASP-2 peptide).

To detect such inhibition, it was necessary to synthesize an analogue of the CLASP2peptide AA2L that (1) retained similar DLG1 binding properties and (2) would not itself generate a signal in the assay selected to measure inhibition. Because most molecular interactions between PDZ proteins and their ligands involve only the C-terminal 6 amino acids of the ligand, an eight amino acid variant of the CLASP-2 peptide, MTSSSSVV (SEQ ID NO: 191), was anticipated to retain similar DLG1 binding properties as the 20 amino acid AA2L CLASP-2 peptide. This eight amino acid CLASP-2 peptide (lacking a functional label) was therefore synthesized and purified by standard techniques as described supra. When 100 uM of the (functionally unlabeled) eight amino acid CLASP-2 peptide and 20 uM of the biotin-labeled AA2L CLASP-2 peptide were added simultaneously to DLG1 in a variant of the “G” assay (described supra), the binding of the labeled AA2L CLASP-2 peptide was, as predicted, inhibited by greater than 50% (FIG. 3A). An analogous experiment in which the labeled AA2L CLASP-2 peptide was replaced with another labeled DLG1 ligand, labeled AAI3L Fas peptide demonstrated similar inhibition by the eight amino acid CLASP-2 peptide (FIG. 3A). Thus, an effective inhibitor of DLG1-ligand binding (i.e. the eight amino acid CLASP-2 peptide MTSSSSVV, SEQ ID NO: 191) with a known potency range (order of magnitude 21 uM) was designed based on knowledge of the affinity, 21 uM, with which a particular labeled ligand, the CLASP-2 peptide AA2L, bound to DLG1.

### 7.3 Example 3

### Generation of Eukaryotic Expression Constructs Bearing DNA Fragments that Encode PDZ Domain Containing Genes or Portions of PDZ Domain Genes

This example describes the cloning of PDZ domain containing genes or portions of PDZ domain containing genes were into eukaryotic expression vectors in fusion with red fluorescent protein (RFP).

A. Strategy

DNA fragments corresponding to PDZ domain containing genes were generated by RT-PCR from jurkat cell line (transformed T-cells) derived RNA. Primers were designed to create restriction nuclease recognition sites at the PCR fragment's ends, to allow cloning of those fragments into the appropriate vectors. Subsequent to RT-PCR, DNA samples submitted to agarose gel electrophoresis. Bands corresponding in size to the expected size were excised, DNA extracted and treated with appropriate restriction endonuclease. DNA samples were purified once more by gel electrophoresis, and gel extracted DNA fragments were coprecipitated and ligated with the appropriate linearized cloning vector. After transformation into E.coli, bacterial colonies were screened by PCR for the presence and correct orientation of insert. Positive clones were picked for large scale DNA preparation and the insert including the flanking vectors sites were sequenced to ensure correct sequence of fragments and junctions with the vectors and fusionproteins.

B. Vectors:

Cloning vectors were pDsRED1-N1 (purchased from CLONTECH, #6921-1) and pDsRED1-N1(+ATG), a derivative of pDsRED1-N1 generated by recombinant DNA technology.

DNA fragments to clone that contained the ATG-start codon were cloned into pDsRED1-N1. Fragments void of a proper translation initiation codon were cloned into pDsRED1-N-(+ATG), since this vector includes an translation initiation start codon. Vector pDsRED1-N1(+ATG) differs from pDsRED1 only with regard to the multiple cloning sites. The sequence that is unique to pDsRED1-N1(+ATG) is shown below; boundaries with pDsRED1-N1 are printed in lower case and correspond to nucleotides N 633 and N 662 in pDsRED1-N1, respectively.

5′-attGCCACCATGGGAATTCTGGATCCGGGAgat-3′

C. Deduced Amino Acid Linker Sequences:

Linker sequences between the cloned inserts and RFP vary depending on the vectors and on the restriction endonuclease used for cloning. Deduced linker amino acid sequences are listed in the table below; For some constructs, the first N-terminal and/or last C-terminal amino acid corresponds to a linker amino acid introduced by the cloning process but is not represented at that position in the corresponding gene.

TABLE 8

| | |
|---|---|
| pDsRED1-N1, cloning approach: | PDZ domain insert C-term-LEU-GLN-SER- |
| (fragment) Eco RI or Mfe I/Eco RI | THR-VAL-PRO-ARG-ALA-ARG-ASP-PRO-PRO- |
| (vector) | VAL-ALA-THR-red flourecent protein; |
| pDsRED1-N1(+ATG), cloning approach: | Start codon (MET)-GLY-ILE-PDZ domain |
| (fragment) Eco RI/Eco RI (vector) | gene insert-LEU-ASP-PRO-GLY-TYR-PRO- |
| | PRO-VAL-ALA-THR-red flourecent protein; |
| pDsRED1-N1(+ATG), cloning approach: | Start codon (MET)-ARG-ILE-PDZ domain |
| (fragment) Mfe I/Eco RI (vector) | gene insert-LEU-ASP-PRO-GLY-TYR-PRO-PRO- |
| | VAL-ALA-THR-red flourecent protein; |

D. Constructs:

The deduced protein sequence of cloned inserts, primers used to generate DNA fragments by RT-PCR and accession # are given below for each construct. For all constructs, the fusion with RFP was carboxy terminal.

1. Homo Sapiens Dishevelled 1 (DVL1)
Acc #:
NM_{—}004421
GI: 4758213
Cloning sites for all constructs: Eco RI/Eco RI

Construct (N-P) [Covers the methionin start codon and extends over the C-terminal boundary of the DVL1 PDZ domain];
primers: 308 DVF and 311 DVR;

vector: pDsRED1-N1

aa 1-aa 341
MAETKIIYHMDEEETPYLVKLPVAPERVTLADFKNVLSNRPVHAYKFFKS
MDQDFGVVKEEIFDDNAKLPCFNGRVVSWLVLVEGAHSDAGSQGTDSHTD
LPPPLERTGGIGDSRSPSFQPDVASSRDGMDNETGTESMVSHRRDRARRR
NREEAARTNGHPRGDRRRDVGLPPDSASTALSSELESSSFVDSDEDDSTS
RLSSSTEQSTSSRLIRKHKRRRRKQRLRQADRASSFSSMTDSTMSLNIIT
VTLNMERHHFLGICIVGQSNDRGDGGIYIGSIMKGGAVAADGRIEPGDML
LQVNDVNFENMSNDDAVRVLREIVSQTGPISLTVAKCWDPT

Construct (N) [Covers the methionin start codon and extends to the N-terminal boundary of the DVL1 PDZ domain];
primers: 308 DVF and 345 DVR

vector: pDsRED1-N1

aa 1-aa 197
MAETKIIYHMDEEETPYLVKLPVAPERVTLADFKNVLSNRPVHAYKFFFK
SMDQDFGVVKEEIFDDNAKLPCFNGRVVSWLVLVEGAHSDAGSQGTDSHT
DLPPPLERTGGIGDSRSPSFQPDVASSRDGMDNETGTESMVSHRRDRARR
RNREEAARTNGHPRGDRRRDVGLPPDSASTALSSELESSSFVDSDEDG

Construct (P) [Consists of the PDZ domain of DVL1];
primers: 344 DLF and 311 DVR;

vector: pDsRED1-N1(+ATG)

aa 246-aa 341
SLNIITVTLNMERHHFLGICIVGQSNDRGDGGIYIGSIMKGGAVAADGRI
EPGDMLLQVNDVNFENMSNDDAVRVLREIVSQTGPISLTVAKCWDPT

Primers:

308 DYF (N 128-N 155)
5′-TCGGAATTCGTCGCGCCATGGCGGAGAC-3′
311 DVR (N 1004-N 1032)
5′-GGGAATTCGGTCCCAGCACTTGGCCACAG-3′
344 DVF (N 873-N 900)
5′-CCAGAATTCTCAACATCGTCACTGTCAC-3′
345 DYR (N713-N744)
5′-TCGGAATTCCATCCTCGTCCGAGTCCACAAAG-3′

2. KLAA 0751 /41.8 KD
Acc #:
AB018294
GI: 3882222
   Cloning Sites for all Constructs: (Vector) Eco RI/(Fragment) Mfe I

Construct (N-J) [includes the third in frame-methionin (putative start) codon in (GI: 3882222) and extends c-terminal of the PDZ domain to the region on sequence divergency between KIAA 0751 (GI: 3882222) and hypothetical 41.8 Kd protein (AF007156/GI: 3882222)];
primers: 318 KIF and 320 KIR;

vector: pDsRED1-N1

aa 389-aa 803
MMYFGGHSLEEDLEWSEPQIKDSGVDTCSSTTLNEEHSHSDKHPVTWQPS
KDGDRLIGRILLNKRLKDGSVPRDSGAMLGLKVVGGKMTESGRLCAFITK
VKKGSLADTVGHLRPGDEVLEWNGRLLQGATFEEVYNIILESKPEPQVEL
VVSRPIGDIPRIPDSTHAQLESSSSSFESQKMDRPSISVTSPMSPGMLRD
VPQFLSGQLSIKLWFDKVGHQLIVTILGAKDLPSREDGRPRNPYVKIYFL
PDRSDKNKRRTKTVKKTLEPKWNQTFIYSPVHRREFRERMLEITLWDQAR
VREEESEFLGEILIELETALLDDEPHWYKLQTHDVSSLPLPHPSPYMPRR
QLHGESPTRRLQRSKRISDSEVSDYDCDDGIGVVSDYRHDGRDLQSSTLS
VPEQVMSSNHCSPSGSPHRVDVIGRTT

Construct (P) [consists of the PDZ domain of KIAA 0751/41.8 Kd hypothetical protein (GI: 3882222)];
primers: 341 KIF and 319 KIR.

vector pDsRED1-N1(+ATG)

aa 443-aa 534
LKDGSVPRDSGAMLGLKVVGGKMTESGRLCAFITKVKKGSLADTVGHLRP
GDEVLEWNGRLLQGATFEEVYNIILESKPEPQVELVVSRPIA

Primers:

318 KIF (N 1366-N 1393)
5′-AGACAATTGAGGAAATGATGTACTTTGG-3′
319 KIR (N 1830-N 1857)
5′-GAACAATTGCAATAGGCCTTGAAACTAC-3′
320 KIR (N 2640-N 2667)
5′-ACCCAATTGTAGTCCTTCCTATAACATC-3′
341 KIF (N 1567-N 1593)
5′-ATAGAATTCTAAAAGATGGAAGTGTAC-3′

3. Homo Sapiens PAR6
Acc#:
AF265565
GI: 8468608
Cloning sites for all constructs: Eco RI/Eco RI

Construct (N-P) [Covers the methionin start codon and extends over the C-terminal boundary of the PDZ domain];
primers: 322 PAF and 324 PAR;

vector: pDsRED1-N1

aa 1-aa 251
MARPQRTPARSPDSIVEVKSKFDAEFRRFALPRASVSGFQEFSRLLRAV
HQIPGLDVLLGYTDAHGDLLPLTNDDSLHRALASGPPPLRLLVQKREAD
SSGLAFASNSLQRRKKGLLLRPVAPLRTRPPLLISLPQDFRQVSSVIDV
DLLPETHRRVRLHKHGSDRPLGFYIRDGMSVRVAPQGLERVPGIFISRL
VRGGLAESTGLLAVSDEILEVNGIEVAGKTLDQVTDMMVANSHNLIVTV
KPANQR

Construct (N) [Covers the methionin start codon and extends to the N-terminal boundary of the PDZ domain;
primers: 322 PAF and 343 PAR

vector: pDsRED1-N1

aa 1-aa 147
MARPQRTPARSPDSIVEVKSKFDAEFRRFALPRASVSGFQEFSRLLRAVH
QIPGLDVLLGYTDAHGDLLPLTINDDSLHRALASGPPPLRLLVQKREADS
SGLAFASNSLQRRKKGLLLRPVAPLRTRPPLLISLPQDRQVSSVIDV

Construct (P) [Consists of the PDZ domain of PAR6];
primers: 342 PAF and 324 PAR;

vector: pDsRED1-N1(+ATG)

aa 155-aa 251
RRVRLHKHGSDRPLGFYIRDGMSVRVAPQGLERVPGIFISRLVRGGLAES
TGLLAVSDEILEVNGIEVAGKTLDQVTDMMVANSHNLIVTVKPANQR

Primers

322 PAF (N 55-N 82)
5′-CCCGAATTCGCCATGGCCCGGCCGCAGAG-3′
324 PAR (N 798-N 825)
5′-CGTGAATTCGCTGGTTGGCGGGCTTGAC-3′
342 PAF (N 519-N 548)
5′-GAGGAATTCCGACGGGTGCGGCTGCACAAG-3′
343 PAR (N 485-N 516)
5′-GCAGAATTCCCACGTCTATGACTGAGGAAAC-3′

4. Homo Sapiens Post-Synaptic Density Protein 95 (PSD95)
Acc #:
ABU83192
GI: 3318652
Cloning sites for all constructs: Eco RI/Eco RI
Vector: pDsRED1-N1

Construct (N-P3) [Covers the methionin start codon and extends over the C-terminal boundary of PDZ domain 3;

primers: 315 PSF and 304 PSR.

aa 1-aa 442
MSQRPRAPRSALWLLAPPLLRWAPPLLTVLHSDLFQALLDILDYYEASLS
ESQKYRYQDEDTPPLEHSPAHLPNQANSPPVIVNTDTLEAPGYELQVNGT
EGEMEYEEITLERGNSGLGFSIAGGTDNPHIGDDPSIFITKIIPGGAAAQ
DGRLRVNDSILFVNEVDVREVTHSAAVEALKEAGSIVRLYVMRRKPPAEK
VMEIKLIKGPKGLGFSIAGGVGNQHIPGDNSIYVTKIIEGGAAHKDGRLQ
IGDKILAVNSVGLEDVMHEDAVAALKNTYDVVYLKVAKPSNAYLSDSYAP
PDITTSYSQHLDNEISHSSYLGTDYPTAMTPTSPRRYSPVAKDLLGEEDI
PREPRRIVIHRGSTGLGFNIVGGEDGEGIFISFILAGGPADLSGELRKGD
QLLSVNGVDLRNASHEQAAIALKNAGQTVTIIAQYKPEEYSR

primers:

315 PSF (N 847-N 876)
5′-AGAGAATTCAGAGATATGTCCCAGAGACCAAG-3′
304 PSR (N 2161-N 2189)
5′-CGAGAATTCTGTACTCTTCTGGTTTATAC-3′

5. Homo Sapiens hCASK (CASK).
Acc #:
AF032119
GI: 2641548
Cloning sites: Eco RI/Eco RI

Construct (P) [Covers the PDZ domain of hCASK];
Note: The amino acid sequence homology between the human hCASK and the mouse mCASK-B is 100% identical.
primers: 336 CAF and 335 CAR;

vector: pDsRED1-N1(+ATG)

aa 399-aa 572
RLVQFQKNTDEPMGITLKMNELNHCIVARIMHGGMIHRQGTLHVGDEIRE
INGISVANQTVEQLQKMLREMRGSITFKIVPSYR

Primers

336 CAF (N 1484-N 1512)
5′-CCAGAATTCGGCTGGTACAGTTTCAAAAG-3′
325 CAR (N 1722-N 1750)
5′-ACTGAATTCGGTAACTTGGCACAATCTTG-3′

6. Homo Sapiens Membrane Protein, Palmitolated 2 (MPP2/DLG2)
Acc#:
X82895
GI: 939884
Cloning sites for all constructs: Eco RI/Eco RI

Construct (N-SH3) [Covers the methionin start codon, the PDZ domain and extends to the C-terminal boundary of the MPP2 SH3 domain;the construct is a splice variant of the construct annotated under GI:939884. With respect to GI:939884, the DNA portion N 238 to 309 is missing; this DNA stretch corresponds to AA 51-74. The open reading frame is maintained throughout the deletion].
primers: 305 MF and 306 MR;

vector: pDsRED1-N1

aa 1-aa 317
MPVAATNSETAMQQVLDNLGSLPSATGAAELDLIFLRGIMESPIVRSLAK
AHERLEETKLEAVRDNNLELVQEILRDLAQLAEQSSTAAELAHILQEPHF
QSLLETHDSVASKTYETPPPSPGLDPTFSNQPVPPDAVRMVGIRKTAGEH
LGVTFRVEGGELVIARILHGGMVAQQGLLHYGDIIKEVNGQPVGSDPRAL
QELLRNASGSVILKILPSYQEPHLPRQVFVKCHFDYDPARDSLIPCKEAG
LRFNAGDLLQIVNQDDANWWQACHVEGGSAGLIPSQLLEEKRKG

Primers:

| | |
|---|---|
| 305 MF | 5′-AGAGAATTCAGAGCCCTTGCCTCCTTC-3′ |
| (N 58-N 84) | |
| 306 MR | 5′-TGAGAATTCCTTTCCGCTTCTCCTCCAG-3′ |
| (N 798-N 825) | |

7. Homo Sapiens Tax Interaction Protein 1 (TIP1)
Acc #:
AF028823
GI: 2613001
Cloning sites: Eco RI/Bam H1
   (We determined 5′start site and 5′ full length sequence by 5′ RACE)

Construct (N-C);

vector: pDsRed1-N1

aa 3-aa 125
YIPGQPVTAVVQRVEIHKLRQGENLILGFSIGGGIDQDPSQNPFSEDKTD
KGIYVTRVSEGGPAEIAGLQSGDKIMQVNGWDMTMVTHDQARKRLTKRSE
EVVRLLVTRQSLQKAVQQSML

Primer:

| | |
|---|---|
| 1318 TIP R3-1 | 5′-CAGTCCATGCTGTCGGATCCG-3′ |
| (N 336-N 356) | |
| 1317 TIP R5-1* | 5′-GTCGGAATTCCCTACATCCCG-3′ |

*Primer 5′ end corresponds to the nucleotide that is located 29 nucleotides 5′ of N 1; primer sequence corresponds to sequence determined by 5′ RACE; numbering corresponds to genbank sequence entry (GI 2613001).

### 7.4 Example 4

### Expression of PDZ Domain-Containing Proteins in Mammalian Cells

We have identified a number of PDZ domain-containing proteins that are expressed in lymphocytes. To study the biology of these molecules in immunity, we have adopted a dominant-negative approach by over-expressing portions of tagged PRISM molecules in lymphocytes. We have termed cellular studies of gene function as CELLOMICS. We used two standard methods for DNA transfection; DNA precipitation by calcium phosphate (Graham and van der Eb, infra. and Gorman,) and electroporation (Potter, infra). Expression of PDZ fusion proteins was tested in mouse and human lymphocytic cell lines and in human embryonic kidney cells (293 HEK, ATCC CRL-1573) based on detection of red fluorescent protein (RFP).

7.4.1 Materials and Methods

A. Cell Lines used for Transfection of PRISM-Tagged Constructs

Jurkat E6 human T cells (ATCC TIB-152) were maintained and tested in complete IMDM (IMDM medium supplemented with 2 mM glutamine, 100 U/mL penicillin, 100 μg/mL streptomycin, 0.1 mM nonessential amino acids, 1 MM sodium pyruvate (Gibco BRL), 50 μM beta mercaptoethanol (Sigma), and 10% fetal calf serum (Gemini Bio-Products)). 293 HEK cells were maintained and tested in complete DMEM (DMEM medium supplemented with 2 mM glutamine, 10 mM HEPES, 100 U/mL penicillin, 100 μg/mL streptomycin, and 10% fetal calf serum). CH27 mouse B cell lymphoma and 2B4 mouse T cell hybrid lines were maintained and tested in complete RPMI (RPMI 1640 medium supplemented with 2 mM glutamine, 100 U/mL penicillin, 100 μg/mL streptomycin, 0.1 mM nonessential amino acids, 1 mM sodium pyruvate, 10 mM HEPES, 50 μM beta mercaptoethanol, and 10% fetal calf serum).

B. Transfection Methods

DNA precipitation by calcium phosphate was used to transfect 293 HEK cells. At least 2 hrs before transfection, ˜5×10⁵ cells were plated in 5 ml of complete DMEM onto one 60 mm TC-treated dish for each transfection. For each plate, 5-10 μg of DNA was brought to a volume of 110 μl with deionized H₂O. Fifteen μl of 2M CaCl₂ was added to the DNA solution, which was then slowly added to 125 μl of 2× HBS pH 7.0, (2× HBS=1.64% NaCl 1.188 % and Hepes 0.04% Na₂HPO₄). A fine precipitate formed, which was then added dropwise to cells and incubated at 37° C., 5% CO₂. Transfected cells were analyzed for expression of RFP on day 1, 2 and 3 post-transfection using the RFP control plasmid and some of the PRISM-RFP fusion constructs. Since we consistently found maximal expression on day 2, the data described below is from cells 2 days after transfection.

The lymphocytic cell lines (Jurkat E6, CH27, and 2B4) cells were transfected by electroporation and tested for control RFP expression on day 1 and 2 post-transfection. All three lymphocytic cell lines showed that maximal expression of RFP control plasmid and some of the PRISM-RFP constructs on day 1 post-transfection and therefore only day 1 expression data is described below. The BTX ECM830 generator was used to transfect the Jurkat E6 cells as follows: two cuvettes (4 mm electrode gap) containing 5×10⁶ cells in 0.5 ml and 5-30 μg of DNA in serum free-IMDM were electroporated with a single pulse at 260 volts for 50 msec. Cuvettes were immediately placed on ice for 10-15 minutes before being transferred to 100 mm dishes containing 10 ml of complete IMDM. CH27 and 2B4 cells were transfected in cuvettes (4 mm electrode gap) using the BioRad Gene pulser. The protocol using the BioRad Gene pulser is the same as for the BTX ECM830 generator, except they were electroporated in serum free-RPMI at 0.45 kV, 960 μF with unlimited resistance for 38-44 msec.

7.4.2 Analysis of PRISM-Tagged Fusion Protein Expression

A. PDZ-RFP Fusion Protein Constructs

DNA fragments encoding PDZ domain proteins were cloned into DsRED (Clonetech), an RFP fusion vector. To ensure proper folding of inserts, the RFP was placed C-terminal to PRISM. Constructs tested for expression in the DsRED vector are: CASK(P), DLG1(N-P3), DLG1(N), DVL1(N-P), DVL1(N), DVL1(P), MPP2(N-SH3), PAR6(N-P), PAR6(N), PAR6(P), PSD95(N-P3), TIP1(P), TIP1(N-C), KIAA0751(N3-J) and KIAA0751(P), as described in Example 3, supra. The abbreviation N denotes the N-terminus, C denotes the C-terminus, P denotes one PDZ region, P3 denotes three PDZ regions, SH3 denotes the inclusion of the SH3 domain, and J denotes the joining region.

B. PDZ-RFP Fusion Proteins are Expressed in 293 HEK Cells

All of the PDZ-RFP transfected cells were analysis using a Coulter EPICS XL flow cytometer and inverted Nikon Diaphot fluorescent microscope. Each set of transfections included the DsRED plasmid (RFP) as a positive control for transfection efficiency. All of the PDZ-RFP constructs expressed RFP fusion protein in 293 HEK cells with transfection efficiency ranging from 15-60%.

Fluorescent microscopy revealed various expression patterns. The RFP expression from the control DsRED construct was evenly diffused in the cell varying from weak to very brightly fluorescence. For the constructs expressed in 293 HEK cells, the staining patterns are summarized here: CASK(P) some bright and diffuse and some punctated, DLG1(N-P3) differentially speckled, some globular and few diffuse, DLG1(N) differentially speckled and some globular, DVL1(N-P) diffuse and punctated and polarized and globular, DVL1(N) brightly speckled and few polarized and globular, DVL1(P) occasional diffuse and rare speckled, MPP2(N-SH3) polarized and globular and some punctated, PAR6(N-P) speckled and some polarized and globular, PAR6(N) diffuse and punctated, PAR6(P) diffuse and punctated, polarized and globular, PSD95(N-P3) diffuse, TIP1(P) diffuse, speckled, polarized and globular, TIP1(N-C) occasional rings and polarized, KLAA0751(N3-J) punctated and few dim and diffuse, and KIAA0751(P) punctated and few dim and diffuse.

C. PDZ-RFP Fusion Protein Expression is Restricted in Jurkat T Cells

Jurkat E6 cells were screened for expression of PDZ-RFP fusion proteins tested using flow cytometry and a fluorescent microscope on day 1 post-transfection. Although expression of all in 293 HEK cells, several constructs did not expressed in Jurkat E6 cells. The control RFP and all of the expressed PDZ-RFP fusion proteins had a much lower percent of cells expressing RFP as compared to 293 HEK cells. Constructs that were tested but less than 4% of the cells expressed RFP were considered negative, they were: DLG1(N-P3), DLG1(N), DVL1(P), MPP2(N-SH3), PAR6(N-C), PAR6(P), PSD95(N-P3), TIP1(P), and KIAA0751(N3-J). There was weak expression (4-10% RFP positive) for DVL1(N), PAR6(N), and KIAA075(P). CASK(P), DVL1(N-P), PAR(N-P), and TIP1(N-C) expression levels were 10-40% in Jurkat E6 cells. The DsRED plasmid was used as a RFP positive control for each set of transfections.

Fluorescent microscopy of Jurkat E6 was similar to the 293 HEK cells, the control DsRED construct was evenly diffused, varying from weak to very brightly fluorescence. The PRISM-RFP fusion protein staining patterns in Jurkat E6 are summarized as follows: CASK(P) some bright and diffuse and some punctated, DVL1(N-P) punctated and polarized and globular, DVL1(P) occasional diffuse, PAR6(N-P) polarized and globular and a few diffuse, PAR6(P) polarized and globular close to the membrane, TIP1(N-C) occasional rings and polarized, and KIAA0751(P) few dim and diffuse.

D. PDZ-RFP Expression is not Rescued by Stimulation with Anti-TCR and Anti-CD28

This experiment describes expression of for the PDZ-RFP fusion proteins by stimulating the Jurkat E6 cells after transfection (see, e.g., copending U.S. Ser. No 60/240503, incorporated herein by reference). This approach was tested for PDZ-RFP fusion proteins DLG1(N-P3), DVL1(N), MPP2(N-SH3), PAR6(N-P), PAR6(N), PAR6(P), PSD95(N-P3), TIP1(P), TIP1(N-C), KIAA0751(N3-J) and KIAA0751(P) and as control DsRED and HC4 42-1. Jurkat E6 cells were transfected with 25 μg of DNA as described above and stimulated using 2.5 1μg/mL mouse anti-human CD28 monoclonal antibody (PharMingen International catalog number 555726) and a 1:10 dilution of mouse anti-human Jurkat TCR monoclonal antibody supernatant (ATCC CRL-2424 clone C305) 2 hrs after transfection. At 24 hr post-transfection each RFP fusion protein was screened for RFP expression by XL flow cytometer and fluorescent microscopy and compared to the same cells unstimulated. None of the PRISM-RFP fusion proteins showed a significant change in expression.

7.4.3 Discussion

The CELLOMICS approach has been instructive in deducing the role of PDZ-domain proteins in lymphocyte process. The level of PDZ-domain protein expression appears to be highly regulated in mouse and human lymphocytes, but not in human 293 HEK cells. Control RFP and most of the PDZ-RFP contructs are expressed in 293 HEK but only a subset of PDZ-RFP molecules (PAR6, TIP1 and DVL1) are expressed in human and mouse lymphocytes. Regulation of expression may occur at the level of translation or post-translation, possibly affecting a lymphocyte-specific process that is necessary for proper protein expression or continuing cell survival.

In the case of DVL1 and PAR6 we show that for expression and localization 30 both the P and N domains are necessary. Protein localization appears to be controlled by different domains of the protein. Our data for the DVL1 and PAR6 constructs suggest that the PDZ and the N-terminal domains are important for proper stability, sorting and/or compartmentalization in lymphocytes. Diminished expression of DVL1(N) and (P) and PAR6(N) and (P), as compared to DVL1(N-P) and PAR6(N-P) correlates with the loss of these domains. There also appears to be a hierarchy or an order to the successful localization of these molecules with the n-terminal domain being of first importance and the P (PDZ) domain playing a secondary role. In the case of TIP1(P) and TIP(N-C) the additional sequences on both the c- and n-terminal domains rescue lymphocytic expression.

7.4.4 References

Graham, F. and van der Eb, A., (1973). Virology 52:456.

Gorman, C., Science, (1983). 221, 551-553.

Potter, H. (1995). Recombinant DNA Methodology II. Academic Press, Inc. Chapter 31, pg. 467484. Applications of Electroporation in Recombinant DNA Technology.

Reynaud et al. 2000, J. Biol. Chem. 275:33962-333968.

Suzuki et al, 1999, Oncogene 18:5967-72.

### 7.5 Example 5

### TIP1-RFP Overexpression Enchanes Anti-CD95 Induced Apoptosis in Jurkat T Cells

This example shows the use of the assays and PRISM MATRIX described herein to identify a medically significant PDZ-PL interaction.

Human T cell lymphocyte virus type 1 (HTLV-1) is the etiologic agent of neoplasia within human peripheral blood T cells. The most important factor contributing to the initial stages of viral-mediated transformation of T cells after HTLV-1 infection is the viral oncoprotein Tax. Tax has been reported to bind to several proteins containing PDZ domains including TIP1 (Suzuki, T., et al., and Rousset et al., et al.).

Reviewing the PRISM MATRIX (similar to that provided in TABLE 2) we found that TIP1 binds to the CD95 and Tax. As described infra, we also determined that Tax competes for binding to TIP1 1000-fold better than CD95. Therefore we hypothesized TIP1 is a positive regulator of apoptosis and that in HTLV-1 infection, Tax blocks TIP1 binding to CD95 to promote an anti-apoptotic effect. We predicted that overexpression of TIP1-RFP would lower sensitivity to CD95-induced apoptosis and that TAX-1 (sometimes refered to herein as “TAX”) can immortalize host cells by competing with CD95 for TIP-1 to block apoptosis.

A. TIP-1 Interaction with CD95 and TAX-1

As shown in the PRISM MATRIX, both the C-termini of Taxi and of CD95 (Fas) bind to the PDZ domain of Tax1-interacting protein (Tax1-IP, TIP-1). Various concentrations of peptide ligands corresponding to the C-terminal 20 amino acids of Tax1 and CD95 were reacted with the Tax1-IP GST/PDZ fusion protein in a “G assay” format. These experiments revealed that the interaction of Tax1 with Tax1-IP is of higher affinity than the interaction of CD95 with Tax1-IP. We therefore anticipated that a peptide corresponding to the C-terminal 8 amino acids of Tax1 would be able to block efficiently the binding of the 20 amino acid CD95 peptide to Tax1-IP. In addition, we anticipated that a peptide corresponding to the C-terminal 8 amino acids of CD95 would less efficiently block the binding of the 20 amino acid Tax1 peptide to Tax1-IP.

As shown in FIGS. 6 and 7, these predictions were correct: 100 uM of the truncated Tax1 peptide effectively blocked binding of the longer CD95 peptide to Tax1-IP, whereas 500 uM of the truncated CD95 peptide was required to block binding of the longer Tax1 peptide. Thus, information in the PRISM MATRIX, in combination with experiments investigating the affinities of different PDZ-ligand interactions, is useful in the design of inhibitors of PDZ-ligand binding of different potencies.

Additional experiments showed that the CD95-TIP-1 interaction occurs in vivo. using biotynylated peptides corresponding to the C-terminal 20 residues of CD95 or TAX we were able to “pull-down” TIP-1-rfp from a cell lysate from transfected 293 HEK cells. This interaction was blocked in the presence of peptides having the sequence of the C-termimus (i.e., 8 C-terminal residues) of TAX-1 or CD95.

B. Enhancement of Apoptosis

Jurkat E6 T cells were transfected with either 20 μg of the RFP, TIP1-RFP, or PAR6(N-P)-RFP. Twenty-four hours after transfection, cells were treated with 100 ng/ml anti-human CD95 (Immunotech #1504, mouse monoclonal anti-human CD95 clone CH11) for 2hr. at 37° C. To analyze the levels of apoptosis in anti-CD95-treated cells we used the annexin V-FITC flow cytometer assay (Immunotech, Cat. No.2375). We followed the protocol supplied by the manufacturer, except that propidium iodide was omitted because the RFP and propidium iodide emission spectra are overlapping using an Argon Laser (488 nm). To test for apoptosis with annexin V, approximately 5×10⁵ cells were washed once with PBS and resuspended in 500 μl of annexin V binding buffer and 5 μl of annexin V-FITC solution was added. Cells were incubated for 10 minutes on ice, in the dark. Each sample was then analyzed using flow cytometry.

Since the RFP emission spectra is broad, detectable in two of the three channels (FL-2=560-590 and FL-3=605-725), only the green channel (FL-1=505-545) is testing. We circumvented this by doing the experiment in two steps. First we set a forward scatter, side scatter dot plot and then separated live from dead cells using a green viability dye (Sytox, Molecular probes). Secondly, we used the live cell gate to test for apoptotic cells with annexin V-FITC. Since many cells are already dead from electroporation and each construct can vary in percentage of cell death, we only compared live RFP positive cells with live RFP negative cells for annexin V. Additionally there is little cell death since the cells are only treated with anti-CD95 for 2 hrs. Viable cells were analyzed using a FL-1 by FL-3 two-color dot plot, and showed 4 distinct populations: viable transfected cells expressing RFP only, viable non-transfected cells, negative for RFP and annexin V-FITC, apoptotic non-transfected cells positive for annexin V-FITC, and apoptotic transfected cells double positive for RFP and annexin V-FITC [FIG. 5A].

The results showed that cells expressing TIP1-RFP have a 30% increase in apoptosis versus the TIP1-RFP negative cells [FIG. 5B]. In contrast, cells expressing either RFP control or PAR6(N-P)-RFP showed little to no increase in apoptosis, when compared to untransfected cells. The finding that overexpression of TIP1-RFP results in an increased sensitivity to anti CD95-mediated apoptosis demonstrates the physiological relevance of the TIP1/CD95 interaction in vivo. Furthermore, transfected cells expressing higher of TIP1-RFP were more sensitive to apoptosis than cells expressing lower levels of TIP1-RFP, confirming the pro-apoptotic effects of TIP1.

HTLV-1 infection results in adult T cell leukemia and tropical paraparesis (a model for Multiple Sclerosis). Restoration of apoptosis to HTLV-1 infected cells by interfering with TAX-1 interaction with CD95 is a basis for the treatment of HTLV-1. Furthermore, modulation of TIP1-CD95 interactions may be a target for treatment of Multiple Sclerosis.

### 7.6 Example 6

### HPV E6 Oncogene and Prism

This example demonstrates the use of PL sequence motifs identified according the to the invention in the prediction of biological function in an oncogenic virus.

Human papilloma virus (HPV) infection plays a role in development of cervical carcinoma. The oncoprotein responsible for this is the early gene E6 from strains 16, 18 and 31. E6 associates with p53 and shunts this tumor suppressor into the ubiquitin proteosomal pathway to affect transformation. Using the PL motifs disclosed herein, we noted that the E6 from oncogenic strains HPV16, 18 and 31 are PDZ ligands (PLs) with the carboxy-terminal E-T-Q-V/L. Similarly, the E6 of oncogenic strain HPV66 has the carboxy-terminus ESTV, which also matches the consensus PDZ binding motif.

We performed an expanded search of the HPV E6 proteins and discovered HPV70 E6 fits perfectly the described PDZ consensus ETQV, identical to HPV18 and 31. We can thus predict that HPV70 is likely oncogenic on the basis that E6 is a PDZ ligand. Other HPV strains with E6 proteins that are potential PLs (based on motifs) include 57 (RTSH), 2a (RTLH), 63 (LYII). Strains 77 (QSRQ) and 80 (GSIE)may also be PLs, although the motif match is less strong. This information is summarized in TABLE 9.

PDZ targets for carboxy-terminal peptides corresponding to the above described E6s are determined using the methods of the invention (e.g., the G assay and PRISM MATRIX assay). Inhibitors of the interaction of the PDZ and oncogenic E6 PLs are determined are identified using the methods of the invention and are useful for inhibition of E6-mediated transformation. Such inhibitors (e.g., small molecules, peptides or recombinant proteins) are administeres to patients (e.g., by local application to the vaginal vault and the uterine cervix) to treat or prevent cervical carcinoma. Diagnostic assays for oncogenic HPV are carried out using the sequences corresponding to the HPV E6 PL to design polynucleotide (e.g., PCR) or antibody probes that distinguish E6 proteins that are PLs from those that are not PLs.

TABLE 9

HPV E6 C-TERMINAL SEQUENCES

| | | | | |
|---|---|---|---|---|
| Strain | GI | C-TERMINAL E6 SEQUENCES | ONCOGENIC | PDZ LIGAND |

| | | | | |
|---|---|---|---|---|
| 61 | 9628574 | TGPCTARWQP | | NO |
| 60 | 9628566 | RQRSYCRNCIEK | | NO |
| 55 | 9628558 | CWTSCMETILP | | NO |
| 50 | 9628550 | CCRNCYEHEG | NO | NO |
| 48 | 9628542 | CRNCISHEGR | NO | NO |
| 44 | 9628534 | CFHCWTSCMETILP | NO | NO |
| 38 | 9628526 | GNWKGRCRHCKAIE | NO | NO |
| 37 | 9628518 | WKGLCRHCGSIG | NO | NO |
| 66 | 9628582 | TGSCLQCWRHTSRQATESTV | YES | YES |
| 57 | 9626033 | RCMNCAPRCMENAPALRTSH | NO | YES? |
| 2a | 9626032 | HCMNCGSSCTATDPASRTLH | NO | YES? |
| 16 | 4927719 | WTGRCMSCCRSSRTRRETQL | YES | YES |
| 18 | 60995 | HSCCNRARQERLQRRRETQV | YES | YES |
| 31 | 333048 | GRWTGRCIACWRRPRTETQV | YES | YES |
| 33 | | CAACWRSARRRRLQRRRETAL | YES | YES |
| 51 | | CANCWQRTRQRRLQRRNETQV | YES | YES |
| 52 | | CSECWRPTRRPRLQRRRVTQV | YES | YES |
| 58 | | CAVCWRPARRRRLQRRRQTQV | YES | YES |
| 70 | 134508 | RHCWTSNREDRRRIRRETQV | NO | YES |
| 63 | 312092 | VHKVRNKFKAKCSLCRLYII | NO | YES |
| 77 | 2911558 | GHWRGSCLHCWSRCMGQSRQ | | ? |
| 80 | 2911565 | QFHKVRRNWKGLCRHCGSIE | | ? |
| 21 | 9628462 | WKGICRLCKHFQ | | NO |
| 11 | 333026 | WKGRCLHCWTTCMEDLLP | NO | NO |
| 36 | 9628510 | WKGICRQCKHFYNDW | NO | NO |
| 29 | 9628502 | WRGSCLYCWSRCMGQSPR | NO | NO |
| 28 | 9628494 | CQYCWLRCTVRIPQ | NO | NO |
| 24 | 9628486 | KVRRGWKGLCRQCKQI | NO | NO |
| 22 | 9628470 | VRDHWKGRCRHCKAIE | NO | NO |
| 21 | 9628462 | HKVRGSWKGICRLCKHFQ | NO | NO |
| 20 | 9628454 | FYLVRGSWKGICRLCKHFQ | NO | NO |
| 4 | 9626597 | TCYLIRGLWRGYCRNCIRKQ | ND | NO |
| 54 | 1017782 | RRFHCVRGYWKGRCLHCWKP | | |
| 5B | 9626498 | KVRNAWKGICRQCKHFYHDW | | |
| 74 | 1491796 | NTWKGRCFHCWTTCMENILP | | |
| 75 | 2911544 | EFHKVRNRWKGVCRHCRVIE | | |
| 76 | 2911544 | EFHKVRNRWKGVCRHCRVIE | | |
| 47 | 9627136 | KVRNAWKGVCRQCKHFYNDW | ND | NO |
| 65 | 9626613 | ACYLIRGLWRGYCRNCIRKQ | | |

### 7.7 Example 7

### Design of Small Molecule Inhibitors of PDZ-Ligand Interactions, and their Clinical Application

As shown in TABLE 2, both the C-termini of Dock2 and of BLR-1 bind to the PDZ domain of KIAA0807. This example describes the design of small molecule (<600 molecular weight) inhibitors of these interactions. As described supra, chemical entities resembling the C-termini of PDZ ligands that bind to a given PDZ domain (including peptides and mimetics) are effective inhibitors of ligand binding to that PDZ domain. We synthesized and purified 8 amino acid peptides corresponding to the C-termini of Dock2 and BLR-1, for use as inhibitors of KIAA0807-ligand binding. In addition, we synthesized and purified small molecules corresponding to the C-terminal four amino acids of Dock2 and BLR-1, acetylated at the N-terminus. Inhibition of KIAA0807-ligand binding by peptide (8 amino acid) and small molecule (4 amino acid) inhibitors are shown in FIGS. 8 and 9.

In FIG. 8, the bars on the left hand side of the figure show that increasing concentrations of the peptide inhibitor (the C-terminal 8 amino acids of BLR-1) are somewhat effective at blocking binding of 1 uM of the biotinylated C-terminal 20 amino acids of BLR-1 to KIAA0807 GST/PDZ fusion protein. The bars of the right hand side of the figure show that increasing concentrations of the small molecule inhibitor (Acetyl-LTTF) are equally or more effective. In FIG. 9, the bars on the left hand side of the figure show that increasing concentrations of the peptide inhibitor (the C-terminal 8 amino acids of Dock2) are somewhat effective at blocking binding of the 1 uM of the biotinylated C-terminal 20 amino acids of Dock2 to KIAA0807 GST/PDZ fusion protein. The bars on the right hand side of the figure show that increasing concentrations of the small molecule inhibitor (Acetyl-STDL) are equally or more effective. Thus, a general route to producing a small molecule inhibitor of a PDZ-ligand interaction is to synthesize a molecule corresponding to the C-terminal four amino acids of the involved ligand, acetylated at the N-terminus. This compound can subsequently be altered by art known means (e.g., changing its covalent composition to optimize pharmacokinetic properties without grossly altering its molecular structure, especially the molecular structure of the most C-terminal protein).

### 7.8 Example 8

### Inhibition of Individual Interactions found in the Prism Matrix

As shown in the PRISM MATRIX (TABLE 2), the C-terminus of Clasp4 binds to the PDZ domain of KIAA440, and the C-terminus of DNAM-1 binds to the PDZ domain of WWP3. As disclosed herein, it was anticipated that (i) a molecule resembling (i.e., structurally similar to) the C-terminus of Clasp-4 would inhibit the Clasp4/KIAA440 interaction and (ii) a molecule resembling the C-terminus of DNAM-1 would inhibit the DNAM-1/WWP3 interaction.

Peptides corresponding to the C-terminal 8 amino acids of Clasp-4 and DNAM-1 were synthesized and purified. These peptides were then tested for their abilities to inhibit the corresponding interactions. Inhibitor peptide concentrations in the range of 20-200 uM were effective (i.e., at least 50% inhibition) at blocking the interactions of labeled peptides corresponding to the C-termini of Clasp-4 and DNAM-1 with KIAA440 and WWP3, respectively (as measured in a “G assay” format). Thus, as described hereinabove, for each interaction identified in the PRISM MATRIX, it is possible to design or identify (e.g., by library screening) an effective inhibitor of that interaction by synthesizing a molecule (e.g., a peptide, peptide mimetic, or structurally similar small organic molecule) resembling the C-terminus of the PL involved in the interaction.

The present invention is not to be limited in scope by the exemplified embodiments which are intended as illustrations of single aspects of the invention and any sequences which are functionally equivalent are within the scope of the invention. Indeed, various modifications of the invention in addition to those shown and described herein will become apparent to those skilled in the art from the foregoing description and accompanying drawings. Such modifications are intended to fall within the scope of the appended claims.

All publications cited herein are incorporated by reference in their entirety and for all purposes.

1. A method of modulating a biological function of an endothelial cell or hematopoietic cell, comprising introducing into the cell an agent that inhibits binding of a PDZ protein and a PL protein in the cell, thereby modulating the biological function.

2. The method of claim 1, wherein the PL is selected from the group consisting of CD105, VCAM1, CD95, Spectrin β, KV1.3, DNAM1, Neuroligin 3, CD44, CD38, CD3η, LPAP, CD46, CDw128B, DOCK2, PAG, CD34, and BLR-1.

3. The method of claim 2, wherein the PDZ is selected from the group consisting of MPP1, K303, K807, DLG1, PSD95, NeDLG, IP43, LDP, LIM, K545, TIP1, PTN4, CBP, AF6, PDZK1, DLG5, Syntenin, WWP3, and K561.

4. The method of claim 1, wherein
a) the PDZ protein is MPP1 and the PL protein has a carboxy-terminal amino acid motif X-S/T/Y/I-X-V;
b) the PDZ protein is LIMK1 and the PL protein has a carboxy-terminal amino acid motif X-S/T/Y-X-V;
c) the PDZ protein is K303 and the PL protein has a carboxy-terminal amino acid motif X-S-X-V;
d) the PDZ protein is K807 and the PL protein has a carboxy-terminal amino acid motif X1-S/T-X2-V/I/L/F;
e) the PDZ protein is DLG1, PSD95, or NeDLG and the PL protein has a carboxy-terminal amino acid motif X-S/T/Y/A/E-X-V/I/L;
f) the PDZ protein is SNTa1 and the PL protein has a carboxy-terminal amino acid motif X-S/T/Y-D/Y-V/I/L;
g) the PDZ protein is DVL1 and the PL protein has a carboxy-terminal amino acid motif X-S/T/Y-X-V;
h) the PDZ protein is LDP and the PL protein has a carboxy-terminal amino acid motif X-A/S-X2-V/I;
i) the PDZ protein is LIM and the PL protein has a carboxy-terminal amino acid motif X-S/T-X2-A/V;
j) the PDZ protein is K561 and the PL protein has a carboxy-terminal amino acid motif X-S/T/Y-X-V/I/L/F;
k) the PDZ protein is K545 and the PL protein has a carboxy-terminal amino acid motif X-A/S/T/Y-M-A/S/V;
l) the PDZ protein is TAX-IP2 and the PL protein has a carboxy-terminal amino acid motif X-S-D/E-V;
m) the PDZ protein is MPP2 and the PL protein has a carboxy-terminal amino acid motif X-S/T/Y-X-A/V/I;
n) the PDZ protein is TIP-1 and the PL protein has a carboxy-terminal amino acid motif X-S/T-X2-V/I/L;
o) the PDZ protein is PTN-4 and the PL protein has a carboxy-terminal amino acid motif X1-S/T-X-V/F;
p) the PDZ protein is prIL16 and the PL protein has a carboxy-terminal amino acid motif D/E/K/R-V/I/L/F/Y-X-V;
q) the PDZ protein is CBP and the PL protein has a carboxy-terminal amino acid motif X-S/T-F/Y-V;
r) the PDZ protein is protein 41 and the PL protein has a carboxy-terminal amino acid motif X-A/S/T/Y/F-X-A/V/I/L;
s) the PDZ protein is AF6 and the PL protein has a carboxy-terminal amino acid motif X-A/S/T/Y-F/Y-V/I/L;
t) the PDZ protein is RGS12 and the PL protein has a carboxy-terminal amino acid motif X1-S/T/Y-X-V/F;
v) the PDZ protein is PDZK1 and the PL protein has a carboxy-terminal amino acid motif X-T-X-F;
w) the PDZ protein is DLG5 and the PL protein has a carboxy-terminal amino acid motif X-S/T-X-V;
x) the PDZ protein is Synt and the PL protein has a carboxy-terminal amino acid motif X1-V/I/L-X2-V;
y) the PDZ protein is WWP3 and the PL protein has a carboxy-terminal amino acid motif X-S/T-X2-V; and,
z) the PDZ protein is TAX-IP40 and the PL protein has a carboxy-terminal amino acid motif X-Y-X-V;
where X is any amino acid, X1 is any amino acid, X2 is any amino acid.

5. The method of claim 2 wherein the agent is a peptide comprising a sequence of at least the carboxy-terminal two residues of the PL protein.

6. The method of claim 4 wherein the agent is a peptide comprising a sequence of at least the carboxy-terminal three residues of the PL protein.

7. The method of claim 2 wherein the agent is a small molecule or peptide mimetic of the carboxy-terminus of the PL protein.

8. The method of claim 4, wherein the cell is a T cell or a B cell.

9. A method of determining whether a test compound is an inhibitor of binding between a PDZ protein and a PL protein comprising:
a) contacting
i) a PDZ domain polypeptide having a sequence from the PDZ protein, and
ii) a PL peptide, wherein the PL peptide comprises a C-terminal sequence of a PL protein selected from the group consisting of CD105, VCAM1, CD95, Spectrin β, KV1.3, DNAM1,Neuroligin 3, TAX CD44, CD38, CD3η, LPAP, CD46, CDw128B, DOCK2, PAG, CD34, and BLR-1 under conditions in which they form a complex
wherein said contacting is carried out in the presence and in the absence of a test compound;
b) detecting the formation of the complex in the presence and absence of the test compound
wherein less complex formation in the presence of the test compound than in the absence of the compound indicates that the test compound is an inhibitor of a PDZ protein-PL protein binding.

10. An inhibitor identified by the method of claim 9.

11. The inhibitor of claim 9 that is
(a) a peptide comprising a sequence that is from 3 to about 20 residues of a C-terminal sequence of a PL protein selected from CD105, VCAM1, CD95, Spectrin β, KV1.3, DNAM1, Neuroligin 3, TAX CD44, CD38, CD3η, LPAP, CD46, CDw128B, DOCK2, PAG, CD34, and BLR-1;
(b) a peptide mimetic of a peptide of section (a); or
(c) a small organic molecule with a molecular weight less than 1 kD.

12. A pharmaceutical composition comprising an inhibitor of claim 11.

13. A method for treating a disease characterized by leukocyte activation, comprising administering a therapeutically effective amount of an inhibitor of claim 11.

14. The method of claim 13 wherein the disease is characterized by an inflammatory or humoral immune response.

15. The method of claim 14 wherein the disease is an autoimmune disease.

16. A method of modulating a biological function of a cell, comprising introducing into the cell an antagonist that inhibits binding of a PDZ protein and a PL protein in the cell, wherein,
a) the PDZ protein is MPP1 (p55) and the PL is Spectrin β;
b) the PDZ protein is K303 and the PL is Spectrin β;
c) the PDZ protein is K807 and the PL VCAM1, Spectrin β, KV1.3, Neuroligin 3, CD38, CD3η, LPAP, CD46 (form 1), CDw128B, DOCK2, PAG, CD34, or BLR-1;
d) the PDZ protein is DLG1 and the PL is Spectrin;
e) the PDZ protein is PSD95 and the PL is Spectrin β, CD34, or CD38;
f) the PDZ protein is NeDLG and the PL is Spectrin β or CD38;
g) the PDZ protein is TAX IP43 and the PL is Spectrin β, or CD38;
h) the PDZ protein is LDP and the PL is CD38;
i) the PDZ protein is LIM and the PL is CD105;
j) the PDZ protein is K545 and the PL is CD105;
k) the PDZ protein is TIP1 and the PL is CD95, KV1.3, CD3η, LPAP;
l) the PDZ protein is PTN-4 and the PL is Spectrin β;
m) the PDZ protein is CBP and the PL is Spectrin β;
n) the PDZ protein is AF6 and the PL is Spectrin β;
o) the PDZ protein is PDZK1 and the PL is BLR-1;
p) the PDZ protein is DLG5 and the PL is Spectrin;
q) the PDZ protein is Syntenin and the PL is CD44;
r) the PDZ protein is WWP3 and the PL is VCAM1, Spectrin β, DNAM1, Neuroligin 3;
s) the PDZ protein is K561 and the PL is BLR-1.

17. The method of claim 16 wherein the cell is a hematopoietic cell.
